(19) United States
(12) Patent Application Publication
(10) Pub. No.: US 2026/0240840 A1
(43) Pub. Date: Aug. 20, 2026

(54) PREPARATION OF 3,4-DIHYDROQUINOLIN-2(1H)-ONE COMPOUNDS AS ARTEMIS ENDONUCLEASE INHIBITORS

(71) Applicants: CURIA BUFFALO R&D, LLC, Buffalo, NY (US); THE UNITED STATES OF AMERICA, AS REPRESENTED BY THE SECRETARY, DEPARTMENT OF HE, Bethesda, MD (US)

(72) Inventors: William MOORE, Hagerstown, MD (US); Neal GREEN, Newton, MA (US); Mark WOLF, Albany, NY (US); William PAQUETTE, Albany, NY (US); Robb LEWIS, Albany, NY (US); Junho MAENG, Albany, NY (US)

(21) Appl. No.: 19/543,735

(22) Filed: Feb. 18, 2026

Related U.S. Application Data
(60) Provisional application No. 63/759,935, filed on Feb. 18, 2025.

(51) Int. Cl. A61K 31/4995; A61K 31/397; A61K 31/435; A61K 31/444; A61P 35/02; C07D 401/10; C07D 471/04; C07D 471/08; C07D 487/08; C07D 491/107

(52) U.S. Cl. CPC A61K 31/4995; A61K 31/397; A61K 31/435; A61K 31/444; A61P 35/02; C07D 401/10; C07D 471/04; C07D 471/08; C07D 487/08; C07D 491/107

(57) ABSTRACT

The present disclosure is directed to 3,4-dihydroquinolin-2(1H)-one compounds, including compounds of Formula (I)

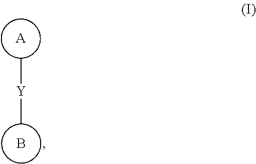

where the substituents

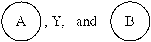

are as described herein. The present disclosure is also directed to methods of using these compounds as Artemis endonuclease inhibitors and modulators, particularly for the treatment of cancer and the treatment of severe combined immunodeficiency.

This application claims the priority benefit of U.S. Provisional Patent Application Ser. No. 63/759,935, filed Feb. 18, 2025, which is hereby incorporated by reference in its entirety.

This invention was made with government support under HHSN261200800001E awarded by National Institutes of Health. The government has certain rights in this invention.

### FIELD

The present invention relates to preparation of 3,4-dihydroquinolin-2(1H)-one compounds and their use as Artemis endonuclease inhibitors or modulators.

### BACKGROUND

Nucleases catalyze the hydrolysis of the phosphodiester bonds in nucleic acids. Nucleases are broadly classified as exonucleases or endonucleases. Exonucleases are often non sequence-specific. On the other hand, endonucleases can be further grouped into sequence-specific endonucleases (e.g., restriction enzymes and structure-selective endonucleases) (Yang, “Nucleases: Diversity of Structure, Function and Mechanism,” Q. Rev. Biophys. 44:1-93 (2011); Yosaatmadja et al., “Structural and Mechanistic Insights Into the Artemis Endonuclease and Strategies for its Inhibition,” Nucleic Acids Research 49(16):9310-9326 (2021)). Artemis (SNM1C or DCLRE1C), SNM1A (DCLRE1A), and SNM1B (Apollo or DCLRE1B) are human nucleases that are members of the extended structural family of metallo-β-lactamase (MBL) fold enzymes (Callebaut et al., “Metallo-Beta-Lactamase Fold Within Nucleic Acids Processing Enzymes: the Beta-CASP Family,” Nucleic Acids Res. 30:3592-3601 (2002); Allerston et al., “The Structures of the SNM1A and SNM1B/Apollo Nuclease Domains Reveal a Potential Basis for Their Distinct DNA Processing Activities,” Nucleic Acids Res. 43:11047-11060 (2015); Yosaatmadja et al., “Structural and Mechanistic Insights Into the Artemis Endonuclease and Strategies for its Inhibition,” Nucleic Acids Research 49(16):9310-9326 (2021)).

SNM1A, SNM1B, and Artemis have similar structures of their core catalytic domains. However, SNM1A, SNM1B, and Artemis have distinct functions and selectivities. While SNM1A and SNM1B are exclusively 5′ to 3′ exonucleases, Artemis is an endonuclease (Malu et al., “Artemis C-Terminal Region Facilitates V(D)J Recombination Through its Interactions with DNA Ligase IV and DNA-pkcs,” J. Exp. Med. 209:955-963 (2012); Niewolik et al., “Autoinhibition of the Nuclease ARTEMIS is Mediated by a Physical Interaction Between its Catalytic and C-Terminal Domains,” J. Biol. Chem. 292:3351-3365 (2017); Yosaatmadja et al., “Structural and Mechanistic Insights Into the Artemis Endonuclease and Strategies for its Inhibition,” Nucleic Acids Research 49(16):9310-9326 (2021)). However, a minor 5′ to 3′ exonuclease activity for Artemis has been reported (Li et al., “Evidence that the DNA endonuclease ARTEMIS also has intrinsic 5′-exonuclease activity,” J. Biol. Chem. 289:7825-7834 (2014); Yosaatmadja et al., “Structural and Mechanistic Insights Into the Artemis Endonuclease and Strategies for its Inhibition,” Nucleic Acids Research 49(16):9310-9326 (2021)).

The Artemis endonuclease complex is an important component in the non-homologous end joining (NHEJ) pathway. NHEJ is required for the DNA end joining phase of V(D)J recombination, the somatic process in developing T- and B-lymphocytes, which is required for their maturation and migration to the periphery. Artemis has the unique activity of opening the sealed DNA hairpin ends formed during RAG endonuclease mediated immunoglobulin (Ig) and T-cell receptor (TCR) gene rearrangement responsible for generating their antigen-recognizing diversity. The key role of Artemis in this process is demonstrated by the fact that individuals with genetic lesions reducing Artemis activity suffer from severe combined immunodeficiency (SCID), similar to effects of DNA-PKcs mutations, as lymphocytes that cannot resolve the RAG-induced Ig or TCR gene double strand breaks (in the form of hairpin DNA ends) undergo apoptosis. In the case of Acute Lymphoblastic Leukemia (ALL), tumor cells are developmentally arrested at the maturation stage, where they undergo continuous rounds of V(D)J recombination.

To facilitate treatment of SCID and ALL, as well as any other disease conditions that implicate Artemis, it would be desired to develop new compounds that can inhibit the activity of Artemis. The present disclosure is directed to overcoming these and other deficiencies in the art.

### SUMMARY

A first aspect of the present disclosure relates to a compound of Formula (I):

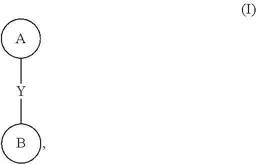

where

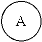

is selected from the group consisting of

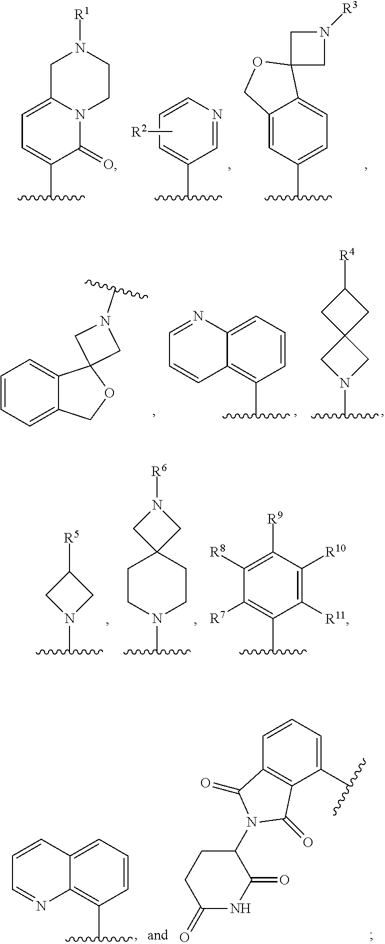

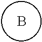

is selected from the group consisting of

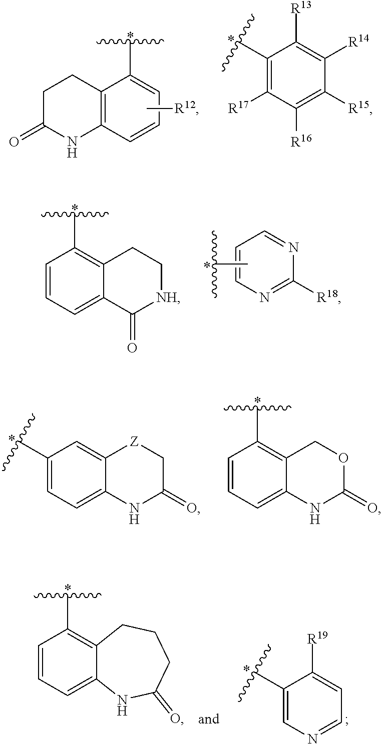

Y is absent, or, if present, is selected from the group consisting of —NH—, —NMe-, and

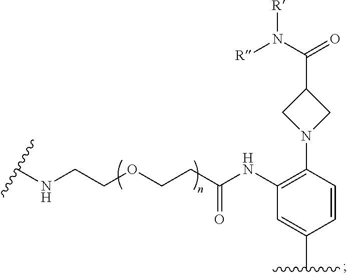

Z is CH₂ or O;
R′ is C₁₋₆ alkyl;
R″ is C₁₋₆ alkyl;
R¹ is selected from the group consisting of H, CN, C₁₋₆ alkyl, C₃₋₈ cycloalkyl, aryl, —C(O)C₁₋₆ alkyl, —C(O)C₂₋₆ alkenyl, —C(O)C₂₋₆ alkynyl, —C(O)aryl, —C(O)(CH₂)ₘbenzyl, —C(O)(CH₂)ₘNHMe, —C(O)(CH₂)ₘNMe₂, —C(O)(CH₂)ₘC₃₋₈ cycloalkyl, —C(O)(CH₂)ₘaryl, —C(O)(CH₂)ₘheteroaryl, —C(O)(CH₂)ₘheterocyclyl, —C(O)nonaromatic bicyclic compound, —C(O)CH(OMe)Ph, —S(O)₂C₁₋₆ alkyl, —S(O)₂C₃₋₈ cycloalkyl, and —S(O)₂aryl, where C₁₋₆ alkyl, C₂₋₆ alkenyl, C₃₋₈ cycloalkyl, aryl, benzyl, heteroaryl, and heterocyclyl, can be optionally substituted 1 to 3 times with a substituent independently selected at each occurrence from the group consisting of halogen, CN, CF₃, ═O, OH, COOH, —C₁₋₆ alkyl, C₃₋₈ cycloalkyl, aryl, heterocyclyl, and heteroaryl, —OC₁₋₆ alkyl, —O-aryl, —S(O)₂C₁₋₆ alkyl, —NHC(O)C₁₋₆ alkyl, —NHC(O)aryl, —C(O)C₁₋₆ alkyl, —C₁₋₆ alkylene-OH, —C₁₋₆ alkylene-C(O)OH, —NR^{a}R^{b}, and —C(O)NH₂;
R² is selected from the group consisting of halogen, —COOH, —C₁₋₆ alkyl, —OC₁₋₆ alkyl, —NR^{a}R^{b}, —NH(CH₂)ₖC(O)NR^{a}R^{b}, —NH(CH₂)ₖC(O)NR^{c}R^{d}, —C(O)NR^{a}R^{b}, and —S(O)₂C₁₋₆ alkyl;
R³ is selected from the group consisting of H, C₁₋₆ alkyl, C₃₋₈ cycloalkyl, C₄₋₁₄ cycloalkylalkyl, aryl, arylalkyl, —C(O)C₁₋₆ alkyl, —C(O)C₂₋₆ alkenyl, —C(O)C₃₋₈ cycloalkyl, —C(O)OC₁₋₆ alkyl, —C(O)(CH₂)_{q}aryl, —C(O)(CH₂)_{q}heteroaryl, —C(O)(CH₂)_{q}heterocyclyl, —S(O)₂C₁₋₆ alkyl, —S(O)₂C₂₋₆ alkenyl, —S(O)₂C₃₋₈ cycloalkyl, —S(O)₂(CH₂)_{q}aryl, —S(O)₂heteroaryl, and —S(O)₂heterocyclyl, where C₁₋₆ alkyl, C₃₋₈ cycloalkyl, aryl, arylalkyl, heteroaryl, and heterocyclyl can be optionally substituted 1 to 3 times with a substituent independently selected at each occurrence from the group consisting of OH, COOH, CF₃, halogen, C₁₋₆ alkyl, aryl, heteroaryl, heterocyclyl, NH₂, —OC₁— alkyl, —C(O)C₁₋₆ alkyl, —CH₂COOH, —CH₂OH, and —C(O)NH₂;
R⁴ is selected from the group consisting of —NR^{a}R^{b}, —NH—(CH₂)ₚ-heteroaryl, —NH—(CH₂)ₚ-heterocyclyl, —NH—C(O)C₁₋₆ alkyl, —NH—C(O)C₂₋₆ alkenyl, —NH—C(O)C₁₋₆ alkyl-aryl, —NH—CH₂—C(O)NR^{a}R^{b}, —NH—S(O)₂C₁₋₆ alkyl, and —NH—S(O)₂C₂₋₆ alkenyl, where C₁₋₆ alkyl, heteroaryl, and heterocyclyl can be substituted 1 to 3 times with C₁₋₆ alkyl;
R⁵ is selected from the group consisting of —NH—C(O)—CH₂—NH—C(O)-aryl, —NH—C(O)—CH₂—NH—C(O)—C₂₋₆ alkenyl, and —NH-heteroarylene-C(O)—NR^{a}R^{b};
R⁶ is selected from the group consisting of H, —C(O)C₁₋₆ alkyl, and —C(O)C₂₋₆ alkyl;
R⁷ is selected from the group consisting of H, CN, C₁₋₆ alkyl, —CH₂OH, and —C(O)NH₂;
R⁸ is selected from the group consisting of H, halogen, —C₁₋₆ alkyl, —CH₂OH, —OC₁₋₆ alkyl, —NMe₂, and —C(O)NHMe;
R⁹ is selected from the group consisting of H, halogen, C₁₋₆ alkyl, —OC₁₋₆ alkyl, heterocyclyl, —NR^{a}R^{b}, and —NH—CH₂—C(O)NH-i-Pr, where C₁₋₆ alkyl and heterocyclyl can be substituted with R²¹;
R¹⁰ is selected from the group consisting of H, C₁₋₆ alkyl, —OC₁₋₆ alkyl, —CH₂OH, —NH₂, —NH—C(O)C₁₋₆ alkyl, and —NH—C(O)OC₁₋₆ alkyl, where C₁₋₆ alkyl can be optionally substituted with —OC₁₋₆ alkyl;
R¹¹ is selected from the group consisting of H, halogen, and —C₁₋₆ alkyl;
R¹² is selected from the group consisting of H, halogen, C₁₋₆ alkyl, and —OC₁₋₆ alkyl;
R¹³ is selected from the group consisting of H, OH, —CH₂OH, and C₁₋₆ alkyl;
R¹⁴ is selected from the group consisting of H, C₁₋₆ alkyl, halogen, OH, —OC₁₋₆ alkyl, and CN;
R¹⁵ is selected from the group consisting of H, C₁₋₆ alkyl, —CH₂OH, CN, and —C(O)NH₂;
R¹⁶ is selected from the group consisting of H, CN, halogen, and —C(O)NH₂;
R¹⁷ is selected from the group consisting of H, OH, C₁₋₆ alkyl, —CH₂OH, and —C(O)NH₂;
R¹⁸ is selected from the group consisting of —OC₁₋₆ alkyl,

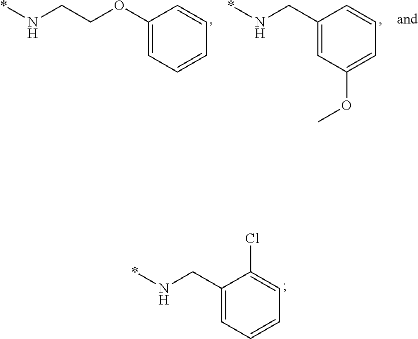

R¹⁹ is C₁₋₆ alkyl or —CH₂OH;
R²⁰ is independently selected at each occurrence from the group consisting of ═O, COOH, C₁₋₆ alkyl, C₃₋₈ cycloalkyl, —C(O)C₁₋₆ alkyl, —C(O)C₂₋₆ alkenyl, —C(O)—C₃₋₈ cycloalkyl, —C(O)aryl, —C(O)heteroaryl, —C(O)heterocyclyl, —C(O)NR^{aa}R^{bb}, —C(O)OC₁₋₆ alkyl, —NH—C(O)OC₁₋₆ alkyl, —NH—C(O)C₂₋₆ alkenyl, —NH—C(O)OC₂₋₆ alkenyl, —C₁₋₆ alkylene-arylene-NH—C(O)C₁₋₆ alkyl, —C₁₋₆ alkylene-arylene-NH—C(O)C₂₋₆ alkenyl, —S(O)₂C₁₋₆ alkyl, —S(O)₂C₂₋₆ alkenyl, —S(O)₂C₃₋₈ cycloalkyl, —S(O)₂(CH₂) aryl, —S(O)₂heterocyclyl, —(CH₂)ᵣheterocyclyl, where C₁₋₆ alkyl, C₁₋₆ alkylene, C₂₋₆ alkenyl, C₃₋₈ cycloalkyl, aryl, arylene, heteroaryl, and heterocyclyl can be optionally substituted 1 to 3 times with a substituent independently selected at each occurrence from the group consisting of halogen, —OH, —COOH, —CN, —C₁₋₆ alkyl, —OC₁₋₆ alkyl, —C₃₋₈ cycloalkyl, aryl, heterocyclyl, —C(O)C₁₋₆ alkyl, —C(O)OC₁₋₆ alkyl, —C(O)OC₁₋₆ alkylene-aryl, and —S(O)₂C₁₋₆ alkyl;
R²¹ is independently selected at each occurrence form the group consisting of —NR^{a}R^{b}, —C(O)C₁₋₆ alkyl, —C(O)OC₁₋₆ alkyl, and —S(O)₂C₁₋₆ alkyl, where —S(O)₂C₁₋₆ alkyl can be optionally substituted with -aryl-COOH;
R²² is independently selected at each occurrence form the group consisting of C₃₋₈ cycloalkyl, aryl, benzyl, —NR^{aa}R^{bb}, heteroaryl, and heterocyclyl, where heteroaryl and heterocyclyl can be optionally substituted 1 to 3 times with C₁₋₆ alkyl;
R^{a} is selected from the group consisting of H, C₁₋₆ alkyl, aryl, and heteroaryl, where C₁₋₆ alkyl, aryl, and heteroaryl can be optionally substituted 1 to 3 times with a substituent independently selected at each occurrence from the group consisting of H, OH, COOH, CF₃, —OC₁₋₆ alkyl, aryl, and —NR^{aa}R^{bb};
R^{b} is H or C₁₋₆ alkyl, where C₁₋₆ alkyl can be optionally substituted 1 to 3 times with —C(O)NR^{aa}R^{bb}; or
R^{a} and R^{b} combine with the nitrogen atom to which they are attached to form piperazine, azetidine, piperidine, pyrrolidine, diazabicyclo[2.2.1]heptane, diazabicyclo[2.2.2]octane, 1,4-diazepane, or 2,7-diazaspiro[3.5]nonane ring, where piperazine, azetidine, piperidine, pyrrolidine, diazabicyclo[2.2.1]heptane, diazabicyclo[2.2.2]octane, 1,4-diazepane, or 2,7-diazaspiro[3.5]nonane ring can be optionally substituted 1 to 3 times with R²⁰,
R^{c} is H, C₁₋₆ alkyl, or C₃₋₈ cycloalkyl, where C₁₋₆ alkyl can be optionally substituted 1 to 3 times with R²²;
R^{d} is H or C₁₋₆ alkyl; or
R^{c} and R^{d} combine with the nitrogen atom to which they are attached to form piperazine, piperidine, pyrrolidine, or morpholine ring, where piperazine or piperidine ring can be optionally substituted 1 to 3 times with a substituent independently selected at each occurrence from the group consisting of C₁₋₆ alkyl, heteroaryl, and heterocyclyl;
R^{aa} is H, C₁₋₆ alkyl, aryl, arylalkyl, or CN, where C₁₋₆ alkyl can be optionally substituted 1 to 3 times with—a substituent independently selected at each occurrence from the group consisting of C(O)OC₁₋₆ alkyl, COOH, and OH;
R^{bb} is H or C₁₋₆ alkyl;
m is 0, 1, 2, or 3;
n is 1, 2, 3, 4, or 5;
p is 1 or 2;
q is 0, 1, or 2;
r is 1;
s is 0 or 1; and
k is 0 or 1;
   or an oxide thereof, a pharmaceutically acceptable salt thereof, a solvate thereof, or a prodrug thereof.

A second aspect of the present disclosure relates to a pharmaceutical composition that includes a therapeutically effective amount of the compound according to the first aspect of the present disclosure and a pharmaceutically acceptable carrier.

A third aspect of the present disclosure relates to a method of treating cancer or neoplastic condition in a subject, which method includes the step of administering a compound according to the first aspect of the present disclosure to the subject in need thereof.

A fourth aspect of the present disclosure relates to a method of inhibiting an Artemis nuclease activity, which method includes the step of contacting an Artemis nuclease with a compound according to the first aspect of the present disclosure under conditions effective to inhibit the Artemis nuclease activity.

A fifth aspect of the present disclosure relates to a method of ameliorating a condition caused by the activity of Artemis nuclease in a subject, which method includes the step of administering a compound according to the first aspect of the present disclosure to the subject.

A sixth aspect of the present disclosure relates to a method of enhancing cancer therapy in a subject, which method includes the step of delivering a compound according to the first aspect of the present disclosure to cancerous cells in the subject in an amount effective to inhibit Artemis nuclease, and treating the subject with a second cancer therapy.

A seventh aspect of the present disclosure relates to a method for the treatment of a severe combined immunodeficiency (SCID), which method includes the step of administering a compound according to the first aspect of the present disclosure to a subject in need thereof.

An eighth aspect of the present disclosure relates to a method of biotherapy in a subject, which method includes the step of administering a compound according to the first aspect of the present disclosure to the subject.

The present application describes the development of first-in-class small molecule inhibitors of Artemis endonuclease, which are expected to modulate this endonuclease by capitalizing on its unique role in V(D)J recombination. These inhibitors of Artemis endonuclease are suitable for the treatment of Acute Lymphoblastic Leukemia (ALL) from both the B- and T-cell lineages. In addition, given the role of Artemis in the DNA double strand break (DSB) repair, via the non-homologous end-joining (NHEJ) pathway, such small molecule inhibitors of Artemis endonuclease can be used to sensitize solid tumors (e.g., breast & lung cancers, sarcomas, neuroblastomas, and germ cell malignancies) as well as several types of lymphoma to DNA DSB-inducing agents such as radiation and topoisomerase inhibitor therapy.

Treatment of individuals having genetic lesions reducing Artemis activity and patients with Acute Lymphoblastic Leukemia (ALL) with an Artemis inhibitor should result in the persistence of unrepaired RAG-mediated DNA DSBs as hair-pinned DNA ends, and subsequent induction of the apoptotic cascade.

Analysis of data generated from samples obtained in the course of clinical trials supports the finding that lower Artemis expression correlates with better overall and relapse-free survival in ALL patients. Although Artemis inhibition will also induce death in normal pre-B and pre-T cells, these will be replaced upon cessation of therapy by stem cells in the patient's immune system. Importantly, the treatment-related temporary block in B- and T-cell maturation would not be a dose-limiting side effect, because the mature lymphocyte compartment present prior to treatment is quite large and will not be affected by Artemis inhibition.

### BRIEF DESCRIPTION OF THE DRAWING

FIG. 1 is a schematic illustration of a High-Throughput Screening (HTS) Assay. Steps 1, 2, and 3 show the fluorescence assay for cutting of the DNA hairpin substrate by Artems:DNA-Kcs. Artemis activity assay is optimized for development of a fluorescent signal from the cleavage of the 6-FAM modified end of hairpin DNA (to release it from proximity to the BHQ quencher) and monitored kinetically on a suitable microplate reader with FITC filters (excitation wavelength=485 nm, emission wavelength=520 nm). Alternative FRET-matched fluorophore-quencher combinations can be selected. A hypothetical readout is illustrated subsequent to Step 3. Compounds are tested in concentration response and the slopes of the enzyme readouts can be converted to percent inhibition fit to a 4-parameter logistic curve to calculate the IC₅₀ of each compound.

### DETAILED DESCRIPTION

One aspect of the present disclosure relates to a compound of Formula (I):

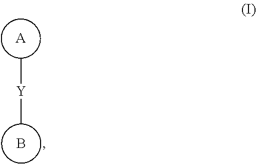

where

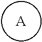

is selected from the group consisting of

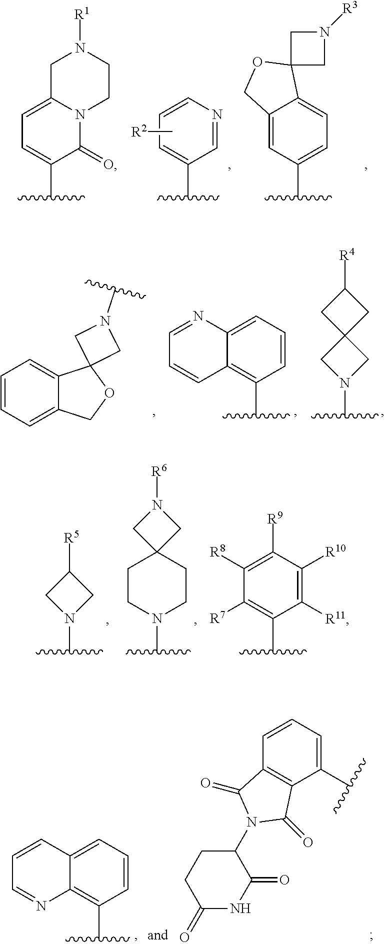

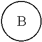

is selected from the group consisting of

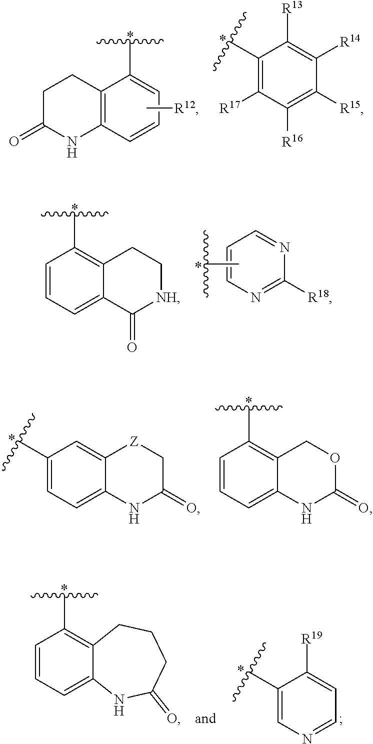

Y is absent, or, if present, is selected from the group consisting of —NH—, —NMe-, and

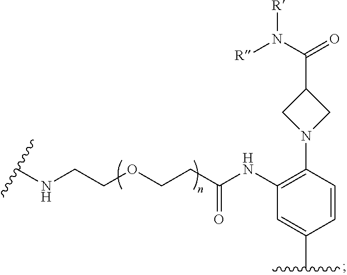

Z is CH₂ or O;
R′ is C₁₋₆ alkyl;
R″ is C₁₋₆ alkyl;
R¹ is selected from the group consisting of H, CN, C₁₋₆ alkyl, C₃₋₈ cycloalkyl, aryl, —C(O)C₁₋₆ alkyl, —C(O)C₂₋₆ alkenyl, —C(O)C₂₋₆ alkynyl, —C(O)aryl, —C(O)(CH₂)ₘbenzyl, —C(O)(CH₂)ₘNHMe, —C(O)(CH₂)ₘNMe₂, —C(O)(CH₂)ₘC₃₋₈ cycloalkyl, —C(O)(CH₂)ₘaryl, —C(O)(CH₂)ₘheteroaryl, —C(O)(CH₂)ₘheterocyclyl, —C(O)nonaromatic bicyclic compound, —C(O)CH(OMe)Ph, —S(O)₂C₁₋₆ alkyl, —S(O)₂C₃₋₈ cycloalkyl, and —S(O)₂aryl, where C₁₋₆ alkyl, C₂₋₆ alkenyl, C₃₋₈ cycloalkyl, aryl, benzyl, heteroaryl, and heterocyclyl, can be optionally substituted 1 to 3 times with a substituent independently selected at each occurrence from the group consisting of halogen, CN, CF₃, ═O, OH, COOH, —C₁₋₆ alkyl, C₃₋₈ cycloalkyl, aryl, heterocyclyl, and heteroaryl, —OC₁₋₆ alkyl, —O-aryl, —S(O)₂C₁₋₆ alkyl, —NHC(O)C₁₋₆ alkyl, —NHC(O)aryl, —C(O)C₁₋₆ alkyl, —C₁₋₆ alkylene-OH, —C₁₋₆ alkylene-C(O)OH, —NR^{a}R^{b}, and —C(O)NH₂;
R² is selected from the group consisting of halogen, —COOH, —C₁₋₆ alkyl, —OC₁₋₆ alkyl, —NR^{a}R^{b}, —NH(CH₂)ₖC(O)NR^{a}R^{b}, —NH(CH₂)ₖC(O)NR^{c}R^{d}, —C(O)NR^{a}R^{b}, and —S(O)₂C₁₋₆ alkyl;
R³ is selected from the group consisting of H, C₁₋₆ alkyl, C₃₋₈ cycloalkyl, C₄₋₁₄ cycloalkylalkyl, aryl, arylalkyl, —C(O)C₁₋₆ alkyl, —C(O)C₂₋₆ alkenyl, —C(O)C₃₋₈ cycloalkyl, —C(O)OC₁₋₆ alkyl, —C(O)(CH₂)_{q}aryl, —C(O)(CH₂)_{q}heteroaryl, —C(O)(CH₂)_{q}heterocyclyl, —S(O)₂C₁₋₆ alkyl, —S(O)₂C₂₋₆ alkenyl, —S(O)₂C₃₋₈ cycloalkyl, —S(O)₂(CH₂)_{q}aryl, —S(O)₂heteroaryl, and —S(O)₂heterocyclyl, where C₁₋₆ alkyl, C₃₋₈ cycloalkyl, aryl, arylalkyl, heteroaryl, and heterocyclyl can be optionally substituted 1 to 3 times with a substituent independently selected at each occurrence from the group consisting of OH, COOH, CF₃, halogen, C₁₋₆ alkyl, aryl, heteroaryl, heterocyclyl, NH₂, —OC₁₋₆ alkyl, —C(O)C₁₋₆ alkyl, —CH₂COOH, —CH₂OH, and —C(O)NH₂;
R⁴ is selected from the group consisting of —NR^{a}R^{b}, —NH—(CH₂)ₚ-heteroaryl, —NH—(CH₂)ₚheterocyclyl, —NH—C(O)C₁₋₆ alkyl, —NH—C(O)C₂₋₆ alkenyl, —NH—C(O)C₁₋₆ alkyl-aryl, —NH—CH₂—C(O)NR^{a}R^{b}, —NH—S(O)₂C₁₋₆ alkyl, and —NH—S(O)₂C₂₋₆ alkenyl, where C₁₋₆ alkyl, heteroaryl, and heterocyclyl can be substituted 1 to 3 times with C₁₋₆ alkyl;
R⁵ is selected from the group consisting of —NH—C(O)—CH₂—NH—C(O)-aryl, —NH—C(O)—CH₂—NH—C(O)—C₂₋₆ alkenyl, and —NH-heteroarylene-C(O)—NR^{a}R^{b};
R⁶ is selected from the group consisting of H, —C(O)C₁₋₆ alkyl, and —C(O)C₂₋₆ alkyl;
R⁷ is selected from the group consisting of H, CN, C₁₋₆ alkyl, —CH₂OH, and —C(O)NH₂;
R⁸ is selected from the group consisting of H, halogen, —C₁₋₆ alkyl, —CH₂OH, —OC₁₋₆ alkyl, —NMe₂, and —C(O)NHMe;
R⁹ is selected from the group consisting of H, halogen, C₁₋₆ alkyl, —OC₁₋₆ alkyl, heterocyclyl, —NR^{a}R^{b}, and —NH—CH₂—C(O)NH-i-Pr, where C₁₋₆ alkyl and heterocyclyl can be substituted with R²¹;
R¹⁰ is selected from the group consisting of H, C₁₋₆ alkyl, —OC₁₋₆ alkyl, —CH₂OH, —NH₂, —NH—C(O)C₁₋₆ alkyl, and —NH—C(O)OC₁₋₆ alkyl, where C₁₋₆ alkyl can be optionally substituted with —OC₁₋₆ alkyl;
R¹¹ is selected from the group consisting of H, halogen, and —C₁₋₆ alkyl;
R¹² is selected from the group consisting of H, halogen, C₁₋₆ alkyl, and —OC₁₋₆ alkyl;
R¹³ is selected from the group consisting of H, OH, —CH₂OH, and C₁₋₆ alkyl;
R¹⁴ is selected from the group consisting of H, C₁₋₆ alkyl, halogen, OH, —OC₁₋₆ alkyl, and CN;
R¹⁵ is selected from the group consisting of H, C₁₋₆ alkyl, —CH₂OH, CN, and —C(O)NH₂;
R¹⁶ is selected from the group consisting of H, CN, halogen, and —C(O)NH₂;
R¹⁷ is selected from the group consisting of H, OH, C₁₋₆ alkyl, —CH₂OH, and —C(O)NH₂;
R¹⁸ is selected from the group consisting of —OC₁₋₆ alkyl,

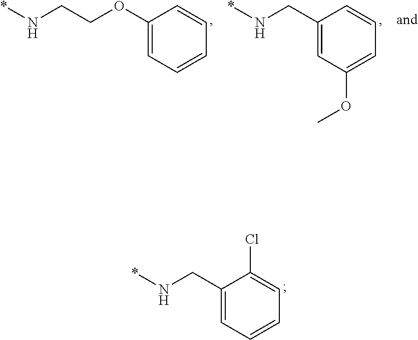

R¹⁹ is C₁₋₆ alkyl or —CH₂OH;
R²⁰ is independently selected at each occurrence from the group consisting of ═O, COOH, C₁₋₆ alkyl, C₃₋₈ cycloalkyl, —C(O)C₁₋₆ alkyl, —C(O)C₂₋₆ alkenyl, —C(O)—C₃₋₈ cycloalkyl, —C(O)aryl, —C(O)heteroaryl, —C(O)heterocyclyl, —C(O)NR^{aa}R^{bb}, —C(O)OC₁₋₆ alkyl, —NH—C(O)OC₁₋₆ alkyl, —NH—C(O)C₂₋₆ alkenyl, —NH—C(O)OC₂₋₆ alkenyl, —C₁₋₆ alkylene-arylene-NH—C(O)C₁₋₆ alkyl, —C₁₋₆ alkylene-arylene-NH—C(O)C₂₋₆ alkenyl, —S(O)₂C₁₋₆ alkyl, —S(O)₂C₂₋₆ alkenyl, —S(O)₂C₃₋₈ cycloalkyl, —S(O)₂(CH₂)ₛaryl, —S(O)₂heterocyclyl, —(CH₂)ᵣheterocyclyl, where C₁₋₆ alkyl, C₁₋₆ alkylene, C₂₋₆ alkenyl, C₃₋₈ cycloalkyl, aryl, arylene, heteroaryl, and heterocyclyl can be optionally substituted 1 to 3 times with a substituent independently selected at each occurrence from the group consisting of halogen, —OH, —COOH, —CN, —C₁₋₆ alkyl, —OC₁₋₆ alkyl, —C₃₋₈ cycloalkyl, aryl, heterocyclyl, —C(O)C₁₋₆ alkyl, —C(O)OC₁₋₆ alkyl, —C(O)OC₁₋₆ alkylene-aryl, and —S(O)₂C₁₋₆ alkyl;
R²¹ is independently selected at each occurrence form the group consisting of —NR^{a}R^{b}, —C(O)C₁₋₆ alkyl, —C(O)OC₁₋₆ alkyl, and —S(O)₂C₁₋₆ alkyl, where —S(O)₂C₁₋₆ alkyl can be optionally substituted with -aryl-COOH;
R²² is independently selected at each occurrence form the group consisting of C₃₋₈ cycloalkyl, aryl, benzyl, —NR^{aa}R^{bb}, heteroaryl, and heterocyclyl, where heteroaryl and heterocyclyl can be optionally substituted 1 to 3 times with C₁₋₆ alkyl;
R^{a} is selected from the group consisting of H, C₁₋₆ alkyl, aryl, and heteroaryl, where C₁₋₆ alkyl, aryl, and heteroaryl can be optionally substituted 1 to 3 times with a substituent independently selected at each occurrence from the group consisting of H, OH, COOH, CF₃, —OC₁₋₆ alkyl, aryl, and —NR^{aa}R^{bb};
R^{b} is H or C₁₋₆ alkyl, where C₁₋₆ alkyl can be optionally substituted 1 to 3 times with —C(O)NR^{aa}R^{bb}; or
R^{a} and R^{b} combine with the nitrogen atom to which they are attached to form piperazine, azetidine, piperidine, pyrrolidine, diazabicyclo[2.2.1]heptane, diazabicyclo[2.2.2]octane, 1,4-diazepane, or 2,7-diazaspiro[3.5]nonane ring, where piperazine, azetidine, piperidine, pyrrolidine, diazabicyclo[2.2.1]heptane, diazabicyclo[2.2.2]octane, 1,4-diazepane, or 2,7-diazaspiro[3.5]nonane ring can be optionally substituted 1 to 3 times with R²⁰,
R^{c} is H, C₁₋₆ alkyl, or C₃₋₈ cycloalkyl, where C₁₋₆ alkyl can be optionally substituted 1 to 3 times with R²²;
R^{d} is H or C₁₋₆ alkyl; or
R^{c} and R^{d} combine with the nitrogen atom to which they are attached to form piperazine, piperidine, pyrrolidine, or morpholine ring, where piperazine or piperidine ring can be optionally substituted 1 to 3 times with a substituent independently selected at each occurrence from the group consisting of C₁₋₆ alkyl, heteroaryl, and heterocyclyl;
R^{aa} is H, C₁₋₆ alkyl, aryl, arylalkyl, or CN, where C₁₋₆ alkyl can be optionally substituted 1 to 3 times with—a substituent independently selected at each occurrence from the group consisting of C(O)OC₁₋₆ alkyl, COOH, and OH;
R^{bb} is H or C₁₋₆ alkyl;
m is 0, 1, 2, or 3;
n is 1, 2, 3, 4, or 5;
p is 1 or 2;
q is 0, 1, or 2;
r is 1;
s is 0 or 1; and
k is 0 or 1;
   or an oxide thereof, a pharmaceutically acceptable salt thereof, a solvate thereof, or a prodrug thereof.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “alkyl” means an aliphatic hydrocarbon group which may be straight or branched having about 1 to about 12 carbon atoms in the chain. Particular alkyl groups have 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl. The term “alkylene” refers to a divalent group formed from an alkane by removal of two hydrogen atoms. Exemplary alkylene groups include, but are not limited to, methylene, ethylene, propylene, and other divalent groups derived from the alkanes described above.

The term “alkenyl” means an aliphatic hydrocarbon group containing a carbon-carbon double bond and which may be straight or branched having about 2 to about 6 carbon atoms in the chain. Particular alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. The term “alkenyl” may also refer to a hydrocarbon chain having 2 to 6 carbons containing at least one double bond and at least one triple bond.

The term “alkynyl” means an aliphatic hydrocarbon group containing a carbon-carbon triple bond and which may be straight or branched having about 2 to about 6 carbon atoms in the chain. Particular alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl.

The term “cycloalkyl” means a non-aromatic mono- or multicyclic ring system of about 3 to about 12 carbon atoms, preferably of about 3 to about 8 carbon atoms. Exemplary monocyclic cycloalkyls include cyclopentyl, cyclohexyl, cycloheptyl, bicyclo[1.1.1]pentyl, and the like.

The term “cycloalkylalkyl” means a cycloalkyl-alkyl-group in which the cycloalkyl and alkyl are as defined herein. Exemplary cycloalkylalkyl groups include cyclopropylmethyl, cyclobutylmethyl, cyclopentylmethyl, cyclopropylethyl, cyclobutylethyl, and cyclopentylethyl. The alkyl radical and the cycloalkyl radical may be optionally substituted as defined herein.

The term “aryl” means an aromatic monocyclic or multicyclic ring system of 6 to about 14 carbon atoms, preferably of 6 to about 10 carbon atoms. Representative aryl groups include phenyl and naphthyl. The term “arylene” refers to a group obtained by removal of a hydrogen atom from an aryl group. Non-limiting examples of arylene include phenylene and naphthylene.

The term “arylalkyl” or “alkylaryl” means an alkyl substituted with one or more aryl groups, wherein the alkyl and aryl groups are as herein described. One particular example is an arylmethyl or arylethyl group, in which a single or a double carbon spacer unit is attached to an aryl group, where the carbon spacer and the aryl group can be optionally substituted as described herein. Representative arylalkyl groups include

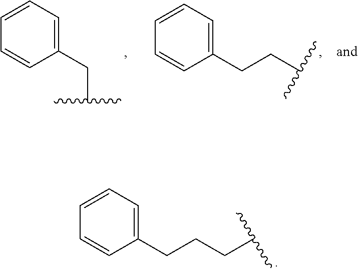 [SMILES: *Cc1ccccc1 ; *CCc1ccccc1 ; *CCCc1ccccc1]

The term “heteroaryl” means an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen, or sulfur. In the case of multicyclic ring system, only one of the rings needs to be aromatic for the ring system to be defined as “Heteroaryl”. Preferred heteroaryls contain about 5 to 6 ring atoms. The prefix aza, oxa, thia, or thio before heteroaryl means that at least a nitrogen, oxygen, or sulfur atom, respectively, is present as a ring atom. A nitrogen atom of a heteroaryl is optionally oxidized to the corresponding N-oxide. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, phthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like. The term “heteroarylene” refers to a group obtained by removal of a hydrogen atom from a heteroaryl group. Exemplary heteroarylene groups include, but are not limited to, groups derived from the heteroaryl groups described above.

As used herein, “heterocyclyl” refers to a stable 3- to 18-membered ring (radical) which consists of carbon atoms and from one to five heteroatoms selected from the group consisting of nitrogen, oxygen and sulfur. For purposes of this application, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. For purposes of this application, when the heterocycle is a polycyclic ring system, one of the rings can be an aromatic ring and another one is non-aromatic. Examples of heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The term “nonaromatic bicyclic compound” means a non-aromatic bicyclic system containing 6 to 14 atoms, preferably 7 to about 10 carbon atoms. Representative bicyclic compounds include 2,3-dihydro-1H-indenyl, 1,2,3,4-tetrahydronaphthalenyl, 6,7,8,9-tetrahydro-5H-benzo[7]annulenyl and the like.

The term “monocyclic” used herein indicates a molecular structure having one ring.

The term “polycyclic” or “multi-cyclic” used herein indicates a molecular structure having two or more rings, including, but not limited to, fused, bridged, or spiro rings.

The term “phenyl” means a phenyl group as shown below

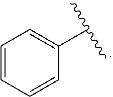 [SMILES: *c1ccccc1]

The term “benzyl” means a benzyl group as shown below

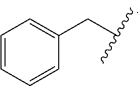 [SMILES: *Cc1ccccc1]

The term “2,7-diazaspiro[3.5]nonane” means a 2,7-diazaspiro[3.5]nonane group as shown below

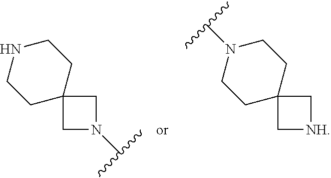 [SMILES: *N1CC2(CCNCC2)C1 ; *N1CCC2(CC1)CNC2]

The term “diazabicyclo[2.2.1]heptane” means a diazabicyclo[2.2.1]heptane group as shown below

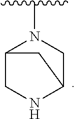 [SMILES: *N1CC2CC1CN2]

The term “diazabicyclo[2.2.2]octane” means a diazabicyclo[2.2.2]octane group as shown below

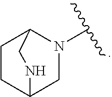 [SMILES: *N1CC2CCC1CN2]

The term “1,4-diazepane” means a 1,4-diazepane group as shown below

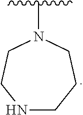 [SMILES: *N1CCCNCC1]

Terminology related to “protecting”, “deprotecting,” and “protected” functionalities occurs throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes described herein, the person of ordinary skill can readily envision those groups that would be suitable as “protecting groups.” Suitable groups for that purpose are discussed in standard textbooks in the field of chemistry, such as Greene, Protective Groups in Organic Synthesis, John Wiley & Sons, New York (1991), which is hereby incorporated by reference in its entirety.

A compound with a hydroxy group drawn next to a nitrogen on a heterocycle can exist as the “keto” form. For example, 3-(2-hydroxy-[1,2,4]triazolo[1,5-a]pyridin-6-yl)propanoic acid can exist as 3-(2-oxo-2,3-dihydro-[1,2,4]triazolo[1,5-a]pyridin-6-yl)propanoic acid.

The term “halogen” means fluoro, chloro, bromo, or iodo.

The term “substituted” or “substitution” of an atom means that one or more hydrogen on the designated atom is replaced with a selection from the indicated group, provided that the designated atom's normal valency is not exceeded.

“Unsubstituted” atoms bear all of the hydrogen atoms dictated by their valency. When a substituent is keto (i.e., ═O), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, lower alkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The term “method of treating” means amelioration or relief from the symptoms and/or effects associated with the disorders described herein. As used herein, reference to “treatment” of a patient is intended to include prophylaxis. Treatment duration can be of limited duration, e.g., for a specific period of time, or may be for an extended duration such as for the remainder of the patient's life.

The term “compounds of the invention”, and equivalent expressions, are meant to embrace compounds of general Formula (I), Formula (IA), Formula (IB), Formula (IC), Formula (ID), Formula (IE), Formula (IF), Formula (IG), Formula (IH), Formula (IJ), Formula (IK), Formula (IL), Formula (IM), Formula (IN), Formula (IC), Formula (IP), Formula (IQ), Formula (IR), Formula (IS), Formula (IA′), Formula (IB′), Formula (IC′), Formula (ID′), Formula (IE′), and Formula (IF′), as hereinbefore described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The term “pharmaceutically acceptable salts” means the relatively non-toxic, inorganic, and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like (see, for example, Berge et al., “Pharmaceutical Salts,” J. Pharm. Sci., 66:1-9 (1977) and Remington's Pharmaceutical Sciences, 17th ed., Mack Publishing Company, Easton, Pa., 1985, p. 1418, which are hereby incorporated by reference in their entirety). Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include, for example, sodium hydride, sodium hydroxide, potassium hydroxide, calcium hydroxide, aluminum hydroxide, lithium hydroxide, magnesium hydroxide, and zinc hydroxide. Suitable amine base addition salts are prepared from amines which have sufficient basicity to form a stable salt, and preferably include those amines which are frequently used in medicinal chemistry because of their low toxicity and acceptability for medical use, such as ammonia, ethylenediamine, N-methylglucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, dicyclohexylamine, and the like.

The term “pharmaceutically acceptable prodrugs” as used herein means those prodrugs of the compounds useful according to the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” means compounds that are rapidly transformed in vivo to yield the parent compound of the above formula, for example by hydrolysis in blood. Functional groups which may be rapidly transformed, by metabolic cleavage, in vivo form a class of groups reactive with the carboxyl group of the compounds of this invention. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier (1985); Methods in Enzymology, K. Widder et al, Ed., Academic Press, 42, p. 309-396 (1985); A Textbook of Drug Design and Development, Krogsgaard-Larsen and H. Bundgaard, ed., Chapter 5; “Design and Applications of Prodrugs” p. 113-191 (1991); Advanced Drug Delivery Reviews, H. Bundgard, 8, p. 1-38 (1992); J. Pharm. Sci., 77:285 (1988); Nakeya et al, Chem. Pharm. Bull., 32:692 (1984); Higuchi et al., “Pro-drugs as Novel Delivery Systems,” Vol. 14 of the A.C.S. Symposium Series, and Bioreversible Carriers in Drug Design, Edward B. Roche, ed., American Pharmaceutical Association and Pergamon Press (1987), which are incorporated herein by reference in their entirety. Examples of prodrugs include, but are not limited to, acetate, formate, and benzoate derivatives of alcohol and amine functional groups in the compounds of the invention.

The term “solvate” refers to a compound of Formula (I), Formula (IA), Formula (IB), Formula (IC), Formula (ID), Formula (IE), Formula (IF), Formula (IG), Formula (IH), Formula (IJ), Formula (IK), Formula (IL), Formula (IM), Formula (IN), Formula (IO), Formula (IP), Formula (IQ), Formula (IR), Formula (IS), Formula (IA′), Formula (IB′), Formula (IC′), Formula (ID′), Formula (IE′), and Formula (IF′) in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The term “therapeutically effective amounts” is meant to describe an amount of compound of the present invention effective to produce the desired therapeutic effect. Such amounts generally vary according to a number of factors well within the purview of ordinarily skilled artisans given the description provided herein to determine and account for. These include, without limitation: the particular subject, as well as its age, weight, height, general physical condition, and medical history; the particular compound used, as well as the carrier in which it is formulated and the route of administration selected for it; and the nature and severity of the condition being treated.

The term “pharmaceutical composition” means a composition comprising a compound of Formula (I), Formula (IA), Formula (IB), Formula (IC), Formula (ID), Formula (IE), Formula (IF), Formula (IG), Formula (IH), Formula (IJ), Formula (IK), Formula (IL), Formula (IM), Formula (IN), Formula (IO), Formula (IP), Formula (IQ), Formula (IR), Formula (IS), Formula (IA′), Formula (IB′), Formula (IC′), Formula (ID′), Formula (IE′), and Formula (IF′) and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The term “pharmaceutically acceptable” means it is, within the scope of sound medical judgement, suitable for use in contact with the cells of humans and lower animals without undue toxicity, irritation, allergic response and the like, and are commensurate with a reasonable benefit/risk ratio.

The term “pharmaceutically acceptable dosage forms” means dosage forms of the compound of the invention, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

This technology also envisions the “quaternization” of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

In the characterization of some of the substituents, it is recited that certain substituents may combine to form rings. Unless stated otherwise, it is intended that such rings may exhibit various degrees of unsaturation (from fully saturated to fully unsaturated), may include heteroatoms and may be substituted with lower alkyl or alkoxy.

In some embodiments, R¹ is

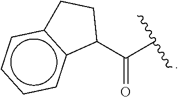

In some embodiments, the compound has the Formula (IA):

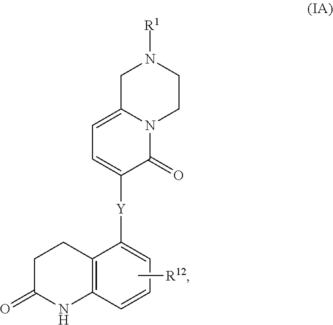

where Y is absent, or, if present, is —NH— or —NMe-.

In some embodiments, the compound has the Formula (IA′):

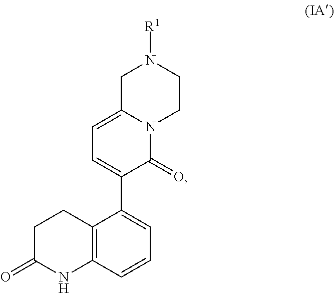

where R¹ is selected from the group consisting of C₃₋₈ cycloalkyl, —C(O)aryl, —S(O)₂aryl, —C(O)heteroaryl, and —C(O)C₃₋₈ cycloalkyl, where aryl and heteroaryl can be optionally substituted 1 to 3 times with a substituent independently selected at each occurrence from the group consisting of H, halogen, —C₁₋₆ alkyl, and —OC₁₋₆ alkyl.

In some embodiments, R¹ is selected from the group consisting of

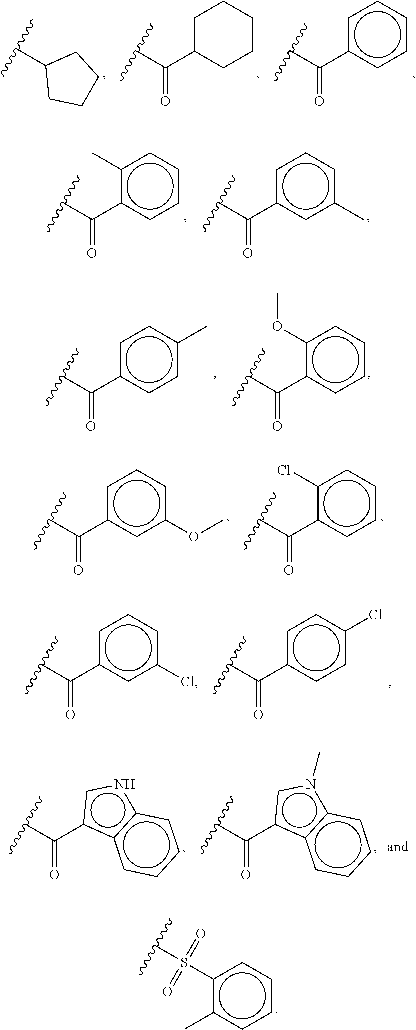

In some embodiments, the compound has the Formula (IB):

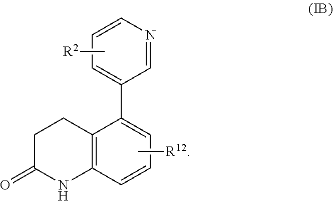

In some embodiments, the compound has the Formula (IB′):

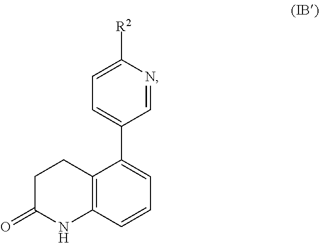

where
R² is —NR^{a}R^{b};
R^{a} and R^{b} combine with the nitrogen atom to which they are attached to form piperazine, azetidine, diazabicyclo[2.2.1]heptane, and diazabicyclo[2.2.2]octane, where piperazine, azetidine, diazabicyclo[2.2.1]heptane, and diazabicyclo[2.2.2]octane can be optionally substituted 1 to 3 times with R²⁰;
R²⁰ is independently selected at each occurrence form the group consisting of H, C₁₋₆ alkyl, C₃₋₈ cycloalkyl, —C(O)C₁₋₆ alkyl, —C(O)C₂₋₆ alkenyl, —C(O)—C₃₋₈ cycloalkyl, —C(O)aryl, —C(O)heteroaryl, —C(O)heterocyclyl, —C(O)NR^{aa}R^{bb}, —C(O)OC₁₋₆ alkyl, —S(O)₂C₁₋₆ alkyl, —S(O)₂C₂₋₆ alkenyl, —S(O)₂C₃₋₈ cycloalkyl, —S(O)₂aryl, and —S(O)₂heterocyclyl, where C₁₋₆ alkyl, C₂₋₆ alkenyl, C₃₋₈ cycloalkyl, aryl, and heteroaryl can be optionally substituted 1 to 3 times with a substituent independently selected at each occurrence from the group consisting of H, halogen, —COOH, —C₁₋₆ alkyl, —OC₁₋₆ alkyl, C₃₋₈ cycloalkyl, and aryl;
R^{aa} is selected at each occurrence form the group consisting of H, C₁₋₆ alkyl, and arylalkyl; and
R^{bb} is H or C₁₋₆ alkyl.

In some embodiments, R² is selected from the group consisting of

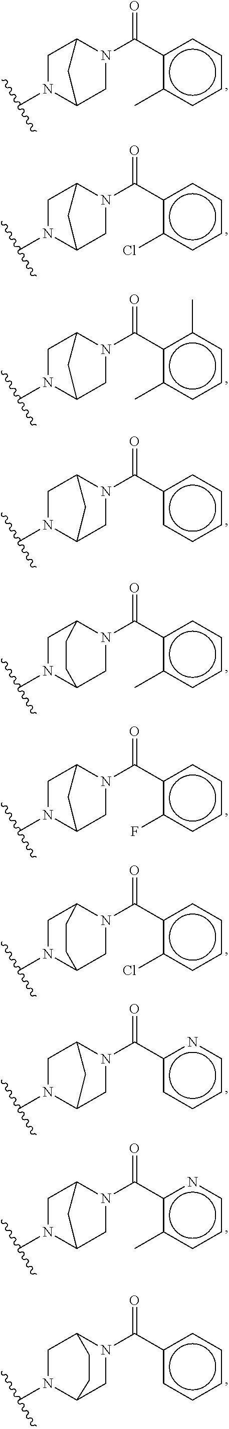

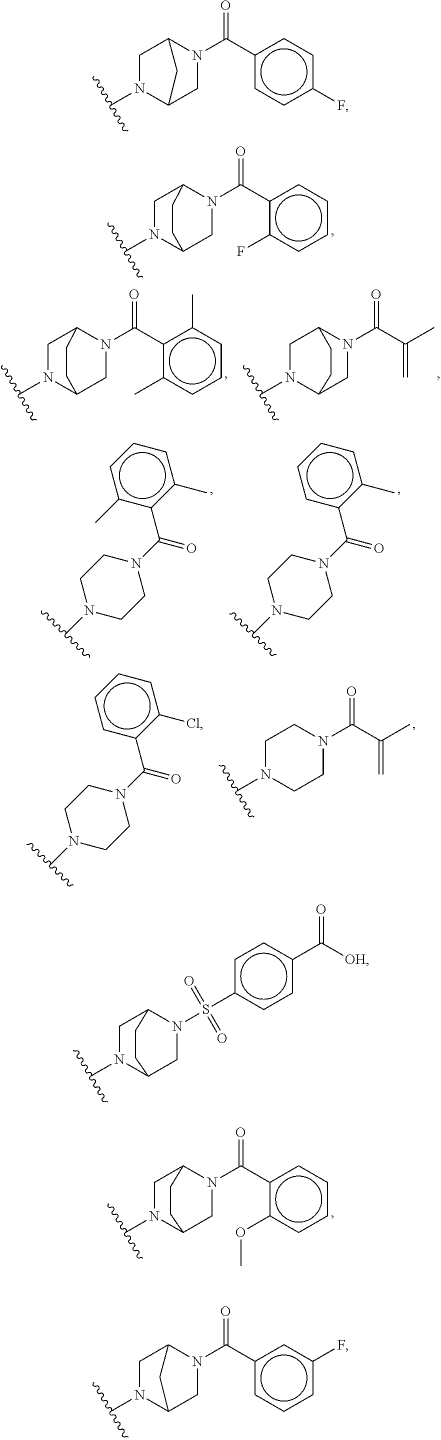

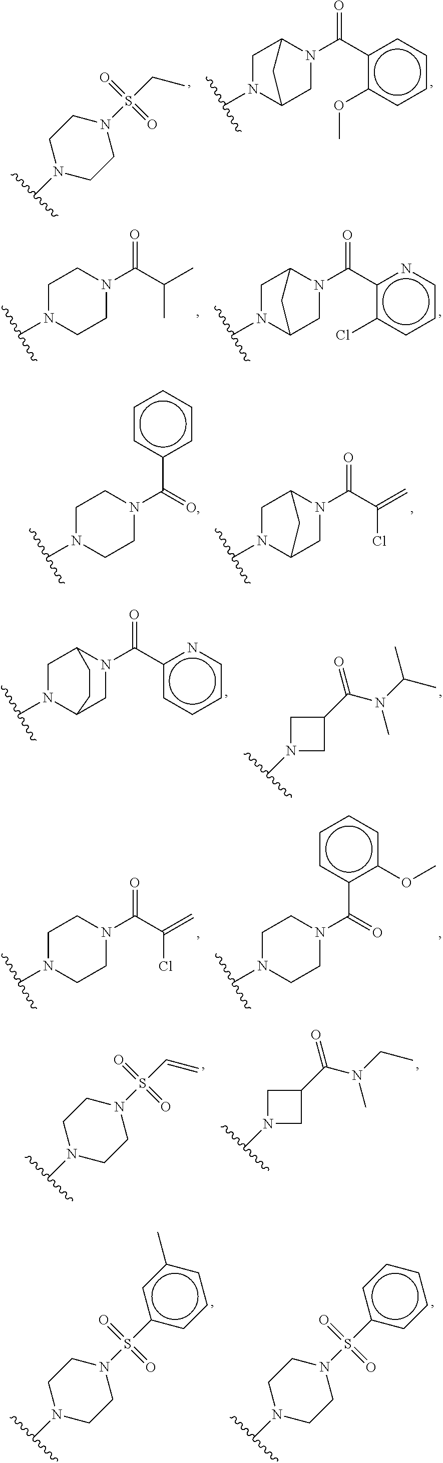

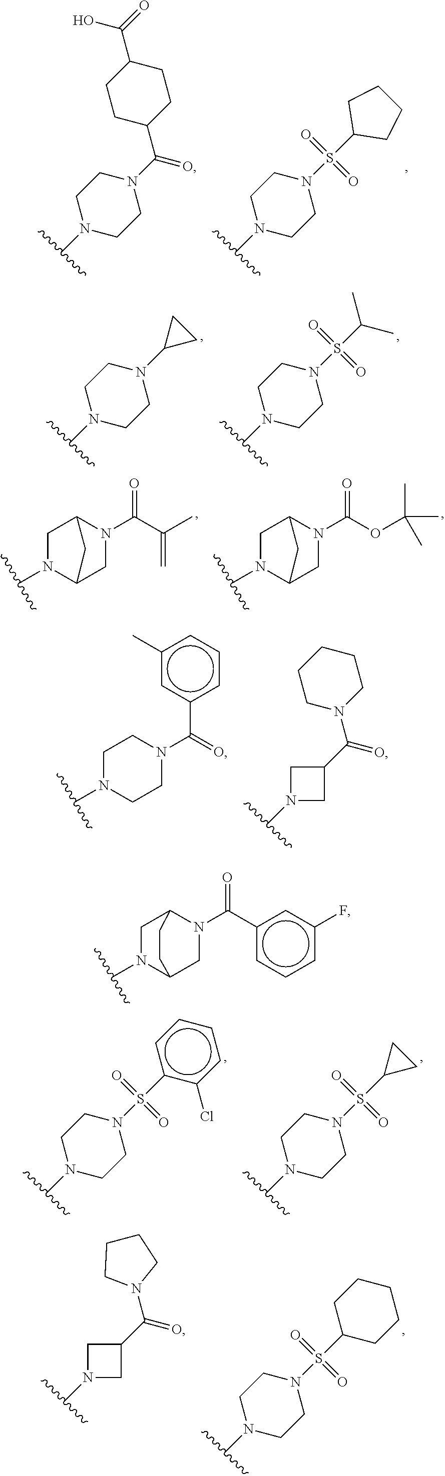

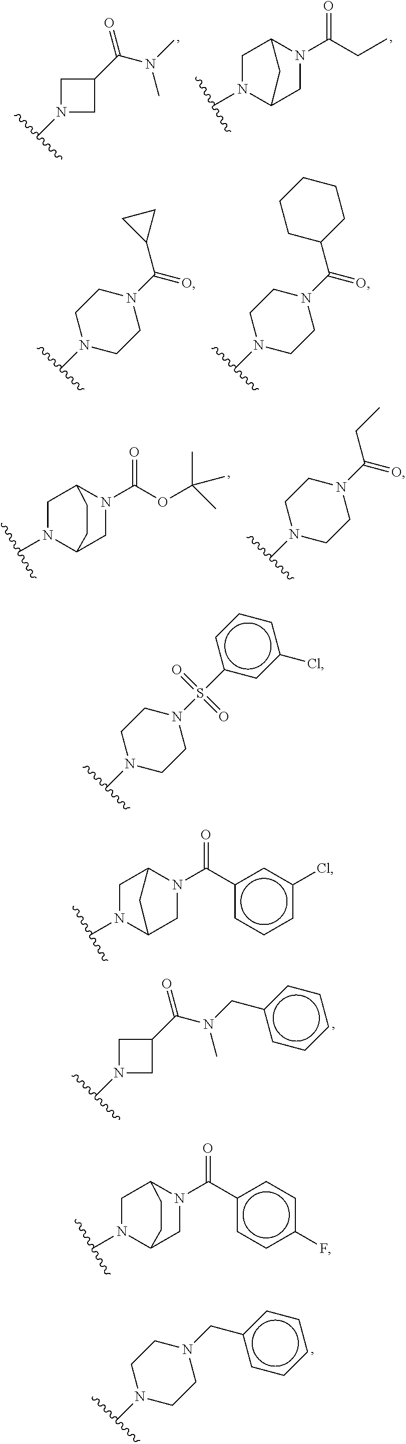

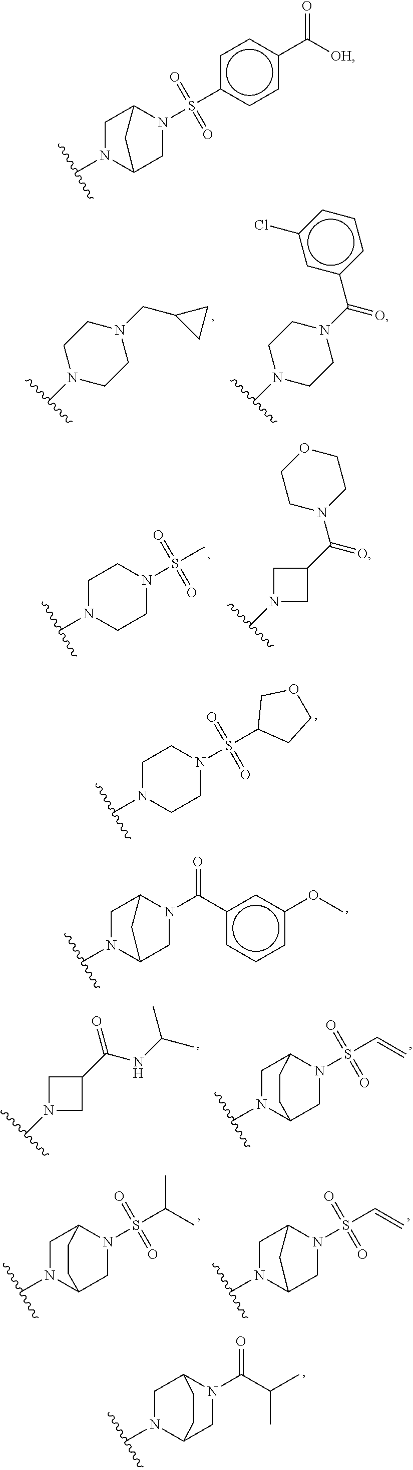

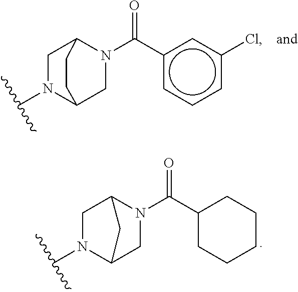

In some embodiments, R² is

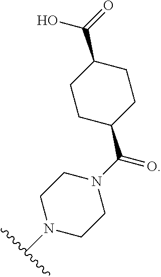 [SMILES: *N1CCN(C(=O)[C@H]2CC[C@@H](C(=O)O)CC2)CC1]

In some embodiments, the compound has the Formula (IC):

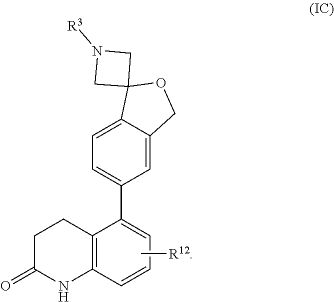

In some embodiments, the compound has the Formula (IC′):

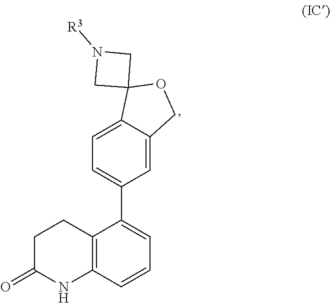

where R³ is selected from the group consisting of —C(O)C₁₋₆ alkyl, —C(O)C₂₋₆ alkenyl, —C(O)C₃₋₈ cycloalkyl, —C(O)aryl, and —S(O)₂aryl, where C₃₋₈ cycloalkyl and aryl can be optionally substituted 1 to 3 times with COOH.

In some embodiments, R³ is selected from the group consisting of

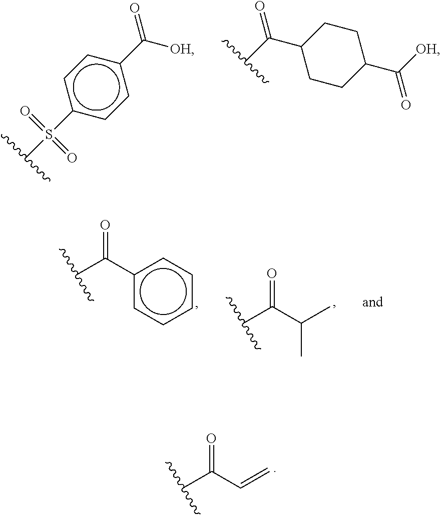

In some embodiments, R³ is

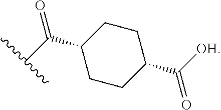 [SMILES: *C(=O)[C@H]1CC[C@@H](C(=O)O)CC1]

In some embodiments, the compound has the Formula (ID):

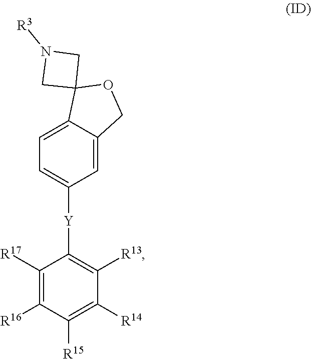

where Y is absent, or, if present, is —NH— or —NMe-.

In some embodiments, the compound has the Formula (ID′):

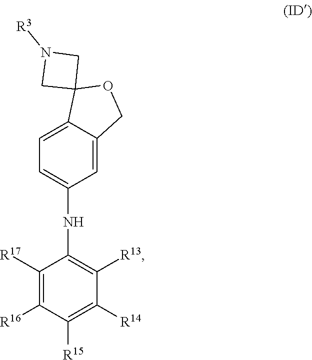

where R³ is —S(O)₂aryl, where aryl can be optionally substituted 1 to 3 times with —COOH.

In some embodiments, R³ is

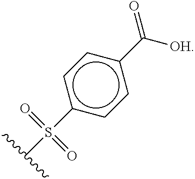

In some embodiments, R¹³ is CH₃, R¹⁴ is H, Ru is H, R¹⁶ is CN, and R¹⁷ is H.

In some embodiments, the compound has the Formula (IE):

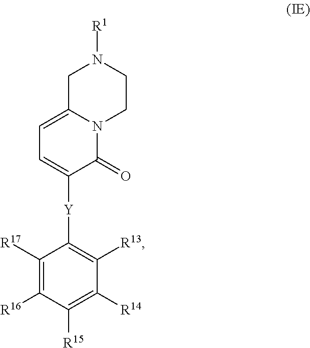

where Y is absent, or, if present, is —NH— or —NMe-.

In some embodiments, the compound has the Formula (IE′):

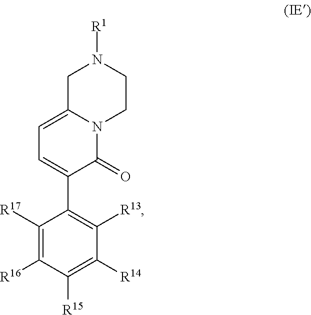

where R¹ is —C(O)aryl, where aryl can be optionally substituted 1 to 3 times with —C₁₋₆ alkyl.

In some embodiments, R¹ is

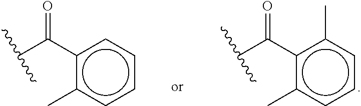

In some embodiments, R¹³ is H, R¹⁴ is F, R¹⁵ is H, R¹⁶ is CN, and R¹⁷ is H.

In some embodiments, the compound has the Formula (IF):

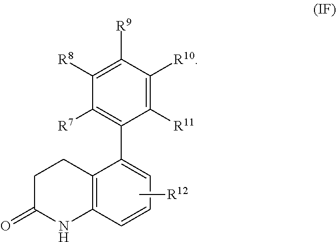

In some embodiments, the compound has the Formula (IF′):

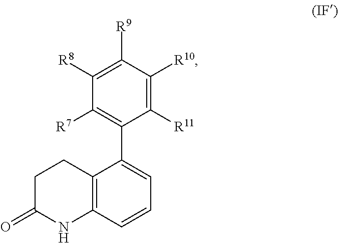

where
R⁹ is monocyclic heterocyclyl or —NR^{a}R^{b}, where monocyclic heterocyclyl can be optionally substituted 1 time with —C(O)C₁₋₆ alkyl, —C(O)OC₁₋₆ alkyl, or —S(O)₂C₁₋₆ alkyl;
R^{a} is heteroaryl optionally substituted 1 to 3 times with a substituent independently selected at each occurrence from the group consisting of H, OH, and CF₃;
R^{b} is H; or
R^{a} and R^{b} combine with the nitrogen atom to which they are attached to form azetidine optionally substituted 1 time with —C(O)NR^{aa}R^{bb};
R^{aa} is C₁₋₆ alkyl; and
R^{bb} is C₁₋₆ alkyl.

In some embodiments, R^{a} is pyridinyl substituted with OH and CF₃.

In some embodiments, R⁹ is selected from the group consisting of

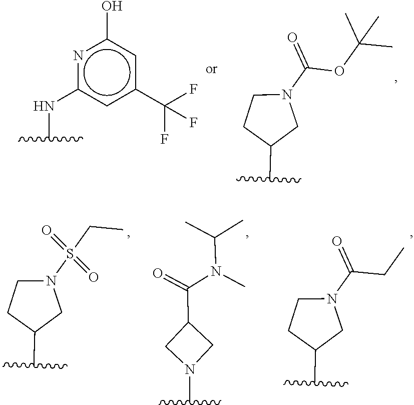

In some embodiments, R⁷ is H, R⁸ is H, R¹⁰ is H or NH₂, and R¹¹ is H.

In some embodiments, the compound has the Formula (IG):

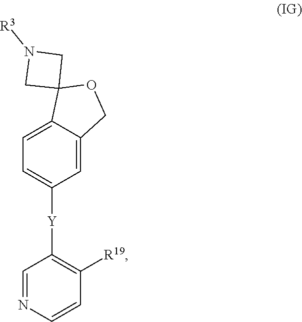

where Y is absent, or, if present, is —NH— or —NMe-.

In some embodiments, the compound has the Formula (IH):

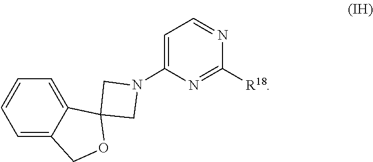

In some embodiments, the compound has the Formula (IJ):

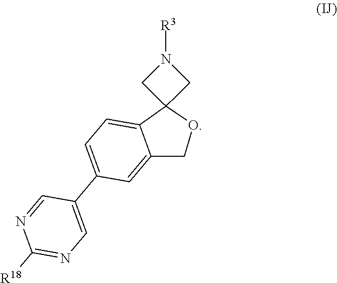

In some embodiments, the compound has the Formula (IK):

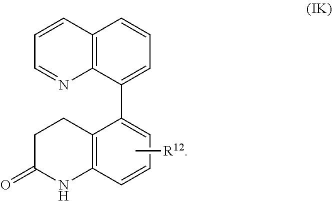

In some embodiments, the compound has the Formula (IL):

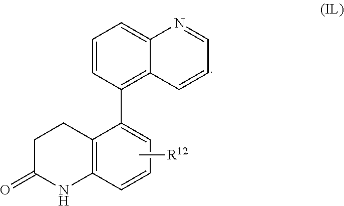

In some embodiments, the compound has the Formula (IM):

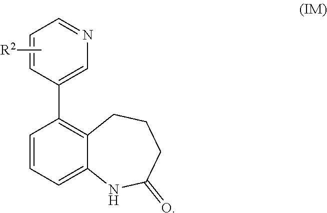

In some embodiments, the compound has the Formula (IN):

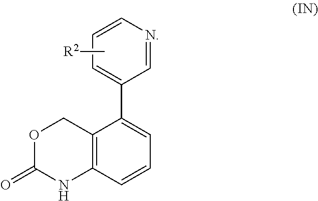

In some embodiments, the compound has the Formula (IO):

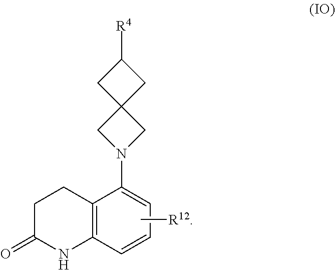

In some embodiments, the compound has the Formula (IP):

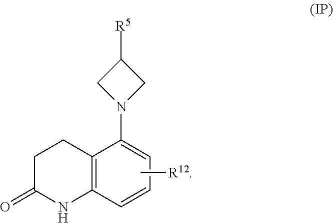

In some embodiments, the compound has the Formula (IQ):

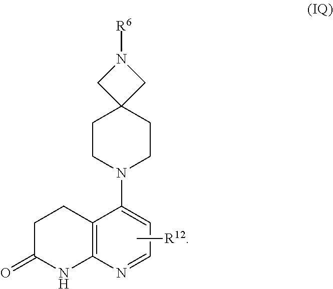

In some embodiments, the compound has the Formula (IR):

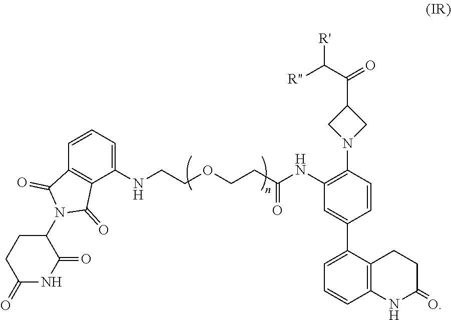

In some embodiments, the compound has the Formula (IS):

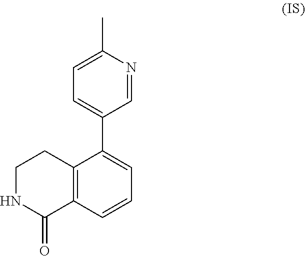 [SMILES: Cc1ccc(-c2cccc3c2CCNC3=O)cn1]

Compounds of the present invention can be produced according to the general schemes outlined below (Schemes 1-13). For example, compounds of Formula (I) can be prepared according to Scheme 1 below.

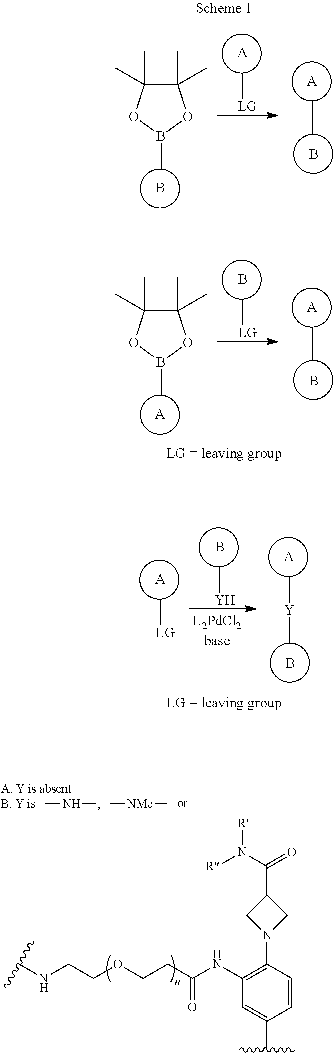

Compounds of Formula (I), where Y is absent, can be prepared by reacting a boronic ester with

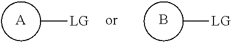

(Scheme 1A) in a presence of a suitable catalyst. Catalysts that can be used include, but are not limited to, dichloro[1,1′-bis(diphenylphosphino) ferrocene]palladium (II) dichloromethane adduct and tetrakis(triphenylphosphine)palladium (0). This reaction can be carried out in a variety of solvents, such as 1,4-dioxane, water, DMF, or other such solvents or in the mixture of such solvents. Compounds of Formula (I), where Y is of —NH—, —NMe-, or

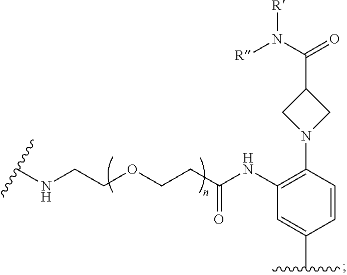

can be prepared by reacting an amine

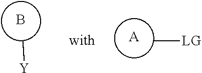

in the presence of base and palladium catalyst (Scheme 1B). Suitable compounds of formula

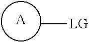

that can be used include for example aryl halides and trifluoromethanesulfonates.

Compounds of Formula (IA) can be prepared according to Schemes 2 and 3 below.

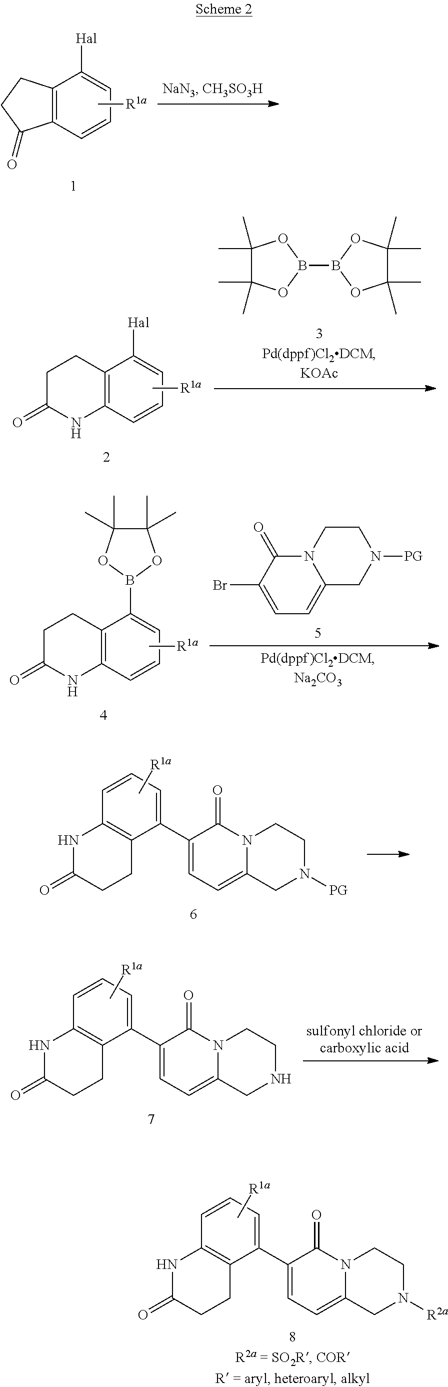

Compound 1 can be reacted with NaN₃ in methanesulfonic acid to produce 3,4-dihydroquinolin-2(1H)-one (2), which can be reacted with bis(pinacolato)diborane (3) in the presence of dichloro[1,1′-bis(diphenylphosphino)ferrocene]palladium (II) dichloromethane adduct and potassium acetate to form corresponding boronic ester 4. This reaction can be carried out in a variety of solvents, such as 1,4-dioxane, DMF, or other such solvents or in the mixture of such solvents. Boronic ester 4 can be reacted with compound 5 in the presence of dichloro[1,1′-bis(diphenylphosphino)ferrocene]palladium (II) dichloromethane adduct and sodium carbonate to form compound 6. This reaction can be carried out in a variety of solvents, such as 1,4-dioxane, water, DMF, or other such solvents or in the mixture of such solvents. Compound 6 can be deprotected to form compound 7 using any suitable deprotecting agent. A detailed description of the protecting groups and conditions for their removal is contained in The Peptides, Vol. 3, Gross and Meinenhofer, Eds., Academic Press, New York, 1981, which is hereby incorporated by reference in its entirety. This reaction can be carried out in a variety of solvents, such as methylene chloride (CH₂Cl₂), tetrahydrofuran (THF), dimethylformamide (DMF), or other such solvents or in the mixture of such solvents. Compound 7 can be treated with a sulfonyl chloride or coupled with a carboxylic acid to prepare compound (8). Sulfonamide 8 can be prepared by reacting compound 7 with the corresponding sulfonyl chloride in the presence of a base. Any suitable base can be used, for example, triethylamine or N,N-diisopropylethylamine. This reaction can be carried out in a variety of solvents, such as dichloromethane, dichloroethane, or other such solvents or in the mixture of such solvents. Amide 8 can be prepared by reacting compound 7 with the corresponding carboxylic acid in the presence of a base and a coupling agent. Any suitable base can be used, for example, triethylamine or N,N-diisopropylethylamine. Any suitable coupling agent can be used, for example, HATU, HOBT, PyBOP, HBTU, TBTU, HOAT, and PyBroP. The reaction can be carried out in a variety of solvents, for example in methylene chloride (CH₂Cl₂), tetrahydrofuran (THF), dimethylformamide (DMF), or other such solvents or in the mixture of such solvents.

Alternatively, compounds of Formula (IA) can be prepared by reacting compound 7 with an aldehyde or ketone in the presence of a reducing agent (Scheme 3). In some instances, an aldehyde derivative can be used and the aldehyde is formed during the reaction. Any reducing agent can be used, for example, NaBH₃CN, LiAlH₄, or NaBH(OAc)₃. This reaction can be carried out in a variety of solvents, such as dichloromethane, dichloroethane, methanol, ethanol, THF, diethyl ether, or other such solvents or a mixture of these solvents.

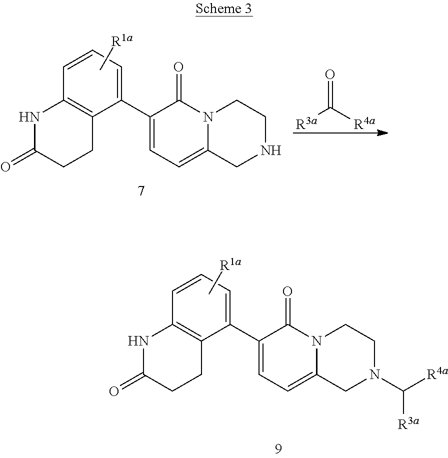

Compounds of Formula (IB) can be prepared according to the Schemes 4-7 below.

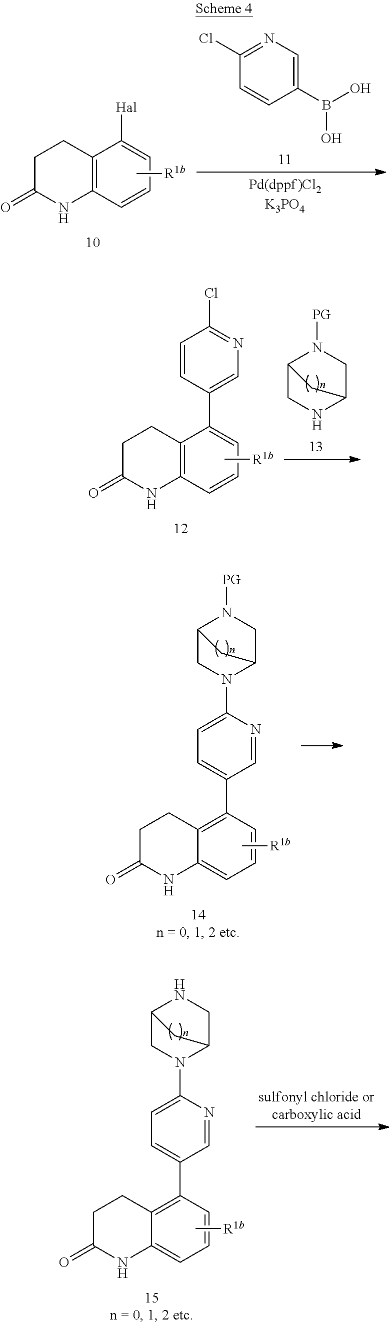

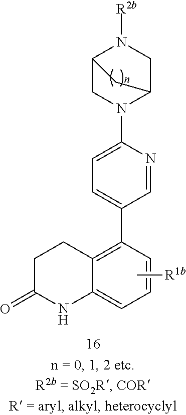

Compound 10 can be reacted with (6-chloropyridin-3-yl)boronic acid (11) to form 3,4-dihydroquinolin-2(1H)-one (12) in the presence of dichloro[1,1′-bis(diphenylphosphino) ferrocene]palladium(II) acetone adduct and potassium phosphate. This reaction can be carried out in a variety of solvents, such as 1,4-dioxane, DMF, or other such solvents or in the mixture of such solvents. 3,4-Dihydroquinolin-2(1H)-one (12) can be reacted with a suitable amine (13) to form compound 14. This reaction can be carried out in a variety of solvents, such as DMSO, DMF, or other such solvents or in the mixture of such solvents. Compound 14 can be deprotected to form compound 15 using any suitable deprotecting agent. A detailed description of the protecting groups and conditions for their removal is contained in The Peptides, Vol. 3, Gross and Meinenhofer, Eds., Academic Press, New York, 1981, which is hereby incorporated by reference in its entirety. This reaction can be carried out in a variety of solvents, such as methylene chloride (CH₂Cl₂), tetrahydrofuran (THF), dimethylformamide (DMF), or other such solvents or in the mixture of such solvents. Compound 15 can be treated with a sulfonyl chloride or coupled with a carboxylic acid to prepare compound (16). Sulfonamide (16) can be prepared by reacting compound 15 with the corresponding sulfonyl chloride in the presence of a base. Any suitable base can be used, for example, triethylamine or N,N-diisopropylethylamine. This reaction can be carried out in a variety of solvents, such as dichloromethane, dichloroethane, or other such solvents or in the mixture of such solvents. Amide (16) can be prepared by reacting compound 15 with the corresponding carboxylic acid in the presence of a base and a coupling agent. Any suitable base can be used, for example, triethylamine or N,N-diisopropylethylamine. Any suitable coupling agent can be used, for example, HATU, HOBT, PyBOP, HBTU, TBTU, HOAT, and PyBroP. The reaction can be carried out in a variety of solvents, for example in methylene chloride (CH₂Cl₂), tetrahydrofuran (THF), dimethylformamide (DMF), or other such solvents or in the mixture of such solvents.

Alternatively, compounds of Formula (IB) can be prepared by reacting compound 15 with an aldehyde in a presence of a reducing agent (Scheme 5). In some instances an aldehyde derivative can be used and the aldehyde is formed during the reaction. Any reducing agent can be used, for example, NaBH₃CN, LiAlH₄, and NaBH(OAc)₃. This reaction can be carried out in a variety of solvents, such as dichloromethane, dichloroethane, methanol, ethanol, THF, diethyl ether, or other such solvents or a mixture of these solvents.

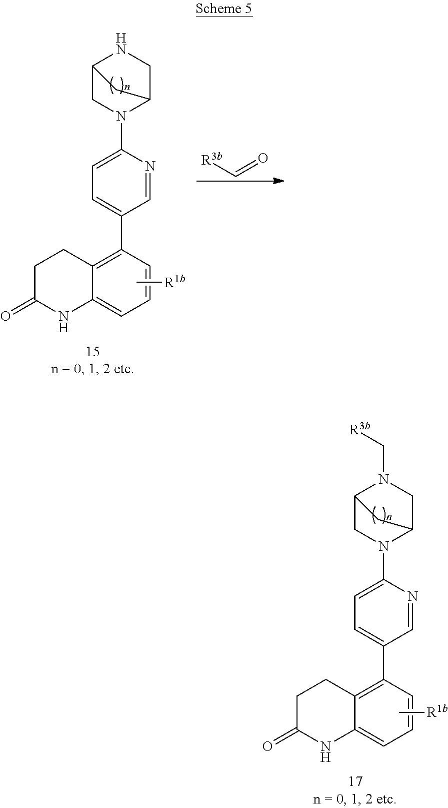

Alternatively, compounds of Formula (IB) can be prepared by reacting acid 20 with an amine in a presence of a base and a coupling agent (Scheme 6).

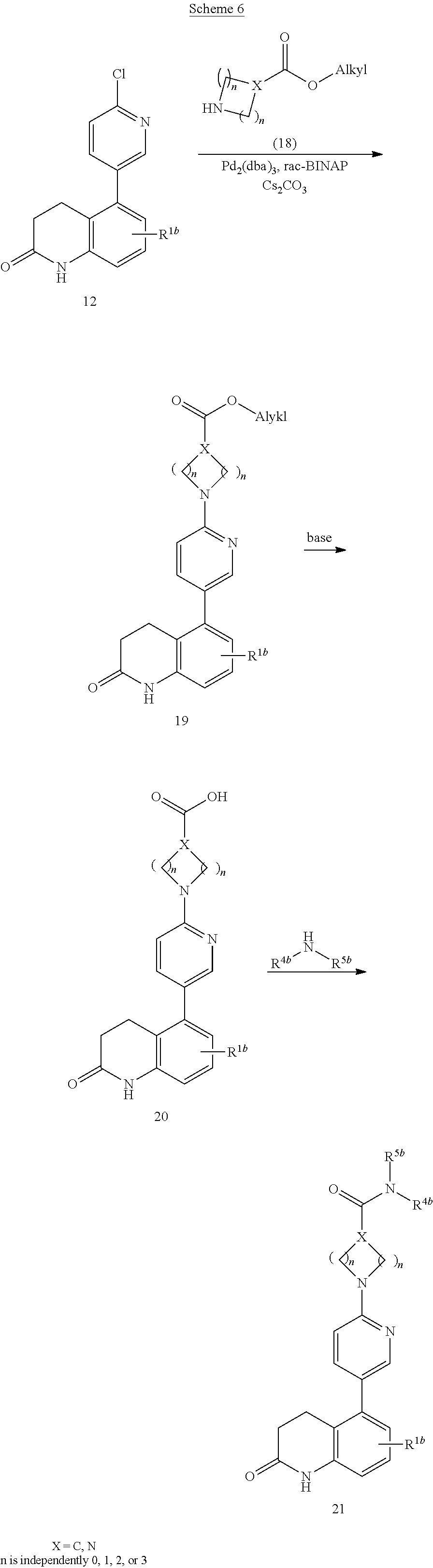

3,4-Dihydroquinolin-2(1H)-one (12) can be reacted with a suitable amine (18) in the presence of rac-2,2′-bis(diphenylphosphino)-1,1′-binaphthyl, tris(dibenzylideneacetone)-dipalladium(0), and cesium carbonate to form compound 19. This reaction can be carried out in a variety of solvents, such as 1,4-dioxane, THF, DMF, or other such solvents or a mixture of these solvents. Compound 19 can be hydrolyzed using a base to form acid 20. Any suitable base can be used, such as LiOH, NaOH, or KOH. This reaction can be carried out in a variety of solvents, such as THF, MeOH, EtOH, iPrOH, or other such solvents or a mixture of these solvents. Compound 20 can be treated with an amine in the presence of a base and a coupling agent to prepare compound 21. Any suitable base can be used, for example, triethylamine or N,N-diisopropylethylamine. Any suitable coupling agent can be used, for example, HATU, HOBT, PyBOP, HBTU, TBTU, HOAT, and PyBroP. The reaction can be carried out in a variety of solvents, for example in methylene chloride (CH₂Cl₂), tetrahydrofuran (THF), dimethylformamide (DMF), or other such solvents or in the mixture of such solvents.

Alternatively, compounds of Formula (IB) can be prepared by reacting compound 15 with an alkylating agent in the presence of a base (Scheme 7). Any suitable base can be used, for example, triethylamine or N,N-diisopropylethylamine. Suitable alkylating agents include, but are not limited to alkyl halides, arylalkyl halides, alkyl triflates, arylalkyl triflates, and other similar reagents. The reaction can be carried out in a variety of solvents, for example in methylene chloride (CH₂Cl₂), tetrahydrofuran (THF), dimethylformamide (DMF), or other such solvents or in the mixture of such solvents.

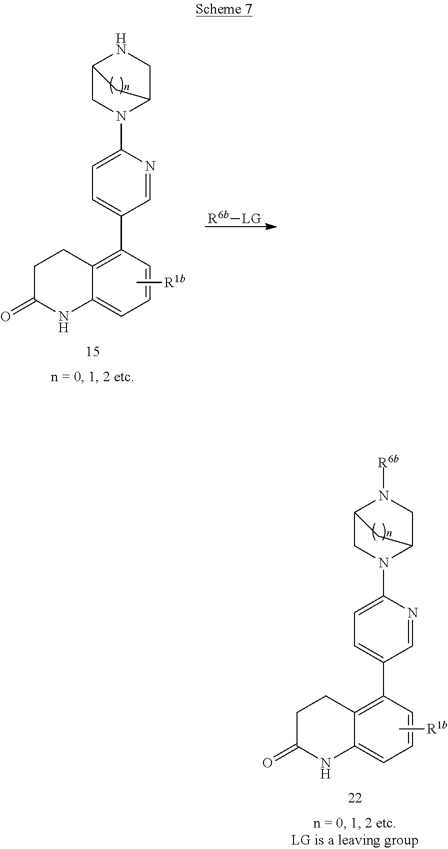

Compounds of Formula (IC) can be prepared according to Scheme 8 below.

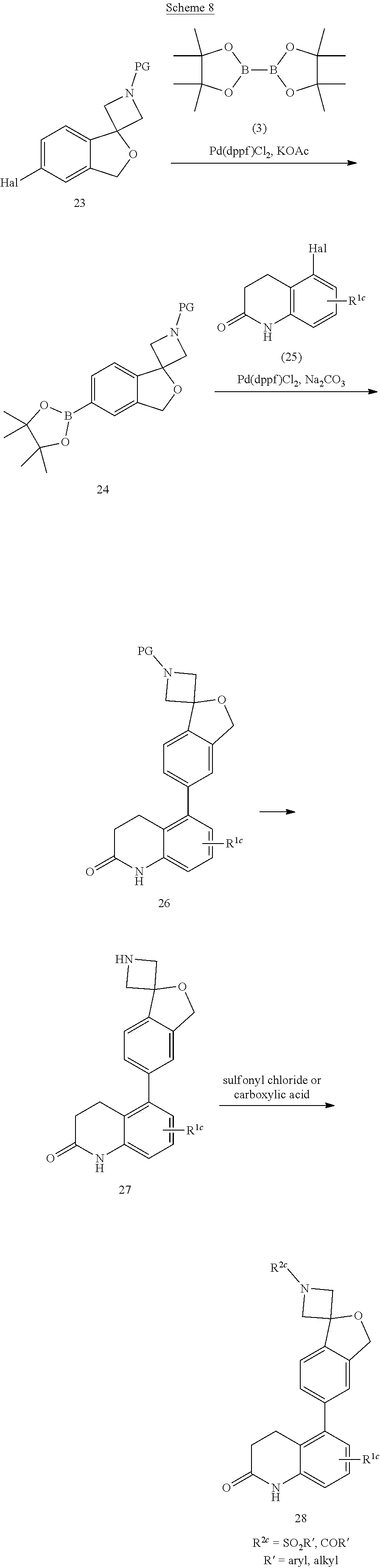

Compound 23 can be reacted with bis(pinacolato)diboron (3) in the presence of dichloro[1,1′-bis(diphenylphosphino)ferrocene]palladium (II) or dichloro[1,1′-bis(diphenylphosphino)ferrocene]palladium (II) dichloromethane adduct and potassium acetate to form compound 24. This reaction can be carried out in a variety of solvents, such as 1,4-dioxane, THF, DMF, or other such solvents or a mixture of these solvents. Compound 24 can be treated with 3,4-dihydroquinolin-2(1H)-one (25) in the presence of dichloro[1,1′-bis(diphenylphosphino)ferrocene]palladium (II) dichloromethane adduct and sodium carbonate to prepare compound 26. This reaction can be carried out in a variety of solvents, such as 1,4-dioxane, water, THF, DMF, or other such solvents or a mixture of these solvents. Compound 26 can be deprotected to form compound 27 using any suitable deprotecting agent. A detailed description of the protecting groups and conditions for their removal is contained in The Peptides, Vol. 3, Gross and Meinenhofer, Eds., Academic Press, New York, 1981, which is hereby incorporated by reference in its entirety. This reaction can be carried out in a variety of solvents, such as methylene chloride (CH₂Cl₂), tetrahydrofuran (THF), dimethylformamide (DMF), or other such solvents or in the mixture of such solvents. Compound 27 can be treated with a sulfonyl chloride or coupled with a carboxylic acid to prepare compound 28. Sulfonamide 28 can be prepared by reacting compound 27 with the corresponding sulfonyl chloride in the presence of a base. Any suitable base can be used, for example, triethylamine or N,N-diisopropylethylamine. This reaction can be carried out in a variety of solvents, such as dichloromethane, dichloroethane, or other such solvents or in the mixture of such solvents. Amide 28 can also be prepared by reacting compound 27 with the corresponding carboxylic acid in the presence of a base and a coupling agent. Any suitable base can be used, for example, triethylamine or N,N-diisopropylethylamine. Any suitable coupling agent can be used, for example, HATU, HOBT, PyBOP, HBTU, TBTU, HOAT, and PyBroP. The reaction can be carried out in a variety of solvents, for example in methylene chloride (CH₂Cl₂), tetrahydrofuran (THF), dimethylformamide (DMF), or other such solvents or in the mixture of such solvents.

Compounds of Formula (ID) can be prepared according to Scheme 9 below.

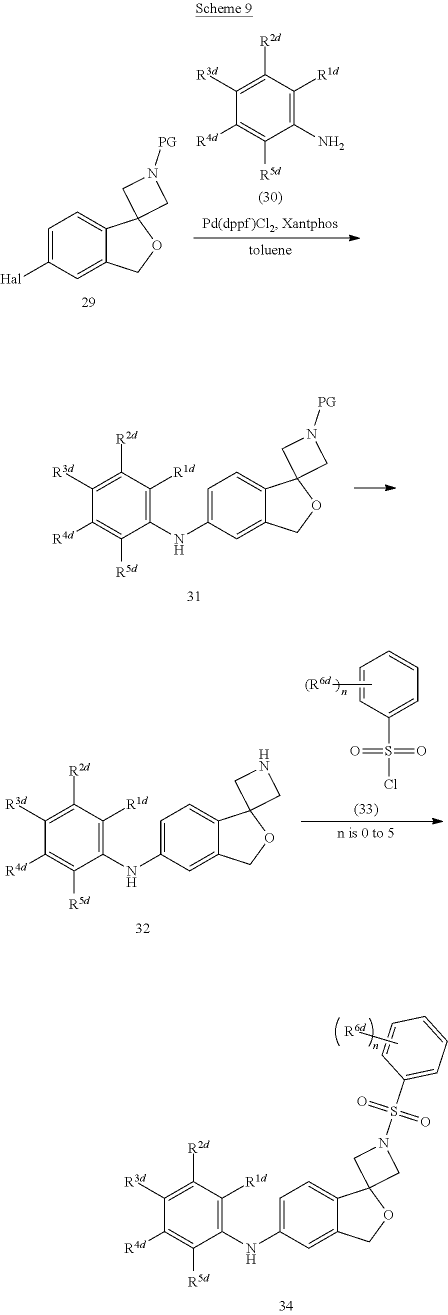

Compound 29 can be reacted with an amine (30) in the presence of 9,9-dimethyl-4,5-bis(diphenylphosphino)xanthene and dichloro[1,1′-bis(diphenylphosphino)ferrocene]palladium (II) or dichloro[1,1′-bis(diphenylphosphino)ferrocene]palladium (II) dichloromethane adduct in toluene to form compound 31. Compound 31 can be deprotected to form compound 32 using any suitable deprotecting agent. A detailed description of the protecting groups and conditions for their removal is contained in The Peptides, Vol. 3, Gross and Meinenhofer, Eds., Academic Press, New York, 1981, which is hereby incorporated by reference in its entirety. This reaction can be carried out in a variety of solvents, such as methylene chloride (CH₂Cl₂), tetrahydrofuran (THF), dimethylformamide (DMF), or other such solvents or in the mixture of such solvents. Compound 32 can be reacted with the corresponding sulfonyl chloride in the presence of a base to form sulfonamide 34. Any suitable base can be used, for example, triethylamine or N,N-diisopropylethylamine. This reaction can be carried out in a variety of solvents, such as dichloromethane, dichloroethane, or other such solvents or in the mixture of such solvents.

Compounds of Formula (IE) can be prepared according to the Scheme 10 below.

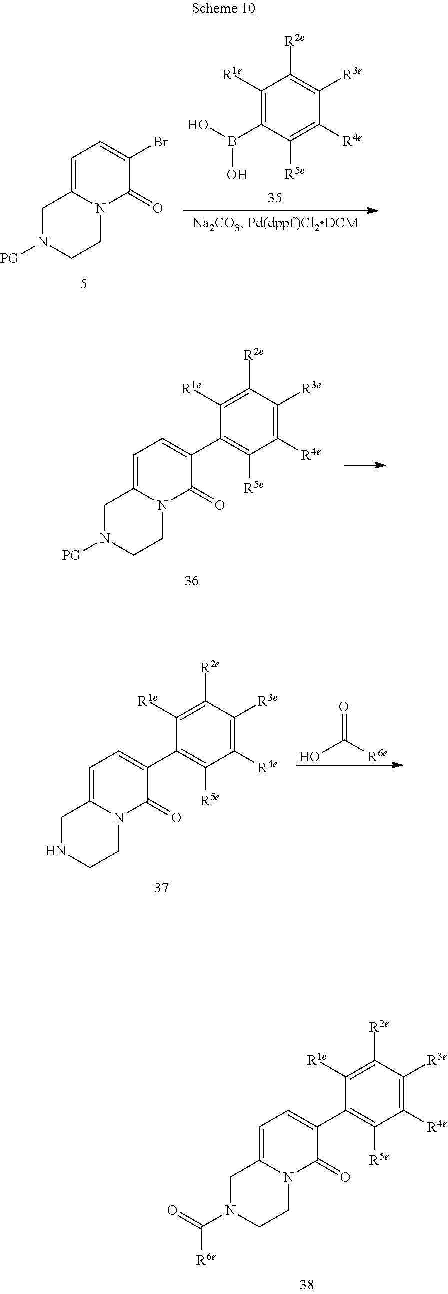

Boronic ester 35 can be reacted with compound 5 in the presence of dichloro[1,1′-bis(diphenylphosphino)ferrocene]palladium (II) dichloromethane adduct and sodium carbonate to form compound 36. This reaction can be carried out in a variety of solvents, such as 1,4-dioxane, water, THF, DMF, or other such solvents or a mixture of these solvents. Compound 36 can be deprotected to form compound 37 using any suitable deprotecting agent. A detailed description of the protecting groups and conditions for their removal is contained in The Peptides, Vol. 3, Gross and Meinenhofer, Eds., Academic Press, New York, 1981, which is hereby incorporated by reference in its entirety. This reaction can be carried out in a variety of solvents, such as methylene chloride (CH₂Cl₂), tetrahydrofuran (THF), dimethylformamide (DMF), or other such solvents or in the mixture of such solvents. Amide 38 can be prepared by reacting compound 37 with the corresponding carboxylic acid in the presence of a base and a coupling agent. Any suitable base can be used, for example, triethylamine or N,N-diisopropylethylamine. Any suitable coupling agent can be used, for example, propylphosphonic anhydride (T₃P), HATU, HOBT, PyBOP, HBTU, TBTU, HOAT, and PyBroP. The reaction can be carried out in a variety of solvents, for example in methylene chloride (CH₂Cl₂), tetrahydrofuran (THF), dimethylformamide (DMF), or other such solvents or in the mixture of such solvents.

Compounds of Formula (IF) can be prepared according to the Schemes 11-13 below.

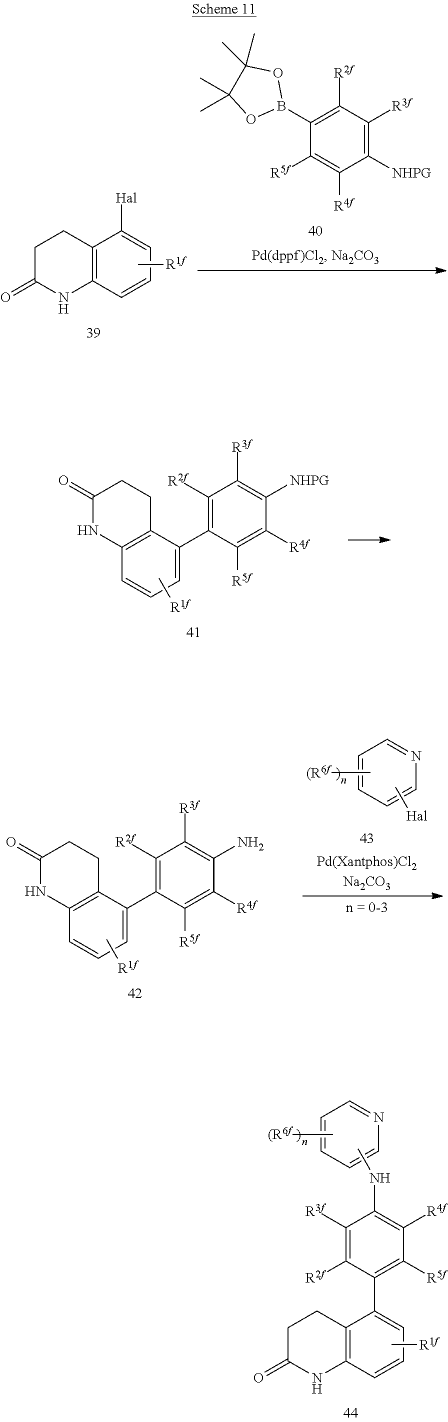

Compound 39 can be reacted with boronic ester 40 to form compound 41 in the presence of dichloro[1,1′-bis(diphenylphosphino)ferrocene]palladium (II) dichloromethane adduct and sodium carbonate. This reaction can be carried out in a variety of solvents, such as 1,4-dioxane, water, DMF, or other such solvents or in the mixture of such solvents. Compound 41 can be deprotected to form compound 42 using any suitable deprotecting agent. A detailed description of the protecting groups and conditions for their removal is contained in The Peptides, Vol. 3, Gross and Meinenhofer, Eds., Academic Press, New York, 1981, which is hereby incorporated by reference in its entirety. This reaction can be carried out in a variety of solvents, such as methylene chloride (CH₂Cl₂), tetrahydrofuran (THF), dimethylformamide (DMF), or other such solvents or in the mixture of such solvents. Compound 42 can be reacted with compound 43 to form compound 44 in the presence of dichloro[9,9-dimethyl-4,5-bis(diphenylphosphino)xanthene]palladium(II) and sodium carbonate. This reaction can be carried out in a variety of solvents, such as 1,4-dioxane, water, DMF, or other such solvents or in the mixture of such solvents.

Alternatively, compounds of Formula (IF) can be prepared according to Scheme 12 below.

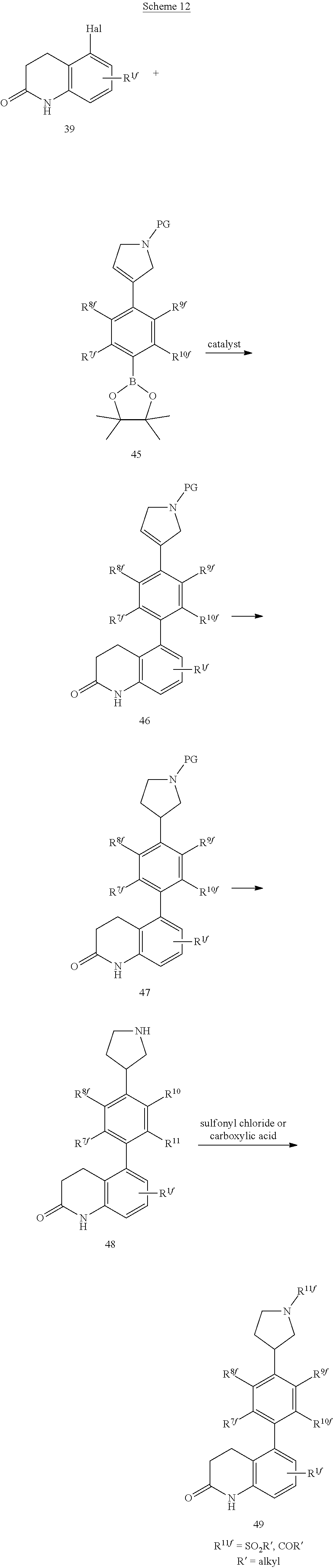

Compound 39 can be reacted with boronic ester 45 to form compound 46 in the presence of a suitable catalyst. Catalysts that can be used include, but are not limited to dichloro[1,1′-bis(diphenylphosphino)ferrocene]palladium (II) dichloromethane adduct and tetrakis(triphenylphosphine)palladium (0). This reaction can be carried out in a variety of solvents, such as 1,4-dioxane, water, DMF, or other such solvents or in the mixture of such solvents. Compound 46 can be reduced to form compound 47 using any suitable reducing agent capable of reducing a double bond. Compound 47 can be deprotected to form compound 48 using any suitable deprotecting agent. A detailed description of the protecting groups and conditions for their removal is contained in The Peptides, Vol. 3, Gross and Meinenhofer, Eds., Academic Press, New York, 1981, which is hereby incorporated by reference in its entirety. This reaction can be carried out in a variety of solvents, such as methylene chloride (CH₂Cl₂), tetrahydrofuran (THF), dimethylformamide (DMF), or other such solvents or in the mixture of such solvents. Compound 48 can be treated with a sulfonyl chloride or coupled with a carboxylic acid to prepare compound 49. Sulfonamide 49 can be prepared by reacting compound 48 with the corresponding sulfonyl chloride in the presence of a base. Any suitable base can be used, for example, triethylamine or N,N-diisopropylethylamine. This reaction can be carried out in a variety of solvents, such as dichloromethane, dichloroethane, or other such solvents or in the mixture of such solvents. Amide 49 can be prepared by reacting compound 48 with the corresponding carboxylic acid in the presence of a base and a coupling agent. Any suitable base can be used, for example, triethylamine or N,N-diisopropylethylamine. Any suitable coupling agent can be used, for example, HATU, HOBT, PyBOP, HBTU, TBTU, HOAT, and PyBroP. The reaction can be carried out in a variety of solvents, for example in methylene chloride (CH₂Cl₂), tetrahydrofuran (THF), dimethylformamide (DMF), or other such solvents or in the mixture of such solvents.

Alternatively, compounds of Formula (IF) can be prepared according to Scheme 13 below.

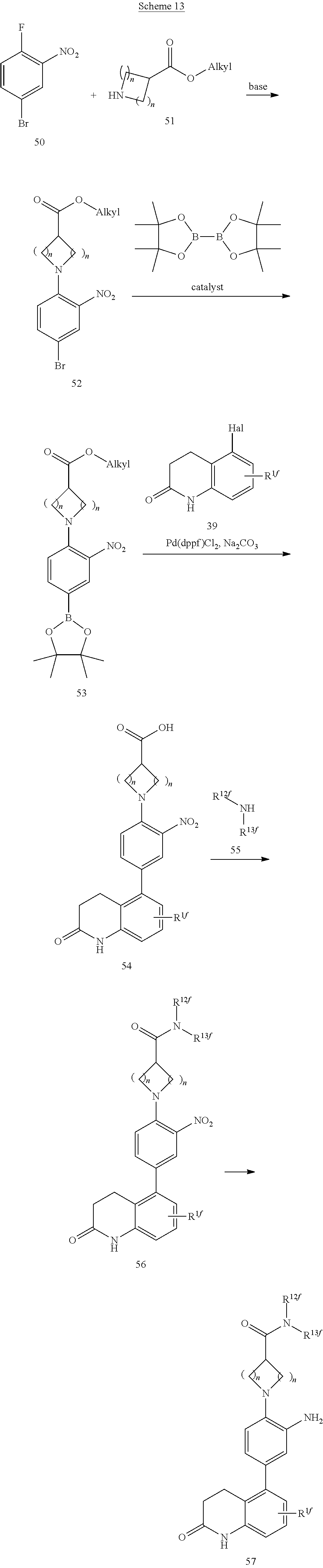

Compound 50 can be reacted with a suitable amine (51) in the presence of a base to form compound 52. Any suitable base can be used, for example, triethylamine or N,N-diisopropylethylamine. This reaction can be carried out in dimethyl sulfoxide (DMSO), tetrahydrofuran (THF), dimethylformamide (DMF), or other such solvents or in the mixture of such solvents. Compound 52 can be reacted with bis(pinacolato)diborane in the presence of a suitable catalyst to form corresponding boronic ester 53. Catalysts that can be used include, but are not limited to dichloro[1,1′-bis(diphenylphosphino)ferrocene]palladium (II) dichloromethane adduct and tetrakis(triphenylphosphine)palladium (0). This reaction can be carried out in a variety of solvents, such as 1,4-dioxane, DMF, or other such solvents or in the mixture of such solvents. Compound 53 can be reacted with compound 39 in the presence of dichloro[1,1′-bis(diphenylphosphino)ferrocene]palladium (II) dichloromethane adduct and sodium carbonate form compound 54. This reaction can be carried out in a variety of solvents, such as 1,4-dioxane, water, DMF, or other such solvents or in the mixture of such solvents. Compound 54 can be reacted with amine 55 and a coupling agent to prepare amide 56. Any suitable coupling agent can be used, for example, HATU, HOBT, PyBOP, HBTU, TBTU, HOAT, and PyBroP. The reaction can be carried out in a variety of solvents, for example in methylene chloride (CH₂Cl₂), tetrahydrofuran (THF), dimethylformamide (DMF), or other such solvents or in the mixture of such solvents. Compound 56 can be reduced to form compound 57. Any suitable methods for reducing an aryl nitro group to an aryl amine can be employed, for example, hydrogenation using Pd/C. This reaction can be carried out in a variety of solvents, such as methanol, ethanol, isopropanol, tetrahydrofuran (THF), methylene chloride (CH₂Cl₂) or other such solvents or in the mixture of such solvents.

While it may be possible for compounds of Formula (I), Formula (IA), Formula (IB), Formula (IC), Formula (ID), Formula (IE), Formula (IF), Formula (IG), Formula (IH), Formula (IJ), Formula (IK), Formula (IL), Formula (IM), Formula (IN), Formula (IO), Formula (IP), Formula (IQ), Formula (IR), Formula (IS), Formula (IA′), Formula (IB′), Formula (IC′), Formula (ID′), Formula (IE′), and Formula (IF′), to be administered as raw chemicals, it will often be preferable to present them as a part of a pharmaceutical composition. Accordingly, another aspect of the present invention is a pharmaceutical composition containing a therapeutically effective amount of the compound of Formula (I) such as any one or more compounds of Formula (IA), Formula (IB), Formula (IC), Formula (ID), Formula (IE), Formula (IF), Formula (IG), Formula (IH), Formula (IJ), Formula (IK), Formula (IL), Formula (IM), Formula (IN), Formula (IO), Formula (TP), Formula (IQ), Formula (IR), Formula (IS), Formula (IA′), Formula (IB′), Formula (IC′), Formula (ID′), Formula (IE′), and/or Formula (IF′), or a pharmaceutically acceptable salt or solvate thereof, and a pharmaceutically acceptable excipient, diluent, or carrier. The excipient, diluent, or carrier must be “acceptable” in the sense of being compatible with the other ingredients of the formulation and not deleterious to the recipient thereof.

Compounds and pharmaceutical compositions suitable for use in the present invention include those wherein the compound can be administered in an effective amount to achieve its intended purpose. Administration of the compound described in more detail below.

Suitable pharmaceutical formulations can be determined by the skilled artisan depending on the route of administration and the desired dosage. See, e.g., Remington's Pharmaceutical Sciences, 1435-712 (18th ed., Mack Publishing Co, Easton, Pennsylvania, 1990). Formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data obtainable through animal or human clinical trials.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable” includes any and all solvents, dispersion media, coatings, antibacterial and antifungal agents, isotonic and absorption delaying agents and the like. The use of such excipients, diluents, and carriers for pharmaceutically active substances is well known in the art. Except insofar as any conventional media or agent is incompatible with the therapeutic compositions, its use in therapeutic compositions is contemplated. Supplementary active ingredients also can be incorporated into the compositions. In exemplary embodiments, the formulation may comprise corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cystine dihydrochloride, L-tryptophan, L-aspartic acid, choline chloride, taurine, m-inositol, ferrous sulfate, ascorbyl palmitate, zinc sulfate, L-carnitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D3 and cyanocobalamin.

The compounds can be present in a pharmaceutical composition as a pharmaceutically acceptable salt. As used herein, “pharmaceutically acceptable salts” include, for example base addition salts and acid addition salts.

Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′-dibenzylethylenediamine, chloroprocaine, choline, diethanolamine, dicyclohexylamine, ethylenediamine, N-methylglucamine, and procaine.

Pharmaceutically acceptable acid addition salts include inorganic or organic acid salts. Examples of suitable acid salts include the hydrochlorides, formates, acetates, citrates, salicylates, nitrates, phosphates. Other suitable pharmaceutically acceptable salts are well known to those skilled in the art and include, for example, formic, acetic, citric, oxalic, tartaric, or mandelic acids, hydrochloric acid, hydrobromic acid, sulfuric acid or phosphoric acid with organic carboxylic, sulfonic, sulfo or phospho acids or N-substituted sulfamic acids, for example acetic acid, trifluoroacetic acid (TFA), propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane 1,2-disulfonic acid, benzenesulfonic acid, 4-methylbenzenesulfonic acid, naphthalene 2-sulfonic acid, naphthalene 1,5-disulfonic acid, 2- or 3-phosphoglycerate, glucose 6-phosphate, N-cyclohexylsulfamic acid (with the formation of cyclamates), or with other acid organic compounds, such as ascorbic acid.

Pharmaceutical compositions containing the compounds disclosed herein can be manufactured in a conventional manner, e.g., by conventional mixing, dissolving, granulating, dragee-making, levigating, emulsifying, encapsulating, entrapping, or lyophilizing processes. Proper formulation is dependent upon the route of administration chosen.

For oral administration, suitable compositions can be formulated readily by combining a compound disclosed herein with pharmaceutically acceptable excipients such as carriers well known in the art. Such excipients and carriers enable the present compounds to be formulated as tablets, pills, dragees, capsules, liquids, gels, syrups, slurries, suspensions and the like, for oral ingestion by a patient to be treated. Pharmaceutical preparations for oral use can be obtained by adding a compound as disclosed herein with a solid excipient, optionally grinding a resulting mixture, and processing the mixture of granules, after adding suitable auxiliaries, if desired, to obtain tablets or dragee cores. Suitable excipients include, for example, fillers and cellulose preparations. If desired, disintegrating agents can be added.

Pharmaceutically acceptable ingredients are well known for the various types of formulation and may be, for example, binders (e.g., natural or synthetic polymers), lubricants, surfactants, sweetening and flavoring agents, coating materials, preservatives, dyes, thickeners, adjuvants, antimicrobial agents, antioxidants, and carriers for the various formulation types.

When a therapeutically effective amount of a compound disclosed herein is administered orally, the composition typically is in the form of a solid (e.g., tablet, capsule, pill, powder, or troche) or a liquid formulation (e.g., aqueous suspension, solution, elixir, or syrup).

When administered in tablet form, the composition can additionally contain a functional solid and/or solid carrier, such as a gelatin or an adjuvant. The tablet, capsule, and powder can contain about 1 to about 95% compound, and preferably from about 15 to about 90% compound.

When administered in liquid or suspension form, a functional liquid and/or a liquid carrier such as water, water, petroleum, petroleum, or oils of animal or oils or plant of animal or origin plant can be added. Origin. The liquid form of the composition can further contain physiological saline solution, sugar alcohol solutions, dextrose or other saccharide solutions, or glycols. When administered in liquid or suspension form, the composition can contain about 0.5 to about 90% by weight of a compound disclosed herein, and preferably about 1 to about 50% of a compound disclosed herein. In one embodiment contemplated, the liquid carrier is non-aqueous or substantially non-aqueous. For administration in liquid form, the composition may be supplied as a rapidly-dissolving solid formulation for dissolution or suspension immediately prior to administration.

When a therapeutically effective amount of a compound disclosed herein is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to a compound disclosed herein, an isotonic vehicle. Such compositions may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations can optionally contain a preservative to prevent the growth of microorganisms.

Injectable compositions can include sterile aqueous solutions, suspensions, or dispersions and sterile powders for the extemporaneous preparation of sterile injectable solutions, suspensions, or dispersions. In all embodiments the form must be sterile and must be fluid to the extent that easy syringability exists. It must be stable under the conditions of manufacture and storage and must resist the contaminating action of microorganisms, such as bacteria and fungi, by optional inclusion of a preservative. The carrier can be a solvent or dispersion medium containing, for example, water, ethanol, polyol (e.g., glycerol, propylene glycol, and liquid polyethylene glycol, and the like), suitable mixtures thereof, and vegetable oils. In one embodiment contemplated, the carrier is non-aqueous or substantially non-aqueous. The proper fluidity can be maintained, for example, by the use of a coating, such as lecithin, by the maintenance of the required particle size of the compound in the embodiment of dispersion and by the use of surfactants. The prevention of the action of microorganisms can be brought about by various antibacterial and antifungal agents, for example, parabens, chlorobutanol, phenol, sorbic acid, thimerosal, and the like. In many embodiments, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the embodiment of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In addition to the formulations described previously, the agents may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

Slow release or sustained release formulations may also be prepared in order to achieve a controlled release of the active compound in contact with the body fluids in the GI tract, and to provide a substantially constant and effective level of the active compound in the blood plasma. For example, release can be controlled by one or more of dissolution, diffusion, and ion-exchange. In addition, the slow release approach may enhance absorption via saturable or limiting pathways within the GI tract. For example, the compound may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

For administration by inhalation, compounds of the present invention are conveniently delivered in the form of an aerosol spray presentation from pressurized packs or a nebulizer, with the use of a suitable propellant. In the embodiment of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

The compounds disclosed herein can be formulated for parenteral administration by injection (e.g., by bolus injection or continuous infusion). Formulations for injection can be presented in unit dosage form (e.g., in ampules or in multidose containers), with an added preservative. The compositions can take such forms as suspensions, solutions, or emulsions in oily or aqueous vehicles, and can contain formulatory agents such as suspending, stabilizing, and/or dispersing agents.

Intraperitoneal or intrathecal administration of the agents of the present invention can also be achieved using infusion pump devices such as those described by Medtronic, Northridge, CA. Such devices allow continuous infusion of desired compounds avoiding multiple injections and multiple manipulations.

Pharmaceutical formulations for parenteral administration include aqueous solutions of the compounds in water-soluble form. Additionally, suspensions of the compounds can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle (e.g., sterile pyrogen-free water) before use.

Compounds disclosed herein also can be formulated in rectal compositions, such as suppositories or retention enemas (e.g., containing conventional suppository bases). In addition to the formulations described previously, the compounds also can be formulated as a depot preparation. Such long-acting formulations can be administered by implantation (e.g., subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In particular, a compound disclosed herein can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A compound also can be injected parenterally, for example, intravenously, intramuscularly, subcutaneously, intracoronarily, intradermally, intraperitoneally, or intratumorally. For parenteral administration, the compound is best used in the form of a sterile aqueous solution which can contain other substances, for example, salts, or sugar alcohols, such as mannitol, or glucose, to make the solution isotonic with blood.

For veterinary use, a compound disclosed herein is administered as a suitably acceptable formulation in accordance with normal veterinary practice. The veterinarian can readily determine the dosing regimen and route of administration that is most appropriate for a particular animal.

In some embodiments, the pharmaceutical composition contains a compound as disclosed herein either alone or in combination with a second agent traditionally used for the treatment of cancer. Alternatively, the two agents may be packaged into a kit. Specifically, the present invention provides a kit for use in the treatment of cancer comprising a packaged set of medicaments that include the compound disclosed herein as well as buffers and other components for preparing deliverable forms of said medicaments, and/or devices for delivering such medicaments, and/or any agents that are used in combination therapy with the compound disclosed herein, and/or instructions for the treatment of cancer packaged with the medicaments. The instructions may be fixed in any tangible medium, such as printed paper, or a computer readable magnetic or optical medium, or instructions to reference a remote computer data source such as a world wide web page accessible via the internet.

As used herein, a “therapeutically effective amount” means an amount effective to treat or to prevent development of the cancer being treated, or to alleviate the existing symptoms of cancer in the subject being treated. Determination of the effective amounts is well within the capability of those skilled in the art, especially in light of the detailed disclosure provided herein. Generally, a “therapeutically effective dose” refers to that amount of the compound that results in achieving the desired effect, which can be assessed with respect to either: (i) reduction of Artemis endonuclease activity, or (ii) shrinkage of tumor size for solid tumors or reduction of the tumor load for blood cancers. For example, in one preferred embodiment, a therapeutically effective amount of a compound disclosed herein decreases Artemis endonuclease activity by at least 5%, compared to control, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

The amount of compound administered can be dependent on the subject being treated, on the subject's age, health, sex, and weight, the kind of concurrent treatment (if any), severity (or stage) of cancer being treated, the nature of the effect desired, the manner and frequency of treatment, and the judgment of the prescribing physician. The frequency of dosing also can be dependent on pharmacodynamic effects on arterial oxygen pressures. While individual needs vary, determination of optimal ranges of effective amounts of the compound is within the skill of the art. Such doses may be administered in a single dose or it may be divided into multiple doses.

The percentage of active ingredient in the compositions of the present invention may be varied. Obviously, several unit dosage forms may be administered at about the same time. The dose employed will be determined by the physician, and depends upon the desired therapeutic effect, the route of administration and the duration of the treatment, and the condition of the patient. In the adult, the doses are generally from about 0.01 to about 100 mg/kg body weight, preferably about 0.01 to about 10 mg/kg body weight per day by inhalation, from about 0.01 to about 100 mg/kg body weight, preferably 0.1 to 70 mg/kg body weight, more especially 0.1 to 10 mg/kg body weight per day by oral administration, and from about 0.01 to about 50 mg/kg body weight, preferably 0.01 to 10 mg/kg body weight per day by intravenous administration. In each particular case, the doses will be determined in accordance with the factors distinctive to the subject to be treated, such as age, weight, general state of health, and other characteristics which can influence the efficacy of the medicinal product. Effective doses of the compositions of the present invention for the treatment of cancer vary depending upon many different factors, including type and stage of cancer, means of administration, target site, physiological state of the patient, other medications or therapies administered, and physical state of the patient relative to other medical complications. Treatment dosages need to be titrated to optimize safety and efficacy.

The products according to the present invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient.

The compounds and pharmaceutical compositions of the present invention are particularly useful in several methods.

According to one embodiment, the compounds and pharmaceutical compositions of the present invention are useful in a method of inhibiting an Artemis nuclease activity. This method includes the step of contacting an Artemis nuclease with a compound described in the present disclosure under conditions effective to inhibit the Artemis nuclease activity. This method can be carried out in vitro in a cell free assay, in an isolated cell such as a primary isolate or a cell line (e.g., cancer cell line), or in vivo upon administration of the compound or composition to an individual as discussed herein.

Screening assays for detecting inhibitors of Artemis include a high throughput screening assay utilizing a fluorescence resonance energy transfer (FRET)-based DNA cleavage assay. Briefly, a ssDNA substrate containing a 5′-fluorophore-conjugated end and a 3′ BHQ-1 (black hole quencher)-conjugated end can be used. As the fluorophore and BHQ-1 are located proximal to one another, prior to endonucleolytic incision, the intact substrate does not fluoresce. Following endonucleolytic incision by SNM1C/Artemis, uncoupling of fluorophore from BHQ-1 causes an increase in fluorescence. See Yosaatmadja et al., “Structural and Mechanistic Insights Into the Artemis Endonuclease and Strategies for its Inhibition,” Nucleic Acids Research 49(16):9310-9326 (2021); Ogana et al., “Artemis Inhibition as a Therapeutic Strategy for Acute Lymphoblastic Leukemia,” Front Cell Dev Biol. 11:1134121 (2023), each of which is hereby incorporated by reference in its entirety.

According to another embodiment, the compounds and pharmaceutical compositions of the present invention are useful in a method of ameliorating a condition caused by the activity of Artemis nuclease in a subject. This method includes the step of administering to a subject a compound or a pharmaceutical composition as described in the present disclosure. As used herein, ameliorating the condition includes both reducing the severity of symptoms as well as eliminating symptoms of the condition, and reducing the rate of symptom deterioration, that is the rate at which the severity of symptoms progress over time.

According to a further embodiment, the compounds and pharmaceutical compositions of the present invention are useful in a method of treating cancer or neoplastic condition in a subject. This method comprises administering to the subject in need thereof a compound or a pharmaceutical composition as described in the present disclosure.

As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and blood-borne cancers. The term “cancer” encompasses diseases of skin, tissues, organs, bone, cartilage, blood, and vessels. The term “cancer” further encompasses primary and metastatic cancers.

Non-limiting examples of solid tumors that can be treated with the disclosed Artemis inhibitors include pancreatic cancer; bladder cancer; colorectal cancer; breast cancer, including metastatic breast cancer; prostate cancer, including androgen-dependent and androgen-independent prostate cancer; renal cancer including, e.g., metastatic renal cell carcinoma; hepatocellular cancer; lung cancer including, e.g., non-small cell lung cancer (NSCLC), bronchioloalveolar carcinoma (BAC), and adenocarcinoma of the lung; ovarian cancer including, e.g., progressive epithelial or primary peritoneal cancer; cervical cancer; gastric cancer; esophageal cancer; head and neck cancer including, e.g., squamous cell carcinoma of the head and neck; melanoma; neuroendocrine cancer including metastatic neuroendocrine tumors; brain tumors including, e.g., glioma, anaplastic oligodendroglioma, adult glioblastoma multiforme, and adult anaplastic astrocytoma; bone cancer; and soft tissue sarcoma.

Non-limiting examples of blood-borne cancers that can be treated with the disclosed Artemis inhibitors include lymphomas (e.g., Hodgkins and non-Hodgkins lymphomas, including both B-cell and T-cell lymphomas) and leukemias (e.g., hairy cell leukemia, acute myeloid leukemia, acute lymphoblastic leukemia, acute promyelocytic leukemia, chronic lymphocytic leukemia, chronic myelogenous leukemia, myeloproliferative neoplasm, and systemic mastocytosis).

In some embodiments, the compound is administered in combination with radiotherapy or another anti-cancer agent. Exemplary anticancer agents include, without limitation, alkylating agents that cause direct DNA damage, antibiotics or antimetabolites that disrupt DNA synthesis of RNA transcription, camptothecin analogs, antibiotics and antimetabolites that disrupt DNA-related proteins, deacetylase inhibitors, PARP inhibitors, HIF2 inhibitors, chaperone inhibitors, cyclin inhibitors, vinka alkaloids and taxanes, pathway inhibitors selected from TKIs of BRAF, BTK, KRAS, MEK, mTOR, NTRK, and/or PI3K, antiestrogens, androgen receptor antagonists, immunoconjugates and monoclonal antibodies that target overexpressed receptors, VEGF and VEGFR inhibitors, checkpoint inhibitors, CAR-T or TCR therapies, BiTE or immTAC therapies, thalidomide derivatives, BCG and imiquimod immunomodulators, aromatase inhibitors, GnRH analogs and antagonists, and 17-alpha-hydroxylase inhibitors.

According to a further embodiment, the compounds and pharmaceutical compositions of the present invention are useful in a method of enhancing cancer therapy in a subject. This method includes the step of delivering a compound described in the present disclosure to cancerous cells in the subject in an amount effective to inhibit Artemis nuclease, and treating the subject with a second cancer therapy. The second cancer therapy can be any known cancer therapy such as those described above.

According to yet another embodiment, the compounds and pharmaceutical compositions of the present invention are useful in a method for the treatment of a subject having severe combined immunodeficiency (SCID). This method includes the step of administering to the subject a compound as described in the present disclosure. By treatment of SCID, it is contemplated that the degree of immunodeficiency can be lessened, such that an individual undergoing treatment will be less susceptible to infection by common pathogens. This treatment can be used, for example, up until a successful stem cell transplant is implemented.

According to another embodiment, the compounds and pharmaceutical compositions of the present invention are useful in a method of biotherapy in a subject. This method includes the step of administering to the subject a compound described in the present disclosure.

The above disclosure is general. A more specific description is provided below in the following examples. The examples are described solely for the purpose of illustration and are not intended to limit the scope of the present application. Changes in form and substitution of equivalents are contemplated as circumstances suggest or render expedient. Although specific terms have been employed herein, such terms are intended in a descriptive sense and not for purposes of limitation.

### EXAMPLES

The following Examples are presented to illustrate various aspects of the present disclosure, but are not intended to limit the scope of the claimed application.

### Materials and Instruments for Examples 1-4

Unless otherwise noted, reagents and solvents were used as received from commercial suppliers. All non-aqueous reactions were carried out under an atmosphere of dry nitrogen (unless otherwise noted). Flash column chromatography was carried out using a Teledyne Isco CombiFlash Companion Unit with RediSep® Rf silica gel columns. If needed, products were purified by reverse phase chromatography, using a Teledyne Isco CombiFlash Companion Unit with RediSep® Gold C18 reverse phase column. Nuclear magnetic resonance spectra were obtained on a Bruker AVANCE 500 spectrometer at 500 MHz or on a Bruker AVANCE 300 spectrometer at 300 MHz. Spectra are given in ppm (δ) and coupling constants, J values, are reported in hertz (Hz). Tetramethylsilane was used as an internal standard for proton nuclear magnetic resonance. Mass spectra and LCMS analyses were obtained using a Waters Acquity SQD (ESI, UPLC-MS) or Acquity SQD2 instrument.

### Example 1—Synthesis of 5-(Pyridin-3-yl)-3,4-dihydroquinolin-2(1H)-one Derivatives

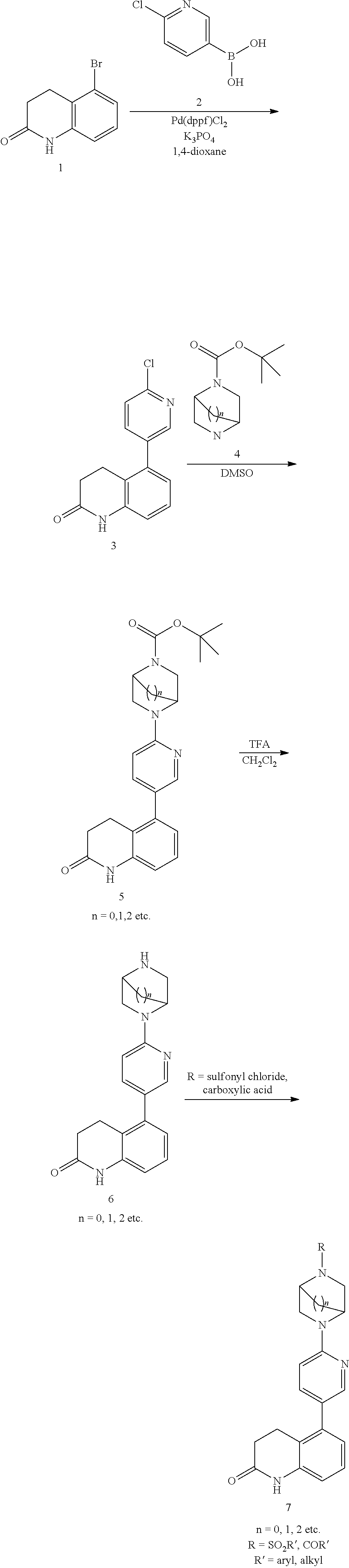

### General Protocol 1: Sulfonamide Formation

To a solution the appropriate amine (1 eq.) in dichloromethane (0.05-0.2 M) was added, triethylamine or N,N-diisopropylethylamine (3-5 eq.), followed by the appropriate sulfonyl chloride (1.2-1.5 eq.) at room temperature. Reactions were monitored by LC-MS and after completion, the reaction mixture was diluted with dichloromethane and washed with saturated aqueous NaHCO₃. The aqueous layer was extracted with dichloromethane and the combined organic layers were dried over MgSO₄ or Na₂SO₄, filtered, and concentrated. The product was purified by normal phase silica gel chromatography and/or reverse phase chromatography, if necessary.

General Protocol 2: Amide Coupling with HATU

To a solution the appropriate amine (1 eq.) in DMF or THF (0.1-0.2 M) was added the appropriate carboxylic acid (1-1.5 eq.), N,N-diisopropylethylamine or triethylamine (2-3 eq.), and HATU (1-1.5 eq.) at room temperature. Reactions were monitored by LC-MS and after completion, the reaction mixture was diluted with EtOAc and washed with water. The aqueous layer was extracted with EtOAc and the combined organic layers were dried over MgSO₄ or Na₂SO₄, filtered, and concentrated. The product was purified by normal phase silica gel chromatography and/or reverse phase chromatography, if necessary.

### Synthesis of Tert-Butyl 5-(5-(2-oxo-1,2,3,4-tetrahydroquinolin-5-yl)pyridin-2-yl)-2,5-diazabicyclo[2.2.2]octane-2-carboxylate

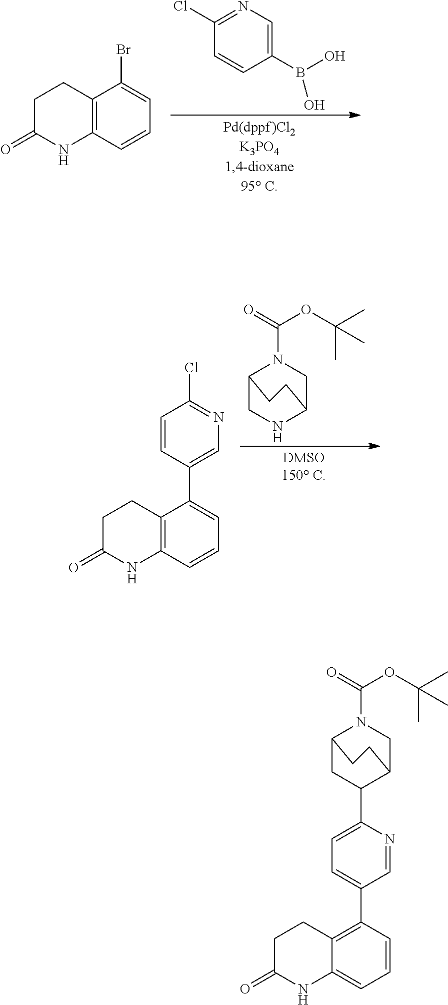

### Synthesis of 5-(6-Chloropyridin-3-yl)-3,4-dihydroquinolin-2(1H)-one

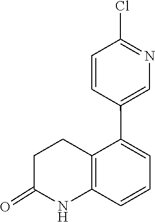 [SMILES: O=C1CCc2c(cccc2-c2ccc(Cl)nc2)N1]

A solution of 5-bromo-3,4-dihydroquinolin-2(1H)-one (4.88 g, 21.6 mmol), (6-chloropyridin-3-yl)boronic acid (4.08 g, 25.9 mmol), potassium phosphate hydrate (32.4 mL, 64.8 mmol, 2M) in 1,4-dioxane (98 mL) was sparged with argon gas for 15 minutes. Dichloro[1,1′-bis(diphenylphosphino)ferrocene]palladium(II) acetone adduct (1.71 g, 2.16 mmol) was added and the reaction mixture was stirred at 95° C. overnight. The reaction was cooled to ambient temperature, diluted with ethyl acetate, filtered through Celite, washed with ethyl acetate, and concentrated. The crude residue was purified by normal phase column chromatography (silica gel, 0-40% ethyl acetate/heptane) to afford 5-(6-chloropyridin-3-yl)-3,4-dihydroquinolin-2(1H)-one (2.0 g, 36% yield) as a white solid. ¹H NMR (300 MHz, DMSO-d₆) δ 10.23 (s, 1H), 8.42 (d, J=2.4 Hz, 1H), 7.88 (dd, J=8.2, 2.5 Hz, 1H), 7.61 (d, J=8.4 Hz, 1H), 7.26 (dd, J=7.8, 7.8 Hz, 1H), 6.97-6.93 (m, 2H), 2.79 (t, J=7.5 Hz, 2H), 2.38 (t, J=7.5 Hz, 2H). ESI MS m/z 259 [C₁₄H₁₁ClN₂O+H]⁺.

### Synthesis of Tert-Butyl 5-(5-(2-axo-1,2,3,4-tetrahydroquinolin-5-yl)pyridin-2-yl)-2,5-diazabicyclo[2.2.2]octane-2-carboxylate

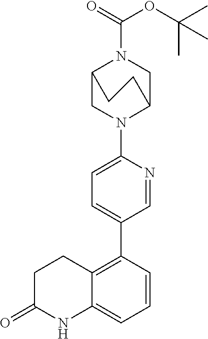 [SMILES: CC(C)(C)OC(=O)N1CC2CCC1CN2c1ccc(-c2cccc3c2CCC(=O)N3)cn1]

To a microwave vial was added 5-(6-chloropyridin-3-yl)-3,4-dihydroquinolin-2(1H)-one (472 mg, 1.82 mmol) and tert-butyl 2,5-diazabicyclo[2.2.2]octane-2-carboxylate (581 mg, 2.74 mmol) in DMSO (1.71 mL). The vial was sealed and reaction mixture was stirred at 150° C. overnight. After this time, the reaction mixture was cooled to ambient temperature and the residue was purified by normal phase column chromatography (silica gel, 0-20% methanol/methylene chloride). Semi-pure fractions were collected and concentrated. The material was dissolved in methanol and purified by C18 reverse phase separation (30 g, 5-100% water/acetonitrile). Clean fractions were collected and lyophilized (acetonitrile and water) to afford tert-butyl 5-(5-(2-oxo-1,2,3,4-tetrahydroquinolin-5-yl)pyridin-2-yl)-2,5-diazabicyclo[2.2.2]octane-2-carboxylate (3.8 mg, 0.5%) as an off-white solid. ¹H NMR (300 MHz, DMSO-d₆) ¹H NMR (300 MHz, CDCl3) δ 10.14 (s, 1H), 8.06 (d, J=2.1 Hz, 1H), 7.54 (dd, J=8.7, 2.4 Hz, 1H), 7.19 (dd, J=7.8, 7.8 Hz, 1H), 6.88-6.85 (m, 2H), 6.59 (d, J=9.0 Hz, 1H), 4.89-4.81 (m, 1H), 4.28-4.22 (m, 1H), 3.61-3.43 (m, 4H), 2.82 (t, J=7.5 Hz, 2H), 2.36 (t, J=7.3 Hz, 2H), 1.96-1.75 (m, 4H), 1.42 (s, 9H). ESI MS m/z 435 [C₂₅H₃₀N₄O₃+H]⁺.

### Synthesis of 4-((5-(5-(2-Oxo-1,2,3,4-tetrahydroquinolin-5-yl)pyridin-2-y)-2,5-diazabicyclo[2.2.2]octan-2-yl)sulfonyl)benzoic Acid

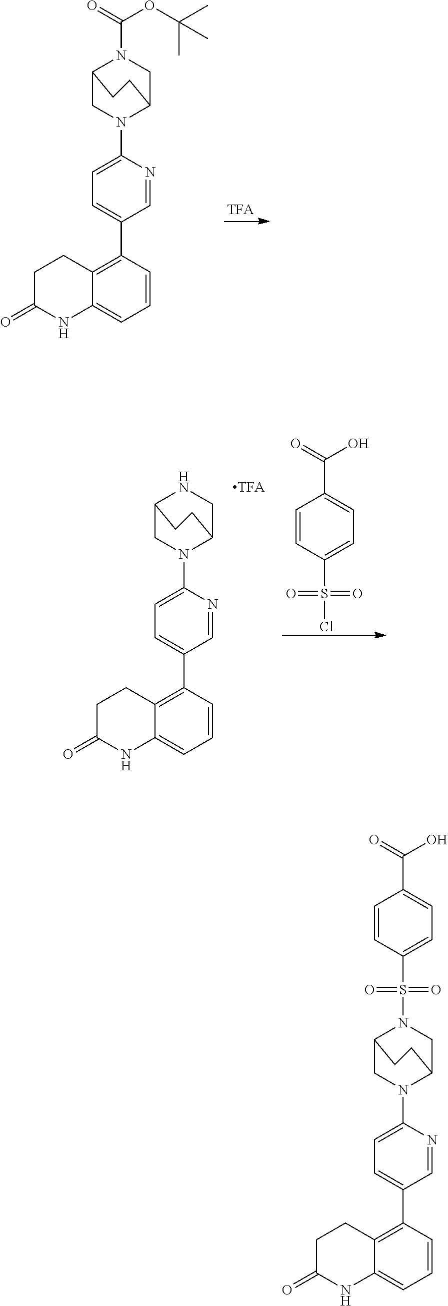 [SMILES: CC(C)(C)OC(=O)N1CC2CCC1CN2c1ccc(-c2cccc3c2CCC(=O)N3)cn1 ; O=C1CCc2c(cccc2-c2ccc(N3CC4CCC3CN4)nc2)N1 ; O=C(O)c1ccc(S(=O)(=O)Cl)cc1 ; O=C1CCc2c(cccc2-c2ccc(N3CC4CCC3CN4S(=O)(=O)c3ccc(C(=O)O)cc3)nc2)N1]

### Synthesis of 5-(6-(2,5-Diazabicyclo[2.2.2]octan-2-yl)pyridin-3-yl)-3,4-dihydroquinolin-2(1H)-one 2,2,2-Trifluoroacetate

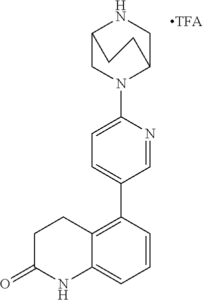 [SMILES: O=C1CCc2c(cccc2-c2ccc(N3CC4CCC3CN4)nc2)N1]

To a solution of tert-butyl 5-(5-(2-oxo-1,2,3,4-tetrahydroquinolin-5-yl)pyridin-2-yl)-2,5-diazabicyclo[2.2.2]octane-2-carboxylate (440 mg, 1.01 mmol) dissolved in methylene chloride (3 mL) was added 2,2,2-trifluoroacetic acid (0.780 ml, 10.1 mmol) at room temperature. The reaction was stirred for 1 hour and then concentrated under reduced pressure to afford 5-(6-(2,5-diazabicyclo[2.2.2]octan-2-yl)pyridin-3-yl)-3,4-dihydroquinolin-2(1H)-one 2,2,2-trifluoroacetate which was used without further purification (339 mg, >99%). ESI MS m/z 335 [C₂₀H₂₂N₄O+H]⁺.

### Synthesis of 4-((5-(5-(2-Oxo-1,2,3,4-tetrahydroquinolin-5-yl)pyridin-2-yl)-2,5-diazabicyclo[2.2.2]octan-2-yl)sulfonyl)benzoic Acid

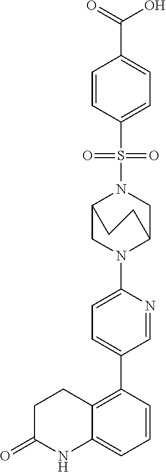 [SMILES: O=C1CCc2c(cccc2-c2ccc(N3CC4CCC3CN4S(=O)(=O)c3ccc(C(=O)O)cc3)nc2)N1]

4-((5-(5-(2-Oxo-1,2,3,4-tetrahydroquinolin-5-yl)pyridin-2-yl)-2,5-diazabicyclo[2.2.2]octan-2-yl)sulfonyl)benzoic acid was prepared according to General Protocol 1 using 5-(6-(2,5-diazabicyclo[2.2.2]octan-2-yl)pyridin-3-yl)-3,4-dihydroquinolin-2(1H)-one 2,2,2-trifluoroacetate and 4-(chlorosulfonyl)benzoic acid to afford 4-((5-(5-(2-oxo-1,2,3,4-tetrahydroquinolin-5-yl)pyridin-2-yl)-2,5-diazabicyclo[2.2.2]octan-2-yl)sulfonyl)benzoic acid (8.0 mg, 23%) as an off-white solid. ¹H NMR (500 MHz, DMSO-d₆) δ 10.10 (s, 1H), 8.04 (d, J=8.5 Hz, 2H), 8.00 (d, J=2.5 Hz, 1H), 7.87 (d, J=8.5 Hz, 2H), 7.46 (dd, J=8.5, 2.5 Hz, 1H), 7.17 (t, J=7.7 Hz, 1H), 6.85 (dd, J=7.8, 2.3 Hz, 2H), 6.43 (d, J=8.5 Hz, 1H), 4.82 (s, 1H), 4.18 (s, 1H), 3.54-3.47 (m, 2H), 3.37-3.35 (m, 2H), 2.79 (t, J=7.8 Hz, 2H), 2.35 (t, J=7.8 Hz, 2H), 1.83-1.69 (m, 4H). ESI MS m/z 519 [C₂₇H₂₆N₄O₅S+H]⁺.

### Synthesis of 5-(6-(4-(Cyclopropylmethyl)piperazin-1-yl)pyridin-3-yl)-3,4-dihydroquinolin-2(1H)-one

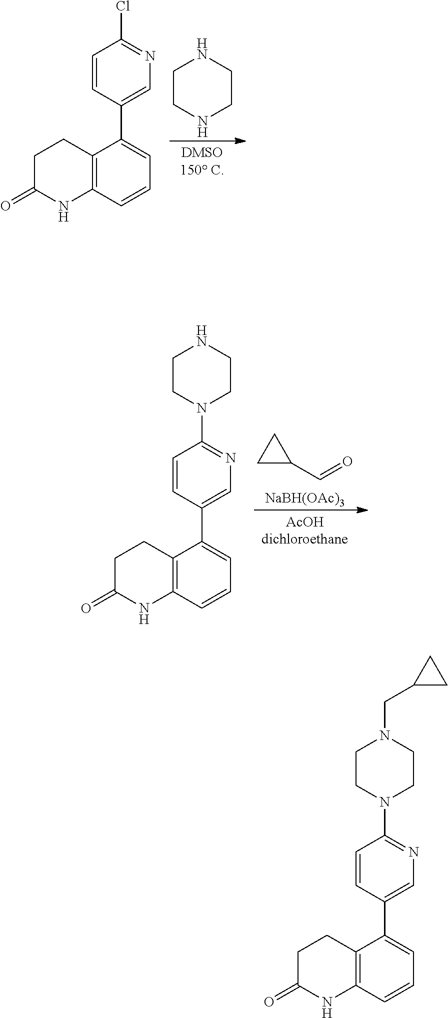

### Synthesis of 5-(6-(Piperazin-1-yl)pyridin-3-yl)-3,4-dihydroquinolin-2(1H)-one

In a microwave vial, 5-(6-chloropyridin-3-yl)-3,4-dihydroquinolin-2(1H)-one (300 mg, 1.16 mmol) and piperazine (200 mg, 2.32 mmol) were dissolved in DMSO (0.5 mL). The vial was sealed and reaction mixture was heated to 150° C. and stirred for 1 hour. The reaction mixture was cooled to room temperature and then diluted with acetonitrile and methylene chloride, sonicated, and the solid was collected by suction filtration and dried under high vacuum to afford 5-(6-(piperazin-1-yl)pyridin-3-yl)-3,4-dihydroquinolin-2(1H)-one (170 mg, 43%) as a white solid. ESI MS m/z 309 [C₁₈H₂₀N₄O+H]⁺.

### Synthesis of 5-(6-(4-(Cyclopropylmethyl)piperazin-1-yl)pyridin-3-yl)-3,4-dihydroquinolin-2(1H)-one

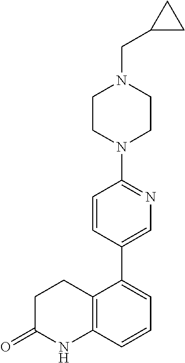 [SMILES: O=C1CCc2c(cccc2-c2ccc(N3CCN(CC4CC4)CC3)nc2)N1]

To a solution of 5-(6-(piperazin-1-yl)pyridin-3-yl)-3,4-dihydroquinolin-2(1H)-one (50 mg, 0.16 mmol) and cyclopropanecarboxaldehyde (0.013 ml, 0.18 mmol) in dichloroethane (3 mL) and acetic acid (0.93 μl, 0.016 mmol) was added sodium triacetoxyborohydride (52 mg, 0.24 mmol) at room temperature and stirred for 48 hours. The solvent was removed under reduced pressure and the residue was partitioned with 1 N NaOH and ethyl acetate. The layers were separated and the aqueous layer was extracted with ethyl acetate (2×) and the combined organic extracts were washed with brine, dried over sodium sulfate, filtered, and concentrated. The reaction mixture was purified by normal phase column chromatography (silica gel, 0-25% MeOH/methylene chloride) to afford 5-(6-(4-(cyclopropylmethyl)piperazin-1-yl)pyridin-3-yl)-3,4-dihydroquinolin-2(1H)-one (40 mg, 68%) as a white solid. ¹H NMR (300 MHz, CDCl₃) δ 8.16 (d, J=2.1 Hz, 1H), 7.82 (s, 1H), 7.46 (dd, J=8.7, 2.4 Hz, 1H), 7.23-7.21 (m, 1H), 6.97 (dd, J=7.7, 1.0 Hz, 1H), 6.77-6.71 (m, 2H), 3.67 (s, 4H), 2.95 (t, J=7.3 Hz, 2H), 2.70 (s, 4H), 2.54 (t, J=7.5 Hz, 2H), 2.31 (s, 2H), 0.95 (s, 1H), 0.59-0.57 (m, 2H), 0.17-0.16 (m, 2H). ESI MS m/z 363 [C₂₂H₂₆N₄O+H]⁺.

### Synthesis of (1s,4s)-4-(4-(5-(2-Oxo-1,2,3,4-tetrahydroquinolin-5-yl)pyridin-2-yl)piperazine-1-carbonyl)clohexane-1-carboxylic Acid

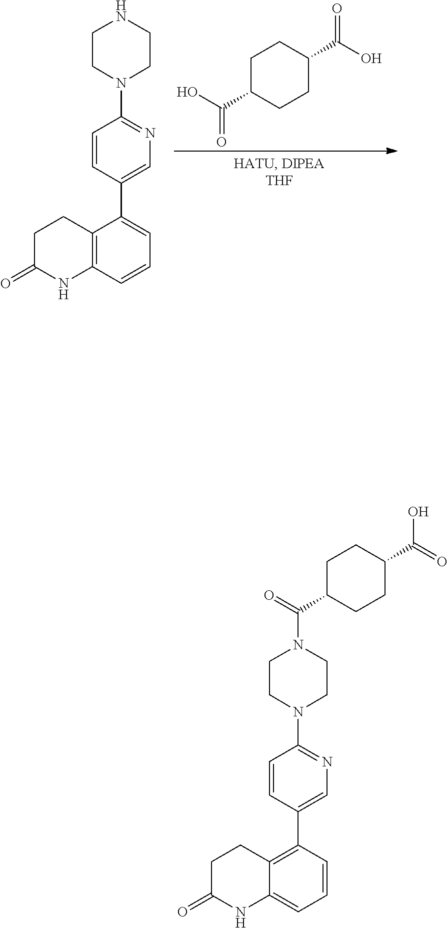 [SMILES: O=C1CCc2c(cccc2-c2ccc(N3CCNCC3)nc2)N1 ; O=C(O)[C@H]1CC[C@@H](C(=O)O)CC1 ; O=C1CCc2c(cccc2-c2ccc(N3CCN(C(=O)[C@H]4CC[C@@H](C(=O)O)CC4)CC3)nc2)N1]

(1s,4s)-4-(4-(5-(2-Oxo-1,2,3,4-tetrahydroquinolin-5-yl)pyridin-2-yl)piperazine-1-carbonyl)cyclohexane-1-carboxylic acid was prepared according to General Protocol 2 using 5-(6-(piperazin-1-yl)pyridin-3-yl)-3,4-dihydroquinolin-2(1H)-one and cis-1,4-cyclohexanedicarboxylic acid to afford (1s,4s)-4-(4-(5-(2-oxo-1,2,3,4-tetrahydroquinolin-5-yl)pyridin-2-yl)piperazine-1-carbonyl)cyclohexane-1-carboxylic acid (170 mg, 43%) as an off-white solid. ¹H NMR (500 MHz, DMSO-d₆) δ 12.10 (s, 1H), 10.13 (s, 1H), 8.12 (d, J=2.5 Hz, 1H), 7.58 (dd, J=8.7, 2.2 Hz, 1H), 7.20 (dd, J=7.8, 7.8 Hz, 1H), 6.92-9.87 (m, 3H), 3.61-3.51 (m, 8H), 2.82 (t, J=7.5 Hz, 2H), 2.70 (s, 1H), 2.38-2.35 (m, 3H), 2.01-2.00 (m, 2H), 1.58-1.53 (m, 6H). ESI MS m/z 463 [C₂₆H₃₀N₄O₄+H]⁺.

### Synthesis of N,N-Dimethyl-1-(5-(2-oxo-1,2,3,4-tetrahydroquinolin-5-yl)pyridin-2-yl)azetidine-3-carboxamide

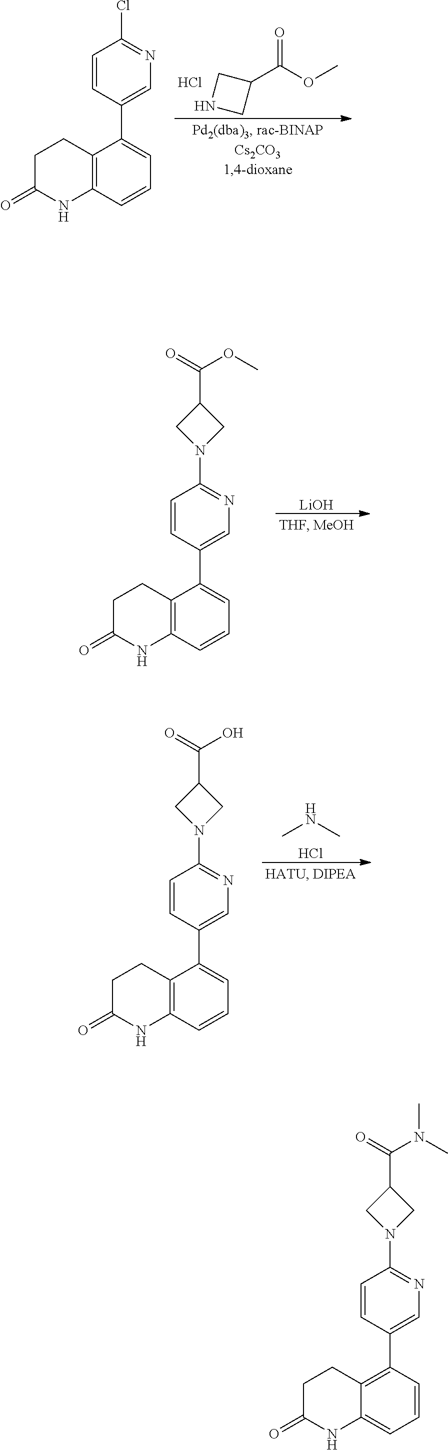

### Synthesis of Methyl 1-(5-(2-oxo-1,2,3,4-tetrahydroquinolin-5-yl)pyridin-2-yl)azetidine-3-carboxylate

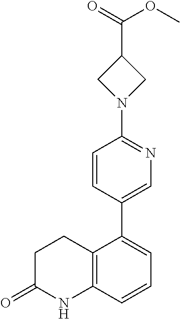 [SMILES: COC(=O)C1CN(c2ccc(-c3cccc4c3CCC(=O)N4)cn2)C1]

In a microwave vial, a solution of 5-(6-chloropyridin-3-yl)-3,4-dihydroquinolin-2(1H)-one (100 mg, 0.390 mmol), methyl azetidine-3-carboxylate hydrochloride (64.5 mg, 0.425 mmol), and cesium carbonate (378 mg, 1.16 mmol) in 1,4-dioxane (3 mL), was degassed with argon gas for 10 min then rac-2,2′-bis(diphenylphosphino)-1,1′-binaphthyl (96 mg, 0.16 mmol) and tris(dibenzylideneacetone)dipalladium(0) (35 mg, 0.039 mmol) were added. The vial was sealed and the reaction was heated at 100° C. overnight. The reaction was cooled to room temperature, diluted with ethyl acetate, filtered through Celite, and rinsed with ethyl acetate. The filtrate was concentrated and purified by normal phase column chromatography (silica gel, 0-100% ethyl acetate/heptane) to afford methyl 1-(5-(2-oxo-1,2,3,4-tetrahydroquinolin-5-yl)pyridin-2-yl)azetidine-3-carboxylate (83 mg, 64%) as a yellow solid. ESI MS m/z 338 [C₁₉H₁₉N₃O₃+H]⁺.

### Synthesis of 1-(5-(2-Oxo-1,2,3,4-tetrahydroquinolin-5-yl)pyridin-2-yl)azetidine-3-carboxylic Acid

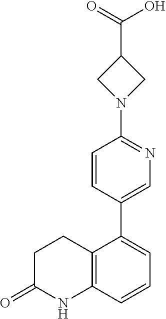 [SMILES: O=C1CCc2c(cccc2-c2ccc(N3CC(C(=O)O)C3)nc2)N1]

To a solution of methyl 1-(5-(2-oxo-1,2,3,4-tetrahydroquinolin-5-yl)pyridin-2-yl)azetidine-3-carboxylate (83 mg, 0.25 mmol) in THF (1 mL) and MeOH (1 mL) was added lithium hydroxide monohydrate (0.5 mL, 1.0 mmol, 2 M) and the reaction mixture was stirred for 1 hour. The reaction was concentrated to remove volatiles then acidified to pH 3 with 1 N HCl. The resulting solid was filtered and the filtrate was concentrated to a minimum volume and purified by C18 reverse phase column chromatography (0-100% acetonitrile/water). Clean fractions were collected and lyophilized (acetonitrile/water) to afford 1-(5-(2-oxo-1,2,3,4-tetrahydroquinolin-5-yl)pyridin-2-yl)azetidine-3-carboxylic acid (53 mg, 66%) as a yellow-white solid. ESI MS m/z 324 [C₁₈H₁₇N₃O₃+H]⁺.

### Synthesis of N,N-Dimethyl-1-(5-(2-oxo-1,2,3,4-tetrahydroquinolin-5-yl)pyridin-2-yl)azetidine-3-carboxamide

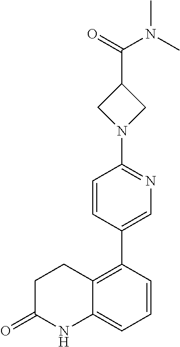 [SMILES: CN(C)C(=O)C1CN(c2ccc(-c3cccc4c3CCC(=O)N4)cn2)C1]

N,N-Dimethyl-1-(5-(2-oxo-1,2,3,4-tetrahydroquinolin-5-yl)pyridin-2-yl)azetidine-3-carboxamide was prepared according to General Protocol 2 using 1-(5-(2-oxo-1,2,3,4-tetrahydroquinolin-5-yl)pyridin-2-yl)azetidine-3-carboxylic acid and dimethylamine hydrochloride to afford N,N-dimethyl-1-(5-(2-oxo-1,2,3,4-tetrahydroquinolin-5-yl)pyridin-2-yl)azetidine-3-carboxamide (4.4 mg, 17%) as an off-white solid. ¹H NMR (500 MHz, DMSO-d₆) δ 10.12 (s, 1H), 8.06 (d, J=2.5 Hz, 1H), 7.53 (dd, J=8.5, 2.0 Hz, 1H), 7.19 (t, J=7.8 Hz, 1H), 6.86 (dd, J=7.7, 2.2 Hz, 2H), 6.47 (d, J=8.5 Hz, 1H), 4.16 (t, J=8.3 Hz, 2H), 4.04 (t, J=7.2 Hz, 2H), 2.91 (s, 3H), 2.86 (s, 3H), 2.80 (t, J=7.5 Hz, 2H), 2.37-2.34 (m, 3H). ESI MS m/z 351 [C₂₀H₂₂N₄O₂+H]⁺.

### Synthesis of 5-(6-(4-(Ethylsulfonyl)piperazin-1-yl)pyridin-3-yl)-3,4-dihydroquinolin-2(1H)-one

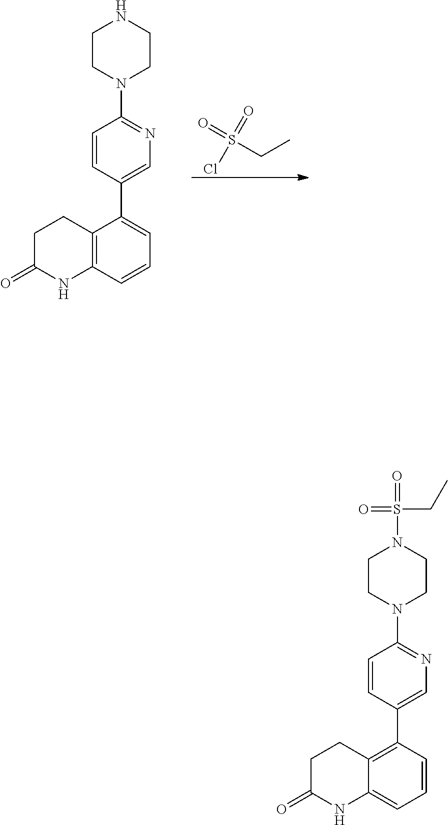

5-(6-(4-(Ethylsulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and ethanesulfonyl chloride to afford 5-(6-(4-(ethylsulfonyl)piperazin-1-yl)pyridin-3-yl)-3,4-dihydroquinolin-2(1H)-one (21 mg, 32%) as an off-white solid. ¹H NMR (500 MHz, DMSO-d₆) δ 10.13 (s, 1H), 8.13 (d, J=2.5 Hz, 1H), 7.59 (dd, J=9.0, 2.5 Hz, 1H), 7.21-7.18 (m, 1H), 6.95 (d, J=8.5 Hz, 1H), 6.88 (d, J=8.0 Hz, 2H), 3.65-3.63 (m, 4H), 3.29-3.27 (m, 4H), 3.10 (q, J=7.3 Hz, 2H), 2.82 (t, J=7.5 Hz, 2H), 2.36 (t, J=7.5 Hz, 2H), 1.23 (t, J=7.5 Hz, 3H). ESI MS m/z 401 [C₂₀H₂₄N₄O₃S+H]⁺.

### Synthesis of 5-(6-(4-(m-Tolylsulfonyl)piperazin-1-yl)pyridin-3-yl)-3,4-dihydroquinolin-2(1H)-one

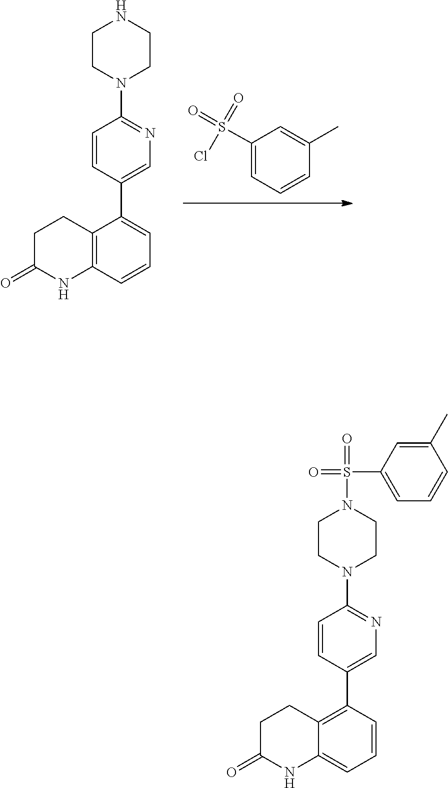 [SMILES: O=C1CCc2c(cccc2-c2ccc(N3CCNCC3)nc2)N1 ; Cc1cccc(S(=O)(=O)Cl)c1 ; Cc1cccc(S(=O)(=O)N2CCN(c3ccc(-c4cccc5c4CCC(=O)N5)cn3)CC2)c1]

5-(6-(4-(m-Tolylsulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and 3-methylbenzenesulfonyl chloride to afford 5-(6-(4-(m-tolylsulfonyl)piperazin-1-yl)pyridin-3-yl)-3,4-dihydroquinolin-2(1H)-one (49 mg, 66%) as an off-white solid. ¹H NMR (500 MHz, DMSO-d₆) δ 10.12 (s, 1H), 8.07 (d, J=2.0 Hz, 1H), 7.58-7.54 (m, 5H), 7.19-7.16 (m, 1H), 6.90-6.84 (m, 3H), 3.66-3.63 (m, 4H), 3.00-2.98 (m, 4H), 2.77 (t, J=7.5 Hz, 2H), 2.42 (s, 3H), 2.37-2.30 (m, 2H). ESI MS m/z 463 [C₂₅H₂₆N₄O₃S+H]⁺.

### Synthesis of 5-(6-(4-(3-Methylbenzoyl)piperazin-1-yl)pyridin-3-yl)-3,4-dihydroquinolin-2(1H)-one

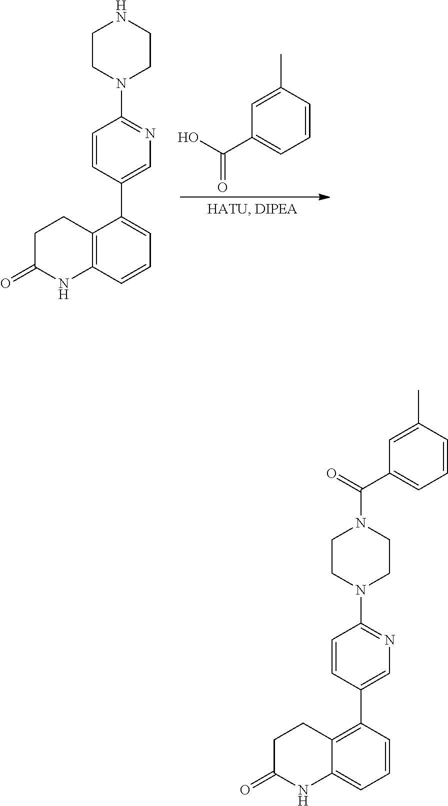

5-(6-(4-(3-Methylbenzoyl)piperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(1H)-one and 3-methylbenzoic acid to afford 5-(6-(4-(3-methylbenzoyl)piperazin-1-yl)pyridin-3-yl)-3,4-dihydroquinolin-2(1H)-one (22 mg, 31%) as an off-white solid. ¹H NMR (500 MHz, DMSO-d₆) δ 10.13 (s, 1H), 8.12 (d, J=2.5 Hz, 1H), 7.58 (dd, J=8.5, 2.5 Hz, 1H), 7.37-7.34 (m, 1H), 7.30-7.18 (m, 4H), 6.93-6.87 (m, 3H), 3.71-3.57 (m, 8H), 2.82 (t, J=7.5 Hz, 2H), 2.38-2.35 (m, 5H). ESI MS m/z 427 [C₂₆H₂₆N₄O₂+H]⁺.

### Synthesis of 5-(6-(4-(2-Methylbenzoyl)piperazin-1-yl)pyridin-3-yl)-3,4-dihydroquinolin-2(1H)-one

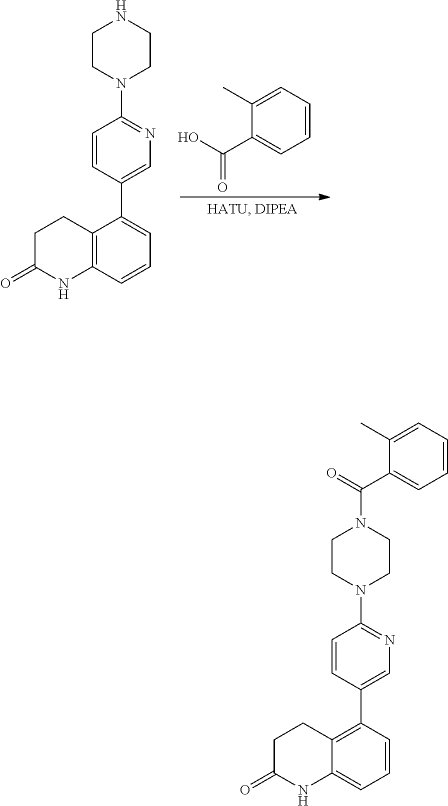

5-(6-(4-(2-Methylbenzoyl)piperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(1H)-one and 2-methylbenzoic acid to afford 5-(6-(4-(2-methylbenzoyl)piperazin-1-yl)pyridin-3-yl)-3,4-dihydroquinolin-2(1H)-one (22 mg, 31%) as an off-white solid. ¹H NMR (500 MHz, DMSO-d₆) δ 10.13 (s, 1H), 8.12 (d, J=2.5 Hz, 1H), 7.58 (dd, J=9.0, 2.5 Hz, 1H), 7.35-7.18 (m, 5H), 6.92-6.91 (m, 3H), 3.78 (s, 2H), 3.67 (s, 2H), 3.51-3.49 (m, 2H), 3.28-3.26 (m, 2H), 2.81 (t, J=7.2 Hz, 2H), 2.36 (t, J=7.5 Hz, 2H), 2.24 (s, 3H). ESI MS m/z 427 [C₂₆H₂₆N₄O₂+H]⁺.

### Synthesis of 5-(6-(4-Benzoylpiperazin-1-yl)pyridin-3-yl)-3,4-dihydroquinolin-2(1H)-one

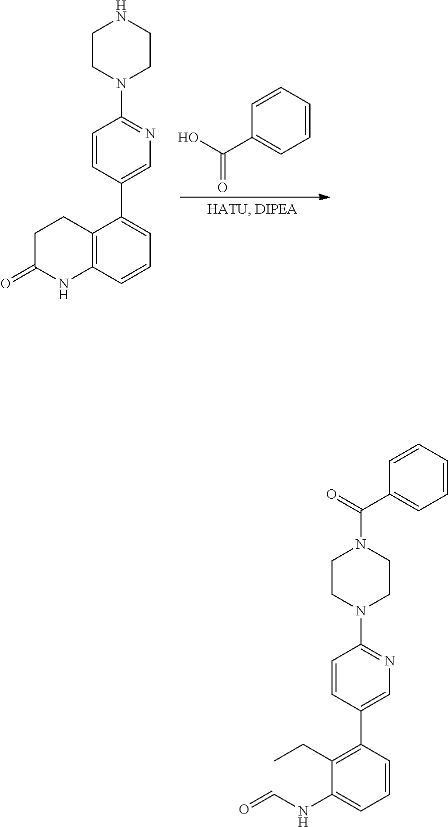

5-(6-(4-Benzoylpiperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(1H)-one and benzoic acid to afford 5-(6-(4-benzoylpiperazin-1-yl)pyridin-3-yl)-3,4-dihydroquinolin-2(1H)-one (17 mg, 25%) as an off-white solid. ¹H NMR (500 MHz, DMSO-d₆) δ 10.12 (s, 1H), 8.12 (d, J=2.0 Hz, 1H), 7.58 (dd, J=8.5, 2.5 Hz, 1H), 7.48-7.44 (m, 5H), 7.19 (t, J=7.7 Hz, 1H), 6.93-6.87 (m, 3H), 3.81-3.40 (m, 8H), 2.82 (t, J=7.5 Hz, 2H), 2.36 (t, J=7.5 Hz, 2H). ESI MS m/z 413 [C₂₅H₂₄N₄O₂+H]⁺.

### Synthesis of 5-(6-(4-(Cyclopropanecarbonyl)piperazin-1-yl)pyridin-3-y)-3,4-dihydroquinolin-2(1H)-one

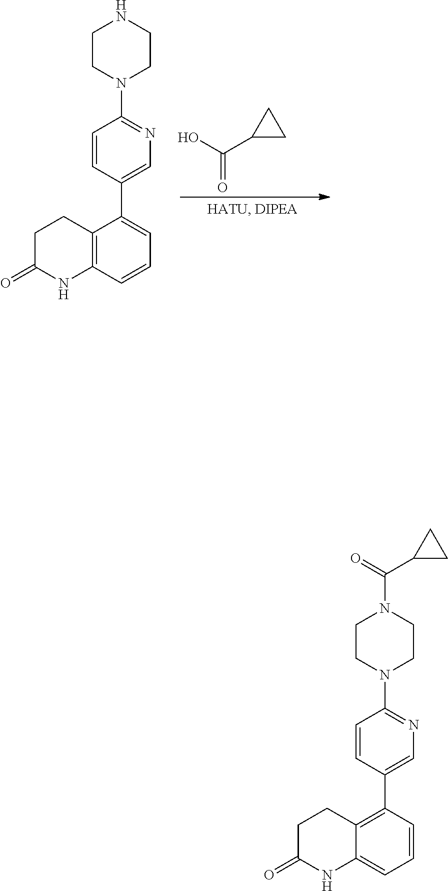

5-(6-(4-(Cyclopropanecarbonyl)piperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(1H)-one and cyclopropanecarboxylic acid to afford 5-(6-(4-(cyclopropanecarbonyl)piperazin-1-yl)pyridin-3-yl)-3,4-dihydroquinolin-2(1H)-one (37 mg, 60%) as an off-white solid. ¹H NMR (500 MHz, DMSO-d₆) δ 10.13 (s, 1H), 8.12 (d, J=2.0 Hz, 1H), 7.58 (dd, J=8.5, 2.5 Hz, 1H), 7.20 (dd, J=7.7, 7.7 Hz, 1H), 6.92 (d, J=9.0 Hz, 1H), 6.89-6.87 (m, 2H), 3.81-3.54 (m, 8H), 2.82 (t, J=7.5 Hz, 2H), 2.36 (t, J=7.5 Hz, 2H), 0.79-0.71 (m, 4H). ESI MS m/z 377 [C₂₂H₂₄N₄O₂+H]⁺.

### Synthesis of 5-(6-(4-(Phenylsulfonyl)piperazin-1-yl)pyridin-3-yl)-3,4-dihydroquinolin-2(1H)-one

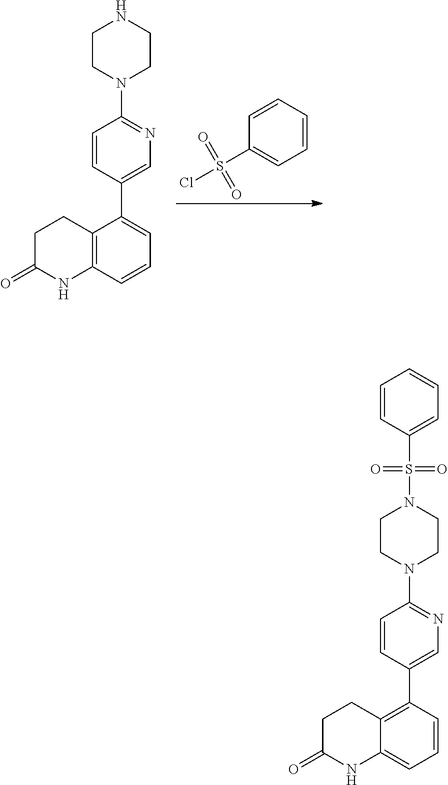

5-(6-(4-(Phenylsulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and benzenesulfonyl chloride to afford 5-(6-(4-(phenylsulfonyl)piperazin-1-yl)pyridin-3-yl)-3,4-dihydroquinolin-2(1H)-one (22 mg, 30%) as an off-white solid. ¹H NMR (500 MHz, DMSO-d₆) δ 10.11 (s, 1H), 8.07 (d, J=2.5 Hz, 1H), 7.79-7.74 (m, 2H), 7.74-7.72 (m, 1H), 7.68-7.65 (m, 2H), 7.54 (dd, J=9.0, 2.5 Hz, 1H), 7.17 (t, J=7.7 Hz, 1H), 6.88-6.83 (m, 3H), 3.65-3.63 (m, 4H), 3.01-2.99 (m, 4H), 2.77 (t, J=7.5 Hz, 2H), 2.34 (t, J=7.5 Hz, 2H). ESI MS m/z 449 [C₂₄H₂₄N₄O₃S+H]⁺.

### Synthesis of 5-(6-(4-(Isopropylsulfonyl)piperazin-1-yl)pyridin-3-yl)-3,4-dihydroquinolin-2(1H)-one

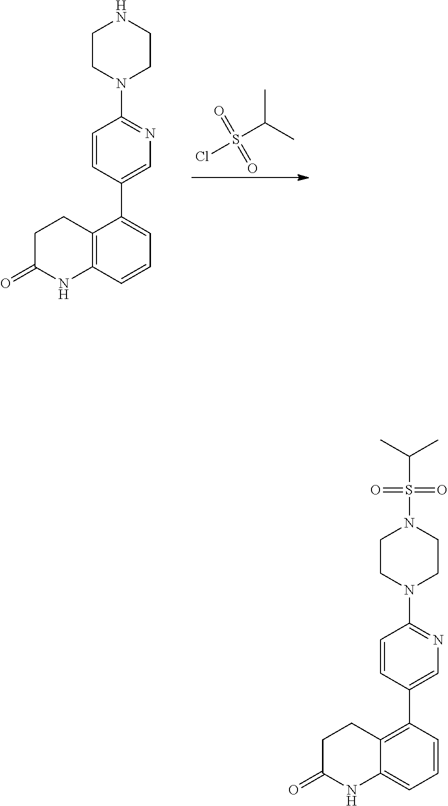

5-(6-(4-(Isopropylsulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and propane-2-sulfonyl chloride to afford 5-(6-(4-(isopropylsulfonyl) piperazin-1-yl)pyridin-3-yl)-3,4-dihydroquinolin-2(1H)-one (20 mg, 30%) as an off-white solid. ¹H NMR (500 MHz, DMSO-d₆) δ ¹H NMR (500 MHz, CDCl3) δ 10.12 (s, 1H), 8.12 (d, J=2.5 Hz, 1H), 7.59-7.57 (m, 1H), 7.20-7.18 (m, 1H), 6.94 (d, J=9.0 Hz, 1H), 6.88 (d, J=7.5 Hz, 2H), 3.62-3.60 (m, 4H), 3.40-3.38 (m, 1H), 3.37-3.36 (m, 4H), 2.82 (t, J=7.5 Hz, 2H), 2.36 (t, J=7.2 Hz, 2H), 1.25 (d, J=7.0 Hz, 6H). ESI MS m/z 415 [C₂₁H₂₆N₄O₃S+H]⁺.

### Synthesis of 5-(6-(4-((3-Chlorophenyl)sulfonyl)piperazin-1-yl)pyridin-3-y)-3,4-dihydroquinolin-2(1H)-one

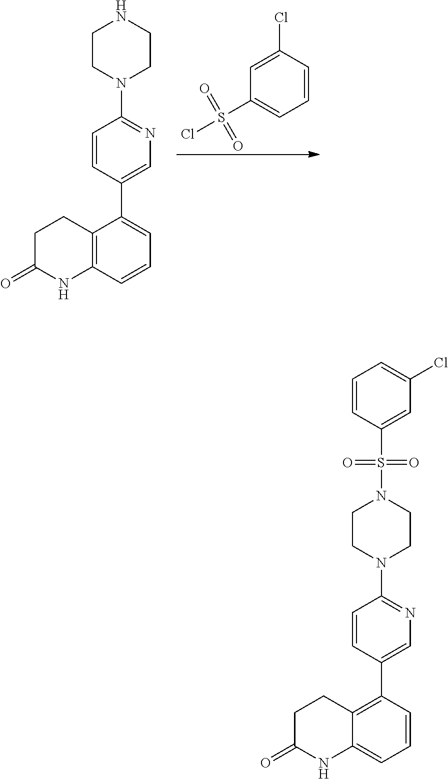

5-(6-(4-((3-Chlorophenyl)sulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and 3-chlorobenzenesulfonyl chloride to afford 5-(6-(4-((3-chlorophenyl)sulfonyl)piperazin-1-yl)pyridin-3-yl)-3,4-dihydroquinolin-2(1H)-one (54 mg, 69%) as an off-white solid. ¹H NMR (500 MHz, DMSO-d₆) δ ¹H NMR (500 MHz, CDCl3) δ 10.11 (s, 1H), 8.08 (s, 1H), 7.83-7.68 (m, 4H), 7.55 (d, J=8.5 Hz, 1H), 7.18 (t, J=7.7 Hz, 1H), 6.90-6.84 (m, 3H), 3.65 (s, 4H), 3.06 (s, 4H), 2.78 (t, J=7.5 Hz, 2H), 2.34 (t, J=7.2 Hz, 2H). ESI MS m/z 483 [C₂₄H₂₃ClN₄O₃S+H]⁺.

### Synthesis of 5-(6-(4-((2-Chlorophenyl)sulfonyl)piperazin-1-yl)pyridin-3-y)-3,4-dihydroquinolin-2(1H)-one

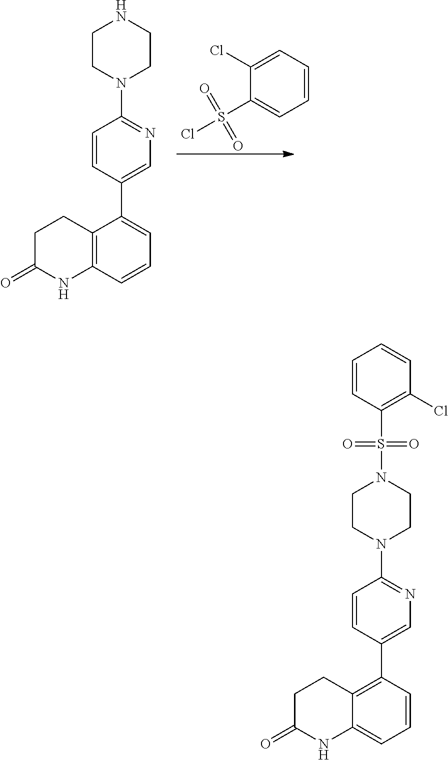

5-(6-(4-((2-Chlorophenyl)sulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and 2-chlorobenzenesulfonyl chloride to afford 5-(6-(4-((2-chlorophenyl)sulfonyl)piperazin-1-yl)pyridin-3-yl)-3,4-dihydroquinolin-2(1H)-one (17 mg, 22%) as an off-white solid. ¹H NMR (500 MHz, DMSO-d₆) δ ¹H NMR (500 MHz, CDCl3) δ 10.12 (s, 1H), 8.10 (d, J=2.5 Hz, 1H), 8.03-8.01 (m, 1H), 7.74-7.68 (m, 2H), 7.61-7.55 (m, 2H), 7.19 (t, J=7.7 Hz, 1H), 6.91 (d, J=9.0 Hz, 1H), 6.88-6.85 (m, 2H), 3.64-3.62 (m, 4H), 3.29-3.26 (m, 4H), 2.79 (t, J=7.5 Hz, 2H), 2.35 (t, J=7.5 Hz, 2H). ESI MS m/z 483 [C₂₄H₂₃ClN₄O₃S+H]⁺.

### Synthesis of 5-(6-(4-(Cyclopropylsulfonyl)piperazin-1-yl)pyridin-3-yl)-3,4-dihydroquinolin-2(1H)-one

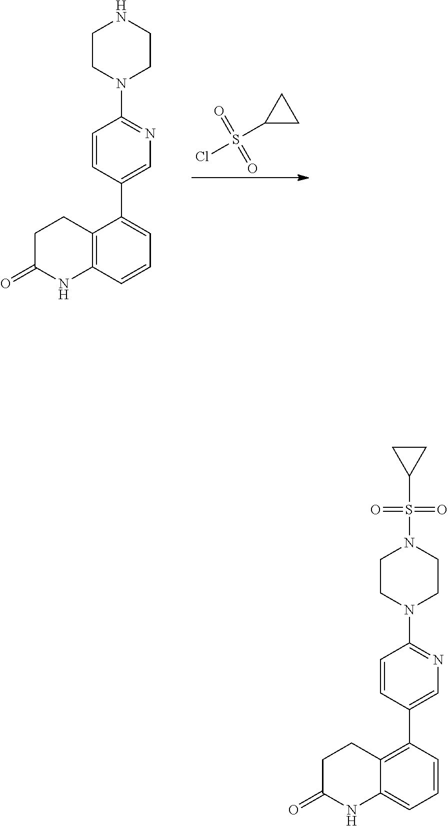 [SMILES: O=C1CCc2c(cccc2-c2ccc(N3CCNCC3)nc2)N1 ; O=S(=O)(Cl)C1CC1 ; O=C1CCc2c(cccc2-c2ccc(N3CCN(S(=O)(=O)C4CC4)CC3)nc2)N1]

5-(6-(4-(Cyclopropylsulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and cyclopropanesulfonyl chloride to afford 5-(6-(4-(cyclopropylsulfonyl)piperazin-1-yl)pyridin-3-yl)-3,4-dihydroquinolin-2(1H)-one (29 mg, 44%) as an off-white solid. ¹H NMR (500 MHz, DMSO-d₆) δ ¹H NMR (500 MHz, CDCl3) δ 10.13 (s, 1H), 8.13 (d, J=2.5 Hz, 1H), 7.59 (dd, J=8.5, 2.5 Hz, 1H), 7.20 (t, J=7.7 Hz, 1H), 6.96 (d, J=9.0 Hz, 1H), 6.88 (d, J=7.5 Hz, 2H), 3.67-3.65 (m, 4H), 3.23-3.22 (m, 4H), 2.82 (t, J=7.5 Hz, 2H), 2.36 (t, J=7.5 Hz, 2H), 1.01-0.94 (m, 4H). ESI MS m/z 413 [C₂₁H₂₄N₄O₃S+H]⁺.

### Synthesis of 5-(6-(4-(Vinylsulfonyl)piperazin-1-yl)pyridin-3-yl)-3,4-dihydroquinolin-2(1H)-one

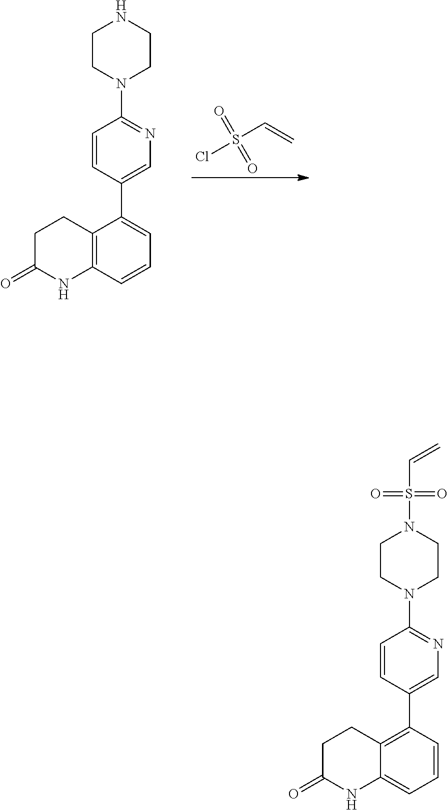

5-(6-(4-(Vinylsulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and ethenesulfonyl chloride to afford 5-(6-(4-(vinylsulfonyl)piperazin-1-yl)pyridin-3-yl)-3,4-dihydroquinolin-2(1H)-one (21 mg, 17%) as a white solid. ¹H NMR (500 MHz, DMSO-d₆) δ ¹H NMR (500 MHz, CDCl3) δ 10.13 (s, 1H), 8.12 (d, J=2.0 Hz, 1H), 7.59 (dd, J=9.0, 2.5 Hz, 1H), 7.20 (t, J=7.8 Hz, 1H), 6.95 (d, J=8.5 Hz, 1H), 6.88-6.83 (m, 3H), 6.21-6.14 (m, 2H), 3.68-3.66 (m, 4H), 3.15-3.13 (m, 4H), 2.81 (t, J=7.5 Hz, 2H), 2.36 (t, J=7.5 Hz, 2H). ESI MS m/z 399 [C₂₀H₂₂N₄O₃S+H]⁺.

### Synthesis of 5-(6-(4-(Cyclohexanecarbonyl)piperazin-1-yl)pyridin-3-yl)-3,4-dihydroquinolin-2(1H)-one

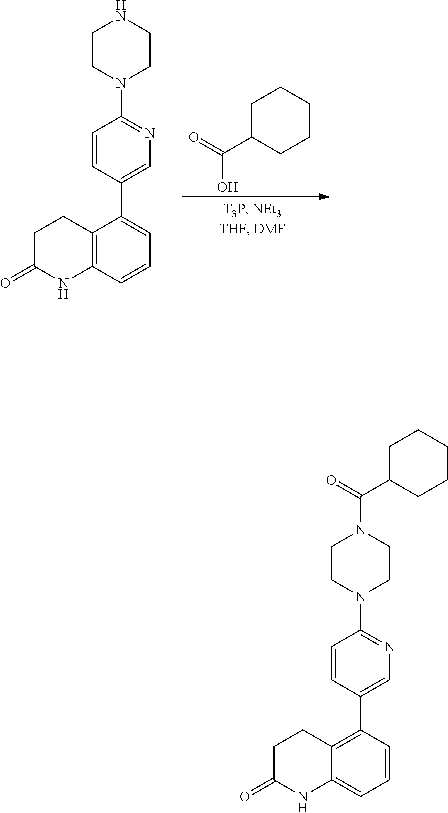

To a solution of 5-(6-(piperazin-1-yl)pyridin-3-yl)-3,4-dihydroquinolin-2(1H)-one (0.05 g, 0.16 mmol) in DMF (3 mL) and THF (3 mL) was added cyclohexanecarboxylic acid (0.062 g, 0.49 mmol), triethylamine (0.068 mL, 0.496 mmol) followed by dropwise addition of 2,4,6-tripropyl-1,3,5,2,4,6-trioxatriphosphinane 2,4,6-trioxide (T₃P, 0.15 mL, 0.24 mmol, 50 wt. % in ethyl acetate) at room temperature. The reaction was stirred for 2 hours then diluted with dichloromethane and washed with water (3×) then brine. The organic layer was collected, dried over sodium sulfate, and concentrated. The residue was purified by column chromatography (silica gel, 0-100% EtOAc/hexanes) to afford 5-(6-(4-(cyclohexanecarbonyl)piperazin-1-yl)pyridin-3-yl)-3,4-dihydroquinolin-2(1H)-one (17 mg, 25%) as an off-white solid. ¹H NMR (300 MHz, DMSO-d₆) δ 10.15 (s, 1H), 8.12 (d, J=2.4 Hz, 1H), 7.58 (dd, J=8.7, 2.4 Hz, 1H), 7.20 (t, J=7.8 Hz, 1H), 6.93-6.86 (m, 3H), 3.58-3.52 (m, 8H), 2.82 (t, J=7.5 Hz, 2H), 2.63-2.62 (m, 1H), 2.36 (t, J=7.3 Hz, 2H), 1.72-1.66 (m, 5H), 1.36-1.18 (m, 5H). ESI MS m/z 419 [C₂₃H₃₀N₄O₂+H]⁺.

### Synthesis of 5-(6-(4-(Methylsulfonyl)piperazin-1-yl)pyridin-3-yl)-3,4-dihydroquinolin-2(1H)-one

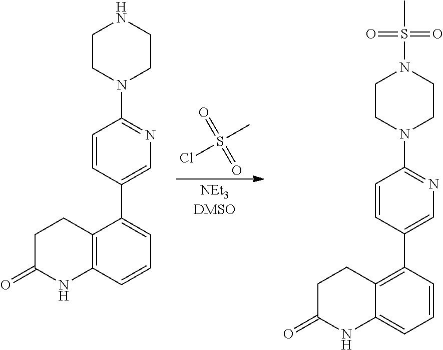

5-(6-(4-(Methylsulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and methanesulfonyl chloride to afford 5-(6-(4-(methylsulfonyl)piperazin-1-yl)pyridin-3-yl)-3,4-dihydroquinolin-2(1H)-one (20 mg, 19%) as an off-white solid. ¹H NMR (300 MHz, DMSO-d₆) δ 10.16 (s, 1H), 8.13 (d, J=2.4 Hz, 1H), 7.60 (dd, J=8.9, 2.6 Hz, 1H), 7.20 (t, J=7.8 Hz, 1H), 6.97 (d, J=9.0 Hz, 1H), 6.88 (d, J=8.1 Hz, 2H), 3.69-3.66 (m, 4H), 3.23-3.20 (m, 4H), 2.90 (s, 3H), 2.82 (t, J=7.5 Hz, 2H), 2.36 (t, J=7.3 Hz, 2H). ESI MS m/z 387 [C₁₉H₂₂N₄O₃S+H]⁺.

### Synthesis of 5-(6-(4-((Tetrahydrofuran-3-yl)sulfonyl)piperazin-1-yl)pyridin-3-y)-3,4-dihydroquinolin-2(1H)-one

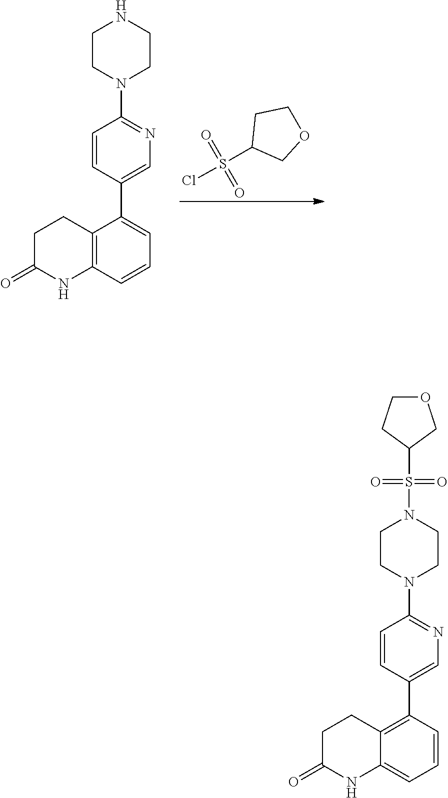

5-(6-(4-((Tetrahydrofuran-3-yl)sulfonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and tetrahydrofuran-3-sulfonyl chloride with DMSO as the solvent to afford 5-(6-(4-((tetrahydrofuran-3-yl)sulfonyl)piperazin-1-yl)pyridin-3-yl)-3,4-dihydroquinolin-2(1H)-one (11 mg, 11%) as an off-white solid. ¹H NMR (300 MHz, DMSO-d₆) δ 10.16 (s, 1H), 8.13 (d, J=2.4 Hz, 1H), 7.59 (dd, J=8.9, 2.6 Hz, 1H), 7.20 (t, J=7.8 Hz, 1H), 6.95 (d, J=8.7 Hz, 1H), 6.88 (d, J=7.8 Hz, 2H), 4.07-3.79 (m, 4H), 3.70-3.62 (m, 5H), 3.32 (br s, 4H), 2.82 (t, J=7.3 Hz, 2H), 2.36 (t, J=7.5 Hz, 2H), 2.28-2.08 (m, 2H). ESI MS m/z 443 [C₂₂H₂₆N₄O₄S+H]⁺.

### Synthesis of 5-(6-(4-Propionylpiperazin-1-yl)pyridin-3-yl)-3,4-dihydroquinolin-2(1H)-one

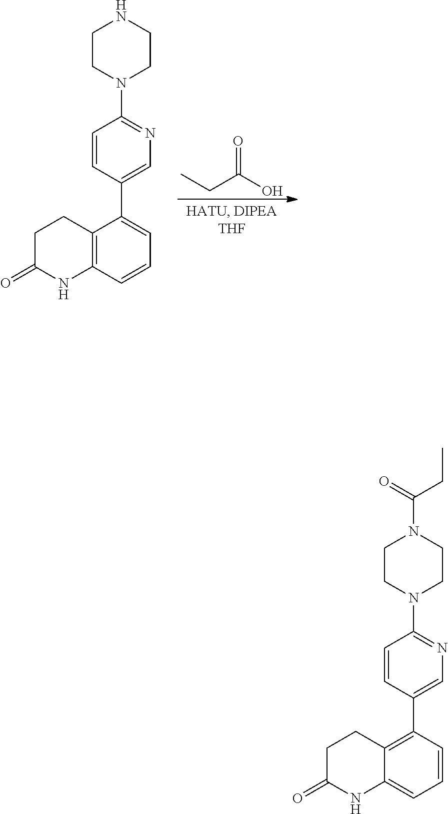

5-(6-(4-Propionylpiperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(11)-one and propionic acid to afford 5-(6-(4-propionylpiperazin-1-yl)pyridin-3-yl)-3,4-dihydroquinolin-2(11)-one (29.7 mg, 50%) as an off-white solid. ¹H NMR (500 MHz, DMSO-d₆) δ 10.13 (s, 1H), 8.12 (d, J=2.5 Hz, 1H), 7.57 (dd, J=8.5, 2.5 Hz, 1H), 7.19 (t, J=8.0 Hz, 1H), 6.91 (d, J=8.5 Hz, 1H), 6.87 (d, J=7.5 Hz, 2H), 3.58-3.52 (m, 8H), 2.82 (t, J=7.0 Hz, 2H), 2.39-2.34 (m, 4H), 1.02 (t, J=7.5 Hz, 3H). ESI MS m/z 365 [C₂₁H₂₄N₄O₂+H]⁺.

### Synthesis of 5-(6-(4-(3-Chlorobenzoyl)piperazin-1-yl)pyridin-3-yl)-3,4-dihydroquinolin-2(1H)-one

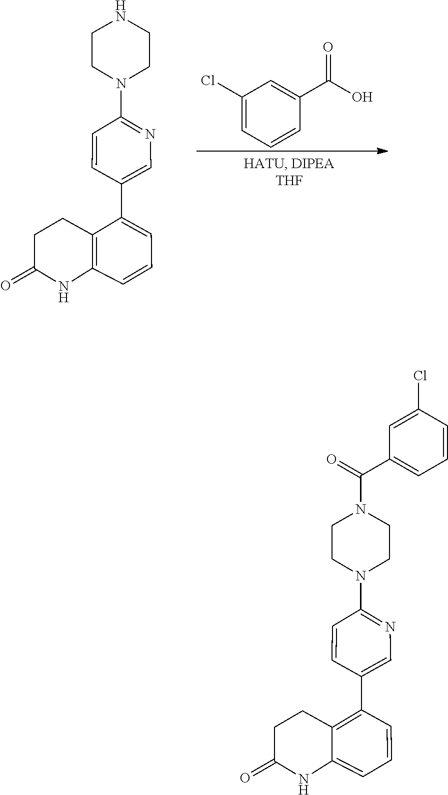 [SMILES: O=C1CCc2c(cccc2-c2ccc(N3CCNCC3)nc2)N1 ; O=C(O)c1cccc(Cl)c1 ; O=C1CCc2c(cccc2-c2ccc(N3CCN(C(=O)c4cccc(Cl)c4)CC3)nc2)N1]

5-(6-(4-(3-Chlorobenzoyl)piperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(1H)-one and 3-chlorobenzoic acid to afford 5-(6-(4-(3-chlorobenzoyl)piperazin-1-yl)pyridin-3-yl)-3,4-dihydroquinolin-2(1H)-one (43.1 mg, 59%) as an off-white solid. ¹H NMR (500 MHz, DMSO-d₆) δ 10.12 (s, 1H), 8.12 (d, J=2.5 Hz, 1H), 7.59-7.49 (m, 4H), 7.43-7.40 (m, 1H), 7.20 (t, J=7.8 Hz, 1H), 6.92 (d, J=8.5 Hz, 1H), 6.88 (d, J=8.0 Hz, 2H), 3.73-3.44 (m, 8H), 2.82 (t, J=7.5 Hz, 2H), 2.36 (t, J=7.5 Hz, 2H). ESI MS m/z 447 [C₂₅H₂₃ClN₄O₂+H]⁺.

### Synthesis of 5-(6-(4-((Cyclohexylsulfonyl)carbonyl)piperazin-1-yl)pyridin-3-y)-3,4-dihydroquinolin-2(1H)-one

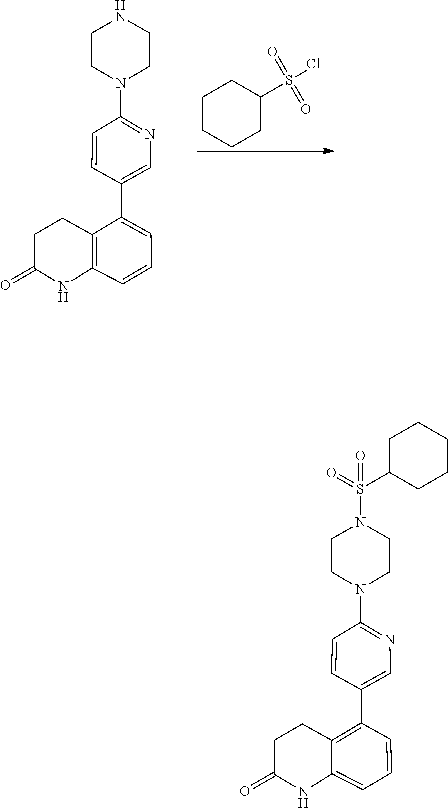 [SMILES: O=C1CCc2c(cccc2-c2ccc(N3CCNCC3)nc2)N1 ; O=S(=O)(Cl)C1CCCCC1 ; O=C1CCc2c(cccc2-c2ccc(N3CCN(S(=O)(=O)C4CCCCC4)CC3)nc2)N1]

5-(6-(4-((Cyclohexylsulfonyl)carbonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and cyclohexanesulfonyl chloride to afford 5-(6-(4-((cyclohexylsulfonyl)carbonyl)piperazin-1-yl)pyridin-3-yl)-3,4-dihydroquinolin-2(1H)-one (26.9 mg, 30%) as an off-white solid. ¹H NMR (500 MHz, DMSO-d₆) δ 10.13 (s, 1H), 8.12 (d, J=2.0 Hz, 1H), 7.58 (dd, J=8.5, 2.5 Hz, 1H), 7.20 (t, J=7.7 Hz, 1H), 6.93 (d, J=8.5 Hz, 1H), 6.88 (d, J=7.5 Hz, 2H), 3.61 (t, J=4.7 Hz, 4H), 3.36-3.26 (m, 4H), 3.20-3.13 (m, 1H), 2.82 (t, J=7.2 Hz, 2H), 2.36 (t, J=7.5 Hz, 2H), 2.02 (d, J=11.0 Hz, 2H), 1.78 (d, J=12.5 Hz, 2H), 1.62 (d, J=12.5 Hz, 1H), 1.43-1.24 (m, 4H), 1.18-1.15 (m, 1H). ESI MS m/z 455 [C₂₄H₃₀N₄O₃S+H]⁺.

### Synthesis of 5-(6-(4-((Cyclopentylsulfonyl)carbonyl)piperazin-1-yl)pyridin-3-y)-3,4-dihydroquinolin-2(1H)-one

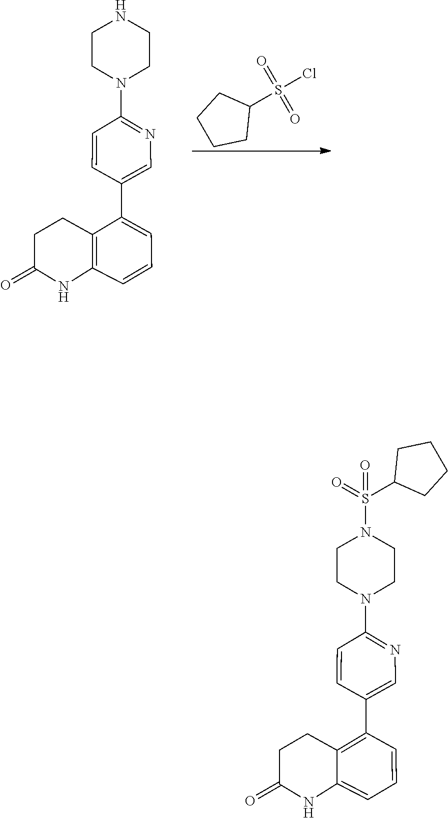 [SMILES: O=C1CCc2c(cccc2-c2ccc(N3CCNCC3)nc2)N1 ; O=S(=O)(Cl)C1CCCC1 ; O=C1CCc2c(cccc2-c2ccc(N3CCN(S(=O)(=O)C4CCCC4)CC3)nc2)N1]

5-(6-(4-((Cyclopentylsulfonyl)carbonyl)piperazin-1-yl)pyridin-3-yl)-3,4-dihydroquinolin-2(1H)-one was prepared according to General Protocol 1 using 5-(6-(piperazin-1-yl)pyridin-3-yl)-3,4-dihydroquinolin-2(1H)-one and cyclopentanesulfonyl chloride to afford 5-(6-(4-((cyclohexylsulfonyl)carbonyl)piperazin-1-yl)pyridin-3-yl)-3,4-dihydroquinolin-2(1H)-one (17.3 mg, 17%) as an off-white solid. ¹H NMR (500 MHz, DMSO-d₆) δ 10.13 (s, 1H), 8.12 (d, J=2.5 Hz, 1H), 7.58 (dd, J=9.0, 2.5 Hz, 1H), 7.20 (t, J=7.7 Hz, 1H), 6.94 (d, J=9.0 Hz, 1H), 6.88 (d, J=7.5 Hz, 2H), 3.70-3.57 (m, 5H), 3.33-3.30 (m, 4H), 2.81 (t, J=7.5 Hz, 2H), 2.36 (t, J=7.5 Hz, 2H), 1.99-1.93 (m, 2H), 1.86-1.79 (m, 2H), 1.69-1.66 (m, 2H), 1.57-1.54 (m, 2H). ESI MS m/z 441 [C₂₃H₂₈N₄O₃S+H]⁺.

### Synthesis of 5-(6-(4-(Cyclopentanecarbonyl)piperazin-1-yl)pyridin-3-y)-3,4-dihydroquinolin-2(1H)-one

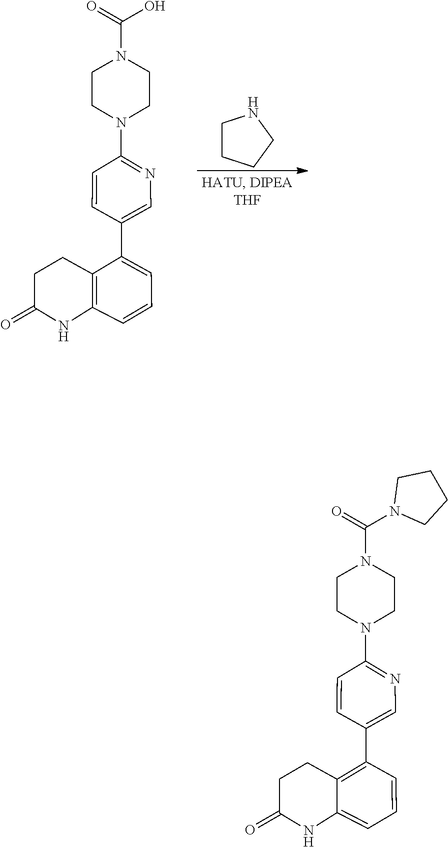 [SMILES: O=C1CCc2c(cccc2-c2ccc(N3CCN(C(=O)O)CC3)nc2)N1 ; C1CCNC1 ; O=C1CCc2c(cccc2-c2ccc(N3CCN(C(=O)N4CCCC4)CC3)nc2)N1]

5-(6-(4-(Cyclopentanecarbonyl)piperazin-1-yl)pyridin-3-yl)-3,4-dihydroquinolin-2(1H)-one was prepared according to General Protocol 2 using 4-(5-(2-oxo-1,2,3,4-tetrahydroquinolin-5-yl)pyridin-2-yl)piperazine-1-carboxylic acid and pyrrolidine to afford 5-(6-(4-(cyclopentanecarbonyl)piperazin-1-yl)pyridin-3-yl)-3,4-dihydroquinolin-2(11)-one (15.5 mg, 18%) as an off-white solid. ¹H NMR (500 MHz, DMSO-d₆) δ 10.12 (s, 1H), 8.06 (d, J=1.5 Hz, 1H), 7.53 (dd, J=1.0, 2.5 Hz, 1H), 7.19 (t, J=8.0 Hz, 1H), 6.86 (dd, J=1.0, 1.7 Hz, 2H), 6.47 (d, J=8.5 Hz, 1H), 4.14 (t, J=8.2 Hz, 2H), 4.04 (t, J=7.0 Hz, 2H), 3.78-3.74 (m, 1H), 3.37-3.30 (m, 4H), 2.80 (t, J=7.5 Hz, 2H), 2.36 (t, J=7.2 Hz, 2H), 1.91-1.87 (m, 2H), 1.80-1.77 (m, 2H). ESI MS m/z 377 [C₂₂H₂₄N₄O₂+H]⁺.

### Synthesis of 5-(6-(4-(Morpholine-4-carbonyl)piperazin-1-yl)pyridin-3-y)-3,4-dihydroquinolin-2(1H)-one

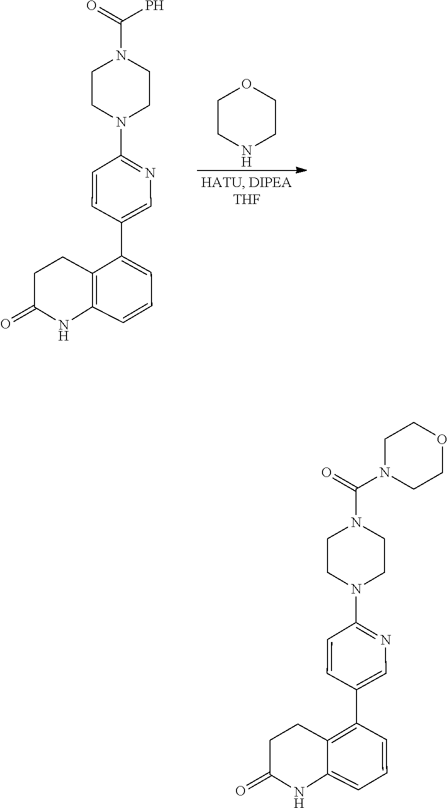

5-(6-(4-(Morpholine-4-carbonyl)piperazin-1-yl)pyridin-3-yl)-3,4-dihydroquinolin-2(1H)-one was prepared according to General Protocol 2 using 4-(5-(2-oxo-1,2,3,4-tetrahydroquinolin-5-yl)pyridin-2-yl)piperazine-1-carboxylic acid and morpholine to afford 5-(6-(4-(morpholine-4-carbonyl)piperazin-1-yl)pyridin-3-yl)-3,4-dihydroquinolin-2(1H)-one (13.4 mg, 15%) as an off-white solid. ¹H NMR (500 MHz, DMSO-d₆) δ 10.12 (s, 1H), 8.06 (d, J=2.0 Hz, 1H), 7.54 (dd, J=1.0, 2.5 Hz, 1H), 7.19 (t, J=7.7 Hz, 1H), 6.86 (dt, J=8.0, 2.0 Hz, 2H), 6.48 (d, J=8.5 Hz, 1H), 4.16 (t, J=8.5 Hz, 2H), 4.07 (t, J=7.2 Hz, 2H), 3.59-3.56 (m, 4H), 3.48 (t, J=5.0 Hz, 2H), 3.35 (t, J=4.7 Hz, 2H), 2.80 (t, J=7.5 Hz, 2H), 2.36 (t, J=7.5 Hz, 2H). ESI MS m/z 393 [C₂₂H₂₄N₄O₃+H]⁺.

### Synthesis of N-isopropyl-4-(5-(2-oxo-1,2,3,4-tetrahydroquinolin-5-yl)pyridin-2-yl)piperazine-1-carboxamide

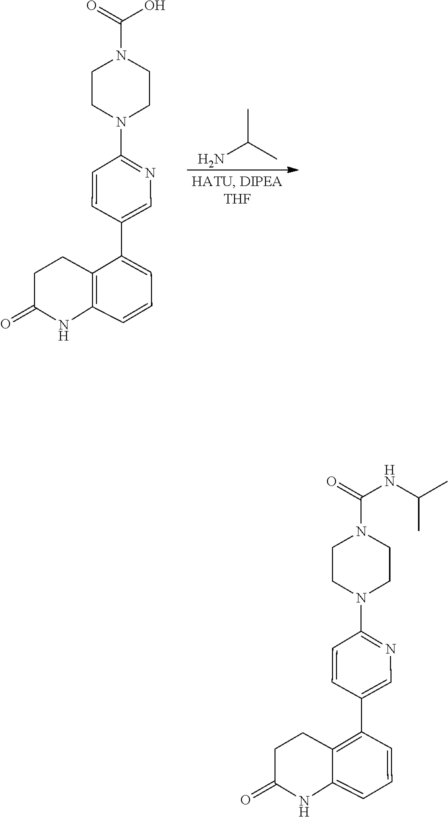

N-Isopropyl-4-(5-(2-oxo-1,2,3,4-tetrahydroquinolin-5-yl)pyridin-2-yl)piperazine-1-carboxamide was prepared according to General Protocol 2 using 4-(5-(2-oxo-1,2,3,4-tetrahydroquinolin-5-yl)pyridin-2-yl)piperazine-1-carboxylic acid and isopropylamine to afford N-isopropyl-4-(5-(2-oxo-1,2,3,4-tetrahydroquinolin-5-yl)pyridin-2-yl)piperazine-1-carboxamide (21.0 mg, 25%) as an off-white solid. ¹H NMR (500 MHz, DMSO-d₆) δ 10.12 (s, 1H), 8.05 (dd, J=1.0, 0.5 Hz, 1H), 7.87 (d, J=8.0 Hz, 1H), 7.53 (dd, J=1.0, 2.0 Hz, 1H), 7.19 (t, J=7.8 Hz, 1H), 6.86 (dd, J=1.0, 1.5 Hz, 2H), 6.45 (d, J=8.5 Hz, 1H), 4.07 (t, J=8.0 Hz, 2H), 3.96 (dd, J=1.0, 6.3 Hz, 2H), 3.89-3.83 (m, 1H), 3.45-3.41 (m, 1H), 2.81 (t, J=7.5 Hz, 2H), 2.36 (dd, J=1.0, 6.7 Hz, 2H), 1.06 (d, J=6.5 Hz, 6H). ESI MS m/z 365 [C₂₁H₂₄N₄O₂+H]⁺.

### Synthesis of N-Benzyl-N-methyl-4-(5-(2-oxo-1,2,3,4-tetrahydroquinolin-5-yl)pyridin-2-yl)piperazine-1-carboxamide

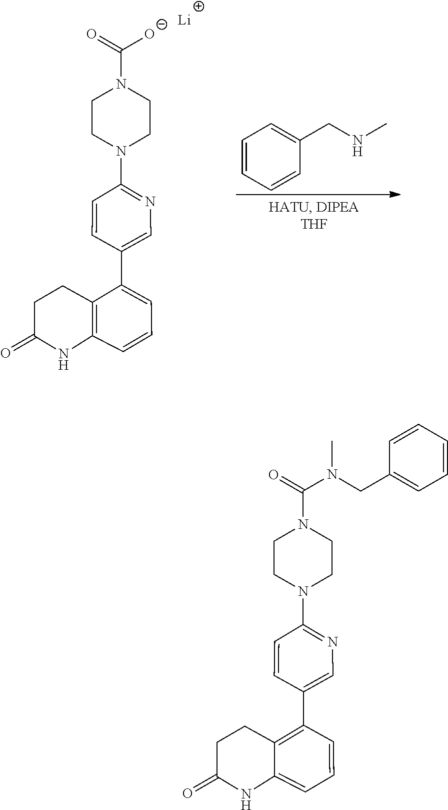

N-Benzyl-N-methyl-4-(5-(2-oxo-1,2,3,4-tetrahydroquinolin-5-yl)pyridin-2-yl)piperazine-1-carboxamide was prepared according to General Protocol 2 using lithium 1-(5-(2-oxo-1,2,3,4-tetrahydroquinolin-5-yl)pyridin-2-yl)azetidine-3-carboxylate and N-benzylmethylamine to afford N-benzyl-N-methyl-4-(5-(2-oxo-1,2,3,4-tetrahydroquinolin-5-yl)pyridin-2-yl)piperazine-1-carboxamide (65.3 mg, 72%) as an off-white solid. ¹H NMR (500 MHz, DMSO-d₆), mixture of rotamers) δ 10.12 (s, 1H), 8.07 (dd, J=2.5, 0.5 Hz, 0.65H), 8.04 (dd, J=2.0, 0.5 Hz, 0.35H), 7.56-7.52 (m, 1H), 7.41-7.17 (m, 7H), 6.88-6.85 (m, 2H), 6.50 (d, J=11.5 Hz, 0.65H), 6.45 (d, J=8.5 Hz, 0.35H), 4.54-4.53 (m, 2H), 4.20 (t, J=8.3 Hz, 1.35H), 4.10-4.02 (m, 2.65H), 3.99-3.95 (m, 1H), 2.87-2.78 (m, 5H), 2.38-2.35 (m, 2H). ESI MS m/z 427 [C₂₆H₂₆N₄O₂+H]⁺.

### Synthesis of N-Ethyl-N-methyl-4-(5-(2-oxo-1,2,3,4-tetrahydroquinolin-5-yl)pyridin-2-yl)piperazine-1-carboxamide

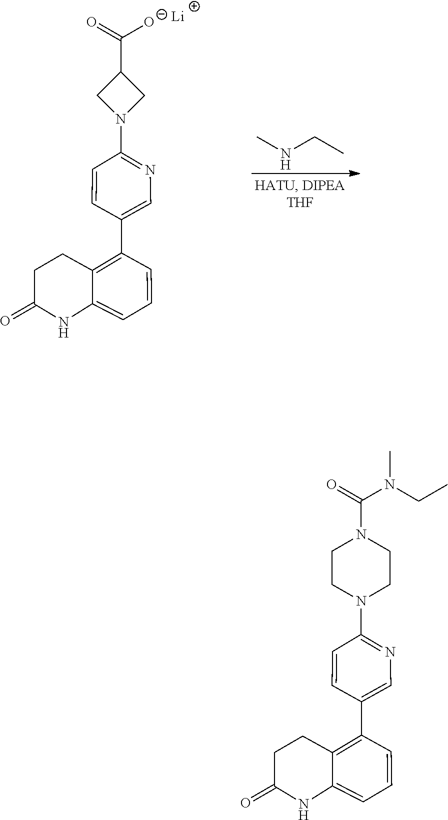

N-Ethyl-N-methyl-4-(5-(2-oxo-1,2,3,4-tetrahydroquinolin-5-yl)pyridin-2-yl)piperazine-1-carboxamide was prepared according to General Protocol 2 using lithium 1-(5-(2-oxo-1,2,3,4-tetrahydroquinolin-5-yl)pyridin-2-yl)azetidine-3-carboxylate and N-ethylmethylamine to afford N-ethyl-N-methyl-4-(5-(2-oxo-1,2,3,4-tetrahydroquinolin-5-yl)pyridin-2-yl)piperazine-1-carboxamide (33.5 mg, 50%) as an off-white solid. ¹H NMR (500 MHz, DMSO-d₆), mixture of rotamers) δ 10.11 (s, 1H), 8.06 (dd, J=1.0, 0.5 Hz, 1H), 7.53 (dd, J=1.0, 2.5 Hz, 1H), 7.19 (t, J=7.8 Hz, 1H), 6.86 (dd, J=1.0, 2.7 Hz, 2H), 6.48 (d, J=8.5 Hz, 1H), 4.16-4.12 (m, 2H), 4.05-4.01 (m, 2H), 3.88-3.72 (m, 1H), 3.36-3.24 (m, 2H), 2.88-2.79 (m, 5H), 2.37-2.34 (m, 2H), 1.11 (t, J=7.0 Hz, 1.35H), 1.02 (t, J=7.0 Hz, 1.65H). ESI MS m/z 365 [C₂₁H₂₄N₄O₂+H]⁺.

### Synthesis of 5-(6-(4-(2-chlorobenzoyl)piperazin-1-yl)pyridin-3-yl)-3,4-dihydroquinolin-2(1H)-one

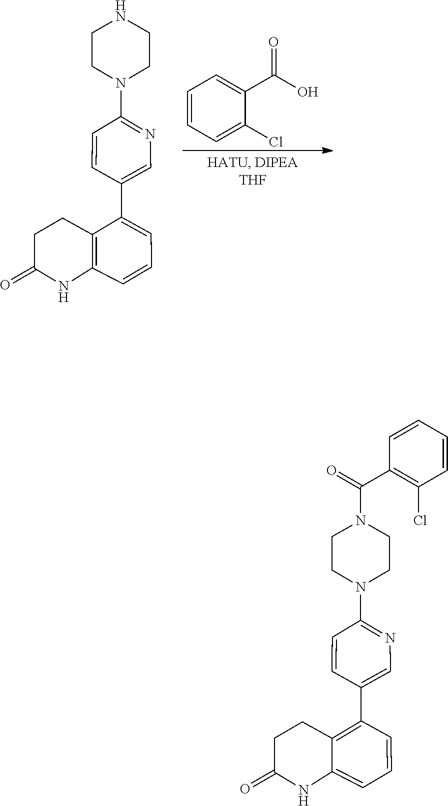

5-(6-(4-(2-Chlorobenzoyl)piperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(11)-one and 2-chlorobenzoic acid to afford 5-(6-(4-(2-chlorobenzoyl)piperazin-1-yl)pyridin-3-yl)-3,4-dihydroquinolin-2(1H)-one (44.2 mg, 61%) as an off-white solid. ¹H NMR (500 MHz, DMSO-d₆) δ 10.12 (s, 1H), 8.12 (d, J=2.5 Hz, 1H), 7.59-7.55 (m, 2H), 7.49-7.42 (m, 3H), 7.19 (t, J=8.0 Hz, 1H), 6.92 (d, J=9.0 Hz, 1H), 6.87 (d, J=8.5 Hz, 2H), 3.78 (t, J=5.0 Hz, 2H), 3.69-3.66 (m, 2H), 3.56-3.53 (m, 2H), 3.27-3.24 (m, 2H), 2.81 (t, J=7.0 Hz, 2H), 2.37-2.34 (m, 2H). ESI MS m/z 447 [C₂₅H₂₃ClN₄O₂+H]⁺.

### Synthesis of 5-(6-(4-Methacryloylpiperazin-1-yl)pyridin-3-yl)-3,4-dihydroquinolin-2(1H)-one

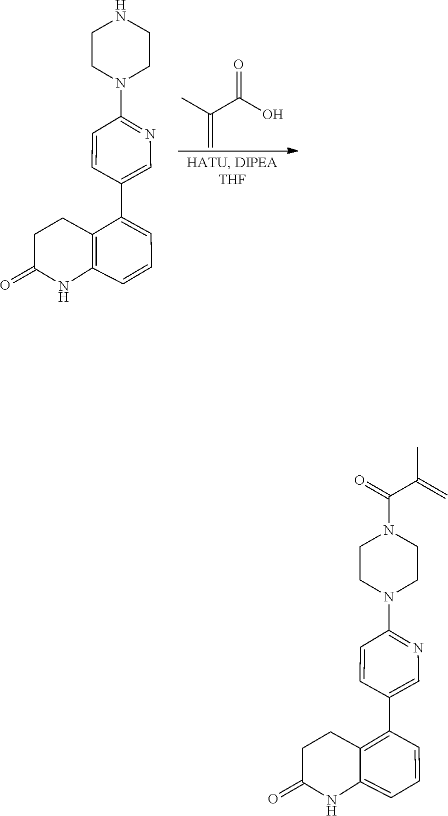

5-(6-(4-Methacryloylpiperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(1H)-one and methacrylic acid to afford 5-(6-(4-methacryloylpiperazin-1-yl)pyridin-3-yl)-3,4-dihydroquinolin-2(1H)-one (31.1 mg, 51%) as an off-white solid. ¹H NMR (500 MHz, DMSO-d₆) δ 10.13 (s, 1H), 8.12 (d, J=2.0 Hz, 1H), 7.58 (dd, J=1.0, 2.5 Hz, 1H), 7.20 (t, J=7.8 Hz, 1H), 6.93 (d, J=9.0 Hz, 1H), 6.88 (d, J=7.5 Hz, 2H), 5.23 (t, J=1.5 Hz, 1H), 5.04 (s, 1H), 3.59-3.57 (m, 8H), 2.82 (t, J=7.5 Hz, 2H), 2.36 (dd, J=8.3, 6.8 Hz, 2H), 1.89 (s, 3H). ESI MS m/z 377 [C₂₂H₂₄N₄O₂+H]⁺.

### Synthesis of 5-(6-(4-Isobutyrylpiperazin-1-yl)pyridin-3-yl)-3,4-dihydroquinolin-2(1H)-one

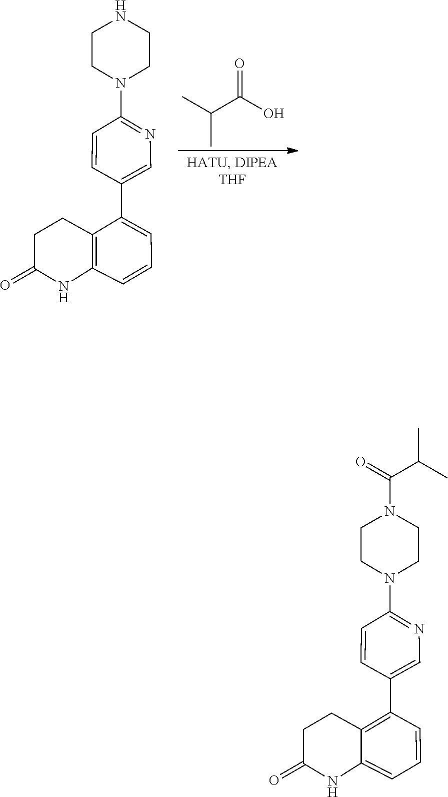

5-(6-(4-Isobutyrylpiperazin-1-yl)pyridin-3-yl)-3,4-dihydroquinolin-2(11)-one was prepared according to General Protocol 2 using 4-(5-(2-oxo-1,2,3,4-tetrahydroquinolin-5-yl)pyridin-2-yl)piperazine-1-carboxylic acid and isobutyric to afford 5-(6-(4-isobutyrylpiperazin-1-yl)pyridin-3-yl)-3,4-dihydroquinolin-2(1H)-one (33.5 mg, 55%) as an off-white solid. ¹H NMR (500 MHz, DMSO-d₆) δ 10.13 (s, 1H), 8.12 (d, J=2.5 Hz, 1H), 7.58 (dd, J=9.0, 2.5 Hz, 1H), 7.20 (t, J=7.8 Hz, 1H), 6.92 (d, J=8.5 Hz, 1H), 6.88 (d, J=8.0 Hz, 2H), 3.62-3.52 (m, 8H), 2.95-2.90 (m, 1H), 2.82 (t, J=7.5 Hz, 2H), 2.36 (dd, J=8.3, 6.8 Hz, 2H), 1.03 (d, J=7.0 Hz, 6H). ESI MS m/z 379 [C₂₂H₂₆N₄O₂+H]⁺.

### Synthesis of 5-(6-(4-(2-Methoxybenzoyl)piperazin-1-yl)pyridin-3-yl)-3,4-dihydroquinolin-2(1H)-one

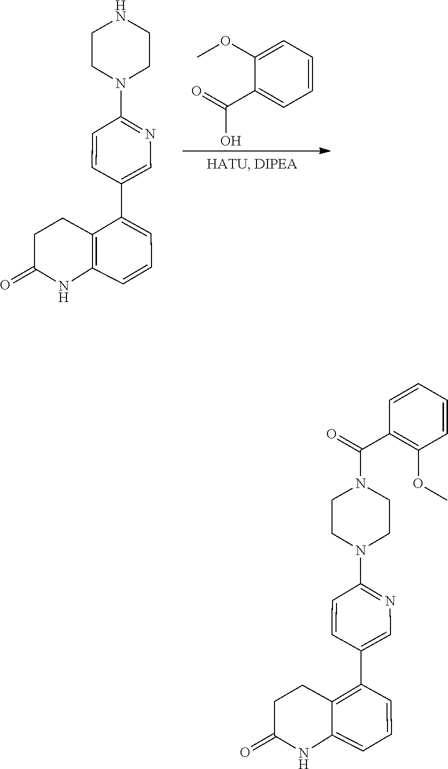 [SMILES: O=C1CCc2c(cccc2-c2ccc(N3CCNCC3)nc2)N1 ; COc1ccccc1C(=O)O ; COc1ccccc1C(=O)N1CCN(c2ccc(-c3cccc4c3CCC(=O)N4)cn2)CC1]

5-(6-(4-(2-Methoxybenzoyl)piperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(1H)-one and o-anisic acid to afford 5-(6-(4-(2-methoxybenzoyl)piperazin-1-yl)pyridin-3-yl)-3,4-dihydroquinolin-2(1H)-one (15 mg, 21%) as a white solid. ¹H NMR (300 MHz, DMSO-d₆) δ 10.15 (s, 1H), 8.12 (d, J=2.3 Hz, 1H), 7.58 (dd, J=8.7, 2.5 Hz, 1H), 7.46-7.38 (m, 1H), 7.25-6.98 (m, 4H), 6.93-6.85 (m, 3H), 3.81 (s, 3H), 3.75-3.72 (m, 2H), 3.70-3.63 (m, 2H), 3.53-3.49 (m, 2H), 3.31-3.23 (m, 2H), 2.85-2.78 (m, 2H), 2.39-2.32 (m, 2H). ESI MS m/z 443 [C₂₆H₂₆N₄O₃+H]⁺.

### Synthesis of 5-(6-(4-Cyclopropylpiperazin-1-yl)pyridin-3-yl)-3,4-dihydroquinolin-2(1H)-one

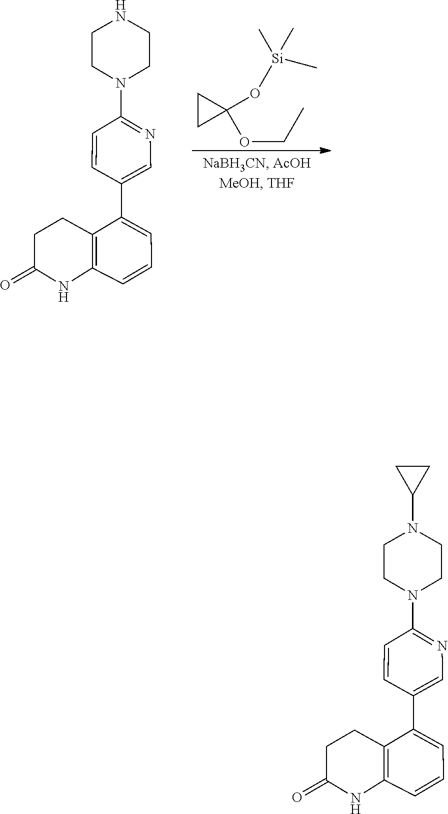 [SMILES: O=C1CCc2c(cccc2-c2ccc(N3CCNCC3)nc2)N1 ; CCOC1(O[Si](C)(C)C)CC1 ; O=C1CCc2c(cccc2-c2ccc(N3CCN(C4CC4)CC3)nc2)N1]

To a mixture of 5-(6-(piperazin-1-yl)pyridin-3-yl)-3,4-dihydroquinolin-2(1H)-one (50 mg, 0.16 mmol) and (1-ethoxycyclopropoxy)trimethylsilane (57 mg, 0.32 mmol) in MeOH (0.2 ml) and THF (2 ml) was added acetic acid (0.15 ml). The reaction was stirred at room temperature for 30 min, then sodium cyanoborohydride (20 mg, 0.32 mmol) was added. The reaction was then heated at 60° C. overnight. The reaction mixture was cooled to room temperature then filtered through Celite and concentrated. The residue was purified by column chromatography (silica gel, 0-20% MeOH/dichloromethane) to afford 5-(6-(4-cyclopropylpiperazin-1-yl)pyridin-3-yl)-3,4-dihydroquinolin-2(1H)-one (10 mg, 21%) as a white solid. ¹H NMR (300 MHz, DMSO-d₆) δ 10.14 (s, 1H), 8.09 (d, J=2.3 Hz, 1H), 7.54 (dd, J=8.7, 2.5 Hz, 1H), 7.19 (t, J=7.8 Hz, 1H), 6.90-6.84 (m, 3H), 3.49 (t, J=4.9 Hz, 4H), 2.82 (t, J=7.4 Hz, 2H), 2.63 (t, J=5.0 Hz, 4H), 2.39-2.32 (m, 2H), 1.68-1.62 (m, 1H), 0.46-0.35 (m, 3H). ESI MS m/z 349 [C₂₁H₂₄N₄O+H]⁺.

### Synthesis of tert-Butyl 5-(5-(2-oxo-1,2,3,4-tetrahydroquinolin-5-yl)pyridin-2-yl)-2,5-diazabicyclo[2.2.1]heptane-2-carboxylate

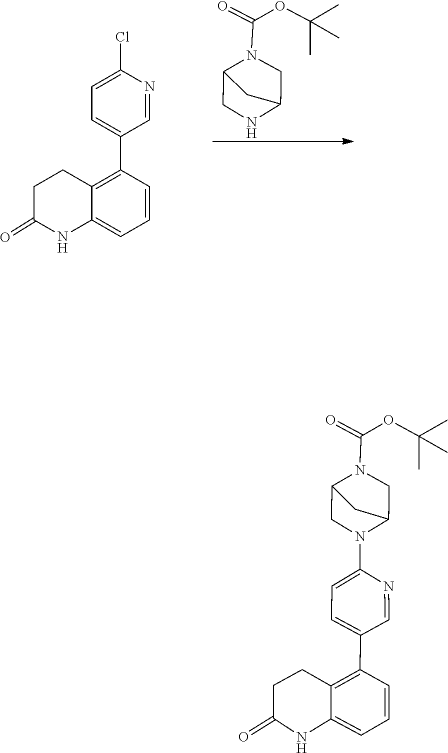 [SMILES: O=C1CCc2c(cccc2-c2ccc(Cl)nc2)N1 ; CC(C)(C)OC(=O)N1CC2CC1CN2 ; CC(C)(C)OC(=O)N1CC2CC1CN2c1ccc(-c2cccc3c2CCC(=O)N3)cn1]

A mixture of 5-(6-chloropyridin-3-yl)-3,4-dihydroquinolin-2(1H)-one (100 mg, 0.390 mmol) and tert-butyl 2,5-diazabicyclo[2.2.1]heptane-2-carboxylate (115 mg, 0.580 mmol) in DMSO (0.5 ml) was heated to 150° C. for 48 hours. The reaction was cooled to room temperature and concentrated. The residue was purified by column chromatography (silica gel, 0-20% MeOH/dichloromethane) to afford tert-butyl 5-(5-(2-oxo-1,2,3,4-tetrahydroquinolin-5-yl)pyridin-2-yl)-2,5-diazabicyclo[2.2.1]heptane-2-carboxylate (15 mg, 9%) as a white solid. ¹H NMR (300 MHz, DMSO-d₆) δ 10.16 (s, 1H), 8.05 (d, J=2.1 Hz, 1H), 7.62-7.55 (m, 1H), 7.20 (t, J=7.8 Hz, 1H), 6.88 (dd, J=7.8, 2.6 Hz, 2H), 6.80-6.66 (m, 1H), 4.87-4.83 (m, 1H), 4.52-4.44 (m, 1H), 3.58-3.51 (m, 2H), 3.30-3.20 (m, 2H), 2.83 (t, J=7.1 Hz, 2H), 2.36 (t, J=7.3 Hz, 2H), 2.00-1.85 (m, 2H), 1.38 (d, J=13.7 Hz, 9H). ESI MS m/z 421 [C₂₄H₂₈N₄O₃+H]⁺.

### Synthesis of 5-(6-(4-Benzylpiperazin-1-yl)pyridin-3-yl)-3,4-dihydroquinolin-2(1H)-one

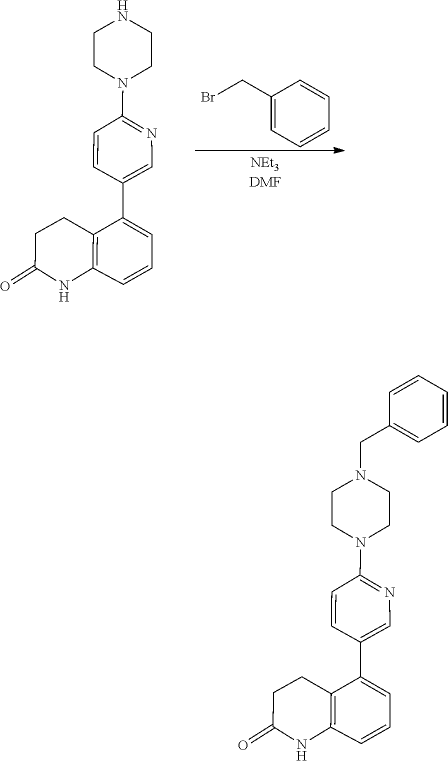 [SMILES: O=C1CCc2c(cccc2-c2ccc(N3CCNCC3)nc2)N1 ; BrCc1ccccc1 ; O=C1CCc2c(cccc2-c2ccc(N3CCN(Cc4ccccc4)CC3)nc2)N1]

To a mixture of 5-(6-(piperazin-1-yl)pyridin-3-yl)-3,4-dihydroquinolin-2(1H)-one (50 mg, 0.16 mmol) and (bromomethyl)benzene (0.029 mL, 0.24 mmol) in DMF (2 ml) was added triethylamine (0.11 ml, 0.81 mmol) at room temperature and the reaction mixture was stirred overnight. The reaction mixture was concentrated and purified by column chromatography (silica gel, 0-20% MeOH/dichloromethane) to afford 5-(6-(4-benzylpiperazin-1-yl)pyridin-3-yl)-3,4-dihydroquinolin-2(1H)-one (15 mg, 23%) as a white solid. ¹H NMR (300 MHz, DMSO-d₆) δ 10.16 (s, 1H), 8.09 (d, J=2.2 Hz, 1H), 7.54 (dd, J=8.8, 2.5 Hz, 1H), 7.40-7.13 (m, 7H), 6.90-6.84 (m, 3H), 3.60-3.40 (m, 6H), 2.90-2.71 (m, 2H), 2.51-2.43 (m, 2H), 2.38-2.33 (m, 2H). ESI MS m/z 399 [C₂₅H₂₆N₄O+H]⁺.

### Synthesis of 4-((5-(5-(2-Oxo-1,2,3,4-tetrahydroquinolin-5-yl)pyridin-2-y)-2,5-diazabicyclo[2.2.1]heptan-2-yl)sulfonyl)benzoic Acid

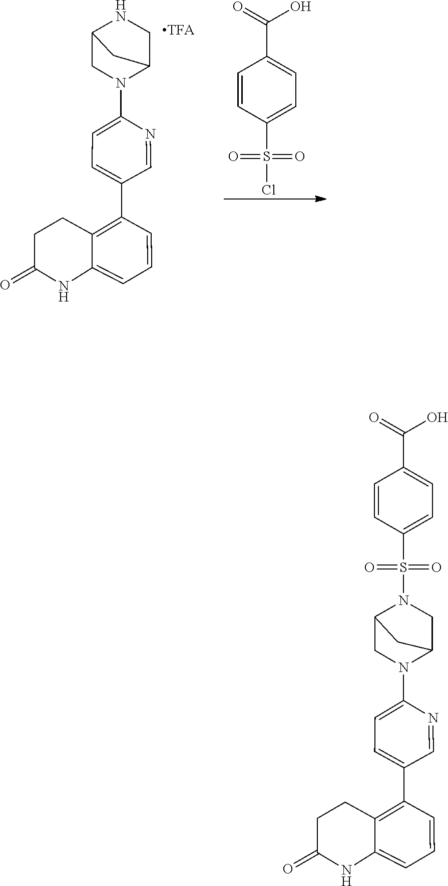 [SMILES: O=C1CCc2c(cccc2-c2ccc(N3CC4CC3CN4)nc2)N1 ; O=C(O)c1ccc(S(=O)(=O)Cl)cc1 ; O=C1CCc2c(cccc2-c2ccc(N3CC4CC3CN4S(=O)(=O)c3ccc(C(=O)O)cc3)nc2)N1]

4-((5-(5-(2-Oxo-1,2,3,4-tetrahydroquinolin-5-yl)pyridin-2-yl)-2,5-diazabicyclo[2.2.1]heptan-2-yl)sulfonyl)benzoic acid was prepared according to General Protocol 1 using 5-(6-(2,5-diazabicyclo[2.2.1]heptan-2-yl)pyridin-3-yl)-3,4-dihydroquinolin-2(1H)-one 2,2,2-trifluoroacetate and 4-(chlorosulfonyl)benzoic acid to afford 4-((5-(5-(2-oxo-1,2,3,4-tetrahydroquinolin-5-yl)pyridin-2-yl)-2,5-diazabicyclo[2.2.1]heptan-2-yl)sulfonyl)benzoic acid (58 mg, 29%) as a white solid. ¹H NMR (300 MHz, DMSO-d₆) δ 13.50 (s, 1H), 10.14 (s, 1H), 8.15-8.05 (m, 2H), 7.99-7.92 (m, 3H), 7.46 (dd, J=8.7, 2.4 Hz, 1H), 7.19 (t, J=7.8 Hz, 1H), 6.86 (d, J=7.8 Hz, 2H), 6.47 (d, J=8.5 Hz, 1H), 4.77 (s, 1H), 4.62 (s, 1H), 3.47-3.42 (m, 1H), 3.32-3.27 (m, 2H), 2.85-2.76 (m, 2H), 2.40-2.34 (m, 2H), 1.85-1.82 (m, 1H), 1.28-1.23 (m, 1H). ESI MS m/z 505 [C₂₆H₂₄N₄O₅S+H]⁺.

### Synthesis of 5-(6-(5-Propionyl-2,5-diazabicyclo[2.2.1]heptan-2-yl)pyridin-3-y)-3,4-dihydroquinolin-2(1H)-one

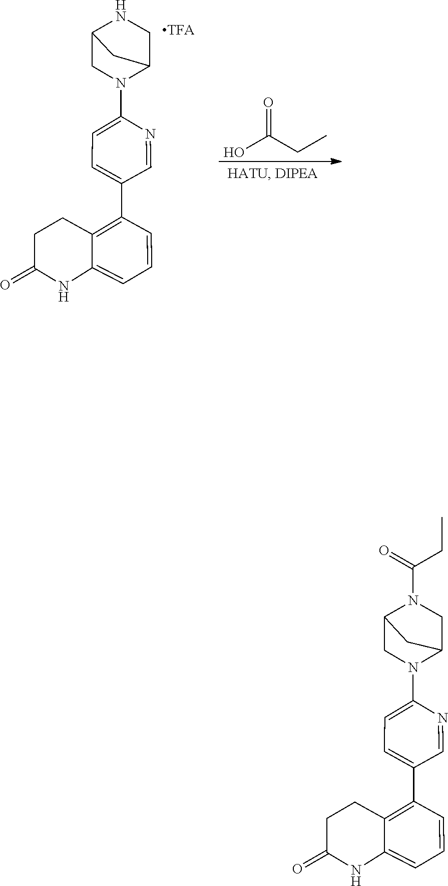

5-(6-(5-Propionyl-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one 2,2,2-trifluoroacetate and propionic acid to afford 5-(6-(5-propionyl-2,5-diazabicyclo[2.2.1]heptan-2-yl)pyridin-3-yl)-3,4-dihydroquinolin-2(1H)-one (15 mg, 35%) as a white solid. ¹H NMR (300 MHz, DMSO-d₆) δ 10.15 (s, 1H), 8.06 (d, J=2.3 Hz, 1H), 7.52 (dt, J=8.6, 2.6 Hz, 1H), 7.19 (t, J=7.8 Hz, 1H), 6.89-6.84 (m, 2H), 6.62-6.58 (m, 1H), 4.82 (t, J=35.5 Hz, 2H), 3.60-3.20 (m, 3H), 2.82 (t, J=7.3 Hz, 2H), 2.50-1.86 (m, 6H), 0.95 (dt, J=18.3, 7.4 Hz, 3H). ESI MS m/z 377 [C₂₂H₂₄N₄O₅+H]⁺.

### Synthesis of 5-(6-(5-Methacryloyl-2,5-diazabicyclo[2.2.1]heptan-2-yl)pyridin-3-y)-3,4-dihydroquinolin-2(1H)-one

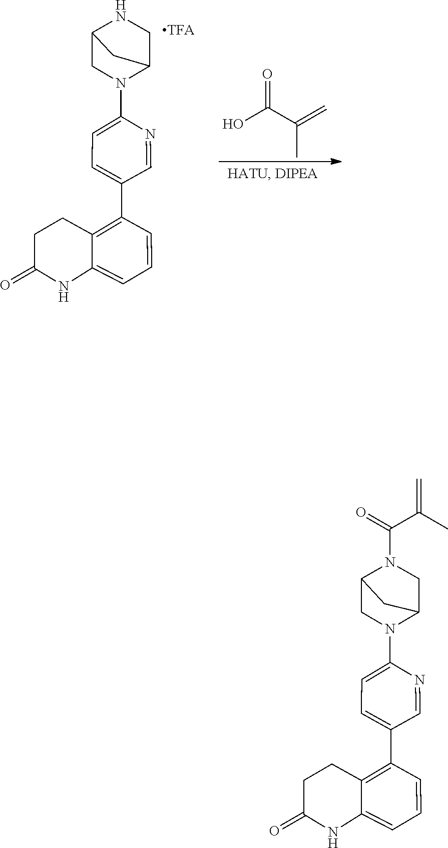

5-(6-(5-Methacryloyl-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one 2,2,2-trifluoroacetate and methacrylic acid to afford 5-(6-(5-methacryloyl-2,5-diazabicyclo[2.2.1]heptan-2-yl)pyridin-3-yl)-3,4-dihydroquinolin-2(1H)-one (20 mg, 45%) as an off-white solid. ¹H NMR (300 MHz, DMSO-d₆) δ ¹H NMR (300 MHz, CDCl3) δ 10.16 (s, 1H), 8.08-8.06 (m, 1H), 7.56-7.50 (m, 1H), 7.19 (t, J=7.6 Hz, 1H), 6.89-6.85 (m, 2H), 6.67-6.58 (m, 1H), 5.31-5.16 (m, 2H), 4.89-4.67 (m, 2H), 3.68-3.40 (m, 3H), 3.28-3.24 (m, 1H), 2.82 (t, J=7.2 Hz, 2H), 2.36 (t, J=7.5 Hz, 2H), 1.97-1.77 (m, 5H). ESI MS m/z 389 [C₂₃H₂₄N₄O₂+H]⁺.

### Synthesis of 5-(6-(3-(Piperidine-1-carbonyl)azetidin-1-yl)pyridin-3-yl)-3,4-dihydroquinolin-2(1H)-one

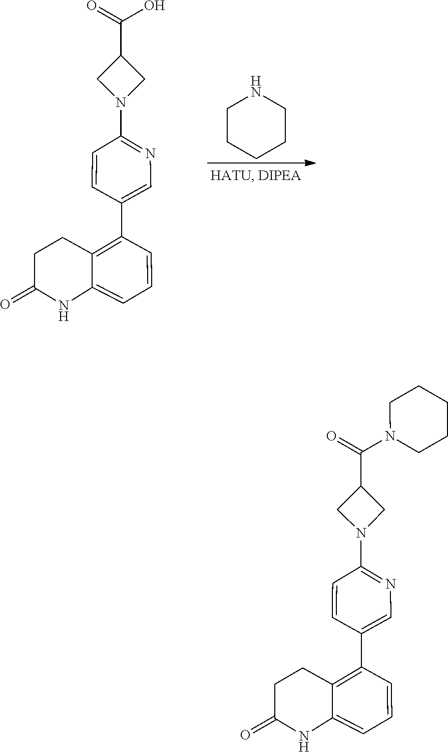 [SMILES: O=C1CCc2c(cccc2-c2ccc(N3CC(C(=O)O)C3)nc2)N1 ; C1CCNCC1 ; O=C1CCc2c(cccc2-c2ccc(N3CC(C(=O)N4CCCCC4)C3)nc2)N1]

5-(6-(3-(Piperidine-1-carbonyl)azetidin-1-yl)pyridin-3-yl)-3,4-dihydroquinolin-2(1H)-one was prepared according to General Protocol 2 using 1-(5-(2-oxo-1,2,3,4-tetrahydroquinolin-5-yl)pyridin-2-yl)azetidine-3-carboxylic acid and piperidine to afford 5-(6-(3-(piperidine-1-carbonyl)azetidin-1-yl)pyridin-3-yl)-3,4-dihydroquinolin-2(1H)-one (13 mg, 24%) as an off-white solid. ¹H NMR (500 MHz, DMSO-d₆) δ ¹H NMR (500 MHz, CDCl3) δ 10.12 (s, 1H), 8.05 (d, J=1.5 Hz, 1H), 7.54-7.53 (m, 1H), 7.19 (t, J=7.7 Hz, 1H), 6.86 (dd, J=8.0, 3.0 Hz, 2H), 6.49 (d, J=9.0 Hz, 1H), 4.16 (t, J=8.2 Hz, 2H), 4.05 (t, J=7.0 Hz, 2H), 3.86-3.81 (m, 1H), 3.46 (t, J=5.5 Hz, 2H), 2.80 (t, J=7.5 Hz, 2H), 2.37-2.34 (m, 3H), 1.59-1.57 (m, 2H), 1.50-1.44 (m, 4H). ESI MS m/z 391 [C₂₃H₂₆N₄O₂+H]⁺.

### Synthesis of 5-(6-(5-(2,6-Dimethylbenzoyl)-2,5-diazabicyclo[2.2.2]octan-2-yl)pyridin-3-yl)-3,4-dihydroquinolin-2(1H ne

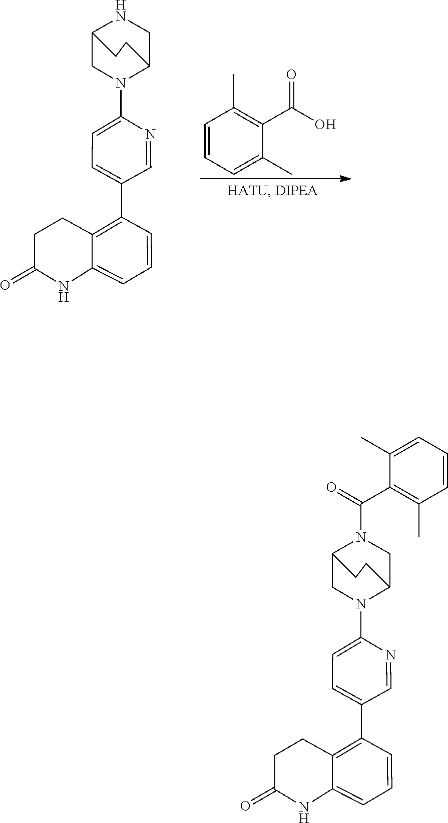 [SMILES: O=C1CCc2c(cccc2-c2ccc(N3CC4CCC3CN4)nc2)N1 ; Cc1cccc(C)c1C(=O)O ; Cc1cccc(C)c1C(=O)N1CC2CCC1CN2c1ccc(-c2cccc3c2CCC(=O)N3)cn1]

5-(6-(5-(2,6-Dimethylbenzoyl)-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and 2,6-dimethylbenzoic acid to afford 5-(6-(5-(2,6-dimethylbenzoyl)-2,5-diazabicyclo[2.2.2]octan-2-yl)pyridin-3-yl)-3,4-dihydroquinolin-2(1H)-one (3.0 mg, 4%) as an off-white solid. ¹H NMR (500 MHz, DMSO-d₆) δ 10.11 (s, 1H), 8.06 (dd, J=16.8, 2.3 Hz, 1H), 7.54 (dd, J=8.7, 2.2 Hz, 1H), 7.22-7.16 (m, 2H), 7.12-7.03 (m, 2H), 6.88-6.85 (m, 2H), 6.63-6.61 (m, 1H), 4.99-4.78 (m, 1H), 3.77-3.74 (m, 2H), 3.69-3.63 (m, 1H), 3.52 (s, 1H), 3.48-3.46 (m, 1H), 3.17-3.11 (m, 1H), 2.84-2.80 (m, 2H), 2.36 (t, J=7.5 Hz, 2H), 2.23-2.17 (m, 5H), 2.04-1.85 (m, 4H). ESI MS m/z 467 [C₂₉H₃₀N₄O₂+H]⁺.

### Synthesis of 5-(6-(4-(2,6-Dimethylbenzoyl)piperazin-1-yl)pyridin-3-yl)-3,4-dihydroquinolin-2(1H)-one

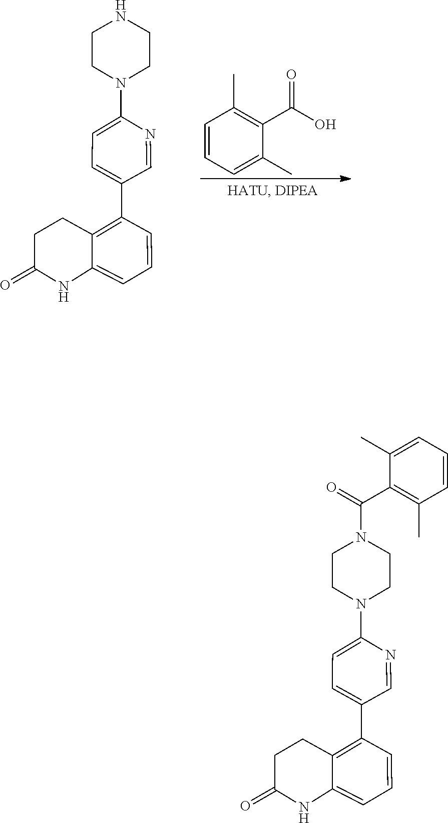 [SMILES: O=C1CCc2c(cccc2-c2ccc(N3CCNCC3)nc2)N1 ; Cc1cccc(C)c1C(=O)O ; Cc1cccc(C)c1C(=O)N1CCN(c2ccc(-c3cccc4c3CCC(=O)N4)cn2)CC1]

5-(6-(4-(2,6-Dimethylbenzoyl)piperazin-1-yl)pyridin-3-yl)-3,4-dihydroquinolin-2(1H)-one was prepared according to General Protocol 2 using 5-(6-(piperazin-1-yl)pyridin-3-yl)-3,4-dihydroquinolin-2(1H)-one and 2,6-dimethylbenzoic acid to afford 5-(6-(4-(2,6-dimethylbenzoyl)piperazin-1-yl)pyridin-3-yl)-3,4-dihydroquinolin-2(1H)-one (17 mg, 16%) as an off-white solid. ¹H NMR (500 MHz, DMSO-d₆) δ 10.12 (s, 1H), 8.12 (d, J=2.0 Hz, 1H), 7.58 (dd, J=9.0, 2.5 Hz, 1H), 7.21-7.18 (m, 2H), 7.10-7.09 (m, 2H), 6.92 (d, J=9.0 Hz, 1H), 6.87 (d, J=8.0 Hz, 2H), 3.82-3.80 (m, 2H), 3.68-3.66 (m, 2H), 3.51-3.49 (m, 2H), 3.22-3.20 (m, 2H), 2.81 (t, J=7.5 Hz, 2H), 2.36 (t, J=7.2 Hz, 2H), 2.19 (s, 6H). ESI MS m/z 441 [C₂₇H₂₈N₄O₂+H]⁺.

### Synthesis of 5-(6-(5-Benzoyl-2,5-diazabicyclo[2.2.2]octan-2-yl)pyridin-3-yl)-3,4-dihydroquinolin-2(1H)-one

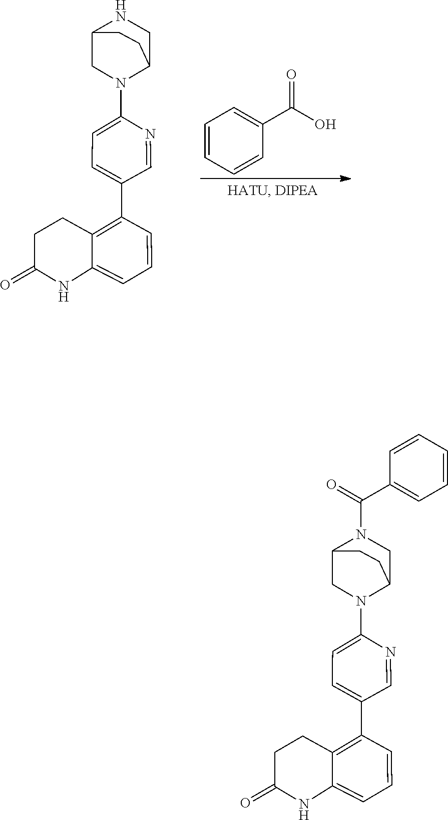 [SMILES: O=C1CCc2c(cccc2-c2ccc(N3CC4CCC3CN4)nc2)N1 ; O=C(O)c1ccccc1 ; O=C1CCc2c(cccc2-c2ccc(N3CC4CCC3CN4C(=O)c3ccccc3)nc2)N1]

5-(6-(5-Benzoyl-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and benzoic acid to afford 5-(6-(5-benzoyl-2,5-diazabicyclo[2.2.2]octan-2-yl)pyridin-3-yl)-3,4-dihydroquinolin-2(1H)-one (38 mg, 32%) as an off-white solid. ¹H NMR (500 MHz, DMSO-d₆) δ ¹H NMR (500 MHz, CDCl3) δ 10.11 (s, 1H), 8.08-8.04 (m, 1H), 7.56-7.54 (m, 1H), 7.49-7.42 (m, 5H), 7.20-7.17 (m, 1H), 6.88-6.85 (m, 2H), 6.62-6.59 (m, 1H), 5.00-4.74 (m, 1H), 3.96 (s, 1H), 3.75-3.44 (m, 4H), 2.83 (t, J=7.7 Hz, 2H), 2.36 (t, J=7.5 Hz, 2H), 2.12-1.74 (m, 4H). ESI MS m/z 439 [C₂₇H₂₆N₄O₂+H]⁺.

### Synthesis of 5-(6-(5-Methacryloyl-2,5-diazabicyclo[2.2.2]octan-2-yl)pyridin-3-y)-3,4-dihydroquinolin-2(1H)-one

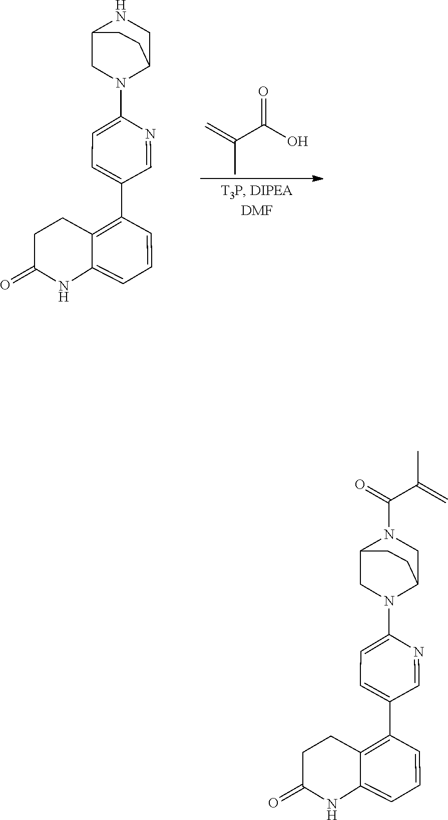

To a solution of 5-(6-(2,5-diazabicyclo[2.2.2]octan-2-yl)pyridin-3-yl)-3,4-dihydroquinolin-2(1H)-one (0.046 g, 0.14 mmol) in DMF (1 mL) was added methacrylic acid (0.014 mL, 0.16 mmol) and N,N-diisopropylethylamine (0.072 mL, 0.41 mmol) followed by addition of 2,4,6-tripropyl-1,3,5,2,4,6-trioxatriphosphinane 2,4,6-trioxide (T₃P, 0.131 g, 0.41 mmol, 50 wt. % in ethyl acetate) at room temperature. The reaction was stirred overnight then purified directly on C18 reverse phase chromatography (15 g, 0-40% acetonitrile:water no TFA) and lyophilized to afford 5-(6-(5-methacryloyl-2,5-diazabicyclo[2.2.2]octan-2-yl)pyridin-3-yl)-3,4-dihydroquinolin-2(1H)-one (14 mg, 25%) as an off-white solid. ¹H NMR (500 MHz, DMSO-d₆) δ 10.11 (s, 1H), 8.07 (d, J=2.0 Hz, 1H), 7.55 (dd, J=9.0, 2.5 Hz, 1H), 7.19 (t, J=8.0 Hz, 1H), 6.88-6.85 (m, 2H), 6.60 (d, J=8.5 Hz, 1H), 5.23-5.22 (m, 1H), 5.06 (s, 1H), 4.94-4.62 (m, 1H), 4.27 (s, 1H), 3.68-3.51 (m, 4H), 2.82 (t, J=7.3 Hz, 2H), 2.36 (t, J=7.5 Hz, 2H), 2.00-1.84 (m, 7H). ESI MS m/z 403 [C₂₄H₂₆N₄O₂+H]⁺.

### Synthesis of 5-(6-(5-Isobutyryl-2,5-diazabicyclo[2.2.2]octan-2-yl)pyridin-3-yl)-3,4-dihydroquinolin-2(1H)-one

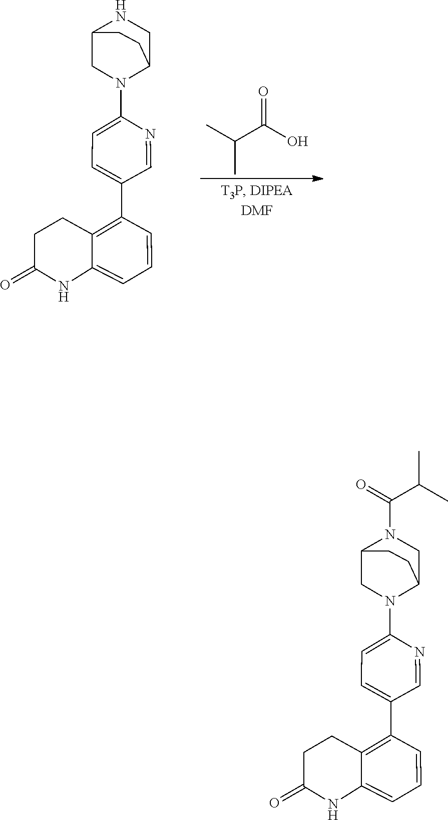

To a solution of 5-(6-(2,5-diazabicyclo[2.2.2]octan-2-yl)pyridin-3-yl)-3,4-dihydroquinolin-2(1H)-one (0.070 g, 0.21 mmol) in DMF (1 mL) was added isobutyric acid (0.022 g 0.25 mmol) and N,N-diisopropylethylamine (0.11 mL, 0.63 mmol) followed by addition of 2,4,6-tripropyl-1,3,5,2,4,6-trioxatriphosphinane 2,4,6-trioxide (T₃P, 0.200 g, 0.63 mmol, 50 wt. % in ethyl acetate) at room temperature. The reaction was stirred for 2 hours then purified directly on C18 reverse phase chromatography (15 g, 0-40% acetonitrile:water no TFA) and lyophilized to afford 5-(6-(5-isobutyryl-2,5-diazabicyclo[2.2.2]octan-2-yl)pyridin-3-yl)-3,4-dihydroquinolin-2(1H)-one (10 mg, 12%) as an off-white solid. ¹H NMR (500 MHz, DMSO-d₆) δ ¹H NMR (500 MHz, CDCl3) δ 10.11 (s, 1H), 8.06 (d, J=2.0 Hz, 1H), 7.55-7.53 (m, 1H), 7.19 (t, J=7.7 Hz, 1H), 6.87-6.85 (m, 2H), 6.59 (d, J=8.5 Hz, 1H), 4.92-4.88 (m, 1H), 4.71 (s, 0.5H), 4.36 (s, 0.5H), 3.74 (s, 1H), 3.64-3.45 (m, 3H), 2.87-2.81 (m, 2H), 2.70-2.61 (m, 1H), 2.37-2.35 (m, 2H), 1.96-1.80 (m, 4H), 1.04-0.96 (m, 6H). ESI MS m/z 405 [C₂₄H₂₈N₄O₂+H]⁺.

### Synthesis of 5-(6-(5-Picolinoyl-2,5-diazabicyclo[2.2.2]octan-2-yl)pyridin-3-yl)-3,4-dihydroquinolin-2(1H)-one

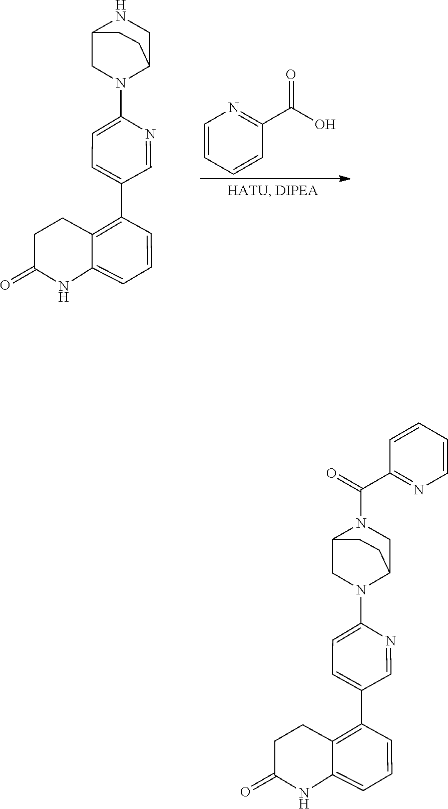 [SMILES: O=C1CCc2c(cccc2-c2ccc(N3CC4CCC3CN4)nc2)N1 ; O=C(O)c1ccccn1 ; O=C1CCc2c(cccc2-c2ccc(N3CC4CCC3CN4C(=O)c3ccccn3)nc2)N1]

5-(6-(5-Picolinoyl-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and picolinic acid to afford 5-(6-(5-picolinoyl-2,5-diazabicyclo[2.2.2]octan-2-yl)pyridin-3-yl)-3,4-dihydroquinolin-2(1H)-one (12 mg, 12%) as an off-white solid. ¹H NMR (500 MHz, DMSO-d₆) δ ¹H NMR (500 MHz, CDCl3) δ 10.11 (s, 1H), 8.64-8.62 (m, 1H), 8.08-8.08 (m, 1H), 7.96-7.94 (m, 1H), 7.68-7.66 (m, 1H), 7.56-7.50 (m, 2H), 7.20-7.17 (m, 1H), 6.89-6.86 (m, 2H), 6.62-6.59 (m, 1H), 5.00 (s, 0.6H), 4.84 (s, 0.6H), 4.36 (s, 1H), 3.83-3.63 (m, 3H), 3.50-3.47 (m, 1H), 2.84-2.81 (m, 2H), 2.37-2.35 (m, 2H), 2.13-1.77 (m, 4H). ESI MS m/z 440 [C₂₆H₂₅NO₂+H]⁺.

### Synthesis of 5-(6-(5-(Isopropylsulfonyl)-2,5-diazabicyclo[2.2.2]octan-2-yl)pyridin-3-yl)-3,4-dihydroquinolin-2(1H)-one

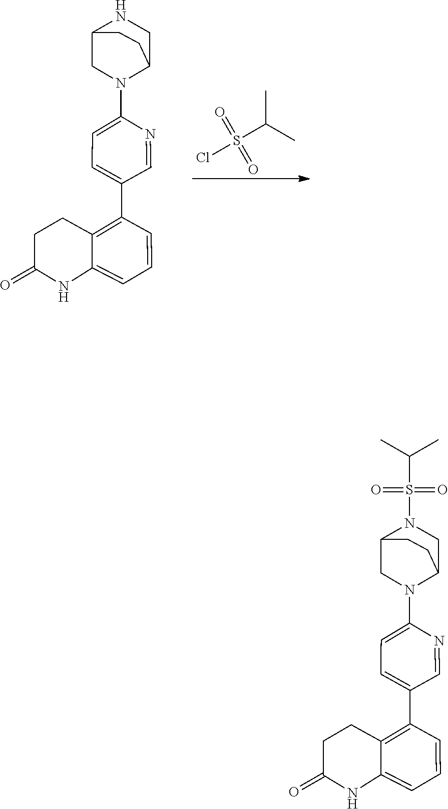

5-(6-(5-(Isopropylsulfonyl)-2,5-diazabicyclo[2.2.2]octan-2-yl)pyridin-3-yl)-3,4-dihydroquinolin-2(1H)-one was prepared according to General Protocol 1 using 5-(6-(2,5-diazabicyclo[2.2.2]octan-2-yl)pyridin-3-yl)-3,4-dihydroquinolin-2(1H)-one and propane-2-sulfonyl chloride to afford 5-(6-(5-(isopropylsulfonyl)-2,5-diazabicyclo[2.2.2]octan-2-yl)pyridin-3-yl)-3,4-dihydroquinolin-2(1H)-one (4 mg, 4%) as an off-white solid. ¹H NMR (500 MHz, DMSO-d₆) δ 10.11 (s, 1H), 8.07 (d, J=2.5 Hz, 1H), 7.79-7.74 (m, 2H), 7.74-7.72 (m, 1H), 7.68-7.65 (m, 2H), 7.54 (dd, J=9.0, 2.5 Hz, 1H), 7.17 (t, J=7.7 Hz, 1H), 6.88-6.83 (m, 3H), 3.65-3.63 (m, 4H), 3.01-2.99 (m, 4H), 2.77 (t, J=7.5 Hz, 2H), 2.34 (t, J=7.5 Hz, 2H). ESI MS m/z 441 [C₂₃H₂₈N₄O₃S+H]⁺.

### Synthesis of 5-(6-(5-(3-chlorobenzoyl)-2,5-diazabicyclo[2.2.2]octan-2-yl)pyridin-3-y)-3,4-dihydroquinolin-2(1H)-one

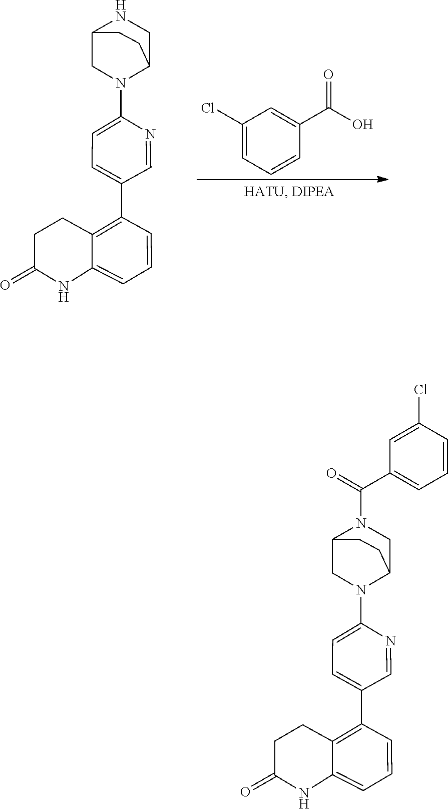 [SMILES: O=C1CCc2c(cccc2-c2ccc(N3CC4CCC3CN4)nc2)N1 ; O=C(O)c1cccc(Cl)c1 ; O=C1CCc2c(cccc2-c2ccc(N3CC4CCC3CN4C(=O)c3cccc(Cl)c3)nc2)N1]

5-(6-(5-(3-Chlorobenzoyl)-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and 3-chlorobenzoic acid to afford 5-(6-(5-(3-chlorobenzoyl)-2,5-diazabicyclo[2.2.2]octan-2-yl)pyridin-3-yl)-3,4-dihydroquinolin-2(1H)-one (26 mg, 18%) as an off-white solid. ¹H NMR (300 MHz, DMSO-d₆) δ 10.15 (s, 1H), 8.08-8.05 (m, 1H), 7.60-7.41 (m, 5H), 7.22-7.17 (m, 1H), 6.89-6.85 (m, 2H), 6.63-6.58 (m, 1H), 5.00 (s, 0.6H), 4.82-4.73 (m, 0.6H), 3.91 (s, 1H), 3.72-3.44 (m, 4H), 2.83 (t, J=7.5 Hz, 2H), 2.36 (t, J=7.3 Hz, 2H), 2.08-1.76 (m, 4H). ESI MS m/z 473 [C₂₇H₂₅ClN₄O₂+H]⁺.

### Synthesis of 5-(6-(5-(2-Chlorobenzoyl)-2,5-diazabicyclo[2.2.2]octan-2-yl)pyridin-3-yl)-3,4-dihydroquinolin-2(1H)-one

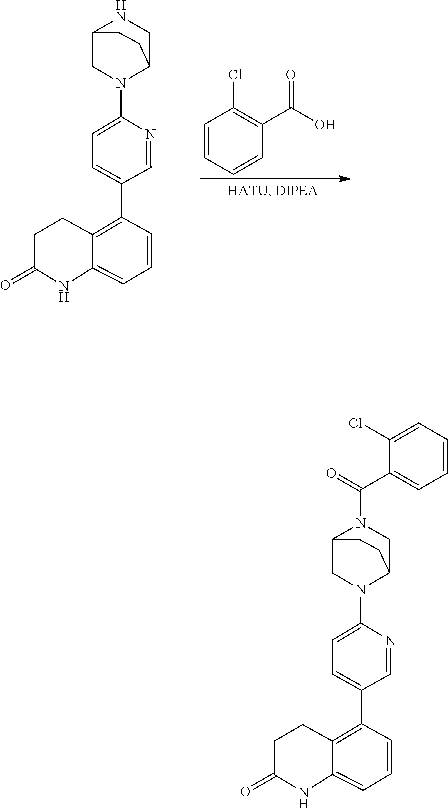 [SMILES: O=C1CCc2c(cccc2-c2ccc(N3CC4CCC3CN4)nc2)N1 ; O=C(O)c1ccccc1Cl ; O=C1CCc2c(cccc2-c2ccc(N3CC4CCC3CN4C(=O)c3ccccc3Cl)nc2)N1]

5-(6-(5-(2-Chlorobenzoyl)-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and 2-chlorobenzoic acid to afford 5-(6-(5-(2-chlorobenzoyl)-2,5-diazabicyclo[2.2.2]octan-2-yl)pyridin-3-yl)-3,4-dihydroquinolin-2(1H)-one (26 mg, 18%) as an off-white solid. ¹H NMR (300 MHz, DMSO-d₆) δ ¹H NMR (300 MHz, CDCl3) δ 10.14 (s, 1H), 8.08-8.08 (m, 1H), 7.56-7.53 (m, 2H), 7.42 (d, J=8.7 Hz, 1H), 7.19-7.17 (m, 1H), 7.03-6.98 (m, 2H), 6.89-6.85 (m, 2H), 6.60 (d, J=9.0 Hz, 1H), 4.98 (s, 0.6H), 4.81-4.68 (m, 0.6H), 4.05 (s, 1H), 3.65-3.44 (m, 4H), 2.83 (t, J=7.5 Hz, 2H), 2.36 (t, J=7.5 Hz, 2H), 2.08-1.75 (m, 4H). ESI MS m/z 473 [C₂₇H₂₅ClN₄O₂+H]⁺.

### Synthesis of 5-(6-(5-(3-fluorobenzoyl)-2,5-diazabicyclo[2.2.1]heptan-2-yl)pyridin-3-yl)-3,4-dihydroquinolin-2(1H)-one

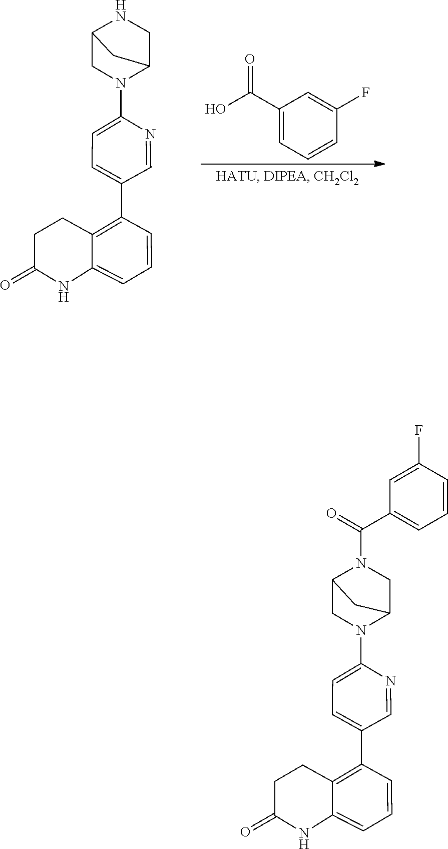 [SMILES: O=C1CCc2c(cccc2-c2ccc(N3CC4CC3CN4)nc2)N1 ; O=C(O)c1cccc(F)c1 ; O=C1CCc2c(cccc2-c2ccc(N3CC4CC3CN4C(=O)c3cccc(F)c3)nc2)N1]

5-(6-(5-(3-Fluorobenzoyl)-2,5-diazabicyclo[2.2.1]heptan-2-yl)pyridin-3-yl)-3,4-dihydroquinolin-2(1H)-one was prepared according to General Protocol 2 using 3-fluorobenzoic acid and 5-(6-(2,5-diazabicyclo[2.2.1]heptan-2-yl)pyridin-3-yl)-3,4-dihydroquinolin-2(1H)-one to afford 5-(6-(5-(3-fluorobenzoyl)-2,5-diazabicyclo[2.2.1]heptan-2-yl)pyridin-3-yl)-3,4-dihydroquinolin-2(1H)-one (10 mg, 15%) as a white solid. ¹H NMR (500 MHz, DMSO-d₆) δ 10.11 (d, J=10.0 Hz, 1H), 8.05 (dd, J=35.8, 2.3 Hz, 1H), 7.59-7.53 (m, 2H), 7.59-7.33 (m, 3H), 7.20-7.17 (m, 1H), 6.90-6.84 (m, 2H), 6.62 (dd, J=35.0, 10.0 Hz, 1H), 4.95-4.87 (m, 1.5H), 4.43 (s, 0.5H), 3.80-3.20 (m, 3H), 2.86-2.75 (m, 2H), 2.40-2.31 (m, 2H), 2.15-1.85 (m, 2H). ESI MS m/z 443 [C₂₆H₂₃FN₄O₂+H]⁺.

### Synthesis of 5-(6-(5-(4-fluorobenzoyl)-2,5-diazabicyclo[2.2.1]heptan-2-yl)pyridin-3-yl)-3,4-dihydroquinolin-2(1H)-one

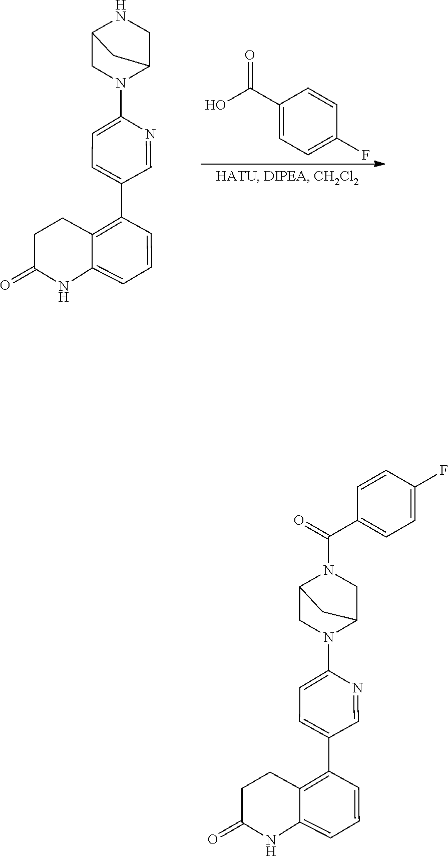 [SMILES: O=C1CCc2c(cccc2-c2ccc(N3CC4CC3CN4)nc2)N1 ; O=C(O)c1ccc(F)cc1 ; O=C1CCc2c(cccc2-c2ccc(N3CC4CC3CN4C(=O)c3ccc(F)cc3)nc2)N1]

5-(6-(5-(4-Fluorobenzoyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one and 4-fluorobenzoic acid to afford 5-(6-(5-(4-fluorobenzoyl)-2,5-diazabicyclo[2.2.1]heptan-2-yl)pyridin-3-yl)-3,4-dihydroquinolin-2(1H)-one (30 mg, 43%) as a white solid. ¹H NMR (500 MHz, DMSO-d₆) δ 10.11 (d, J=9.1 Hz, 1H), 8.05 (dd, J=36.9, 1.9 Hz, 1H), 7.63-7.45 (m, 3H), 7.30 (t, J=8.8 Hz, 1H), 7.25-7.14 (m, 2H), 6.90-6.82 (m, 2H), 6.62 (dd, J=37.1, 8.6 Hz, 1H), 4.93 (s, 1H), 4.89 (s, 0.5H), 4.45 (s, 0.5H), 3.77 (d, J=8.5 Hz, 0.5H), 3.62 (t, J=11.7 Hz, 1H), 3.55 (s, 1H), 3.45 (dd, J=33.2, 10.5 Hz, 1H), 3.26 (d, J=9.1 Hz, 0.5H), 2.87-2.75 (m, 2H), 2.38-2.30 (m, 2H), 2.10-1.91 (m, 2H). ESI MS m/z 443 [C₂₆H₂₃FN₄O₂+H]⁺.

### Synthesis of 5-(6-(5-(4-Fluorobenzoyl)-2,5-diazabicyclo[2.2.2]octan-2-yl)pyridin-3-yl)-3,4-dihydroquinolin-2(1H)-one

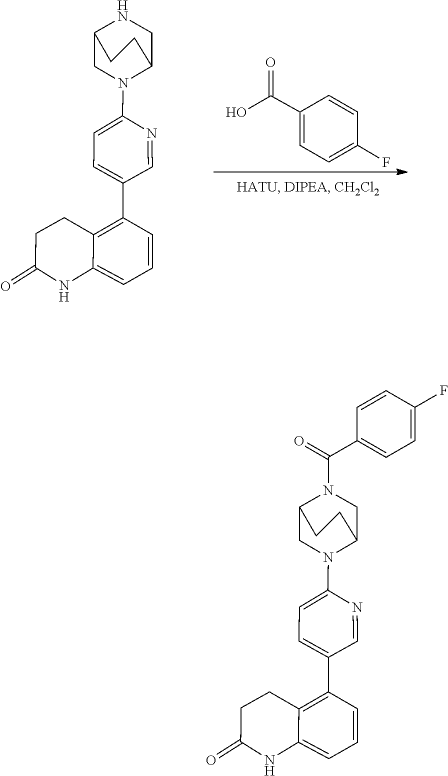 [SMILES: O=C1CCc2c(cccc2-c2ccc(N3CC4CCC3CN4)nc2)N1 ; O=C(O)c1ccc(F)cc1 ; O=C1CCc2c(cccc2-c2ccc(N3CC4CCC3CN4C(=O)c3ccc(F)cc3)nc2)N1]

5-(6-(5-(4-Fluorobenzoyl)-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and 4-fluorobenzoic acid to afford 5-(6-(5-(4-fluorobenzoyl)-2,5-diazabicyclo[2.2.2]octan-2-yl)pyridin-3-yl)-3,4-dihydroquinolin-2(1H)-one (33 mg, 35%) as a white solid. ¹H NMR (500 MHz, DMSO-d₆) δ 10.12 (s, 1H), 8.08 (d, J=2.3 Hz, 0.7H), 8.04 (d, J=2.2 Hz, 0.3H), 7.64-7.49 (m, 3H), 7.34-7.16 (m, 3H), 6.89-6.84 (m, 2H), 6.60 (t, J=8.2 Hz, 1H), 4.99 (s, 0.7H), 4.81 (s, 3H), 4.72 (s, 0.3H), 3.96 (s, 0.7H), 3.80-3.40 (m, 4H), 2.82 (q, J=6.8 Hz, 2H), 2.45 (t, J=7.5 Hz, 2H), 2.20-1.70 (m, 4H). ESI MS m/z 457 [C₂₇H₂₅FN₄O₂+H]⁺.

### Synthesis of 5-(6-(5-(3-Fluorobenzoyl)-2,5-diazabicyclo[2.2.2]octan-2-yl)pyridin-3-yl)-3,4-dihydroquinolin-2(1H)-one

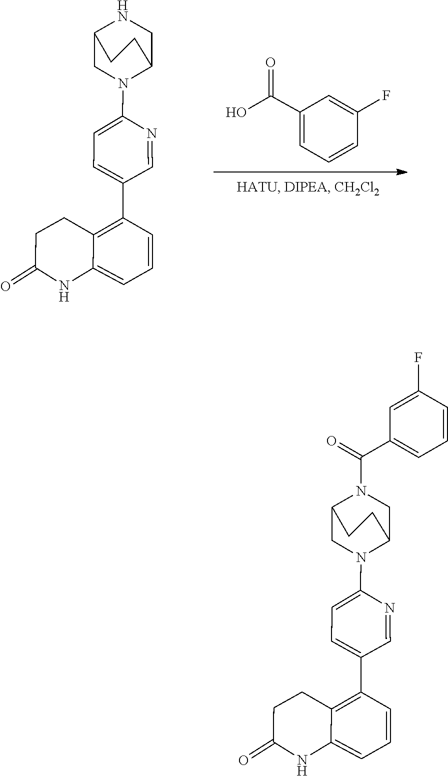 [SMILES: O=C1CCc2c(cccc2-c2ccc(N3CC4CCC3CN4)nc2)N1 ; O=C(O)c1cccc(F)c1 ; O=C1CCc2c(cccc2-c2ccc(N3CC4CCC3CN4C(=O)c3cccc(F)c3)nc2)N1]

5-(6-(5-(3-Fluorobenzoyl)-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and 3-fluorobenzoic acid to afford 5-(6-(5-(3-fluorobenzoyl)-2,5-diazabicyclo[2.2.2]octan-2-yl)pyridin-3-yl)-3,4-dihydroquinolin-2(1H)-one (41.8 mg, 44%) as a white solid. ¹H NMR (500 MHz, DMSO-d₆) δ 10.12 (s, 1H), 8.06 (dd, J=16.4, 2.1 Hz, 1H), 7.56-7.45 (m, 2H), 7.40-7.27 (m, 3H), 7.21-7.16 (m, 1H), 6.89-6.84 (m, 2H), 6.60 (t, J=7.6 Hz, 1H), 5.00-4.80 (m, 1H), 4.74 (s, 0.3H), 3.92 (s, 0.7H), 3.78-3.40 (m, 4H), 2.82 (q, J=7.0 Hz, 2H), 2.39-2.33 (m, 2H), 2.12-1.71 (m, 4H). ESI MS m/z 457 [C₂₇H₂₅FN₄O₂+H]⁺.

### Synthesis of 5-(6-(5-(2-Fluorobenzoyl)-2,5-diazabicyclo[2.2.1]heptan-2-yl)pyridin-3-y)-3,4-dihydroquinolin-2(1H)-one

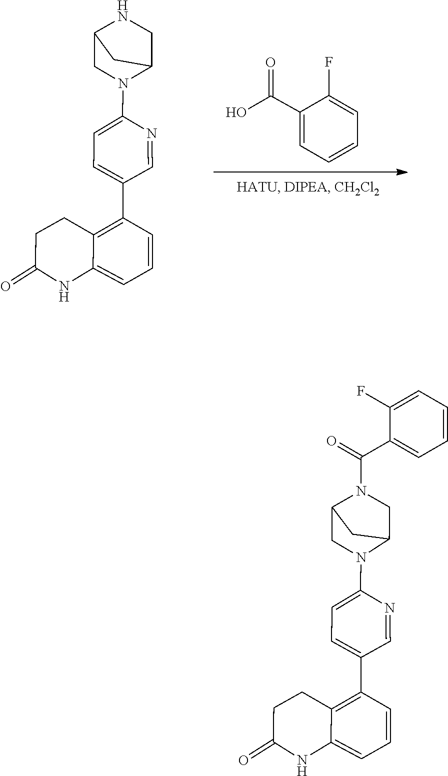 [SMILES: O=C1CCc2c(cccc2-c2ccc(N3CC4CC3CN4)nc2)N1 ; O=C(O)c1ccccc1F ; O=C1CCc2c(cccc2-c2ccc(N3CC4CC3CN4C(=O)c3ccccc3F)nc2)N1]

5-(6-(5-(2-Fluorobenzoyl)-2,5-diazabicyclo[2.2.1]heptan-2-yl)pyridin-3-yl)-3,4-dihydroquinolin-2(1H)-one was prepared according to General Protocol 2 using 2-fluorobenzoic acid and 5-(6-(2,5-diazabicyclo[2.2.1]heptan-2-yl)pyridin-3-yl)-3,4-dihydroquinolin-2(1H)-one to afford 5-(6-(5-(2-fluorobenzoyl)-2,5-diazabicyclo[2.2.1]heptan-2-yl)pyridin-3-yl)-3,4-dihydroquinolin-2(1H)-one (25.5 mg, 37%) as a white solid. ¹H NMR (500 MHz, DMSO-d₆) δ 10.12 (d, J=5.0 Hz, 1H), 8.09 (dd, J=2.4, 0.5 Hz, 0.7H), 8.03 (dd, J=2.5, 0.6 Hz, 0.3H), 7.55-7.18 (m, 6H), 6.90-6.84 (m, 2H), 6.65 (d, J=8.6 Hz, 0.7H), 6.60 (d, J=8.6 Hz, 0.3H), 4.94 (t, J=25.3 Hz, 1.4H), 4.21 (s, 0.6H), 3.65-3.60 (m, 1H), 3.55-3.41 (m, 2H), 3.38 (d, J=9.7 Hz, 0.7H), 3.12 (d, J=9.3 Hz, 0.3H), 2.86-2.78 (m, 2H), 2.40-2.33 (m, 2H), 2.05-1.95 (m, 2H). ESI MS m/z 443 [C₂₆H₂₃FN₄O₂+H]⁺.

### Synthesis of 5-(6-(5-(2-Fluorobenzoyl)-2,5-diazabicyclo[2.2.2]octan-2-yl)pyridin-3-yl)-3,4-dihydroquinolin-2(1H)-one

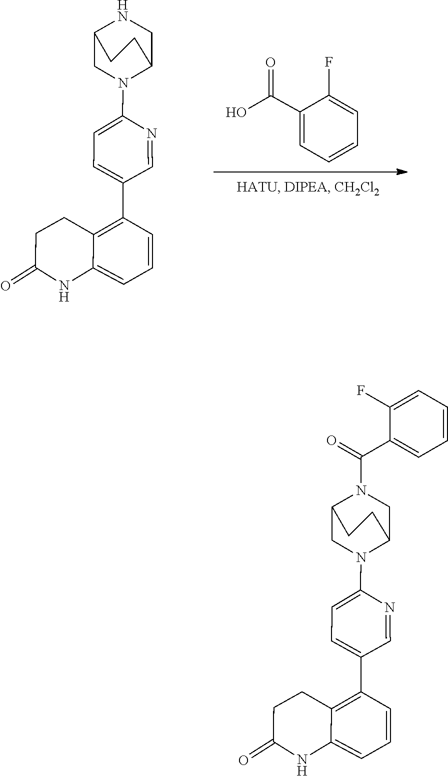 [SMILES: O=C1CCc2c(cccc2-c2ccc(N3CC4CCC3CN4)nc2)N1 ; O=C(O)c1ccccc1F ; O=C1CCc2c(cccc2-c2ccc(N3CC4CCC3CN4C(=O)c3ccccc3F)nc2)N1]

5-(6-(5-(4-Fluorobenzoyl)-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and 2-fluorobenzoic acid to afford 5-(6-(5-(4-fluorobenzoyl)-2,5-diazabicyclo[2.2.2]octan-2-yl)pyridin-3-yl)-3,4-dihydroquinolin-2(1H)-one (16.6 mg, 20%) as a white solid. ¹H NMR (500 MHz, DMSO-d₆) δ 10.12 (s, 1H), 8.06 (dd, J=17.4, 2.1 Hz, 1H), 7.59-7.40 (m, 3H), 7.36-7.26 (m, 2H), 7.22-7.16 (m, 1H), 6.90-6.86 (m, 2H), 6.65-6.58 (m, 1H), 4.91 (d, J=86.9 Hz, 1H), 3.80-3.40 (m, 4H), 2.82 (q, J=6.9 Hz, 2H), 2.39-2.34 (m, 2H), 2.10-1.75 (m, 2H). ESI MS m/z 457 [C₂₇H₂₅FN₄O₂+H]⁺.

### Synthesis of 5-(6-(5-(2-Methoxybenzoyl)-2,5-diazabicyclo[2.2.1]heptan-2-yl)pyridin-3-yl)-3,4-dihydroquinolin-2(1H)-one

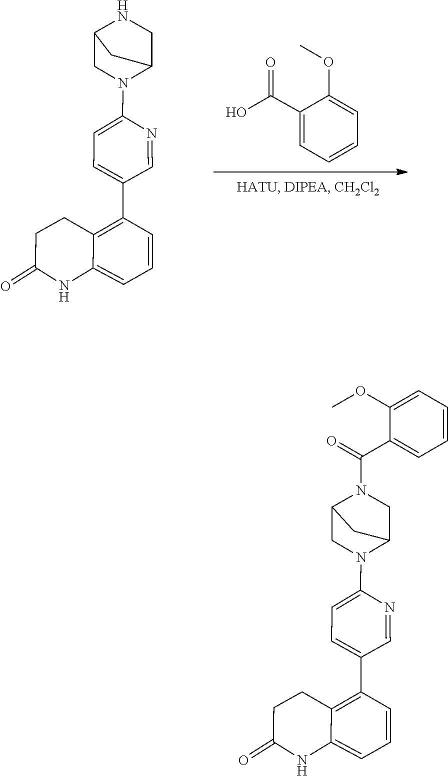 [SMILES: O=C1CCc2c(cccc2-c2ccc(N3CC4CC3CN4)nc2)N1 ; COc1ccccc1C(=O)O ; COc1ccccc1C(=O)N1CC2CC1CN2c1ccc(-c2cccc3c2CCC(=O)N3)cn1]

5-(6-(5-(2-Methoxybenzoyl)-2,5-diazabicyclo[2.2.1]heptan-2-yl)pyridin-3-yl)-3,4-dihydroquinolin-2(1H)-one was prepared according to General Protocol 2 using 2-methoxybenzoic acid and 5-(6-(2,5-diazabicyclo[2.2.1]heptan-2-yl)pyridin-3-yl)-3,4-dihydroquinolin-2(1H)-one to afford 5-(6-(5-(2-methoxybenzoyl)-2,5-diazabicyclo[2.2.1]heptan-2-yl)pyridin-3-yl)-3,4-dihydroquinolin-2(1H)-one (39 mg, 35%) as a white solid. ¹H NMR (500 MHz, DMSO-d₆) δ 10.12 (s, 1H), 8.08 (dd, J=9.7, 2.3 Hz, 1H), 7.54 (dt, J=8.6, 2.4 Hz, 1H), 7.46-7.31 (m, 1H), 7.25 (dd, J=7.4, 1.7 Hz, 0.5H), 7.21-7.16 (m, 1H), 7.14-7.10 (m, 1H), 7.05-6.83 (m, 3.5H), 6.63 (t, J=8.2 Hz, 1H), 4.92 (d, J=10.1 Hz, 1H), 4.78 (s, 0.4H), 4.06 (s, 0.6H), 3.83 (s, 1.7H), 3.64-3.54 (m, 1H), 3.50-3.10 (m, 4.3H), 2.86-2.78 (m, 2H), 2.40-2.33 (m, 2H), 2.10-1.90 (m, 2H). ESI MS m/z 455 [C₂₇H₂₆N₄O₃+H]⁺.

### Synthesis of 5-(6-(5-(2-Chloroacryloyl)-2,5-diazabicyclo[2.2.1]heptan-2-yl)pyridin-3-y)-3,4-dihydroquinolin-2(1H)-one

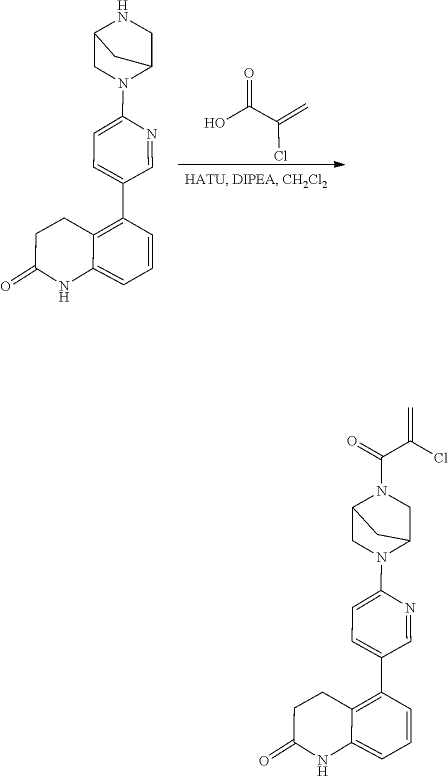

5-(6-(5-(2-Chloroacryloyl)-2,5-diazabicyclo[2.2.1]heptan-2-yl)pyridin-3-yl)-3,4-dihydroquinolin-2(1H)-one was prepared according to General Protocol 2 using 2-chloroacrylic acid and 5-(6-(2,5-diazabicyclo[2.2.1]heptan-2-yl)pyridin-3-yl)-3,4-dihydroquinolin-2(1H)-one to afford 5-(6-(5-(2-chloroacryloyl)-2,5-diazabicyclo[2.2.1]heptan-2-yl)pyridin-3-yl)-3,4-dihydroquinolin-2(1H)-one (20 mg, 26%) as a white solid. ¹H NMR (500 MHz, DMSO-d₆) δ 10.11 (s, 1H), 8.07 (dd, J=9.1, 2.1 Hz, 1H), 7.56-7.50 (m, 1H), 7.19 (t, J=7.8 Hz, 1H), 6.89-6.84 (m, 2H), 6.66-6.59 (m, 1H), 5.92-5.79 (m, 3H), 4.95-4.70 (m, 2H), 3.76-3.42 (m, 3H), 3.40-3.30 (m, 1H), 2.82 (t, J=7.4 Hz, 2H), 2.38-2.33 (m, 2H), 2.05-1.97 (m, 2H). ESI MS m/z 409 [C₂₂H₂₁ClN₄O₂+H]⁺.

### Synthesis of 5-(6-(4-(2-Chloroacryloyl)piperazin-1-yl)pyridin-3-yl)-3,4-dihydroquinolin-2(1H)-one

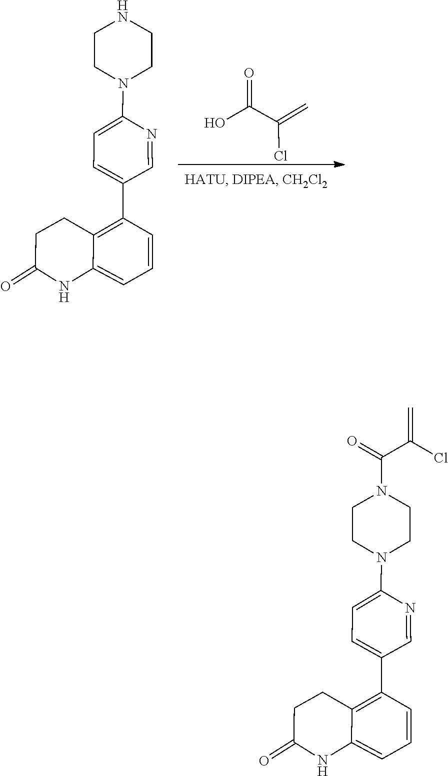

5-(6-(4-(2-Chloroacryloyl)piperazin-1-yl)pyridin-3-yl)-3,4-dihydroquinolin-2(1H)-one was prepared according to General Protocol 2 using 2-chloroacrylic acid and 5-(6-(piperazin-1-yl)pyridin-3-yl)-3,4-dihydroquinolin-2(1H)-one (80 mg, 0.259 mmol to afford 5-(6-(4-(2-chloroacryloyl)piperazin-1-yl)pyridin-3-yl)-3,4-dihydroquinolin-2(1H)-one (10 mg, 10%) as a white solid. ¹H NMR (500 MHz, DMSO-d₆) δ 10.13 (s, 1H), 8.13 (dd, J=2.5, 0.5 Hz, 1H), 7.59 (dd, J=8.7, 2.5 Hz, 1H), 7.20 (t, J=7.8 Hz, 1H), 6.96-6.86 (m, 3H), 5.81 (s, 2H), 3.62 (s, 8H), 2.82 (t, J=7.5 Hz, 2H), 2.39-2.34 (m, 2H). ESI MS m/z 397 [C₂₁H₂₁ClN₄O₂+H]⁺.

### Synthesis of N-Isopropyl-N-methyl-1-(5-(2-oxo-1,2,3,4-tetrahydroquinolin-5-yl)pyridin-2-yl)azetidine-3-carboxamide

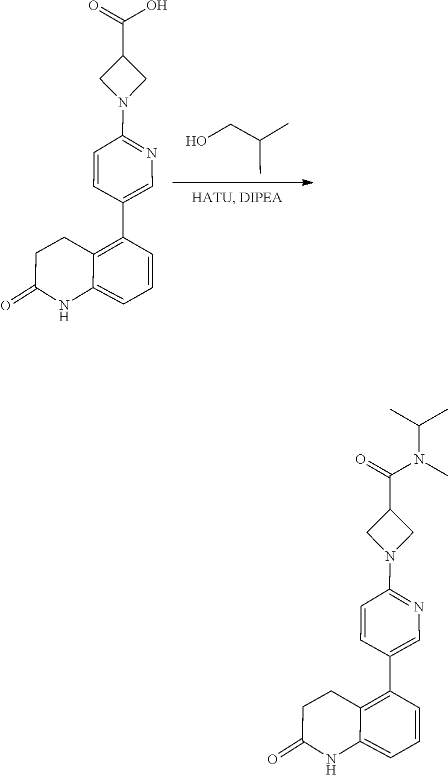

N-Isopropyl-N-methyl-1-(5-(2-oxo-1,2,3,4-tetrahydroquinolin-5-yl)pyridin-2-yl)azetidine-3-carboxamide was prepared according to General Protocol 2 using 1-(5-(2-oxo-1,2,3,4-tetrahydroquinolin-5-yl)pyridin-2-yl)azetidine-3-carboxylic acid and N-methylisopropylamine to afford N-isopropyl-N-methyl-1-(5-(2-oxo-1,2,3,4-tetrahydroquinolin-5-yl)pyridin-2-yl)azetidine-3-carboxamide (58 mg, 42%) as a white solid. ¹H NMR (300 MHz, DMSO-d₆) δ 10.16 (s, 1H), 8.06 (d, J=2.1 Hz, 1H), 7.54 (dd, J=8.7, 2.1 Hz, 1H), 7.19 (t, J=7.8 Hz, 1H), 6.87 (d, J=7.8 Hz, 2H), 6.51-6.47 (m, 1H), 4.19-4.14 (m, 2H), 4.08-4.01 (m, 2H), 3.88-3.82 (m, 2H), 2.80 (t, J=7.3 Hz, 2H), 2.72-2.70 (m, 3H), 2.35 (t, J=7.5 Hz, 2H), 1.14 (d, J=6.3 Hz, 3H), 1.04 (d, J=6.6 Hz, 3H). ESI MS m/z 379 [C₂₂H₂₆N₄O₂+H]⁺.

### Synthesis of 5-(6-(5-(Vinylsulfonyl)-2,5-diazabicyclo[2.2.1]heptan-2-yl)pyridin-3-y)-3,4-dihydroquinolin-2(1H)-one

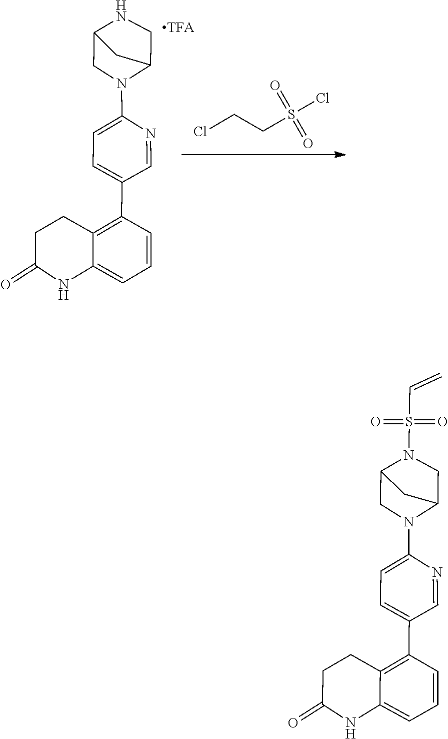 [SMILES: O=C1CCc2c(cccc2-c2ccc(N3CC4CC3CN4)nc2)N1 ; O=S(=O)(Cl)CCCl ; C=CS(=O)(=O)N1CC2CC1CN2c1ccc(-c2cccc3c2CCC(=O)N3)cn1]

5-(6-(5-(Vinylsulfonyl)-2,5-diazabicyclo[2.2.1]heptan-2-yl)pyridin-3-yl)-3,4-dihydroquinolin-2(1H)-one was prepared according to General Protocol 1 using 5-(6-(2,5-diazabicyclo[2.2.1]heptan-2-yl)pyridin-3-yl)-3,4-dihydroquinolin-2(1H)-one 2,2,2-trifluoroacetate and 2-chloroethanesulfonyl chloride to afford 5-(6-(5-(vinylsulfonyl)-2,5-diazabicyclo[2.2.1]heptan-2-yl)pyridin-3-yl)-3,4-dihydroquinolin-2(1H)-one (10 mg, 15%) as a white solid. ¹H NMR (300 MHz, DMSO-d₆) δ 10.15 (s, 1H), 8.07 (d, J=2.4 Hz, 1H), 7.54 (dd, J=8.7, 2.4 Hz, 1H), 7.19 (t, J=7.7 Hz, 1H), 6.97-6.84 (m, 3H), 6.62 (d, J=8.7 Hz, 1H), 6.14-6.08 (m, 2H), 4.90 (s, 1H), 4.43 (s, 1H), 3.55-3.43 (m, 2H), 3.31-3.21 (m, 2H), 2.83 (t, J=7.4 Hz, 2H), 2.36 (t, J=7.5 Hz, 2H), 1.97-1.81 (m, 2H). ESI MS m/z 411 [C₂₁H₂₂N₄O₃S+H]⁺.

### Synthesis of 5-(6-(5-(2,6-Dimethylbenzoyl)-2,5-diazabicyclo[2.2.1]heptan-2-yl)pyridin-3-yl)-3,4-dihydroquinolin-2(1H)-one

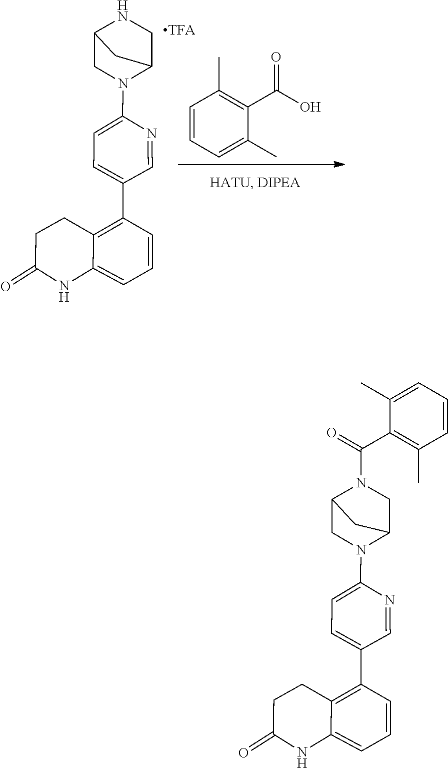

5-(6-(5-(2,6-Dimethylbenzoyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one 2,2,2-trifluoroacetate and 2,6-dimethylbenzoic acid to afford 5-(6-(5-(2,6-dimethylbenzoyl)-2,5-diazabicyclo[2.2.1]heptan-2-yl)pyridin-3-yl)-3,4-dihydroquinolin-2(1H)-one (24 mg, 31%) as a white solid. ¹H NMR (300 MHz, DMSO-d₆) δ 10.16 (s, 1H), 8.11-8.05 (m, 1H), 7.57-7.51 (m, 1H), 7.22-6.86 (m, 6H), 6.68-6.62 (m, 1H), 4.98 (s, 1H), 4.80 (s, 1H), 3.67-3.64 (m, 1H), 3.55-3.41 (m, 2H), 3.29-3.13 (m, 2H), 2.86-2.73 (m, 2H), 2.41-2.34 (m, 2H), 2.20-2.16 (m, 4H), 2.07-1.97 (m, 2H), 1.76 (s, 2H). ESI MS m/z 453 [C₂₈H₂₈N₄O₂+H]⁺.

### Synthesis of 5-(6-(5-Benzoyl-2,5-diazabicyclo[2.2.1]heptan-2-yl)pyridin-3-yl)-3,4-dihydroquinolin-2(1H)-one

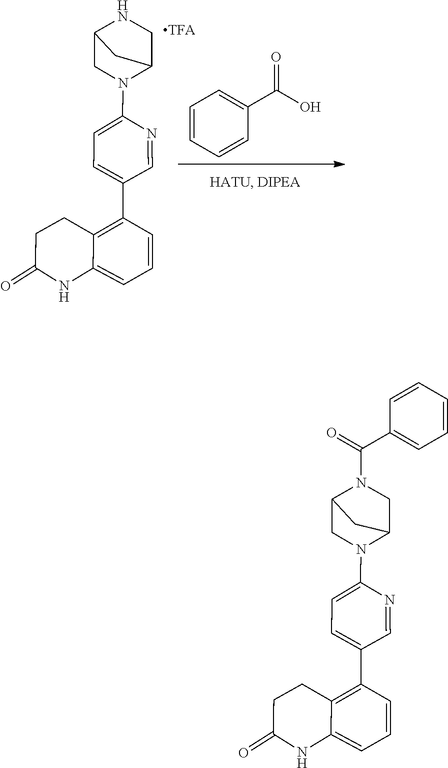

5-(6-(5-(Benzoyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one 2,2,2-trifluoroacetate and benzoic acid to afford 5-(6-(5-(benzoyl)-2,5-diazabicyclo[2.2.1]heptan-2-yl)pyridin-3-yl)-3,4-dihydroquinolin-2(1H)-one (28 mg, 38%) as a white solid. ¹H NMR (300 MHz, DMSO-d₆) δ 10.15-10.13 (m, 1H), 8.10-8.01 (m, 1H), 7.58-7.38 (m, 6H), 7.23-7.14 (m, 1H), 6.90-6.83 (m, 2H), 6.68-6.57 (m, 1H), 4.93 (s, 1H), 4.43 (s, 1H), 3.78-3.44 (m, 4H), 2.86-2.76 (m, 2H), 2.39-2.28 (m, 2H), 2.09-1.93 (m, 2H). ESI MS m/z 425 [C₂₆H₂₄N₄O₂+H]⁺.

### Synthesis of 5-(6-(5-(2-Methylbenzoyl)-2,5-diazabicyclo[2.2.1]heptan-2-yl)pyridin-3-yl)-3,4-dihydroquinolin-2(1H)-one

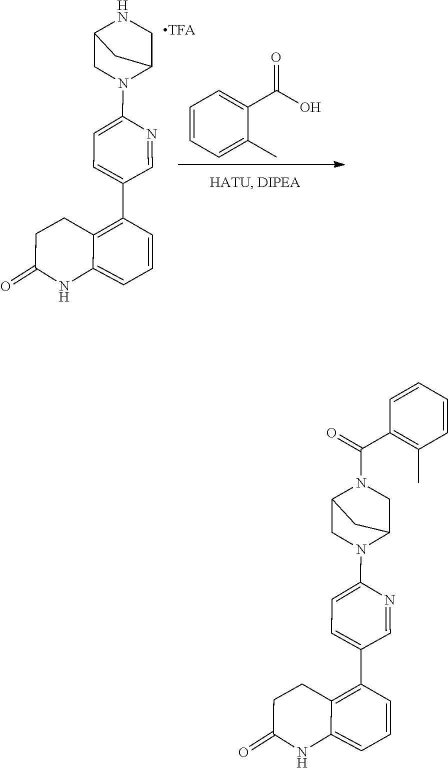

5-(6-(5-(2-Methylbenzoyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one 2,2,2-trifluoroacetate and 2-methylbenzoic acid to afford 5-(6-(5-(2-methylbenzoyl)-2,5-diazabicyclo[2.2.1]heptan-2-yl)pyridin-3-yl)-3,4-dihydroquinolin-2(1H)-one (15 mg, 20%) as a white solid. ¹H NMR (300 MHz, DMSO-d₆) δ 10.16 (s, 1H), 8.07 (dd, J=16.2, 2.1 Hz, 1H), 7.57-7.54 (m, 1H), 7.34-7.13 (m, 5H), 6.90-6.85 (m, 2H), 6.67-6.56 (m, 1H), 4.95-4.81 (m, 1.3H), 4.04 (s, 0.5H), 3.62-3.60 (m, 1H), 3.47-3.30 (m, 3H), 2.86-2.80 (m, 2H), 2.39-2.34 (m, 2H), 2.25 (s, 2H), 2.04-1.96 (m, 3H). ESI MS m/z 439 [C27H₂₆N₄O₂+H]⁺.

### Synthesis of 5-(6-(5-(Cyclohexanecarbonyl)-2,5-diazabicyclo[2.2.1]heptan-2-yl)pyridin-3-yl)-3,4-dihydroquinolin-2(1H)-one

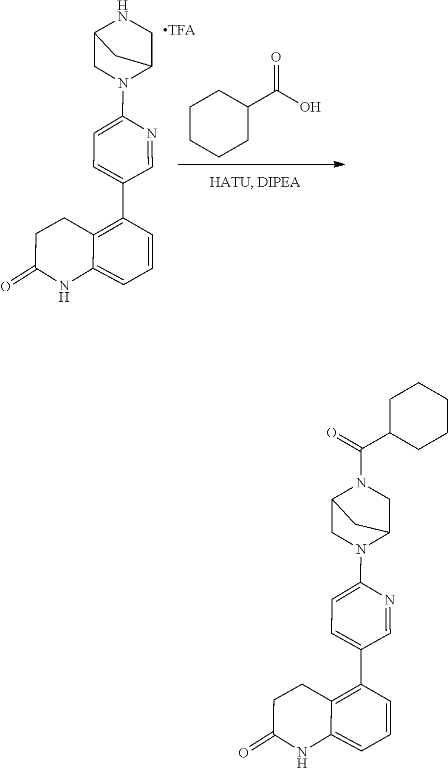

5-(6-(5-(Cyclohexanecarbonyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one 2,2,2-trifluoroacetate and cyclohexanecarboxylic acid to afford 5-(6-(5-(cyclohexanecarbonyl)-2,5-diazabicyclo[2.2.1]heptan-2-yl)pyridin-3-yl)-3,4-dihydroquinolin-2(1H)-one (14 mg, 19%) as a white solid. ¹H NMR (300 MHz, DMSO-d₆) δ 10.16 (s, 1H), 8.06 (s, 1H), 7.52 (dd, J=8.7, 2.4 Hz, 1H), 7.19 (t, J=7.5 Hz, 1H), 6.88-6.85 (m, 2H), 6.63-6.58 (m, 1H), 4.93-4.79 (m, 2H), 3.63-3.43 (m, 3H), 3.32-3.23 (m, 2H), 2.82 (t, J=7.5 Hz, 2H), 2.38-2.27 (m, 2H), 2.00-1.90 (m, 2H), 1.69-1.62 (m, 4H), 1.34-1.16 (m, 6H). ESI MS m/z 431 [C₂₆H₃₀N₄O₂+H]⁺.

### Synthesis of 5-(6-(5-Picolinoyl-2,5-diazabicyclo[2.2.1]heptan-2-yl)pyridin-3-y)-3,4-dihydroquinolin-2(1H)-one

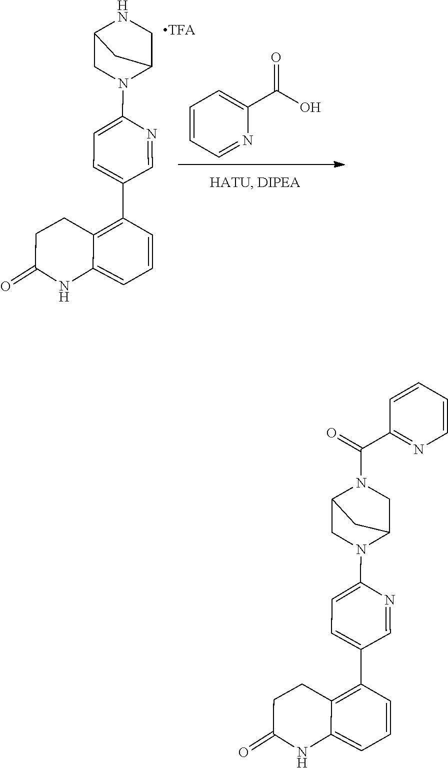

5-(6-(5-Picolinoyl-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one 2,2,2-trifluoroacetate and picolinic acid to afford 5-(6-(5-picolinoyl-2,5-diazabicyclo[2.2.1]heptan-2-yl)pyridin-3-yl)-3,4-dihydroquinolin-2(1H)-one (19 mg, 22%) as an off-white solid. ¹H NMR (300 MHz, DMSO-d₆) δ 10.14 (s, 1H), 8.67-8.56 (m, 1H), 8.09-7.77 (m, 3H), 7.57-7.47 (m, 2H), 7.22-7.15 (m, 1H), 6.89-6.84 (m, 2H), 6.67-6.60 (m, 1H), 5.09-4.91 (m, 2H), 3.96-3.46 (m, 4H), 2.86-2.78 (m, 2H), 2.38-2.31 (m, 2H), 2.02 (s, 2H). ESI MS m/z 426 [C₂₅H₂₃N₅O₂+H]⁺.

### Synthesis of 5-(6-(5-(Vinylsulfonyl)-2,5-diazabicyclo[2.2.2]octan-2-yl)pyridin-3-y)-3,4-dihydroquinolin-2(1H)-one

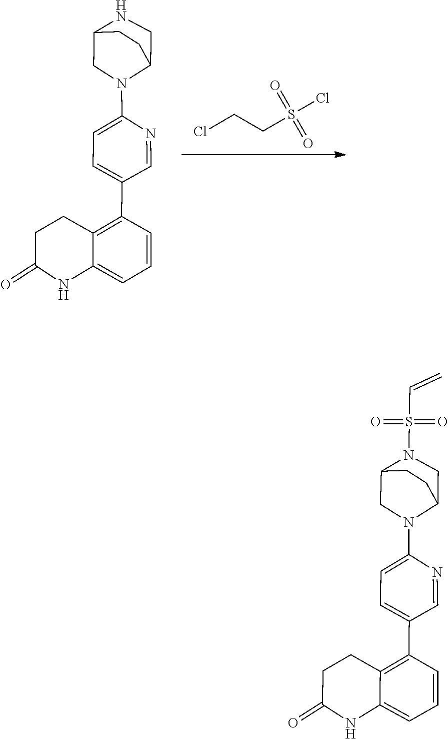 [SMILES: O=C1CCc2c(cccc2-c2ccc(N3CC4CCC3CN4)nc2)N1 ; O=S(=O)(Cl)CCCl ; C=CS(=O)(=O)N1CC2CCC1CN2c1ccc(-c2cccc3c2CCC(=O)N3)cn1]

5-(6-(5-(Vinylsulfonyl)-2,5-diazabicyclo[2.2.2]octan-2-yl)pyridin-3-yl)-3,4-dihydroquinolin-2(1H)-one was prepared according to General Protocol 1 using 5-(6-(2,5-diazabicyclo[2.2.2]octan-2-yl)pyridin-3-yl)-3,4-dihydroquinolin-2(1H)-one and 2-chloroethanesulfonyl chloride to afford 5-(6-(5-(vinylsulfonyl)-2,5-diazabicyclo[2.2.2]octan-2-yl)pyridin-3-yl)-3,4-dihydroquinolin-2(1H)-one (21 mg, 18%) as an off-white solid. ¹H NMR (300 MHz, DMSO-d₆) δ 10.15 (s, 1H), 8.06 (d, J=2.1 Hz, 1H), 7.56 (dd, J=8.7, 2.4 Hz, 1H), 7.19 (t, J=7.8 Hz, 1H), 6.98-6.85 (m, 3H), 6.59 (d, J=8.7 Hz, 1H), 6.13-6.06 (m, 2H), 4.90 (s, 1H), 4.02 (s, 1H), 3.70-3.66 (m, 1H), 3.49-3.37 (m, 3H), 2.82 (t, J=7.5 Hz, 2H), 2.36 (t, J=7.3 Hz, 2H), 2.08-1.80 (m, 4H). ESI MS m/z 425 [C₂₂H₂₄N₄O₃S+H]⁺.

### Synthesis of 5-(6-(5-(2-Chlorobenzoyl)-2,5-diazabicyclo[2.2.1]heptan-2-yl)pyridin-3-yl)-3,4-dihydroquinolin-2(1H)-one

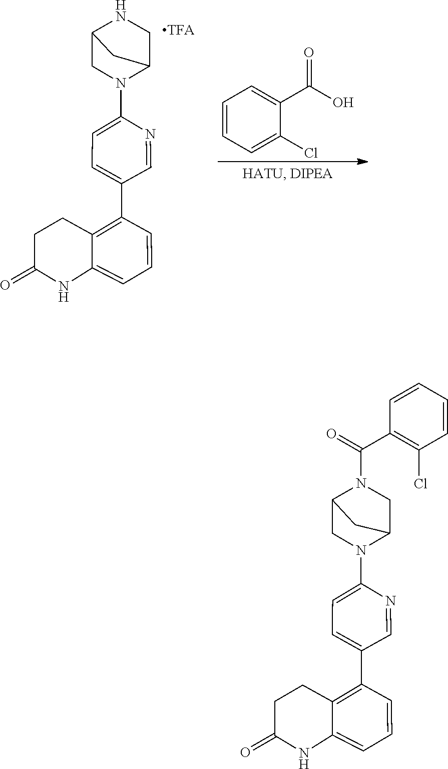 [SMILES: O=C1CCc2c(cccc2-c2ccc(N3CC4CC3CN4)nc2)N1 ; O=C(O)c1ccccc1Cl ; O=C1CCc2c(cccc2-c2ccc(N3CC4CC3CN4C(=O)c3ccccc3Cl)nc2)N1]

5-(6-(5-(2-Chlorobenzoyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one 2,2,2-trifluoroacetate and 2-chlorobenzoic acid to afford 5-(6-(5-(2-chlorobenzoyl)-2,5-diazabicyclo[2.2.1]heptan-2-yl)pyridin-3-yl)-3,4-dihydroquinolin-2(1H)-one (17 mg, 24%) as a white solid. ¹H NMR (300 MHz, DMSO-d₆) δ 10.15 (s, 1H), 8.10-8.04 (m, 1H), 7.59-7.35 (m, 5H), 7.23-7.17 (m, 1H), 6.90-6.85 (m, 2H), 6.71-6.60 (m, 1H), 4.96-4.85 (m, 1.4H), 4.06 (s, 0.5H), 3.64-3.60 (m, 1H), 3.47-3.40 (m, 2H), 3.27-3.04 (m, 1H), 2.86-2.81 (m, 2H), 2.40-2.34 (m, 2H), 2.08-1.99 (m, 2H). ESI MS m/z 459 [C₂₆H₂₃ClN₄O₂+H]⁺.

### Synthesis of 5-(6-(5-(3-Chlorobenzoyl)-2,5-diazabicyclo[2.2.1]heptan-2-yl)pyridin-3-yl)-3,4-dihydroquinolin-2(1H)-one

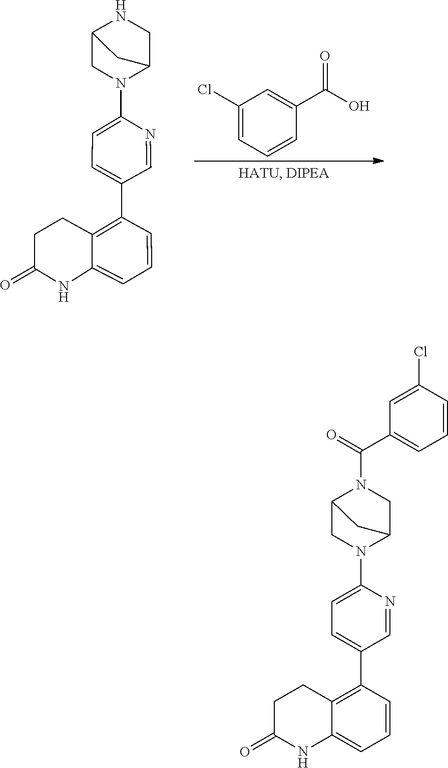 [SMILES: O=C1CCc2c(cccc2-c2ccc(N3CC4CC3CN4)nc2)N1 ; O=C(O)c1cccc(Cl)c1 ; O=C1CCc2c(cccc2-c2ccc(N3CC4CC3CN4C(=O)c3cccc(Cl)c3)nc2)N1]

5-(6-(5-(3-Chlorobenzoyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one and 3-chlorobenzoic acid to afford 5-(6-(5-(3-chlorobenzoyl)-2,5-diazabicyclo[2.2.1]heptan-2-yl)pyridin-3-yl)-3,4-dihydroquinolin-2(1H)-one (17 mg, 24%) as a white solid. ¹H NMR (300 MHz, DMSO-d₆) δ 10.16-10.14 (m, 1H), 8.09-8.02 (m, 1H), 7.57-7.43 (m, 5H), 7.23-7.15 (m, 1H), 6.90-6.83 (m, 2H), 6.67-6.57 (m, 1H), 4.93-4.88 (m, 1.4H), 4.42 (s, 0.6H), 3.77-3.22 (m, 4H), 2.86-2.77 (m, 2H), 2.44-2.34 (m, 2H), 2.10-1.96 (m, 2H). ESI MS m/z 459 [C₂₆H₂₃ClN₄O₂+H]⁺.

### Synthesis of 5-(6-(5-(3-Methoxybenzoyl)-2,5-diazabicyclo[2.2.1]heptan-2-yl)pyridin-3-yl)-3,4-dihydroquinolin-2(1H)-one

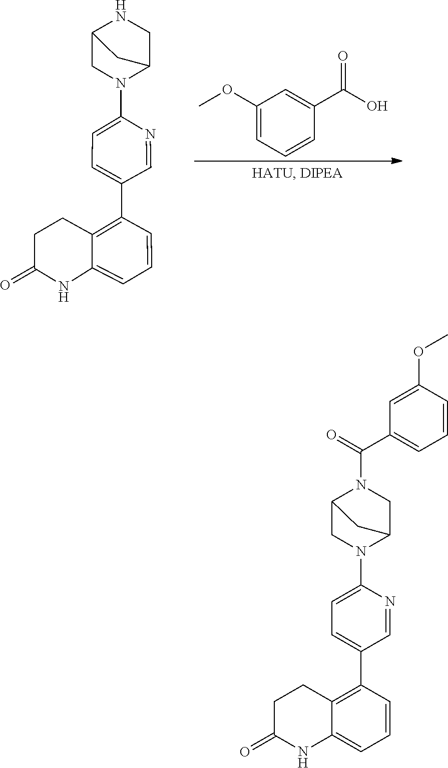 [SMILES: O=C1CCc2c(cccc2-c2ccc(N3CC4CC3CN4)nc2)N1 ; COc1cccc(C(=O)O)c1 ; COc1cccc(C(=O)N2CC3CC2CN3c2ccc(-c3cccc4c3CCC(=O)N4)cn2)c1]

5-(6-(5-(3-Methoxybenzoyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one and 3-methoxybenzoic acid to afford 5-(6-(5-(3-methoxybenzoyl)-2,5-diazabicyclo[2.2.1]heptan-2-yl)pyridin-3-yl)-3,4-dihydroquinolin-2(1H)-one (41 mg, 58%) as an off-white solid. ¹H NMR (300 MHz, DMSO-d₆) δ 10.16-10.14 (m, 1H), 8.10-8.02 (m, 1H), 7.58-7.29 (m, 2H), 7.20-7.17 (m, 1H), 7.08-7.03 (m, 3H), 6.90-6.83 (m, 2H), 6.68-6.58 (m, 1H), 4.93-4.87 (m, 1.4H), 4.44 (s, 0.6H), 3.81-3.75 (m, 3H), 3.64-3.53 (m, 3H), 3.24-3.20 (m, 1H), 2.87-2.79 (m, 2H), 2.39-2.34 (m, 2H), 2.10-1.92 (m, 2H). ESI MS m/z 455 [C₂₇H₂₆N₄O₃+H]⁺.

### Synthesis of 5-(6-(5-(3-Methylpicolinoyl)-2,5-diazabicyclo[2.2.1]heptan-2-yl)pyridin-3-yl)-3,4-dihydroquinolin-2(1H)-one

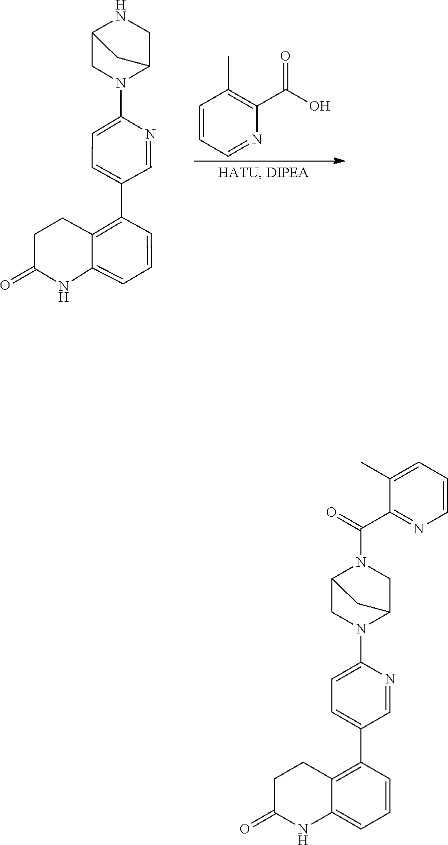

5-(6-(5-(3-Methylpicolinoyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one and 3-methylpicolinic acid to afford 5-(6-(5-(3-methylpicolinoyl)-2,5-diazabicyclo[2.2.1]heptan-2-yl)pyridin-3-yl)-3,4-dihydroquinolin-2(1H)-one (33 mg, 40%) as an off-white solid. ¹H NMR (300 MHz, DMSO-d₆) δ 10.15 (s, 1H), 8.45-8.33 (m, 1H), 8.10-8.04 (m, 1H), 7.78-7.66 (m, 1H), 7.57-7.50 (m, 1H), 7.43-7.31 (m, 1H), 7.22-7.16 (m, 1H), 6.88 (t, J=7.9 Hz, 2H), 6.68-6.65 (m, 1H), 5.02-4.84 (m, 1.4H), 4.15 (s, 0.6H), 3.67-3.61 (m, 1H), 3.49-3.46 (m, 2H), 3.12-3.09 (m, 1H), 2.86-2.78 (m, 2H), 2.39-2.34 (m, 2H), 2.28 (s, 2H), 2.11 (s, 1H), 2.04-1.99 (m, 2H). ESI MS m/z 440 [C₂₆H₂₅N₅O₂+H]⁺.

### Synthesis of 5-(6-(5-(3-Chloropicolinoyl)-2,5-diazabicyclo[2.2.1]heptan-2-yl)pyridin-3-yl)-3,4-dihydroquinolin-2(1H)-one

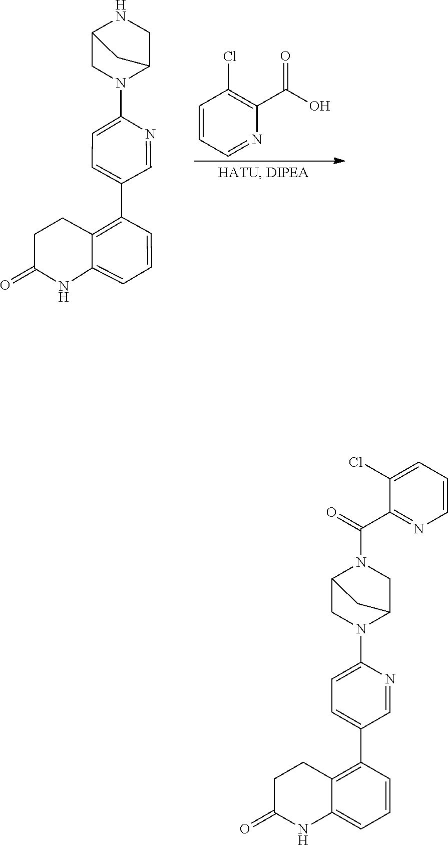 [SMILES: O=C1CCc2c(cccc2-c2ccc(N3CC4CC3CN4)nc2)N1 ; O=C(O)c1ncccc1Cl ; O=C1CCc2c(cccc2-c2ccc(N3CC4CC3CN4C(=O)c3ncccc3Cl)nc2)N1]

5-(6-(5-(3-Chloropicolinoyl)-2,5-diazabicyclo[2.2.1]heptan-2-yl)pyridin-3-yl)-3,4-dihydroquinolin-2(1H)-one was prepared according to General Protocol 2 using 5-(6-(2,5-diazabicyclo[2.2.1]heptan-2-yl)pyridin-3-yl)-3,4-dihydroquinolin-2(1H)-one and 3-chloropicolinic acid to afford 5-(6-(5-(3-chloropicolinoyl)-2,5-diazabicyclo[2.2.1]heptan-2-yl)pyridin-3-yl)-3,4-dihydroquinolin-2(1H)-one (22 mg, 26%) as an off-white solid. ¹H NMR (300 MHz, DMSO-d₆) δ 10.15 (s, 1H), 8.61-8.50 (m, 1H), 8.13-7.99 (m, 2H), 7.59-7.47 (m, 2H), 7.22-7.17 (m, 1H), 6.91-6.85 (m, 2H), 6.68-6.60 (m, 1H), 5.01-4.87 (m, 1.4H), 4.13 (s, 0.6H), 3.68-3.62 (m, 1H), 3.51-3.47 (m, 2H), 3.12-3.09 (m, 1H), 2.86-2.79 (m, 2H), 2.40-2.34 (m, 2H), 2.11-2.01 (m, 2H). ESI MS m/z 460 [C₂₅H₂₂ClN₅O₂+H]⁺.

### Synthesis of 5-(6-(5-(2-Methoxybenzoyl)-2,5-diazabicyclo[2.2.2]octan-2-yl)pyridin-3-y)-3,4-dihydroquinolin-2(1H)-one

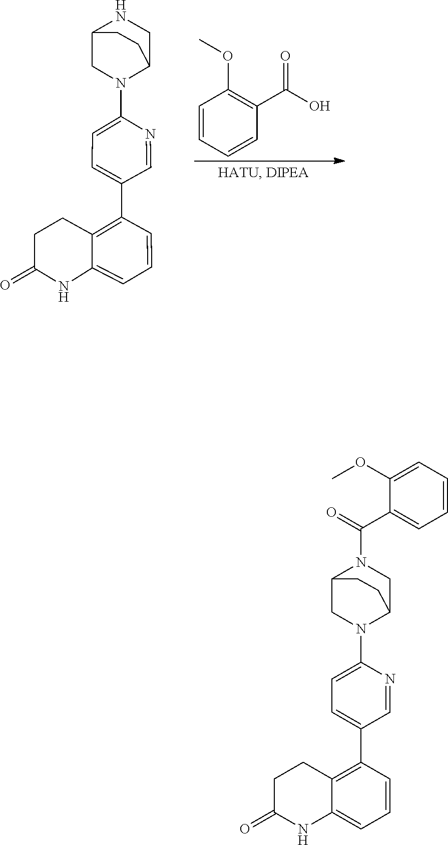 [SMILES: O=C1CCc2c(cccc2-c2ccc(N3CC4CCC3CN4)nc2)N1 ; COc1ccccc1C(=O)O ; COc1ccccc1C(=O)N1CC2CCC1CN2c1ccc(-c2cccc3c2CCC(=O)N3)cn1]

5-(6-(5-(2-Methoxybenzoyl)-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and 2-methoxybenzoic acid to afford 5-(6-(5-(2-methoxybenzoyl)-2,5-diazabicyclo[2.2.2]octan-2-yl)pyridin-3-yl)-3,4-dihydroquinolin-2(1H)-one (27 mg, 39%) as an off-white solid. ¹H NMR (300 MHz, DMSO-d₆) δ 10.15 (s, 1H), 8.08-8.06 (m, 1H), 7.56-7.54 (m, 1H), 7.45-7.39 (m, 1H), 7.25-6.99 (m, 4H), 6.89-6.85 (m, 2H), 6.67-6.55 (m, 1H), 4.98 (s, 0.7H), 4.80 (s, 0.4H), 3.82 (s, 3H), 3.66-3.50 (m, 5H), 2.85-2.81 (m, 2H), 2.39-2.34 (m, 2H), 2.07-1.68 (m, 4H). ESI MS m/z 469 [C₂₈H₂₈N₄O₃+H]⁺.

### Synthesis of 5-(6-(5-(2-Methylbenzoyl)-2,5-diazabicyclo[2.2.2]octan-2-yl)pyridin-3-y)-3,4-dihydroquinolin-2(1H)-one

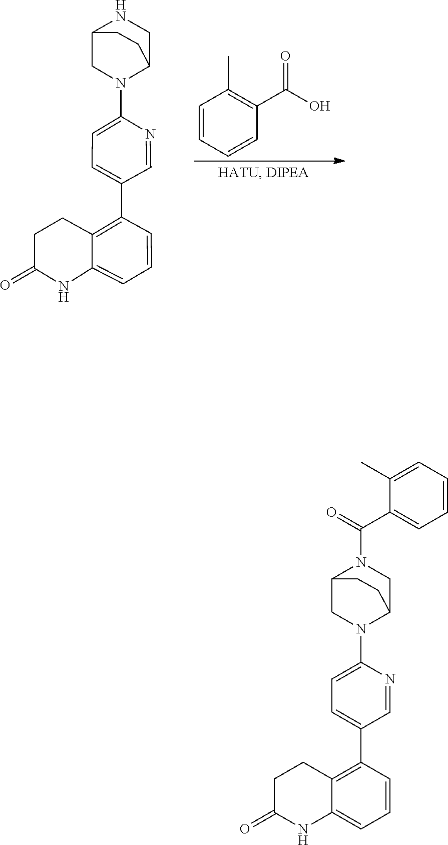

5-(6-(5-(2-Methylbenzoyl)-2,5-diazabicyclo[2.2.2]octan-2-yl)pyridin-3-yl)-3,4-dihydroquinolin-2(1H)-one was prepared according to General Protocol 2 using 5-(6-(2,5-diazabicyclo[2.2.2]octan-2-yl)pyridin-3-yl)-3,4-dihydroquinolin-2(1H)-one and 2-methylbenzoic acid to afford 5-(6-(5-(2-methylbenzoyl)-2,5-diazabicyclo[2.2.2]octan-2-yl)pyridin-3-yl)-3,4-dihydroquinolin-2(1H)-one (24 mg, 36%) as an off-white solid. ¹H NMR (300 MHz, DMSO-d₆) δ ¹H NMR (300 MHz, CDCl₃) δ 10.15 (s, 1H), 8.08-8.05 (m, 1H), 7.55 (dd, J=8.7, 1.8 Hz, 1H), 7.35-7.17 (m, 5H), 6.89-6.85 (m, 2H), 6.61 (d, J=8.1 Hz, 1H), 5.00 (s, 0.7H), 4.83-4.77 (m, 0.6H), 3.77-3.63 (m, 3H), 2.86-2.80 (m, 2H), 2.36 (t, J=7.5 Hz, 2H), 2.25 (s, 3H), 2.06-1.66 (m, 4H). ESI MS m/z 453 [C₂₈H₂₈N₄O₂+H]⁺.

### Example 2—Synthesis of 4-((5′-((5-Cyano-2-methylphenyl)amino)-3′H-spiro[azetidine-3,1′-isobenzofuran]-1-yl)sulfonyl)benzoic Acid

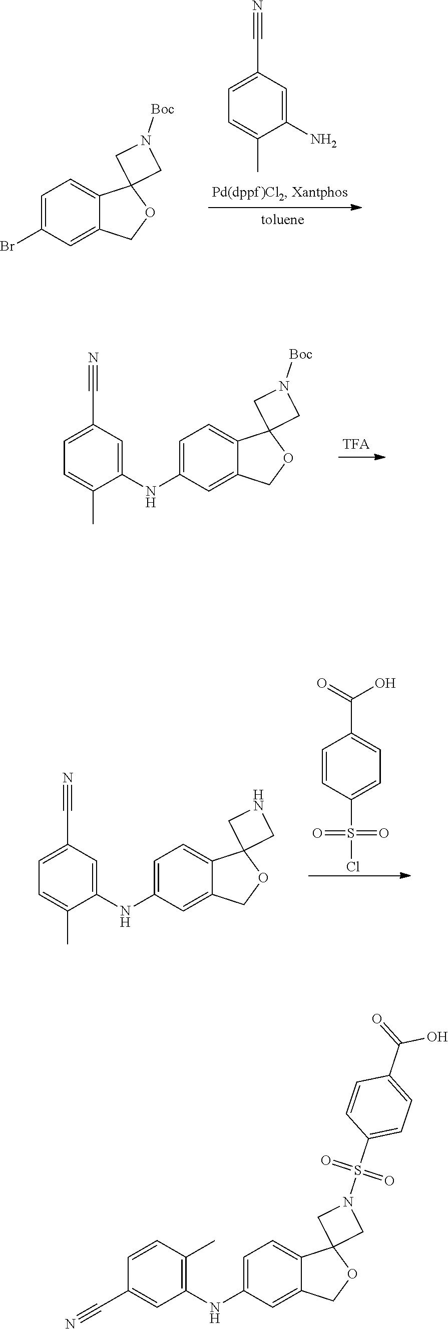

### Preparation of tert-Butyl 5′-((5-cyano-2-methylphenyl)amino)-3′H-spiro[azetidine-3,1′-isobenzofuran]-1-carboxylate

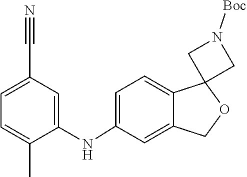 [SMILES: Cc1ccc(C#N)cc1Nc1ccc2c(c1)COC21CN(C(=O)OC(C)(C)C)C1]

A solution of tert-butyl 5′-bromo-3′H-spiro[azetidine-3,1′-isobenzofuran]-1-carboxylate (2.47 g, 7.26 mmol) and 3-amino-4-methylbenzonitrile (1.06 g, 7.99 mmol) in toluene (40 ml) was degassed with N₂ gas for 10 minutes, then sodium tert-butoxide (1.40 g, 14.5 mmol), 9,9-dimethyl-4,5-bis(diphenylphosphino)xanthene (0.29 g, 0.51 mmol), and dichloro[1,1′-bis(diphenylphosphino)ferrocene]palladium (II) dichloromethane adduct (0.119 g, 0.145 mmol) were added. The reaction was heated to 110° C. for 3 hours then cooled to room temperature. The reaction mixture was diluted with EtOAc and washed with water. The aqueous layer was extracted with EtOAc and the combined organics were dried over sodium sulfate, filtered, and concentrated. The crude was purified by normal phase column chromatography (silica gel, 0-50% EtOAc/heptane) to afford tert-butyl 5′-((5-cyano-2-methylphenyl)amino)-3′H-spiro[azetidine-3,1′-isobenzofuran]-1-carboxylate (2.1 g, 74%) as a tan solid. ¹H NMR (300 MHz, CDCl₃) δ 7.42-7.38 (m, 2H), 7.28-7.26 (m, 1H), 7.25-7.18 (m, 1H), 7.03-7.00 (m, 1H), 6.87 (s, 1H), 5.51 (s, 1H), 5.09 (s, 2H), 4.32 (d, J=9.6 Hz, 2H), 4.15 (d, J=9.6 Hz, 2H), 2.32 (s, 3H), 1.49 (s, 9H).

### Preparation of 3-((3′H-Spiro[azetidine-3,1′-isobenzofuran]-5′-yl)amino)-4-methylbenzonitrile

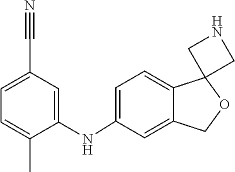 [SMILES: Cc1ccc(C#N)cc1Nc1ccc2c(c1)COC21CNC1]

To a solution of tert-butyl 5′-((5-cyano-2-methylphenyl)amino)-3′H-spiro[azetidine-3,1′-isobenzofuran]-1-carboxylate (1.50 g, 3.83 mmol) dissolved in dichloromethane (25.5 mL) was added 2,2,2-trifluoroacetic acid (5.90 mL, 77.0 mmol) at room temperature. The reaction was stirred for 2 hours then diluted with dichloromethane and washed with sat. aq. NaHCO₃. The aqueous layer was extracted with dichloromethane and the combined organic layers were dried over sodium sulfate, filtered, and concentrated to afford 3-((3′H-spiro[azetidine-3,1′-isobenzofuran]-5′-yl)amino)-4-methylbenzonitrile which was used without further purification (1.30 g, >99%). ESI MS m/z 292 [C₁₈H₁₇N₃O+H]⁺.

### Preparation of 4-((5′-((5-Cyano-2-methylphenyl)amino)-3′H-spiro[azetidine-3,1′-isobenzofuran]-1-yl)sulfonyl)benzoic Acid

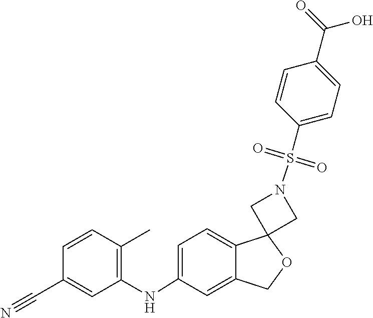 [SMILES: Cc1ccc(C#N)cc1Nc1ccc2c(c1)COC21CN(S(=O)(=O)c2ccc(C(=O)O)cc2)C1]

4-((5′-((5-Cyano-2-methylphenyl)amino)-3′H-spiro[azetidine-3,1′-isobenzofuran]-1-yl)sulfonyl)benzoic acid was prepared according to General Protocol 1 using 3-((3′H-spiro[azetidine-3,1′-isobenzofuran]-5′-yl)amino)-4-methylbenzonitrile and 4-(chlorosulfonyl)benzoic acid to afford 4-((5′-((5-cyano-2-methylphenyl)amino)-3′H-spiro[azetidine-3,1′-isobenzofuran]-1-yl)sulfonyl)benzoic acid (32 mg, 20%) as an off-white solid. ¹H NMR (500 MHz, DMSO-d₆) δ 13.60 (s, 1H), 8.26 (d, J=8.5 Hz, 2H), 8.01 (d, J=8.5 Hz, 2H), 7.76 (s, 1H), 7.39-7.38 (m, 2H), 7.30-7.28 (m, 1H), 6.92-6.88 (m, 1H), 6.82 (s, 1H), 6.76 (d, J=8.5 Hz, 1H), 4.87 (s, 2H), 4.00 (d, J=9.5 Hz, 2H), 3.97 (d, J=9.5 Hz, 2H), 2.25 (s, 3H). ESI MS m/z 474 [C₂₅H₂₁N₃O₅−H]⁻.

### Example 3—Synthesis of 5-(3′H-Spiro[azetidine-3,1′-isobenzofuran]-5′-yl)-3,4-dihydroquinolin-2(1H)-one Derivatives

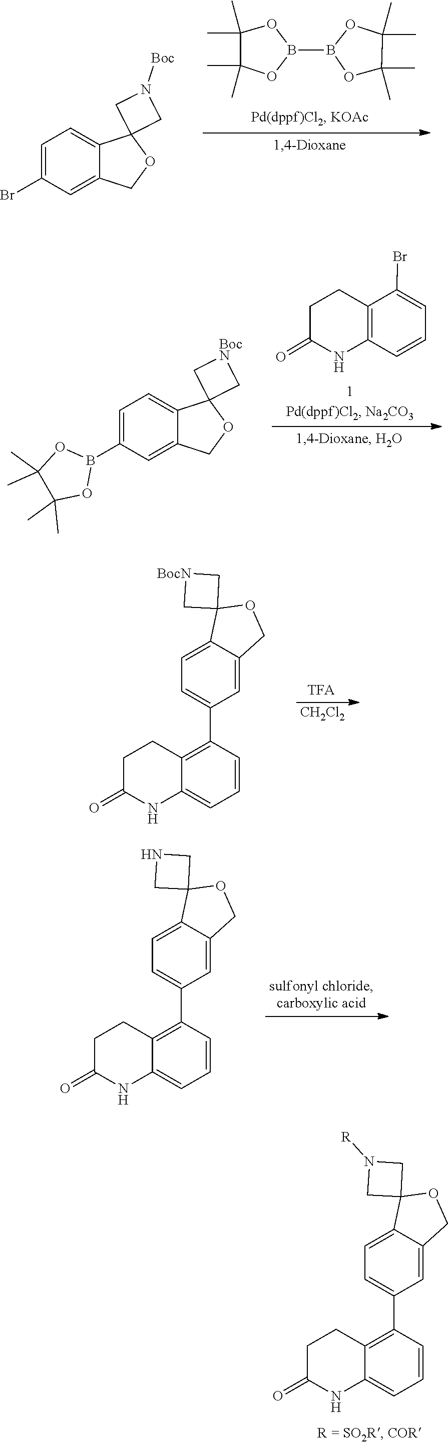

### Preparation of tert-Butyl 5′-(4,4,5,5-tetramethyl-1,3,2-dioxaborolan-2-yl)-3′H-spiro[azetidine-3,1′-isobenzofuran]-1-carboxylate

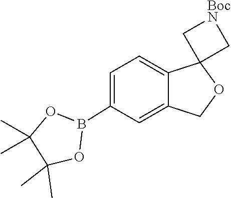 [SMILES: CC(C)(C)OC(=O)N1CC2(C1)OCc1cc(B3OC(C)(C)C(C)(C)O3)ccc12]

A solution of tert-butyl 5′-bromo-3′H-spiro[azetidine-3,1′-isobenzofuran]-1-carboxylate (100 mg, 0.29 mmol), bis(pinacolato)diboron (1.06 g, 7.99 mmol), and potassium acetate (87 mg, 0.88 mmol) in dioxane (3 ml) was degassed with argon for 10 minutes, then dichloro[1,1′-bis(diphenylphosphino)ferrocene]palladium (II) dichloromethane adduct (24 mg, 0.029 mmol) was added. The reaction was heated at 80° C. overnight then cooled to room temperature. The reaction mixture was diluted with EtOAc, washed with water, brine, dried over sodium sulfate, filtered, and concentrated. The crude was purified by normal phase column chromatography (silica gel, 0-20% EtOAc/hexanes) to afford tert-butyl 5′-(4,4,5,5-tetramethyl-1,3,2-dioxaborolan-2-yl)-3′H-spiro[azetidine-3,1′-isobenzofuran]-1-carboxylate (52 mg, 46%) as an off-white solid. ¹H NMR (300 MHz, CDCl₃) δ 7.83 (dd, J=7.5, 0.6 Hz, 1H), 7.67 (s, 1H), 7.49 (d, J=7.5 Hz, 1H), 5.11 (s, 2H), 4.31 (d, J=10.2 Hz, 2H), 4.14 (d, J=10.2 Hz, 2H), 1.33 (s, 9H), 1.26 (s, 12H).

### Preparation of Tert-Butyl 5′-(2-oxo-1,2,3,4-tetrahydroquinolin-5-yl)-3′H-spiro[azetidine-3,1′-isobenzofuran]-1-carboxylate

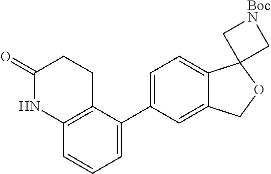 [SMILES: CC(C)(C)OC(=O)N1CC2(C1)OCc1cc(-c3cccc4c3CCC(=O)N4)ccc12]

A solution of tert-butyl 5′-(4,4,5,5-tetramethyl-1,3,2-dioxaborolan-2-yl)-3′H-spiro[azetidine-3,1′-isobenzofuran]-1-carboxylate (52 mg, 0.13 mmol), 5-bromo-3,4-dihydroquinolin-2(1H)-one (36 mg, 0.16 mmol), and sodium carbonate (30 mg, 0.28 mmol) in dioxane (2 mL) and water (0.2 mL) was degassed with argon for 10 minutes. Then dichloro[1,1′-bis(diphenylphosphino)ferrocene]palladium (II) dichloromethane adduct (11 mg, 0.013 mmol) was added and the reaction was heated at 80° C. overnight then cooled to room temperature. The reaction mixture was diluted with EtOAc, washed with water, brine, dried over sodium sulfate, filtered, and concentrated. The crude was purified by normal phase column chromatography (silica gel, 0-100% EtOAc/hexanes) to afford tert-butyl 5′-(2-oxo-1,2,3,4-tetrahydroquinolin-5-yl)-3′H-spiro[azetidine-3,1′-isobenzofuran]-1-carboxylate (27 mg, 50%) as a tan solid. ¹H NMR (300 MHz, CDCl₃) δ 7.62 (s, 1H), 7.53 (d, J=7.8 Hz, 1H), 7.33-7.26 (m, 1H), 7.24-7.22 (m, 1H), 7.15 (s, 1H), 6.98 (dd, J=7.5, 0.9 Hz, 1H), 6.77 (d, J=6.9 Hz, 1H), 5.17 (s, 2H), 4.36 (d, J=9.9 Hz, 2H), 4.19 (d, J=9.9 Hz, 2H), 2.91 (t, J=7.5 Hz, 2H), 2.55 (t, J=7.5 Hz, 2H), 1.49 (s, 9H).

### Preparation of 5-(3′H-Spiro[azetidine-3,1′-isobenzofuran]-5′-yl)-3,4-dihydroquinolin-2(1H)-one 2,2,2-trifluoroacetate

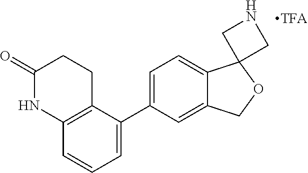 [SMILES: O=C1CCc2c(cccc2-c2ccc3c(c2)COC32CNC2)N1]

To a solution of tert-butyl 5′-(2-oxo-1,2,3,4-tetrahydroquinolin-5-yl)-3′H-spiro[azetidine-3,1′-isobenzofuran]-1-carboxylate (27 mg, 0.066 mmol) dissolved in dichloromethane (2 mL) was added 2,2,2-trifluoroacetic acid (1.0 mL, 135 mmol) at room temperature. The reaction was stirred for 2 hours then concentrated to afford 5-(3′H-spiro [azetidine-3,1′-isobenzofuran]-5′-yl)-3,4-dihydroquinolin-2(1H)-one 2,2,2-trifluoroacetate which was used without further purification (35 mg, >99%). ESI MS m/z 307 [C₁₉H₁₈N₂O₂+H]⁺.

### Preparation of 4-((5′-(2-Oxo-1,2,3,4-tetrahydroquinolin-5-yl)-3′H-spiro[azetidine-3,1′-isobenzofuran]-1-yl)sulfonyl)benzoic Acid

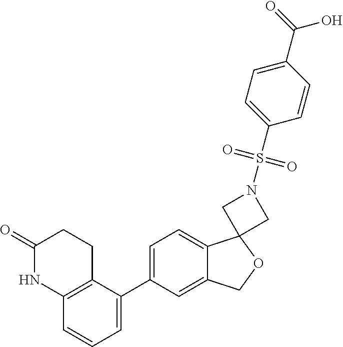 [SMILES: O=C1CCc2c(cccc2-c2ccc3c(c2)COC32CN(S(=O)(=O)c3ccc(C(=O)O)cc3)C2)N1]

4-((5′-(2-Oxo-1,2,3,4-tetrahydroquinolin-5-yl)-3′H-spiro[azetidine-3,1′-isobenzofuran]-1-yl)sulfonyl)benzoic acid was prepared according to General Protocol 1 using 5-(3′H-spiro[azetidine-3,1′-isobenzofuran]-5′-yl)-3,4-dihydroquinolin-2(1H)-one and 4-(chlorosulfonyl)benzoic acid to afford 4-((5′-(2-oxo-1,2,3,4-tetrahydroquinolin-5-yl)-3′H-spiro[azetidine-3,1′-isobenzofuran]-1-yl)sulfonyl)benzoic acid (10 mg, 31%) as an off-white solid. ¹H NMR (500 MHz, DMSO-d₆) δ 13.61 (br s, 1H), 10.15 (s, 1H), 8.27 (d, J=8.0 Hz, 2H), 8.03 (d, J=8.0 Hz, 2H), 7.26-7.18 (m, 2H), 7.06-7.04 (m, 2H), 6.90 (d, J=7.5 Hz, 1H), 6.83 (d, J=7.5 Hz, 1H), 4.99 (s, 2H), 4.09 (d, J=9.0 Hz, 2H), 4.01 (d, J=9.0 Hz, 2H), 2.74 (t, J=7.0 Hz, 2H), 2.34 (t, J=7.5 Hz, 2H). ESI MS m/z 491 [C₂₆H₂₂N₂O₆S+H]⁺.

### Preparation of (1s,4s)-4-(5′-(2-Oxo-1,2,3,4-tetrahydroquinolin-5-yl)-3′H-spiro[azetidine-3,1′-isobenzofuran]-1-carbonyl)clohexane-1-carboxylic Acid

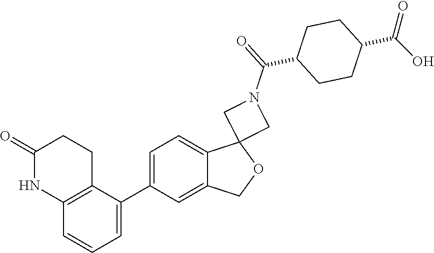 [SMILES: O=C1CCc2c(cccc2-c2ccc3c(c2)COC32CN(C(=O)[C@H]3CC[C@@H](C(=O)O)CC3)C2)N1]

(1s,4s)-4-(5′-(2-Oxo-1,2,3,4-tetrahydroquinolin-5-yl)-3′H-spiro[azetidine-3,1′-isobenzofuran]-1-carbonyl)cyclohexane-1-carboxylic acid was prepared according to General Protocol 2 using 5-(3′H-spiro[azetidine-3,1′-isobenzofuran]-5′-yl)-3,4-dihydroquinolin-2(1H)-one and cis-1,4-cyclohexanedicarboxylic acid to afford (1s,4s)-4-(5′-(2-oxo-1,2,3,4-tetrahydroquinolin-5-yl)-3′H-spiro[azetidine-3,1′-isobenzofuran]-1-carbonyl)cyclohexane-1-carboxylic acid (15 mg, 20%) as an off-white solid. ¹H NMR (300 MHz, DMSO-d₆) δ 12.11 (br s, 1H), 10.19 (s, 1H), 7.59 (d, J=7.8 Hz, 1H), 7.36 (d, J=7.8 Hz, 1H), 7.29 (s, 1H), 7.22 (dd, J=7.8, 7.8 Hz, 1H), 6.90 (dd, J=7.5, 7.5 Hz, 2H), 5.12 (s, 2H), 4.46 (s, 2H), 4.13 (s, 2H), 2.79 (t, J=7.4 Hz, 2H), 2.38-2.33 (m, 4H), 2.01-1.99 (m, 2H), 1.63-1.45 (m, 6H). ESI MS m/z 491 [C₂₆H₂₂N₂O₆S+H]⁺.

### Preparation of 5-(1-Benzoyl-3′H-spiro[azetidine-3,1′-isobenzofuran]-5′-y)-3,4-dihydroquinolin-2(1H)-one

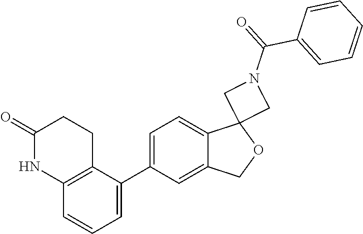 [SMILES: O=C1CCc2c(cccc2-c2ccc3c(c2)COC32CN(C(=O)c3ccccc3)C2)N1]

5-(1-Benzoyl-3′H-spiro[azetidine-3,1′-isobenzofuran]-5′-yl)-3,4-dihydroquinolin-2(1H)-one was prepared according to General Protocol 2 using 5-(3′H-spiro[azetidine-3,1′-isobenzofuran]-5′-yl)-3,4-dihydroquinolin-2(1H)-one and benzoic acid to afford 5-(1-benzoyl-3′H-spiro[azetidine-3,1′-isobenzofuran]-5′-yl)-3,4-dihydroquinolin-2(1H)-one (42 mg, 43%) as a white solid. ¹H NMR (300 MHz, DMSO-d₆) δ 10.20 (s, 1H), 7.75-7.66 (m, 3H), 7.57-7.45 (m, 3H), 7.38-7.35 (m, 1H), 7.31-7.29 (m, 1H), 7.22 (t, J=7.1 Hz, 1H), 7.00-6.85 (m, 2H), 5.14-5.12 (m, 2H), 4.62-4.38 (m, 4H), 2.82-2.76 (m, 2H), 2.37-2.33 (m, 2H). ESI MS m/z 410 [C₂₆H₂₂N₂O₃+H]⁺.

### Preparation of 5-(1-Isobutyryl-3′H-spiro[azetidine-3,1′-isobenzofuran]-5′-yl)-3,4-dihydroquinolin-2(1H)-one

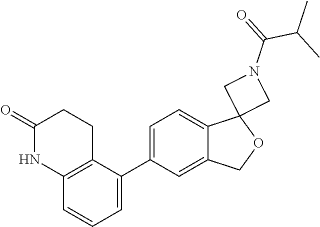 [SMILES: CC(C)C(=O)N1CC2(C1)OCc1cc(-c3cccc4c3CCC(=O)N4)ccc12]

5-(1-Isobutyryl-3′H-spiro[azetidine-3,1′-isobenzofuran]-5′-yl)-3,4-dihydroquinolin-2(1H)-one was prepared according to General Protocol 2 using 5-(3′H-spiro[azetidine-3,1′-isobenzofuran]-5′-yl)-3,4-dihydroquinolin-2(1H)-one and isobutyric acid to afford 5-(1-isobutyryl-3′H-spiro[azetidine-3,1′-isobenzofuran]-5′-yl)-3,4-dihydroquinolin-2(1H)-one (14 mg, 23%) as an off-white solid. ¹H NMR (300 MHz, DMSO-d₆) δ 10.19 (s, 1H), 7.60 (d, J=7.8 Hz, 1H), 7.37 (d, J=8.1 Hz, 1H), 7.29 (s, 1H), 7.22 (t, J=7.8 Hz, 1H), 6.90 (t, J=7.2 Hz, 2H), 5.12 (s, 2H), 4.47 (s, 2H), 4.18 (s, 2H), 2.79 (t, J=7.4 Hz, 2H), 2.60-2.55 (m, 1H), 2.36 (t, J=7.3 Hz, 2H), 1.03 (t, J=6.5 Hz, 6H). ESI MS m/z 377 [C₂₃H₂₄N₂O₃+H]⁺.

### Preparation of 5-(1-Acryloyl-3′H-spiro[azetidine-3,1′-isobenzofuran]-5′-y)-3,4-dihydroquinolin-2(1H)-one

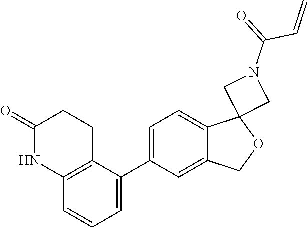 [SMILES: C=CC(=O)N1CC2(C1)OCc1cc(-c3cccc4c3CCC(=O)N4)ccc12]

5-(1-Acryloyl-3′H-spiro[azetidine-3,1′-isobenzofuran]-5′-yl)-3,4-dihydroquinolin-2(1H)-one was prepared according to General Protocol 2 using 5-(3′H-spiro[azetidine-3,1′-isobenzofuran]-5′-yl)-3,4-dihydroquinolin-2(1H)-one and acrylic acid to afford 5-(1-acryloyl-3′H-spiro[azetidine-3,1′-isobenzofuran]-5′-yl)-3,4-dihydroquinolin-2(1H)-one (14 mg, 23%) as a white solid. ¹H NMR (300 MHz, DMSO-d₆) δ 10.19 (s, 1H), 7.64 (d, J=7.8 Hz, 1H), 7.36 (d, J=7.8 Hz, 1H), 7.30 (s, 1H), 7.22 (t, J=7.8 Hz, 1H), 6.92-6.87 (m, 2H), 6.45-6.36 (m, 1H), 6.20-6.14 (m, 1H), 5.72 (dd, J=10.2, 2.1 Hz, 1H), 5.13 (s, 2H), 4.55-4.54 (m, 2H), 4.25-4.24 (m, 2H), 2.79 (t, J=7.5 Hz, 2H), 2.36 (t, J=7.5 Hz, 2H). ESI MS m/z 361 [C₂₂H₂₀N₂O₃+H]⁺.

### Example 4—Synthesis of 5-Phenyl-3,4-dihydroquinolin-2(1H)-one Derivatives

### Synthesis of 5-(4-((6-Hydroxy-4-(trifluoromethyl)pyridin-2-yl)amino)phenyl)-3,4-dihydroquinolin-2(1H)-one

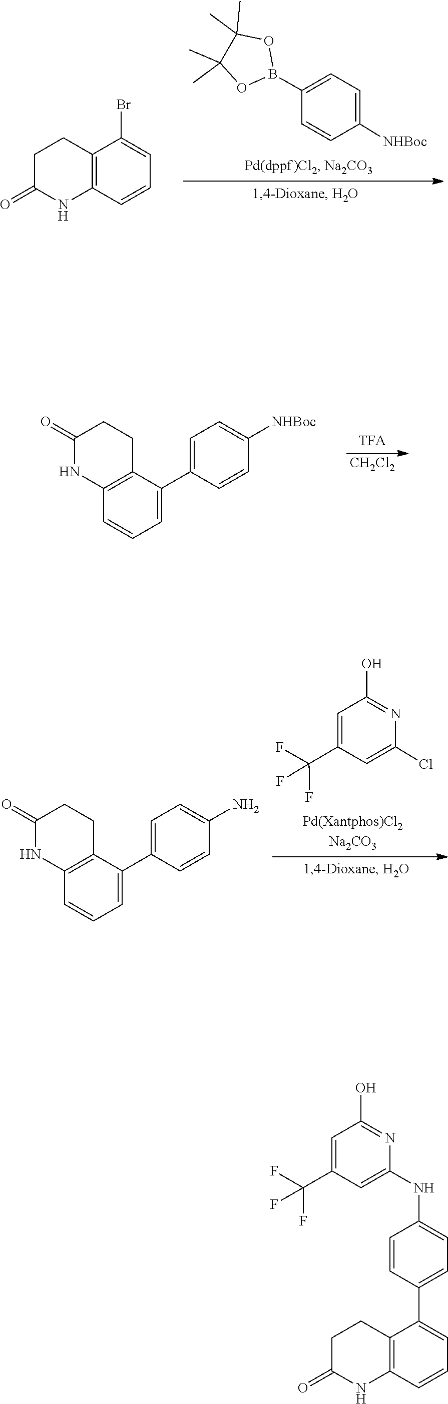

### Preparation of tert-Butyl (4-(2-oxo-1,2,3,4-tetrahydroquinolin-5-yl)phenyl)carbamate

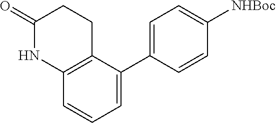 [SMILES: CC(C)(C)OC(=O)Nc1ccc(-c2cccc3c2CCC(=O)N3)cc1]

A solution of 5-bromo-3,4-dihydroquinolin-2(1H)-one (100 mg, 0.44 mmol), tert-butyl (4-(4,4,5,5-tetramethyl-1,3,2-dioxaborolan-2-yl)phenyl)carbamate (169 mg, 0.53 mmol), and sodium carbonate (98 mg, 0.93 mmol) in dioxane (3 mL) and water (0.4 mL) was degassed with argon for 10 minutes, then dichloro[1,1′-bis(diphenylphosphino)ferrocene]palladium (II) dichloromethane adduct (36 mg, 0.044 mmol) was added. The reaction was heated at 80° C. overnight then cooled to room temperature. The reaction mixture was diluted with EtOAc, washed with water, brine, dried over sodium sulfate, filtered, and concentrated. The crude was purified by normal phase column chromatography (silica gel, 0-100% EtOAc/hexanes) to afford tert-butyl (4-(2-oxo-1,2,3,4-tetrahydroquinolin-5-yl)phenyl)carbamate (126 mg, 84%) as a tan solid. ESI MS m/z 339 [C₂₀H₂₂N₂O₃+H]⁺.

### Preparation of 5-(4-Aminophenyl)-3,4-dihydroquinolin-2(1H)-one

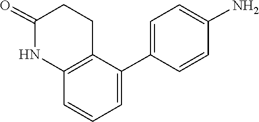 [SMILES: Nc1ccc(-c2cccc3c2CCC(=O)N3)cc1]

To a solution of tert-butyl (4-(2-oxo-1,2,3,4-tetrahydroquinolin-5-yl)phenyl)carbamate (81 mg, 0.24 mmol) dissolved in dichloromethane (3 mL) was added 2,2,2-trifluoroacetic acid (0.8 mL, 0.24 mmol) at room temperature. The reaction was stirred for 1 hour then diluted with dichloromethane, washed with sat., aq. Na₂CO₃, dried over sodium sulfate, and concentrated to afford 5-(4-aminophenyl)-3,4-dihydroquinolin-2(1H)-one which was used without further purification (56 mg, 98%). ESI MS m/z 239 [C₁₉H₁₈N₂O₂+H]⁺.

### Preparation of 5-(4-((6-Hydroxy-4-(trifluoromethyl)pyridin-2-yl)amino)phenyl)-3,4-dihydroquinolin-2(1H)-one

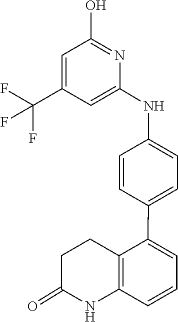 [SMILES: O=C1CCc2c(cccc2-c2ccc(Nc3cc(C(F)(F)F)cc(O)n3)cc2)N1]

A solution of 5-(4-aminophenyl)-3,4-dihydroquinolin-2(1H)-one (56 mg, 0.24 mmol), 6-chloro-4-(trifluoromethyl)pyridin-2-ol (56 mg, 0.28 mmol), and sodium carbonate (52 mg, 0.49 mmol) in dioxane (3 mL) and water (0.3 mL) was degassed with argon for 10 minutes, then dichloro[9,9-dimethyl-4,5-bis(diphenylphosphino)xanthene]palladium(II) (18 mg, 0.024 mmol) was added. The reaction was heated at 100° C. overnight then cooled to room temperature. The reaction mixture was diluted with dichloromethane, washed with water, brine, dried over sodium sulfate, filtered, and concentrated. The crude was purified by normal phase column chromatography (silica gel, 0-100% MeOH/dichloromethane) to afford 5-(4-((6-hydroxy-4-(trifluoromethyl)pyridin-2-yl)amino)phenyl)-3,4-dihydroquinolin-2(1H)-one (9 mg, 10%) as a light yellow solid. ¹H NMR (300 MHz, DMSO-d₆) δ 11.03 (s, 1H), 10.15 (s, 1H), 7.79 (d, J=12.0 Hz, 2H), 7.28-7.17 (m, 4H), 6.91-6.86 (m, 2H), 6.58 (s, 1H), 6.19 (s, 1H), 2.85 (t, J=12.0 Hz, 2H), 2.36 (t, J=12.5 Hz, 2H). ESI MS m/z 400 [C₂₁H₁₆F₃N₃O₂+H]⁺.

### Synthesis of 5-(4-(1-Propionylpyrrolidin-3-yl)phenyl)-3,4-dihydroquinolin-2(1H)-one PGP-195

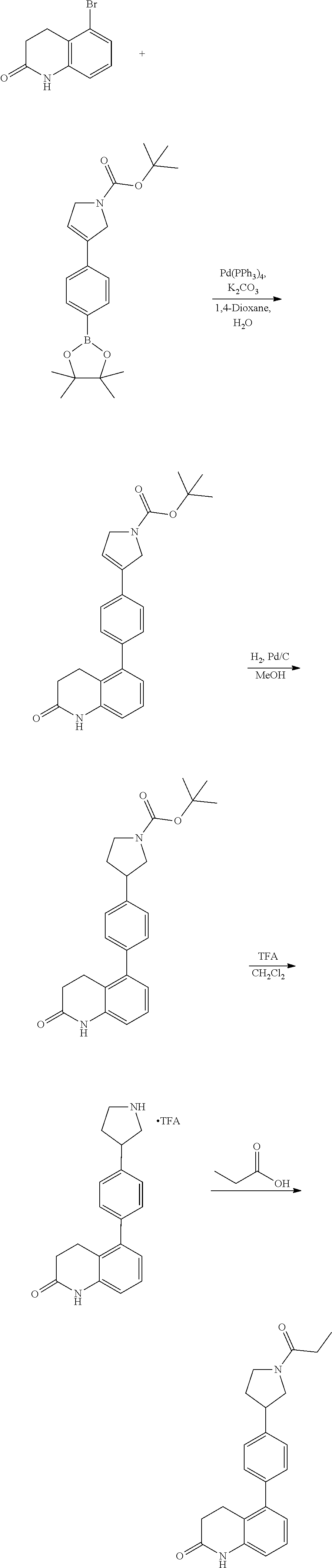

### Preparation of tert-Butyl 3-(4-(2-axo-1,2,3,4-tetrahydroquinolin-5-yl)phenyl)-2,5-dihydro-1H-pyrrole-1-carboxylate

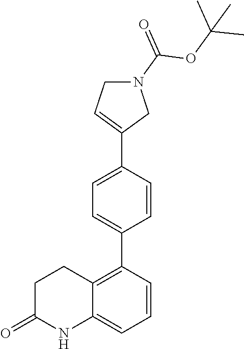 [SMILES: CC(C)(C)OC(=O)N1CC=C(c2ccc(-c3cccc4c3CCC(=O)N4)cc2)C1]

A solution of 5-bromo-3,4-dihydroquinolin-2(1H)-one (239 mg, 1.06 mmol), tert-butyl 3-(4-(4,4,5,5-tetramethyl-1,3,2-dioxaborolan-2-yl)phenyl)-2,5-dihydro-1H-pyrrole-1-carboxylate (410 mg, 0.96 mmol), and potassium carbonate (398 mg, 2.88 mmol) in dioxane (12 mL) and water (3 mL) was degassed with nitrogen for 10 minutes, then tetrakis(triphenyl-phosphine)palladium (0) (56 mg, 0.048 mmol) was added. The reaction was heated at 100° C. overnight then cooled to room temperature. The reaction mixture was diluted with dichloromethane, washed with water, dried over sodium sulfate, filtered, and concentrated. The crude was purified by normal phase column chromatography (silica gel, 0-100% EtOAc/hexanes) to afford tert-butyl 3-(4-(2-oxo-1,2,3,4-tetrahydroquinolin-5-yl)phenyl)-2,5-dihydro-1H-pyrrole-1-carboxylate (300 mg, 64%) as a yellow solid. ESI MS m/z 391 [C₂₄H₂₆N₂O₃+H]⁺.

### Preparation of Tert-Butyl 3-(4-(2-axo-1,2,3,4-tetrahydroquinolin-5-yl)phenyl)pyrrolidine-1-carboxylate

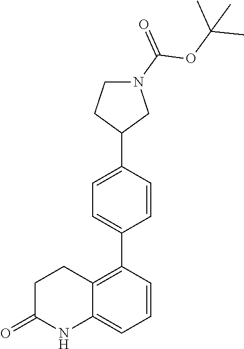 [SMILES: CC(C)(C)OC(=O)N1CCC(c2ccc(-c3cccc4c3CCC(=O)N4)cc2)C1]

A mixture of tert-butyl 3-(4-(2-oxo-1,2,3,4-tetrahydroquinolin-5-yl)phenyl)-2,5-dihydro-1H-pyrrole-1-carboxylate (130 mg, 0.266 mmol) and palladium on carbon (56.7 mg, 0.053 mmol) in MeOH (3 mL) was stirred under H₂ (1 atm) for 2 hours. The reaction mixture was filtered through Celite and concentrated. The crude was purified by normal phase column chromatography (silica gel, 0-100% EtOAc/heptane) to afford tert-butyl 3-(4-(2-oxo-1,2,3,4-tetrahydroquinolin-5-yl)phenyl)pyrrolidine-1-carboxylate (30 mg, 14%) as an off-white solid. ¹H NMR (300 MHz, DMSO-d₆) δ 10.17 (s, 1H), 7.38-7.18 (m, 5H), 6.90-6.87 (m, 2H), 3.77-3.71 (m, 1H), 3.48-3.42 (m, 2H), 3.35-3.31 (m, 2H), 2.79 (t, J=7.4 Hz, 2H), 2.37-2.32 (m, 2H), 2.28-2.18 (m, 1H), 2.03-1.96 (m, 1H), 1.42-1.41 (m, 9H). ESI MS m/z 293 [C₂₄H₂₈N₂O₃+H−C₅H₈O₂]⁺.

### Preparation of 5-(4-(Pyrrolidin-3-yl)phenyl)-3,4-dihydroquinolin-2(1H)-one 2,2,2-Trifluoroacetate

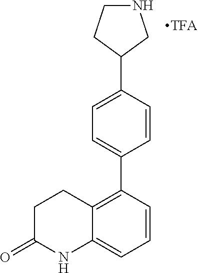 [SMILES: O=C1CCc2c(cccc2-c2ccc(C3CCNC3)cc2)N1 ; "·TFA"]

To a solution of tert-butyl 3-(4-(2-oxo-1,2,3,4-tetrahydroquinolin-5-yl)phenyl)pyrrolidine-1-carboxylate (33 mg, 0.084 mmol) dissolved in dichloromethane (1 mL) was added 2,2,2-trifluoroacetic acid (0.13 mL, 1.68 mmol) at room temperature. The reaction was stirred for 3 hours then concentrated to afford 5-(4-(pyrrolidin-3-yl)phenyl)-3,4-dihydroquinolin-2(1H)-one 2,2,2-trifluoroacetate which was used without further purification (35 mg, >99%). ESI MS m/z 293 [C₁₉H₂₀N₂O+H]⁺.

### Preparation of 5-(4-(1-Propionylpyrrolidin-3-yl)phenyl)-3,4-dihydroquinolin-2(1H)-one

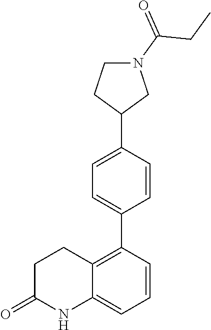 [SMILES: CCC(=O)N1CCC(c2ccc(-c3cccc4c3CCC(=O)N4)cc2)C1]

5-(4-(1-Propionylpyrrolidin-3-yl)phenyl)-3,4-dihydroquinolin-2(1H)-one was prepared according to General Protocol 2 using 5-(4-(pyrrolidin-3-yl)phenyl)-3,4-dihydroquinolin-2(1H)-one 2,2,2-trifluoroacetate and propionic acid to afford 5-(4-(1-propionylpyrrolidine-3-yl)phenyl)-3,4-dihydroquinolin-2(1H)-one (11 mg, 37%) as an off-white solid. ¹H NMR (300 MHz, DMSO-d₆) δ 10.17 (s, 1H), 7.41-7.18 (m, 5H), 6.90-6.87 (m, 2H), 3.92-3.84 (m, 1H), 3.65-3.39 (m, 3H), 3.26-3.23 (m, 1H), 2.79 (t, J=7.3 Hz, 2H), 2.38-2.22 (m, 5H), 2.19-1.89 (m, 1H), 1.03-0.97 (m, 3H). ESI MS m/z 349 [C₂₂H₂₄N₂O₂+H]⁺.

### Preparation of 5-(4-(1-(Ethylsulfonyl)pyrrolidin-3-yl)phenyl)-3,4-dihydroquinolin-2(1H)-one

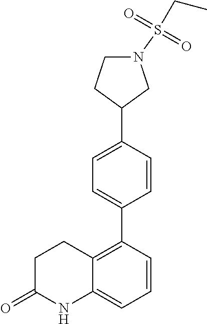 [SMILES: CCS(=O)(=O)N1CCC(c2ccc(-c3cccc4c3CCC(=O)N4)cc2)C1]

5-(4-(1-(Ethylsulfonyl)pyrrolidin-3-yl)phenyl)-3,4-dihydroquinolin-2(1H)-one was prepared according to General Protocol 1 using 5-(4-(pyrrolidin-3-yl)phenyl)-3,4-dihydroquinolin-2(1H)-one 2,2,2-trifluoroacetate and ethanesulfonyl chloride to afford 5-(4-(1-(ethylsulfonyl)pyrrolidin-3-yl)phenyl)-3,4-dihydroquinolin-2(1H)-one (24 mg, 32%) as an off-white solid. ¹H NMR (300 MHz, DMSO-d₆) δ 10.17 (s, 1H), 7.40 (d, J=8.1 Hz, 2H), 7.31 (d, J=8.1 Hz, 2H), 7.21 (dd, J=7.7, 7.7 Hz, 1H), 6.88 (dd, J=7.8, 2.1 Hz, 2H), 3.78-3.73 (m, 1H), 3.55-3.38 (m, 3H), 3.26-3.14 (m, 3H), 2.79 (t, J=7.3 Hz, 2H), 2.38-2.29 (m, 3H), 2.11-1.98 (m, 1H), 1.25 (t, J=7.3 Hz, 3H). ESI MS m/z 385 [C₂₁H₂₄N₂O₃S+H]⁺.

### Synthesis of 1-(2-Amino-4-(2-oxo-1,2,3,4-tetrahydroquinolin-5-yl)phenyl)-N-isopropyl-N-methylazetidine-3-carboxamide

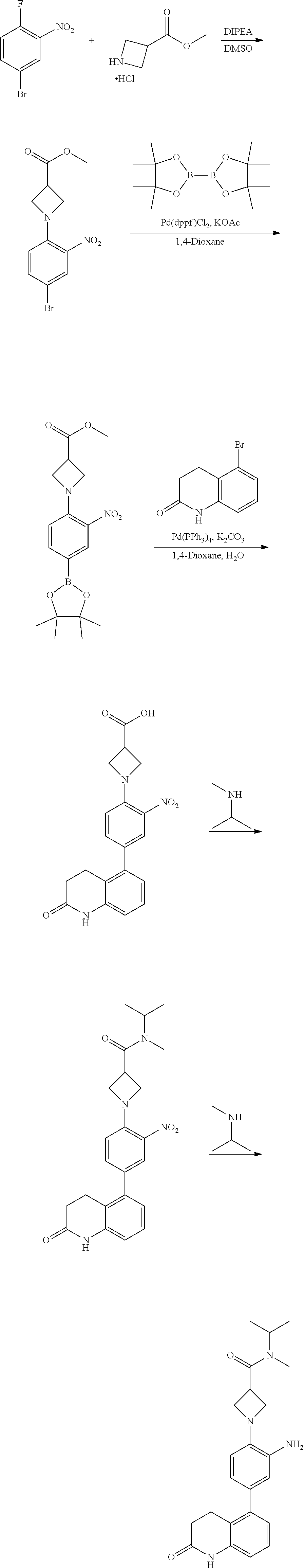

### Preparation of Methyl 1-(4-Bromo-2-nitrophenyl)azetidine-3-carboxylate

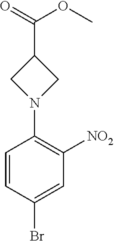 [SMILES: COC(=O)C1CN(c2ccc(Br)cc2[N+](=O)[O-])C1]

A solution of 4-bromo-1-fluoro-2-nitrobenzene (0.81 mL, 6.60 mmol), methyl azetidine-3-carboxylate hydrochloride (1.0 g, 6.60 mmol), and N,N-diisopropylethylamine (1.15 mL, 6.60 mmol) in DMSO (3 ml) was heated at 100° C. overnight. The reaction was stopped and cooled to room temperature and concentrated. The crude was purified by normal phase column chromatography (silica gel, 0-70% EtOAc/heptane) to afford methyl 1-(4-bromo-2-nitrophenyl)azetidine-3-carboxylate (1.6 g, 77%) as an orange solid.

### Preparation of Methyl 1-(2-Nitro-4-(4,4,5,5-tetramethyl-1,3,2-dioxaborolan-2-yl)phenyl)azetidine-3-carboxylate

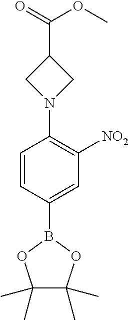 [SMILES: COC(=O)C1CN(c2ccc(B3OC(C)(C)C(C)(C)O3)cc2[N+](=O)[O-])C1]

A mixture of methyl 1-(4-bromo-2-nitrophenyl)azetidine-3-carboxylate (1.90 g, 6.03 mmol), 4,4,4′,4′,5,5,5′,5′-octamethyl-2,2′-bi(1,3,2-dioxaborolane) (2.30 g, 9.04 mmol), and potassium acetate (0.75 mL, 12.1 mmol) in dioxane (30 mL) was degassed with nitrogen gas for 10 min, then 1,1′-bis(diphenylphosphino)ferrocene-palladium(II)dichloride dichloromethane complex (0.492 g, 0.603 mmol) was added. The reaction was heated at 80° C. overnight then purified by normal phase column chromatography (silica gel, 0-100% EtOAc/heptane) to afford methyl 1-(2-nitro-4-(4,4,5,5-tetramethyl-1,3,2-dioxaborolan-2-yl)phenyl)azetidine-3-carboxylate (2.40 g, 99%) as a tan oil. ESI MS m/z 363 [C₁₇H₂₃BN₂O₆+H]⁺.

### Preparation of 1-(2-Nitro-4-(2-oxo-1,2,3,4-tetrahydroquinolin-5-yl)phenyl)azetidine-3-carboxylic Acid

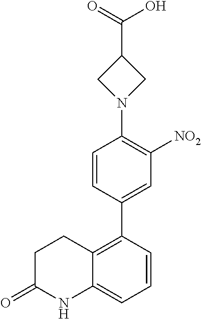 [SMILES: O=C1CCc2c(cccc2-c2ccc(N3CC(C(=O)O)C3)c([N+](=O)[O-])c2)N1]

A mixture of 5-bromo-3,4-dihydroquinolin-2(1H)-one (1.42 g, 6.26 mmol), methyl 1-(2-nitro-4-(4,4,5,5-tetramethyl-1,3,2-dioxaborolan-2-yl)phenyl)azetidine-3-carboxylate (2.40 g, 5.96 mmol), and potassium carbonate (2.47 g, 17.9 mmol) in dioxane (32 ml) and water (8 ml) was degassed with nitrogen for 15 min, then tetrakis(triphenylphosphine)palladium (0) (0.345 g, 0.298 mmol) was added. The reaction was heated at 100° C. overnight then cooled to room temperature. The reaction mixture was diluted with dichloromethane and washed with water. The product was in the aqueous layer so it was concentrated and purified by normal phase chromatography (silica gel, 0-20% MeOH/dichloromethane) to afford 1-(2-nitro-4-(2-oxo-1,2,3,4-tetrahydroquinolin-5-yl)phenyl)azetidine-3-carboxylic acid (1.54 g, 70%) as an orange solid. ESI MS m/z 368 [C₁₉H₁₇N₃O₅+H]⁺.

### Preparation of N-Isopropyl-N-methyl-1-(2-nitro-4-(2-axo-1,2,3,4-tetrahydroquinolin-5-yl)phenyl)azetidine-3-carboxamide

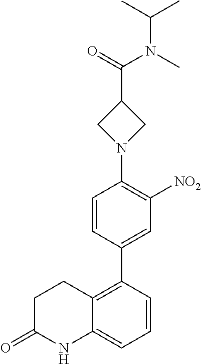 [SMILES: CC(C)N(C)C(=O)C1CN(c2ccc(-c3cccc4c3CCC(=O)N4)cc2[N+](=O)[O-])C1]

N-Isopropyl-N-methyl-1-(2-nitro-4-(2-oxo-1,2,3,4-tetrahydroquinolin-5-yl)phenyl)azetidine-3-carboxamide was prepared according to General Protocol 2 using 1-(2-nitro-4-(2-oxo-1,2,3,4-tetrahydroquinolin-5-yl)phenyl)azetidine-3-carboxylic acid and N-methylisopropylamine to afford N-isopropyl-N-methyl-1-(2-nitro-4-(2-oxo-1,2,3,4-tetrahydroquinolin-5-yl)phenyl)azetidine-3-carboxamide (750 mg, >99%) as a tan oil. ESI MS m/z 423 [C₂₃H₂₆N₄O₄+H]⁺.

### Preparation of 1-(2-Amino-4-(2-oxo-1,2,3,4-tetrahydroquinolin-5-yl)phenyl)-N-isopropyl-N-methylazetidine-3-carboxamide

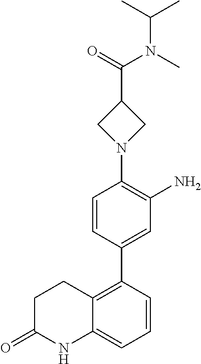 [SMILES: CC(C)N(C)C(=O)C1CN(c2ccc(-c3cccc4c3CCC(=O)N4)cc2N)C1]

A mixture of N-isopropyl-N-methyl-1-(2-nitro-4-(2-oxo-1,2,3,4-tetrahydroquinolin-5-yl)phenyl)azetidine-3-carboxamide (110 mg, 0.234 mmol) and palladium on carbon (125 mg) in MeOH (3 mL) was stirred under H₂ atmosphere (1 atm) for 2 hours. The reaction mixture was filtered through Celite and concentrated. The crude was purified by normal phase chromatography (silica gel, 0-10% MeOH/dichloromethane) followed by reverse phase chromatography (C18, 0-80% CH₃CN/H₂O) to afford 1-(2-amino-4-(2-oxo-1,2,3,4-tetrahydroquinolin-5-yl)phenyl)-N-isopropyl-N-methylazetidine-3-carboxamide (30 mg, 33%) as an off-white solid. ¹H NMR (300 MHz, DMSO-d₆) δ 10.11 (s, 1H), 7.14 (t, J=7.8 Hz, 1H), 6.81 (d, J=7.8 Hz, 2H), 6.58-6.51 (m, 3H), 4.71-4.65 (m, 2H), 4.09-4.03 (m, 2H), 3.92-3.63 (m, 3H), 2.80 (t, J=7.5 Hz, 2H), 2.72-2.68 (m, 3H), 2.36-2.31 (m, 2H), 1.14 (d, J=6.6 Hz, 3H), 1.03 (d, J=6.9 Hz, 3H). ESI MS m/z 393 [C₂₂H₂₈N₄O₂+H]⁺.

### Example 5—Synthesis of 3-Fluoro-5-(6-oxo-1,3,4,6-tetrahydro-2H-pyrido[1,2-a]pyrazin-7-yl)benzonitrile and 7-(2-oxo-1,2,3,4-Tetrahydroquinolin-5-yl)-1,2,3,4-tetrahydro-6H-pyrido[1,2-a]pyrazin-6-one Based Compounds

Unless otherwise noted, reagents and solvents were used as received from commercial suppliers. All non-aqueous reactions were carried out under an atmosphere of dry nitrogen (unless otherwise noted). Proton nuclear magnetic resonance spectra were obtained on a Bruker AVANCE 400 spectrometer at 400 MHz. Spectra are given in ppm (δ) and coupling constants, J values, are reported in hertz (Hz). Tetramethylsilane was used as an internal standard for proton nuclear magnetic resonance. Mass spectra were obtained using a Waters Acquity SQD (ESI, UP-LCMS). Final purifications were performed using a Waters mass triggered auto-purification HPLC system with binary solvent system A (acetonitrile) and B (10 mM ammonium bicarbonate in water) on a Gemini NX-c18 10 μm (30×150 mm) column using a gradient elution with flow rate of 30 mL/min and 20 min run time at ambient temperature. UPLC-MS analyses was obtained on a Waters Acquity UPLC. HPLC analyses was obtained on Agilent 1200 series HPLC system using (a) an Acquity BEH C18 1.7 μm (2.1×30 mm) column eluted according to solvent gradient Method 1, or (b) a X-bridge HILIC 3.5 μm (4.6×150 mm) column eluted according to solvent gradient Method 2. Chiral analyses were obtained on a Waters Acquity UPCC system using (c) a Chiralcel OX-H (4.6*150 mm) 5 μm column eluted according to solvent gradient Method 3.

TABLE 1
Method 1

| Time (min) | Flow (mL/min) | % A | % B |
|---|---|---|---|
| 0.0 | 0.5 | 2 | 98 |
| 0.5 | 0.5 | 2 | 98 |
| 1.5 | 0.5 | 45 | 55 |
| 2.4 | 0.5 | 95 | 5 |
| 3.2 | 0.5 | 95 | 5 |
| 3.3 | 0.5 | 2 | 98 |
| 3.5 | 0.5 | 2 | 98 |

B = Water with 0.05% v/v Trifluoroacetic Acid (or) 10 mM Ammonium formate in Water
A = Acetonitrile

TABLE 2
Method 2

| Time (min) | Flow (mL/min) | % A | % B |
|---|---|---|---|
| 0.0 | 1.0 | 90 | 10 |
| 3.0 | 1.0 | 90 | 10 |
| 8.0 | 1.0 | 70 | 30 |
| 10.0 | 1.0 | 60 | 40 |
| 15.0 | 1.0 | 60 | 40 |
| 15.1 | 1.0 | 90 | 10 |
| 20 | 1.0 | 90 | 10 |

A = 10 mM Ammonium Formate in Water
B = Acetonitrile

### Preparation of 3-Fluoro-5-(6-oxo-1,3,4,6-tetrahydro-2H-pyrido[1,2-a]pyrazin-7-yl)benzonitrile Based Analogs

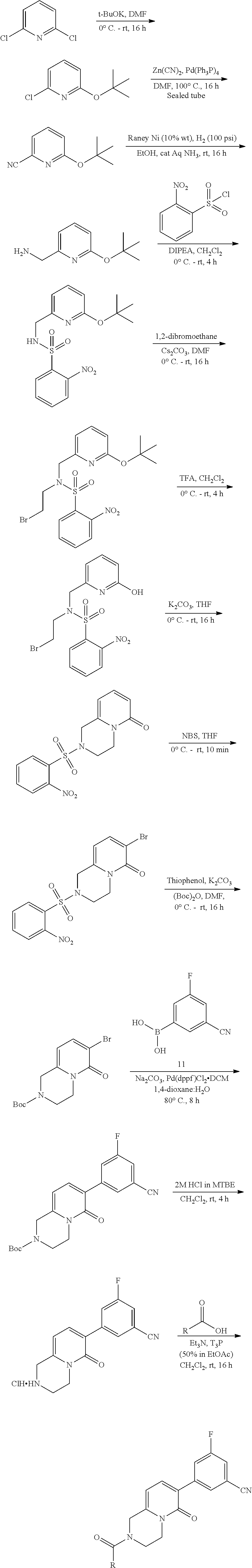

### Preparation of 2-(tert-Butoxy)-6-chloropyridine

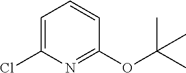

To a stirred solution of 2,6-dichloropyridine (20 g, 135.12 mmol) in DMF (200 mL) at 0° C. was added t-BuOK (22 g, 202.72 mmol) slowly portion wise and the resulting reaction mixture was stirred under nitrogen atmosphere at ambient temperature for 16 hours. After completion of reaction, aq. NH₄Cl (100 mL) solution was added and the resulting mixture was extracted with Hexane (2×200 mL). The combined organic layers were dried over anhydrous sodium sulfate and the drying agent was removed by decantation. The resulting solution was concentrated under reduced pressure and the obtained residue was purified by chromatography (silica 0.6% EtOAc in Hexane). The pure fractions were collected and concentrated under reduced pressure to afford 2-(tert-butoxy)-6-chloropyridine (16 g, 63%) as a colorless liquid. ¹H NMR (400 MHz, CDCl₃) δ 7.43 (t, J=7.6 Hz, 1H), 6.81 (d, J=7.6 Hz, 1H), 6.53 (d, J=8.0 Hz, 1H), 1.58 (s, 9H).

Preparation of 6-(tert-Butoxy) Picolinonitrile

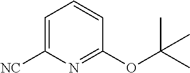

A solution of 2-(tert-butoxy)-6-chloropyridine (17 g, 91.57 mmol) in DMF (170 mL) was taken in a 500 mL sealed tube and charged with Zn(CN)₂ (21.85 g, 183.10 mmol) at room temperature. The reaction mixture was degassed with nitrogen for 10 min and tetrakis(triphenylphosphine) palladium (5.30 g, 4.57 mmol) was added and degassed for 5 more min at room temperature. The resulting reaction mixture was stirred at 100° C. for 16 hours. After completion of reaction by TLC, the reaction mixture was cooled to room temperature, filtered through diatomaceous earth, and washed with EtOAc (200 mL). The filtrate obtained was washed with ice cold water (100 mL) and brine (100 mL) and the combined organic layers were dried over anhydrous sodium sulfate and the drying agent was removed by decantation. The resulting solution was concentrated under reduced pressure and the residue obtained was purified by chromatography (silica 5% EtOAc in Hexane) to afford 6-(tert-butoxy) picolinonitrile (7 g, 43%) as an off-white solid. ¹H NMR (400 MHz, CDCl₃) δ 7.58 (dd, J=8.4, 7.2 Hz, 1H), 7.22 (dd, J=7.2, 0.8 Hz, 1H), 6.83 (dd, J=8.8, 0.8 Hz, 1H), 1.59 (s, 9H).

### Preparation of (6-(tert-Butoxy)pyridin-2-yl)methenamine

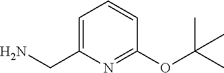

To a stirred solution of 6-(tert-butoxy) picolinonitrile (30 g, 170.4 mmol) in ethanol (300 mL) was added Raney Ni (6 g, 20%) carefully, followed by addition of aq. NH₃(50 mL). The reaction mixture was stirred in 400 mL autoclave under 100 psi hydrogen pressure for 16 hours at room temperature. After completion of reaction, the reaction mixture was filtered through diatomaceous earth and the bed was washed with 10% of MeOH/CH₂Cl₂ (100 mL). The obtained filtrate was concentrated under reduced pressure to afford (6-(tert-butoxy)pyridin-2-yl)methanamine (30 g, crude) as a colorless oil. ¹H NMR (400 MHz, CDCl₃) δ 7.45 (t, J=7.2 Hz, 1H), 6.72 (d, J=6.0 Hz, 1H), 6.50 (d, J=6.0 Hz, 1H), 3.83 (br s, 2H), 1.75 (br s, 2H), 1.59 (s, 9H).

### Preparation of N-((6-(tert-Butoxy)pyridin-2-yl)methyl)-2-nitrobenzenesulfonamide

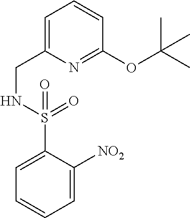 [SMILES: CC(C)(C)Oc1cccc(CNS(=O)(=O)c2ccccc2[N+](=O)[O-])n1]

To a stirred solution of (6-(tert-butoxy)pyridin-2-yl)methenamine (7 g, 38.88 mmol) in dichloromethane (50 mL) and DIPEA (14.33 mL, 116.6 mmol) at 0° C. was added solution of 2-nitrobenzenesulfonyl chloride (9.4 g, 42.76 mmol) in dichloromethane (20 mL) dropwise and the reaction mixture was slowly warmed to room temperature and stirred under nitrogen for 4 hours. After completion of reaction, ice cold water (100 mL) was added and the resulting mixture was extracted with dichloromethane (2×100 mL). The combined organic layers were washed with aq. NaHCO₃ solution (100 mL) followed by brine (50 mL) and dried over anhydrous sodium sulfate and the drying agent was removed by decantation. The resulting solution was concentrated under reduced pressure and the residue obtained was purified by chromatography (silica 5% EtOAc in Hexane) to afford N-((6-(tert-butoxy)pyridin-2-yl)methyl)-2-nitrobenzenesulfonamide (9.7 g, 67%) as a light yellow solid. ¹H NMR (400 MHz, CDCl₃) δ 8.06-8.05 (m, 1H), 8.86-8.82 (m, 1H), 7.71-7.64 (m, 2H), 7.41 (t, J=7.2 Hz, 1H), 6.72 (d, J=7.2 Hz, 1H), 6.49 (d, J=8.4 Hz, 1H), 6.29 (br s, 1H), 4.29 (d, J=3.2 Hz, 1H), 1.53 (s, 9H).

### Preparation of N-(2-Bromoethyl)-N-((6-(tert-butoxy)pyridin-2-yl)methyl)-2-nitrobenzenesulfonamide

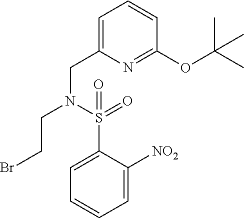 [SMILES: CC(C)(C)Oc1cccc(CN(CCBr)S(=O)(=O)c2ccccc2[N+](=O)[O-])n1]

To a stirred solution of N-((6-(tert-butoxy)pyridin-2-yl)methyl)-2-nitrobenzenesulfonamide (20 g, 54.79 mmol) and Cs₂CO₃ (53.42 g, 164.3 mmol) in DMF (200 mL) at 0° C. was added 1,2-dibromo ethane (8.5 mL, 98.62 mmol) dropwise and the resulting reaction mixture was stirred under nitrogen at ambient temperature for 16 hours. After completion of reaction, the reaction was quenched with ice water (100 mL). The resulting mixture was extracted with EtOAc (2×200 mL), the combined organics were washed with brine solution (100 mL) then dried over anhydrous sodium sulfate, and the drying agent was removed by decantation. The resulting solution was concentrated under reduced pressure and the obtained residue was purified by chromatography (silica 15% EtOAc in Hexane) to afford N-(2-bromoethyl)-N-((6-(tert-butoxy)pyridin-2-yl)methyl)-2-nitrobenzenesulfonamide (8.4 g, 32%) as a light yellow semi solid. ¹H NMR (400 MHz, CDCl₃) δ 7.96 (dd, J=6.8, 0.8 Hz, 1H), 7.721-7.614 (m, 3H), 7.47 (t, J=7.6 Hz, 1H), 6.85 (d, J=7.2 Hz, 1H), 6.56 (d, J=8.4 Hz, 1H), 4.57 (s, 1H), 3.76 (t, J=7.2 Hz, 2H), 3.31 (t, J=8.0 Hz, 2H), 1.53 (s, 9H). ESI MS m/z 473 [C₁₈H₂₂BrN₃O₅S+H]⁺.

### Preparation of N-(2-Bromoethyl)-N-((6-hydroxypyridin-2-yl)methyl)-2-nitrobenzenesulfonamide

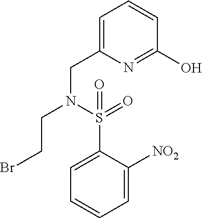 [SMILES: O=[N+]([O-])c1ccccc1S(=O)(=O)N(CCBr)Cc1cccc(O)n1]

To a stirred solution of N-(2-bromoethyl)-N-((6-(tert-butoxy)pyridin-2-yl)methyl)-2-nitrobenzenesulfonamide (1.0 g, 2.12 mmol) at 0° C. was added trifluoroacetic acid (1.0 mL) slowly and the reaction mixture was stirred at ambient temperature for 4 hours. After completion of the reaction, the reaction mixture was concentrated under reduced pressure to afford N-(2-bromoethyl)-N-((6-hydroxypyridin-2-yl)methyl)-2-nitrobenzenesulfonamide (1.0 g, crude) as a light yellow semi solid which was used without further purification: ¹H NMR (400 MHz, CDCl₃) δ 8.07 (dd, J=7.2, 0.8 Hz, 1H), 7.87 (dd, J=8.8, 7.2 Hz, 1H), 7.82-7.72 (m, 3H), 7.01 (d, J=7.2 Hz, 1H), 6.89 (d, J=8.8 Hz, 1H), 4.69 (s, 2H), 3.80 (t, J=7.2 Hz, 2H), 3.40 (t, J=7.2 Hz, 2H). ESI MS m/z 417 [C₁₄H₁₄BrN₃O₅S+H]⁺.

### Preparation of 2-((2-Nitrophenyl)sulfonyl)-1,2,3,4-tetrahydro-6H-pyrido[1,2-a]pyrazin-6-one

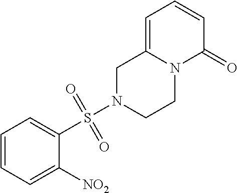 [SMILES: O=c1cccc2n1CCN(S(=O)(=O)c1ccccc1[N+](=O)[O-])C2]

To a stirred solution of N-(2-bromoethyl)-N-((6-hydroxypyridin-2-yl)methyl)-2-nitrobenzenesulfonamide (8.5 g, 20.43 mmol) in THF (85 mL) cooled to 0° C. was added K₂CO₃ (8.4 g, 61.29 mmol) portion wise and the resulting reaction mixture was stirred under nitrogen for 16 hours at ambient temperature. After completion of reaction, the reaction was quenched with ice water (100 mL) solution. The resulting mixture was extracted with EtOAc (2×100 mL), the combined organics were dried over anhydrous sodium sulfate and the drying agent was removed by decantation. The resulting solution was concentrated under reduced pressure to afford 2-((2-nitrophenyl)sulfonyl)-1,2,3,4-tetrahydro-6H-pyrido[1,2-a]pyrazin-6-one (6.5 g, crude) as a yellow solid and used without any further purification. ¹H NMR (400 MHz, CDCl₃) δ 8.06 (d, J=7.2 Hz, 1H), 7.77-7.68 (m, 3H), 7.31 (t, J=7.2 Hz, 1H), 6.51 (d, J=9.2 Hz, 1H), 6.16 (d, J=6.4 Hz, 1H), 4.53 (s, 2H), 4.24 (t, J=5.2 Hz, 2H), 3.72 (t, J=5.2 Hz, 2H). ESI MS m/z 336 [C₁₄H₁₃N₃O₅S+H]⁺.

### Preparation of 7-Bromo-2-((2-nitrophenyl)sulfonyl)-1,2,3,4-tetrahydro-6H-pyrido[1,2-a]pyrazin-6-one

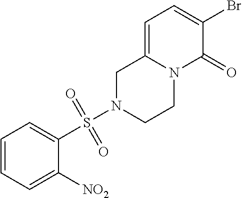 [SMILES: O=c1c(Br)ccc2n1CCN(S(=O)(=O)c1ccccc1[N+](=O)[O-])C2]

To a stirred solution of 2-((2-nitrophenyl) sulfonyl)-1,2,3,4-tetrahydro-6H-pyrido[1,2-a]pyrazin-6-one (44 g, 131.34 mmol) in THF (440 mL) cooled to 0° C. was added NBS (23.3 g, 131.34 mmol) portion wise and the resulting reaction mixture was stirred at room temperature for 10 min. After completion of reaction, the reaction was quenched with ice water (300 mL) solution. The resulting mixture was extracted with EtOAc (2×400 mL), the combined organics were washed with brine solution (200 mL) then dried over anhydrous sodium sulfate and the drying agent was removed by decantation. The resulting solution was concentrated under reduced pressure and the obtained residue was purified by chromatography (silica 20% EtOAc in Hexane) to afford 7-bromo-2-((2-nitrophenyl)sulfonyl)-1,2,3,4-tetrahydro-6H-pyrido[1,2-a]pyrazin-6-one (20 g, 37%) as a light brown solid. ¹H NMR (400 MHz, CDCl₃) δ 8.06 (d, J=7.2, 1.6 Hz, 1H), 7.79-7.68 (m, 4H), 6.08 (d, J=7.6 Hz, 1H), 4.52 (s, 2H), 4.28 (t, J=5.6 Hz, 2H), 3.73 (t, J=5.6 Hz, 2H). ESI MS m/z 415 [C₁₄H₁₂BrN₃O₅S+H]⁺.

### Preparation of tert-Butyl 7-bromo-6-oxo-1,3,4,6-tetrahydro-2H-pyrido[1,2-a]pyrazine-2-carboxylate

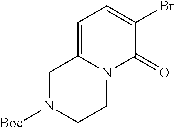 [SMILES: CC(C)(C)OC(=O)N1CCn2c(ccc(Br)c2=O)C1]

To a stirred solution of 7-bromo-2-((2-nitrophenyl)sulfonyl)-1,2,3,4-tetrahydro-6H-pyrido[1,2-a]pyrazin-6-one (28 g, 67.79 mmol) in DMF (280 mL) cooled to 0° C. was added thiophenol (14.9 g, 135.52 mmol) followed by K₂CO₃ (28 g, 203.37 mmol) and the resulting reaction mixture was allowed to stir at room temperature. After 3 hours, Boc anhydride (22.2 g 101.68) was added at room temperature and the reaction mixture was stirred for additional 16 hours. After completion of reaction, the reaction was quenched with ice water (300 mL) solution. The resulting mixture was extracted with EtOAc (2×200 mL), the combined organics were washed with brine solution (150 mL), dried over anhydrous sodium sulfate and the drying agent was removed by decantation. The resulting solution was concentrated under reduced pressure and the residue obtained was purified by chromatography (silica 20% EtOAc in Hexane) to afford tert-butyl 7-bromo-6-oxo-1,3,4,6-tetrahydro-2H-pyrido[1,2-a]pyrazine-2-carboxylate (14 g, 63%) as a light yellow solid. ¹H NMR (400 MHz, CDCl₃) δ 7.69 (d, J=7.6 Hz 1H), 6.02 (d, J=7.2 Hz, 1H), 4.46 (s, 2H), 4.30 (t, J=5.6 Hz, 2H), 3.67 (t, J=5.6 Hz, 2H), 1.49 (s, 9H). ESI MS m/z 327 [C₁₃H₁₇BrN₂O₃−H]⁺.

### Preparation of Tert-Butyl 7-(3-cyano-5-fluorophenyl)-6-oxo-1,3,4,6-tetrahydro-2H-pyrido[1,2-a]pyrazine-2-carboxylate

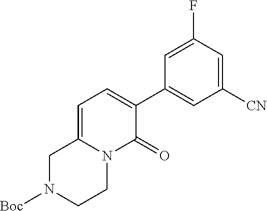 [SMILES: CC(C)(C)OC(=O)N1CCn2c(ccc(-c3cc(F)cc(C#N)c3)c2=O)C1]

To a stirred solution of tert-butyl 7-bromo-6-oxo-1,3,4,6-tetrahydro-2H-pyrido[1,2-a]pyrazine-2-carboxylate (7 g, 21.34 mmol) in acetonitrile: water (3:1) (70 mL) were added (3-cyano-5-fluorophenyl)boronic acid (5.27 g, 32.01 mmol) and solid Na₂CO₃ (6.79 g, 64.02 mmol) at room temperature. The resulting reaction mixture was degassed with nitrogen for 15 min and Pd(dppf)Cl₂·DCM (1.74 g, 2.13 mmol) was added and the reaction mixture was stirred at 80° C. for 8 hours. After completion of reaction, the reaction was quenched with ice water (300 mL) solution. The resulting mixture was extracted with EtOAc (2×200 mL), the combined organics were washed with brine solution (100 mL), then dried over anhydrous sodium sulfate and the drying agent was removed by decantation. The resulting solution was concentrated under reduced pressure and the obtained residue was purified by chromatography (silica 30% EtOAc in Hexane) to afford tert-butyl 7-(3-cyano-5-fluorophenyl)-6-oxo-1,3,4,6-tetrahydro-2H-pyrido[1,2-a]pyrazine-2-carboxylate (5.5 g, 70%) as a light yellow solid. ¹H NMR (400 MHz, CDCl₃) δ 7.81 (s, 1H), 7.77 (d, J=10.0, 1H), 7.52 (d, J=7.2 Hz, 1H), 7.30 (dd, J=8.0, 0.8 Hz, 1H), 6.26 (d, J=7.2 Hz, 1H), 4.56 (s, 2H), 4.32 (t, J=5.6 Hz, 2H), 3.74 (t, J=5.6 Hz, 2H), 1.50 (s, 9H). ESI MS m/z 370 [C₂₀H₂₀FN₃O₃+H]⁺.

### Preparation of 3-Fluoro-5-(6-axo-1,3,4,6-tetrahydro-2H-pyrido[1,2-a]pyrazin-7-yl)benzonitrile Hydrochloride

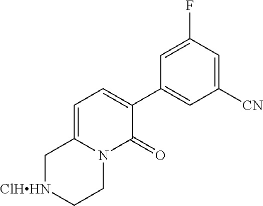

To a stirred solution of tert-butyl 7-(3-cyano-5-fluorophenyl)-6-oxo-1,3,4,6-tetrahydro-2H-pyrido[1,2-a]pyrazine-2-carboxylate (5 g, 13.55 mmol) in CH₂Cl₂ (50 mL) was added solution of 2 M HCl in MTBE (20 mL) at 0° C. The resulting reaction mixture was stirred for 4 hours at ambient temperature. After completion of the reaction, the reaction mixture was concentrated under reduced pressure to afford 3-fluoro-5-(6-oxo-1,3,4,6-tetrahydro-2H-pyrido[1,2-a]pyrazin-7-yl)benzonitrile hydrochloride (4.1 g, crude) as a yellow solid which was used without further purification. ¹H NMR (400 MHz, DMSO-d₆) δ 10.08 (s, 2H), 8.12 (s, 1H), 8.07-8.03 (m, 1H), 7.95 (d, J=7.2 Hz, 1H), 7.82-7.79 (m, 1H), 6.51 (d, J=7.6 Hz, 1H), 4.37 (s, 2H), 4.19 (t, J=5.6 Hz, 2H), 3.53 (t, J=5.6 Hz, 2H). ESI MS m/z 270 [C₁₅H₁₃ClFN₃O+H]⁺.

### Preparation of 3-Fluoro-5-(2-(2-methylbenzoyl)-6-oxo-1,3,4,6-tetrahydro-2H-pyrido[1,2-a]pyrazin-7-yl)benzonitrile

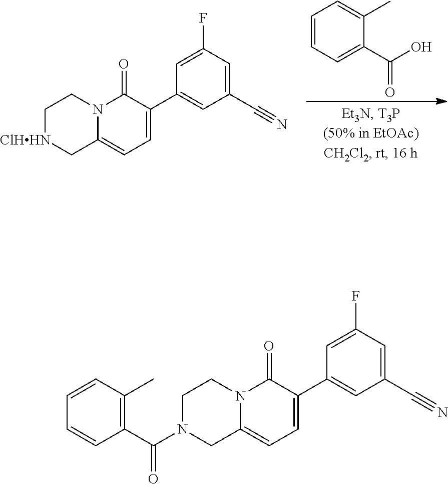

A mixture of 3-fluoro-5-(6-oxo-1,3,4,6-tetrahydro-2H-pyrido[1,2-a]pyrazin-7-yl)benzonitrile·HCl (40 mg, 0.13 mmol), Et₃N (56.1 μL, 0.45 mmol), 2-methylbenzoic acid (21.4 mg, 0.15 mmol), and T₃P (50% in Ethyl acetate) (248.2 μL, 0.45 mmol) in methylene chloride (2 mL) was stirred under nitrogen at ambient temperature for 16 hours. After completion of reaction, aq. NaHCO₃ (2 mL) was added and the resulting mixture was extracted with methylene chloride (3×2 mL). The combined organics were dried over anhydrous sodium sulfate and the drying agent was removed by decantation. The resulting solution was concentrated under reduced pressure and the residue obtained was purified by Mass triggered preparative HPLC to afford 3-fluoro-5-(2-(2-methylbenzoyl)-6-oxo-1,3,4,6-tetrahydro-2H-pyrido[1,2-a]pyrazin-7-yl)benzonitrile (23.3 mg, 46%) as a color less gum. UPLC, method 1: t_{R}=4.06 min, 96.1% (AUC) at 254 nm. ESI MS m/z 388 [C₂₃H₁₈FN₃O₂+H]⁺.

### Preparation of 3-(2-(2,6-Dimethylbenzoyl)-6-oxo-1,3,4,6-tetrahydro-2H-pyrido[1,2-a]pyrazin-7-yl)-5-fluorobenzonitrile

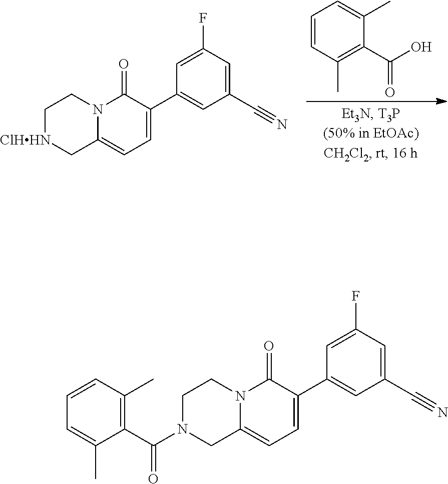

3-(2-(2,6-Dimethylbenzoyl)-6-oxo-1,3,4,6-tetrahydro-2H-pyrido[1,2-a]pyrazin-7-yl)-5-fluorobenzonitrile was prepared using a synthetic procedure similar to the one described above for the synthesis of fluoro-5-(2-(2-methylbenzoyl)-6-oxo-1,3,4,6-tetrahydro-2H-pyrido[1,2-a]pyrazin-7-yl)benzonitrile using 2,6-dimethylbenzoic acid. 3-(2-(2,6-Dimethylbenzoyl)-6-oxo-1,3,4,6-tetrahydro-2H-pyrido[1,2-a]pyrazin-7-yl)-5-fluorobenzonitrile was obtained as an off white solid (18.0 mg, 30%). UPLC, method 2: t_{R}=1.77 min, 97.1% (AUC) at 254 nm. ESI MS m/z 402 [C₂₄H₂₀FN₃O₂+H]⁺.

### Example 6—Synthesis of 7-(2-Oxo-1,2,3,4-tetrahydroquinolin-5-yl)-1,2,3,4-tetrahydro-6H-pyrido[1,2-a]pyrazin-6-one Hydrochloride

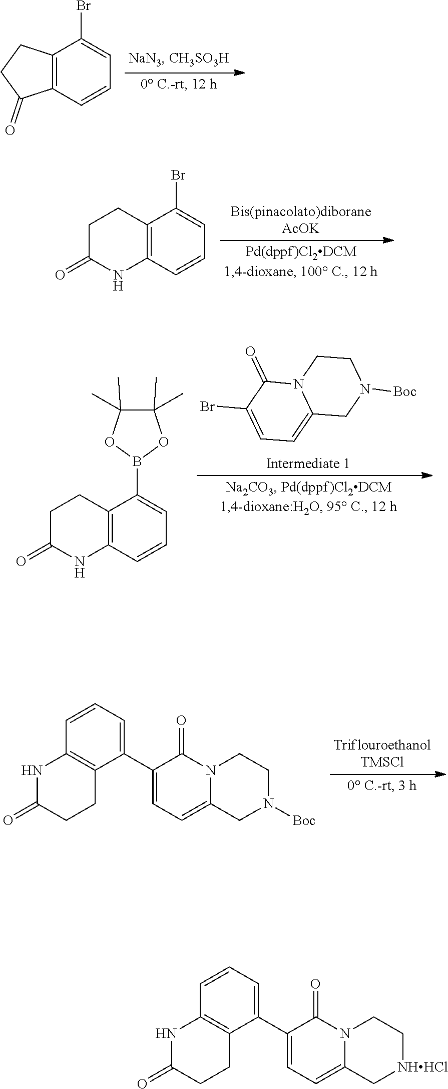

### Preparation of 5-Bromo-3,4-dihydroquinolin-2(1H)-one

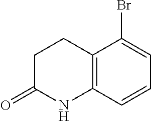 [SMILES: O=C1CCc2c(Br)cccc2N1]

To a stirred solution of 4-bromo-2,3-dihydro-1H-inden-1-one (14 g, 66.35 mmol) in methanesulphonic acid (28 mL) at 0° C. was added NaN₃ (8.60 g, 132.70 mmol) portion wise and the resulting reaction mixture was stirred under nitrogen at ambient temperature for 16 hours. After completion of reaction, the reaction was quenched with aq.NaHCO₃ solution till the solution showed basic pH. The resulting mixture was extracted with EtOAc (2×200 mL), the combined organics were dried over anhydrous sodium sulfate and the drying agent was removed by decantation. The resulting solution was concentrated under reduced pressure and the obtained residue was purified by chromatography (silica 20% EtOAc in Hexane) to afford 5-bromo-3,4-dihydroquinolin-2(1H)-one (8 g, 53%) as a light brown solid. ¹H NMR (400 MHz, CDCl₃) δ 9.31 (br s, 1H) 7.22 (dd, J=8.0, 0.8 Hz, 1H), 7.03 (t, J=8.0 Hz, 1H), 6.81 (d, J=7.6 Hz, 1H), 3.09 (t, J=7.6 Hz, 2H), 2.65 (t, J=7.2 Hz, 2H). ESI MS m/z 226 [C₉H₈BrNO+H]⁺.

### Preparation of 5-(4,4,5,5-tetramethyl-1,3,2-dioxaborolan-2-yl)-3,4-dihydroquinolin-2(1H)-one

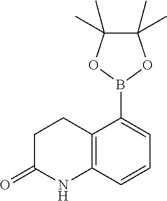 [SMILES: CC1(C)OB(c2cccc3c2CCC(=O)N3)OC1(C)C]

A mixture of 5-bromo-3,4-dihydroquinolin-2(1H)-one (4 g, 17.69 mmol), bis(pinacolato)diborane (7.16 g, 28.31 mmol), and AcOK (5.20 g, 53.07 mmol) in 1,4-dioxane (40 mL) was stirred at room temperature for 10 min. The resulting reaction mixture was degassed with nitrogen for 20 min and Pd(dppf)Cl₂·DCM (1.40 g, 1.76 mmol) was added at room temperature and the resulting reaction mixture was stirred at 100° C. for 12 hours. After completion of reaction, the reaction was quenched with water (100 mL) solution. The resulting mixture was extracted with EtOAc (2×00 mL), the combined organics were washed with brine solution (50 mL), dried over anhydrous sodium sulfate, and the drying agent was removed by decantation. The resulting solution was concentrated under reduced pressure and the obtained residue was used without further purification. ESI MS m/z 274 [C₁₅H₂₀BNO₃+H]⁺.

### Preparation of tert-Butyl 6-oxo-7-(2-oxo-1,2,3,4-tetrahydroquinolin-5-y)-1,3,4,6-tetrahydro-2H-pyrido[1,2-a]pyrazine-2-carboxylate

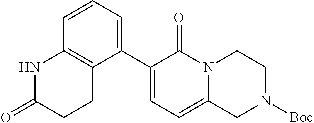 [SMILES: CC(C)(C)OC(=O)N1CCn2c(ccc(-c3cccc4c3CCC(=O)N4)c2=O)C1]

To a stirred solution of tert-butyl 7-bromo-6-oxo-3,4-dihydro-514,615-pyrido[1,6-a]pyrazine-2(1H)-carboxylate (2 g, 6.07 mmol) in acetonitrile: water (3:1) (20 mL) were added 5-(4,4,5,5-tetramethyl-1,3,2-dioxaborolan-2-yl)-3,4-dihydroquinolin-2(1H)-one (2.48 g, 9.11 mmol) and solid Na₂CO₃ (1.93 g, 18.21 mmol) at room temperature. The resulting reaction mixture was degassed with nitrogen for 15 min and Pd(dppf)Cl₂·DCM (0.49 g, 0.60 mmol) was added and stirred at 100° C. for 12 hours. After completion of reaction it was quenched with ice water (50 mL) solution. The resulting mixture was extracted with EtOAc (2×100 mL), the combined organics were washed with brine solution (50 mL), dried over anhydrous sodium sulfate, and the drying agent was removed by decantation. The resulting solution was concentrated under reduced pressure and the obtained residue was purified by chromatography (silica 90% EtOAc in Hexane) to afford tert-butyl 6-oxo-7-(2-oxo-1,2,3,4-tetrahydroquinolin-5-yl)-1,3,4,6-tetrahydro-2H-pyrido[1,2-a]pyrazine-2-carboxylate (1.5 g, 62%) as a light brown solid. ¹H NMR (400 MHz, CDCl₃) δ 8.24 (s, 1H), 7.30 (d, J=7.2 Hz, 1H), 7.18 (t, J=7.6 Hz, 1H), 6.90 (d, J=7.2 Hz, 1H), 6.77 (d, J=8.0 Hz, 1H), 6.22 (d, J=6.8 Hz, 1H), 4.55 (s, 2H), 4.32 (br s, 2H), 3.72 (br s, 2H), 2.94 (br s, 1H), 2.70 (br s, 1H), 2.56 (br s, 2H), 1.50 (s, 9H). ESI MS m/z 370 [C₂₂H₂₅N₃O₄+H]⁺.

### Preparation of 7-(2-Oxo-1,2,3,4-tetrahydroquinolin-5-yl)-1,2,3,4-tetrahydro-6H-pyrido[1,2-a]pyrazin-6-one Hydrochloride

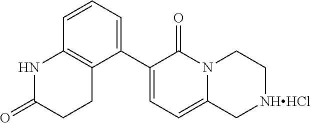

To a stirred solution of tert-butyl 6-oxo-7-(2-oxo-1,2,3,4-tetrahydroquinolin-5-yl)-1,3,4,6-tetrahydro-2H-pyrido[1,2-a]pyrazine-2-carboxylate (1.5 g, 3.79 mmol) in trifluoroethanol (17 mL) was added TMSCl (0.7 mL, 5.68 mmol) carefully at 0° C. and the reaction mixture stirred for 3 hours at ambient temperature. After completion of reaction, the reaction mixture was concentrated under reduced pressure and the crude product obtained was triturated with MTBE to afford 7-(2-oxo-1,2,3,4-tetrahydroquinolin-5-yl)-1,2,3,4-tetrahydro-6H-pyrido[1,2-a]pyrazin-6-one hydrochloride (1.1 g, crude) as a brown solid. ¹H NMR (400 MHz, DMSO-d₆) δ 10.08 (s, 1H), 9.94 (br s, 2H), 7.43 (d, J=7.2 Hz, 1H), 7.16 (t, J=8.0 Hz, 1H), 6.88 (d, J=7.6 Hz, 1H), 6.79 (d, J=7.6 Hz, 1H), 6.42 (d, J=6.8 Hz, 1H), 4.34 (s, 2H), 4.15 (s, 2H), 3.87 (q, J=9.6 Hz, 2H), 3.52 (br s, 2H), 2.34 (t, J=7.6 Hz, 2H). ESI MS m/z 296 [C₁₇H₁₈ClN₃O₂+H]⁺.

### Preparation of 7-(2-Oxo-1,2,3,4-tetrahydroquinolin-5-y)-2-(o-tolylsulfonyl)-1,2,3,4-tetrahydro-6H-pyrido[1,2-a]pyrazin-6-one

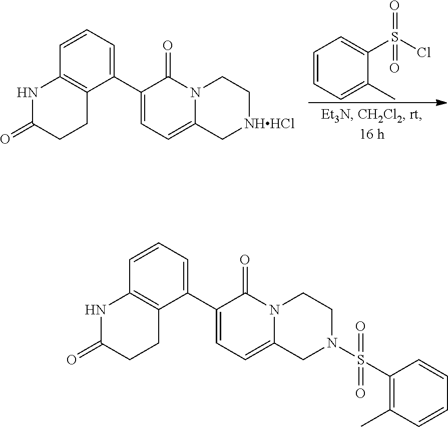

A mixture of 7-(2-oxo-1,2,3,4-tetrahydroquinolin-5-yl)-1,2,3,4-tetrahydro-6H-pyrido[1,2-a]pyrazin-6-one·HCl (50 mg, 0.15 mmol), Et₃N (65 μL, 0.45 mmol), 2-methylbenzenesulfonyl chloride (34.3 mg, 0.18 mmol) in methylene chloride (2 mL) was stirred under nitrogen atmosphere at ambient temperature for 16 hours. After completion of reaction, the reaction was treated with aq. NaHCO₃ (2 mL) and the resulting mixture was extracted with methylene chloride (3×2 mL). The combined organics were dried over anhydrous sodium sulfate and the drying agent was removed by decantation. The resulting solution was concentrated under reduced pressure and the residue obtained was purified by mass triggered preparative HPLC to afford 7-(2-oxo-1,2,3,4-tetrahydroquinolin-5-yl)-2-(o-tolylsulfonyl)-1,2,3,4-tetrahydro-6H-pyrido[1,2-a]pyrazin-6-one (8 mg, 7%) as a brown solid. UPLC, method 2: t_{R}=1.65 min, 98.5% (AUC) at 256 nm. ESI MS m/z 434 [C₂₄H₂₃N₃O₄S+H]⁺.

### Preparation of 2-(3-Chlorobenzoyl)-7-(2-oxo-1,2,3,4-tetrahydroquinolin-5-yl)-1,2,3,4-tetrahydro-6H-pyrido[1,2-a]pyrazin-6-one

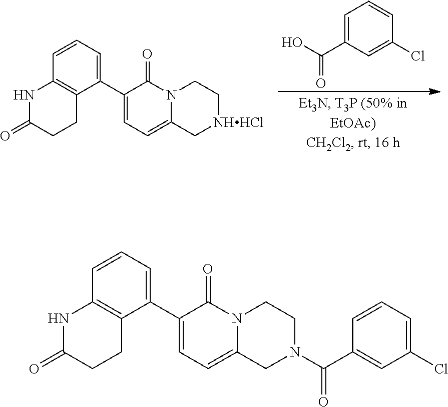

A mixture of 7-(2-oxo-1,2,3,4-tetrahydroquinolin-5-yl)-1,2,3,4-tetrahydro-6H-pyrido[1,2-a]pyrazin-6-one·HCl (50 mg, 0.15 mmol), Et₃N (65 μL, 0.45 mmol), 3-chlorobenzoic acid (28 mg, 0.18 mmol), and T₃P (50 wt % in Ethyl acetate) (288 μL, 0.45 mmol) in methylene chloride (2 mL) was stirred under nitrogen at ambient temperature for 16 hours. After completion of reaction, the reaction was treated with aq.NaHCO₃ (2 mL) and the resulting mixture was extracted with methylene chloride (3×2 mL). The combined organics were dried over anhydrous sodium sulfate and the drying agent was removed by decantation. The resulting solution was concentrated under reduced pressure and the residue obtained was purified by mass triggered preparative HPLC to afford 2-(3-chlorobenzoyl)-7-(2-oxo-1,2,3,4-tetrahydroquinolin-5-yl)-1,2,3,4-tetrahydro-6H-pyrido[1,2-a]pyrazin-6-one (4.9 mg, 8%) as an off-white solid. ¹H NMR (400 MHz, DMSO-d₆) δ 10.09 (s, 1H), 7.59-7.49 (m, 4H), 7.39 (br s, 1H), 7.15 (t, J=7.6 Hz, 1H), 6.86 (d, J=7.6 Hz, 1H), 6.79 (d, J=7.6 Hz, 1H), 6.49-6.27 (m, 1H), 4.76 (br s, 1H), 4.61 (br s, 1H), 4.29 (br s, 1H), 4.20 (br s, 1H), 3.78 (d, J=13.6 Hz, 1H), 3.69 (br s, 1H), 2.66-2.54 (m, 2H), 2.35-2.32 (m, 2H). UPLC, method 1: t_{R}=1.52 min, 97.41% (AUC) at 254 nm. ESI MS m/z 434 [C₂₄H₂₀ClN₃O₃+H]⁺.

### Preparation of 2-(1-Methyl-1H-indole-3-carbonyl)-7-(2-oxo-1,2,3,4-tetrahydroquinolin-5-yl)-1,2,3,4-tetrahydro-6H-pyrido[1,2-a]pyrazin-6-one

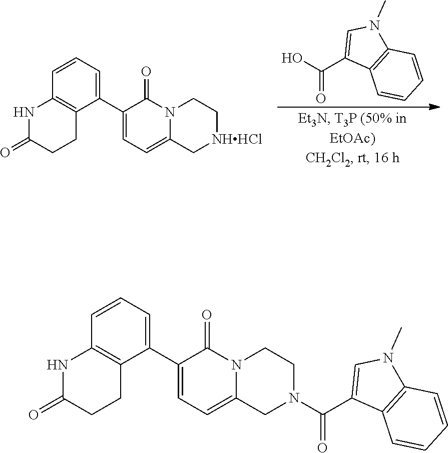

2-(1-Methyl-1H-indole-3-carbonyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(1-Methyl-1H-indole-3-carbonyl)-7-(2-oxo-1,2,3,4-tetrahydroquinolin-5-yl)-1,2,3,4-tetrahydro-6H-pyrido[1,2-a]pyrazin-6-one was obtained as an off-white solid (6.0 mg, 8%). UPLC, method 1: t_{R}=3.44 min, 98.7% (AUC) at 220 nm. ESI MS m/z 453 [C27H₂₄N₄O₃+H]⁺.

### Preparation of 2-Benzoyl-7-(2-oxo-1,2,3,4-tetrahydroquinolin-5-yl)-1,2,3,4-tetrahydro-6H-pyrido[1,2-a]pyrazin-6-one

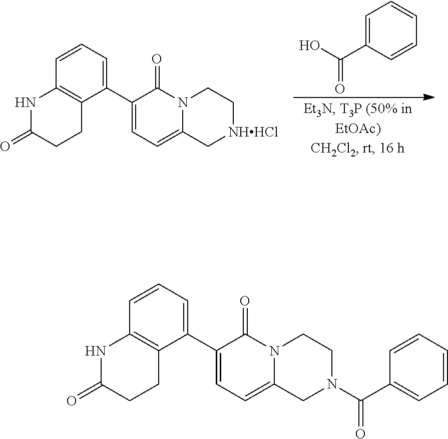

2-Benzoyl-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-Benzoyl-7-(2-oxo-1,2,3,4-tetrahydroquinolin-5-yl)-1,2,3,4-tetrahydro-6H-pyrido[1,2-a]pyrazin-6-one was obtained as an off-white solid (10 mg, 14%). UPLC, method 2: t_{R}=1.21 min, 98.3% (AUC) at 220 nm. ESI MS m/z 400 [C₂₄H₂₁N₃O₃+H]⁺.

### Preparation of 2-(2-Methylbenzoyl)-7-(2-oxo-1,2,3,4-tetrahydroquinolin-5-yl)-1,2,3,4-tetrahydro-6H-pyrido[1,2-a]pyrazin-6-one

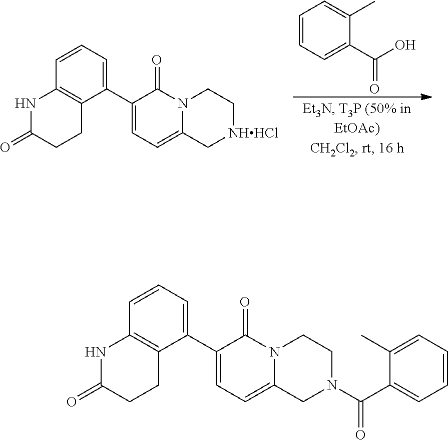

2-(2-Methylbenzoyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(2-Methylbenzoyl)-7-(2-oxo-1,2,3,4-tetrahydroquinolin-5-yl)-1,2,3,4-tetrahydro-6H-pyrido[1,2-a]pyrazin-6-one was obtained as an off-white solid (14.3 mg, 19%). UPLC, method 2: t_{R}=1.28 min, 98.1% (AUC) at 220 nm. ESI MS m/z 414 [C₂₅H₂₃N₃O₃+H]⁺.

### Preparation of 2-(3-Methylbenzoyl)-7-(2-oxo-1,2,3,4-tetrahydroquinolin-5-yl)-1,2,3,4-tetrahydro-6H-pyrido[1,2-a]pyrazin-6-one

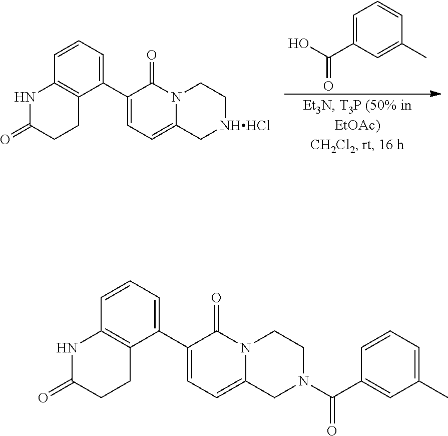

2-(3-Methylbenzoyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(3-Methylbenzoyl)-7-(2-oxo-1,2,3,4-tetrahydroquinolin-5-yl)-1,2,3,4-tetrahydro-6H-pyrido[1,2-a]pyrazin-6-one was obtained as an off-white solid (12.7 mg, 17%). UPLC, method 2: t_{R}=1.34 min, 98.8% (AUC) at 220 nm. ESI MS m/z 414 [C₂₅H₂₃N₃O₃+H]⁺.

### Preparation of 2-(2-Chlorobenzoyl)-7-(2-oxo-1,2,3,4-tetrahydroquinolin-5-yl)-1,2,3,4-tetrahydro-6H-pyrido[1,2-a]pyrazin-6-one

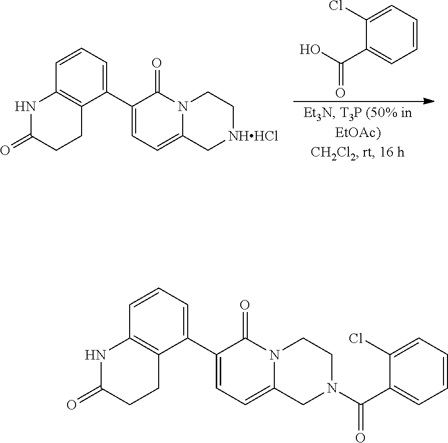

2-(2-Chlorobenzoyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(2-Chlorobenzoyl)-7-(2-oxo-1,2,3,4-tetrahydroquinolin-5-yl)-1,2,3,4-tetrahydro-6H-pyrido[1,2-a]pyrazin-6-one was obtained as an off-white solid (12.8 mg, 18%). UPLC, method 2: t_{R}=1.26 min, 97.4% (AUC) at 220 nm. ESI MS m/z 434 [C₂₄H₂₀ClN₃O₃+H]⁺.

### Preparation of 2-(2-Methoxybenzoyl)-7-(2-oxo-1,2,3,4-tetrahydroquinolin-5-yl)-1,2,3,4-tetrahydro-6H-pyrido[1,2-a]pyrazin-6-one

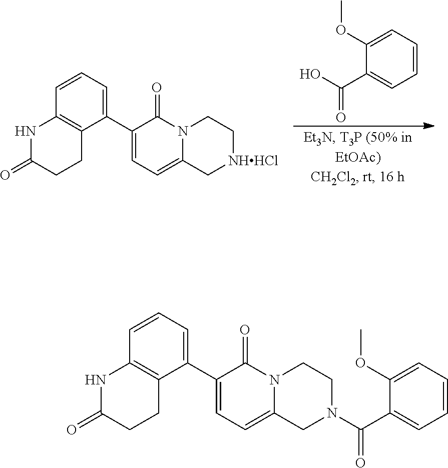

2-(2-Methoxybenzoyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(2-Methoxybenzoyl)-7-(2-oxo-1,2,3,4-tetrahydroquinolin-5-yl)-1,2,3,4-tetrahydro-6H-pyrido[1,2-a]pyrazin-6-one was obtained as an off-white solid (10.3 mg, 14%). UPLC, method 2: t_{R}=1.34 min, 94.4% (AUC) at 220 nm. ESI MS m/z 430 [C₂₅H₂₃N₃O₄+H]⁺.

### Preparation of 2-(3-Methoxybenzoyl)-7-(2-oxo-1,2,3,4-tetrahydroquinolin-5-yl)-1,2,3,4-tetrahydro-6H-pyrido[1,2-a]pyrazin-6-one

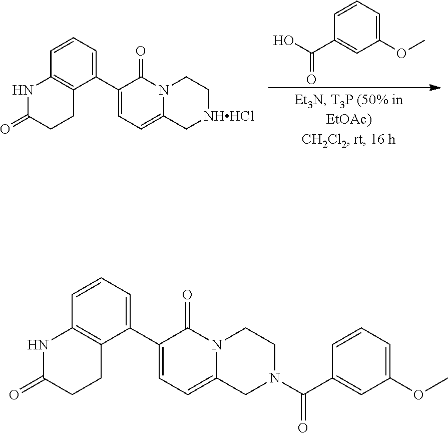

2-(3-Methoxybenzoyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(3-Methoxybenzoyl)-7-(2-oxo-1,2,3,4-tetrahydroquinolin-5-yl)-1,2,3,4-tetrahydro-6H-pyrido[1,2-a]pyrazin-6-one was obtained as an off-white solid (3.8 mg, 5%). UPLC, method 2: t_{R}=1.39 min, 95.2% (AUC) at 245.7 nm. ESI MS m/z 430 [C₂₅H₂₃N₃O₄+H]⁺.

### Preparation of 2-(4-Methylbenzoyl)-7-(2-oxo-1,2,3,4-tetrahydroquinolin-5-yl)-1,2,3,4-tetrahydro-6H-pyrido[1,2-a]pyrazin-6-one

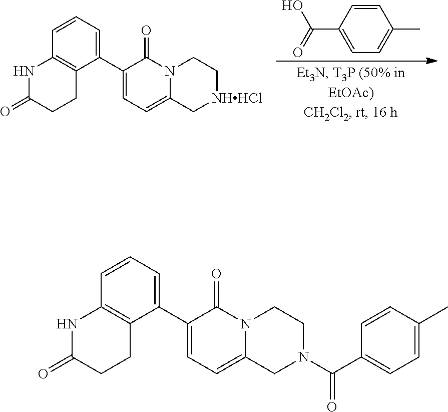

2-(4-Methylbenzoyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(4-Methylbenzoyl)-7-(2-oxo-1,2,3,4-tetrahydroquinolin-5-yl)-1,2,3,4-tetrahydro-6H-pyrido[1,2-a]pyrazin-6-one was obtained as an off-white solid (8.8 mg, 16%). UPLC, method 2: t_{R}=2.95 min, 98.7% (AUC) at 242.7 nm. ESI MS m/z 414 [C₂₅H₂₃N₃O₃+H]⁺.

### Preparation of 2-(4-Chlorobenzoyl)-7-(2-oxo-1,2,3,4-tetrahydroquinolin-5-yl)-1,2,3,4-tetrahydro-6H-pyrido[1,2-a]pyrazin-6-one

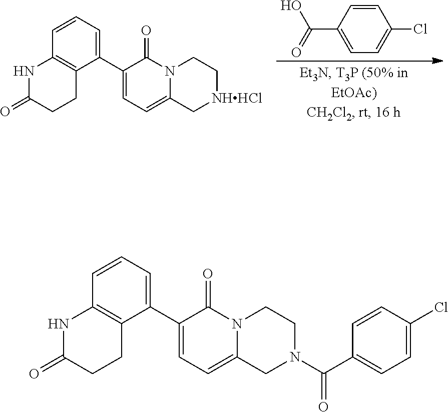

2-(4-Chlorobenzoyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(4-Chlorobenzoyl)-7-(2-oxo-1,2,3,4-tetrahydroquinolin-5-yl)-1,2,3,4-tetrahydro-6H-pyrido[1,2-a]pyrazin-6-one was obtained as an off-white solid (10.5 mg, 18%). UPLC, method 2: t_{R}=3.11 min, >99.5% (AUC) at 242.7 nm. ESI MS m/z 434 [C₂₄H₂₀ClN₃O₃+H]⁺.

### Preparation of 2-(1H-Indole-3-carbonyl)-7-(2-oxo-1,2,3,4-tetrahydroquinolin-5-y)-1,2,3,4-tetrahydro-6H-pyrido[1,2-a]pyrazin-6-one

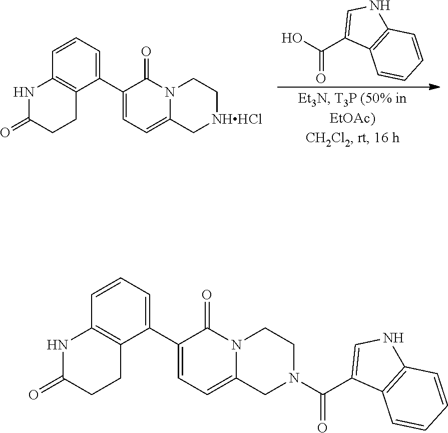

2-(1H-Indole-3-carbonyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(1H-Indole-3-carbonyl)-7-(2-oxo-1,2,3,4-tetrahydroquinolin-5-yl)-1,2,3,4-tetrahydro-6H-pyrido[1,2-a]pyrazin-6-one (5.5 mg, 9%) as an off-white solid. UPLC, method 2: t_{R}=2.71 min, 92.4% (AUC) at 220 nm. ESI MS m/z 439 [C₂₆H₂₂N₄O₃+H]⁺.

### Preparation of 2-(Cyclohexanecarbonyl)-7-(2-oxo-1,2,3,4-tetrahydroquinolin-5-y)-1,2,3,4-tetrahydro-6H-pyrido[1,2-a]pyrazin-6-one

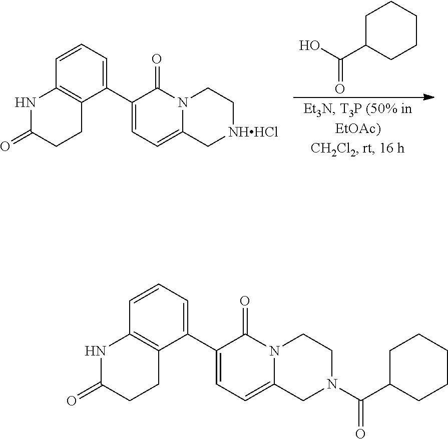

2-(Cyclohexanecarbonyl)-7-(2-oxo-1,2,3,4-tetrahydroquinolin-5-yl)-1,2,3,4-tetrahydro-6H-pyrido[1,2-a]pyrazin-6-one was prepared using a synthetic procedure similar to the one described above for the synthesis of 2-(3-chlorobenzoyl)-7-(2-oxo-1,2,3,4-tetrahydroquinolin-5-yl)-1,2,3,4-tetrahydro-6H-pyrido[1,2-a]pyrazin-6-one. 2-(Cyclohexanecarbonyl)-7-(2-oxo-1,2,3,4-tetrahydroquinolin-5-yl)-1,2,3,4-tetrahydro-6H-pyrido[1,2-a]pyrazin-6-one was obtained as an off-white solid (28.6 mg, 58%). UPLC, method 2: t_{R}=3.08 min, >99.0% (AUC) at 254 nm. ESI MS m/z 406 [C₂₄H₂₇N₃O₃+H]⁺.

### Preparation of 2-Cyclopentyl-7-(2-oxo-1,2,3,4-tetrahydroquinolin-5-yl)-1,2,3,4-tetrahydro-6H-pyrido[1,2-a]pyrazin-6-one

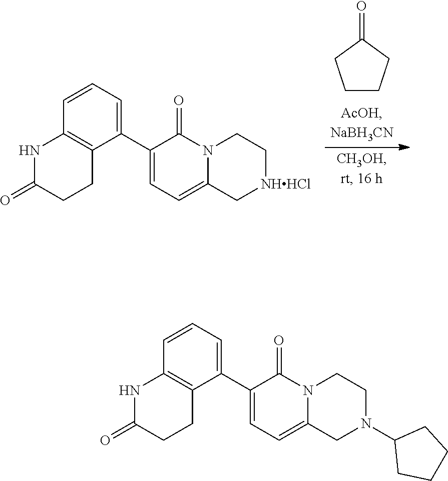

A mixture of 7-(2-oxo-1,2,3,4-tetrahydroquinolin-5-yl)-1,2,3,4-tetrahydro-6H-pyrido[1,2-a]pyrazin-6-one·HCl (50 mg, 0.15 mmol), cyclopentanone (15.2 mg, 0.18 mmol), and acetic acid (catalytic) in MeOH (3 mL) was stirred at room temperature. After 4 hours, NaBH₃CN (19 mg, 0.30 mmol) was added and the reaction mixture was allowed to stir for additional 12 hours at rt. After completion of reaction, the reaction was treated with aq. NaHCO₃ (2 mL) and resulting mixture was extracted with methylene chloride (3×2 mL). The combined organic fractions were dried over anhydrous sodium sulfate and the drying agent was removed by decantation. The resulting solution was concentrated under reduced pressure and the obtained residue was purified by mass triggered preparative HPLC to afford 2-cyclopentyl-7-(2-oxo-1,2,3,4-tetrahydroquinolin-5-yl)-1,2,3,4-tetrahydro-6H-pyrido[1,2-a]pyrazin-6-one (3.5 mg, 6%) as a brown solid. UPLC, method 2: t_{R}=1.34 min, 95.7% (AUC) at 254 nm. ESI MS m/z 364 [C₂₂H₂₅N₃O₂+H]⁺.

### Example 7—Screening Compounds for Artemis Inhibitory Activity

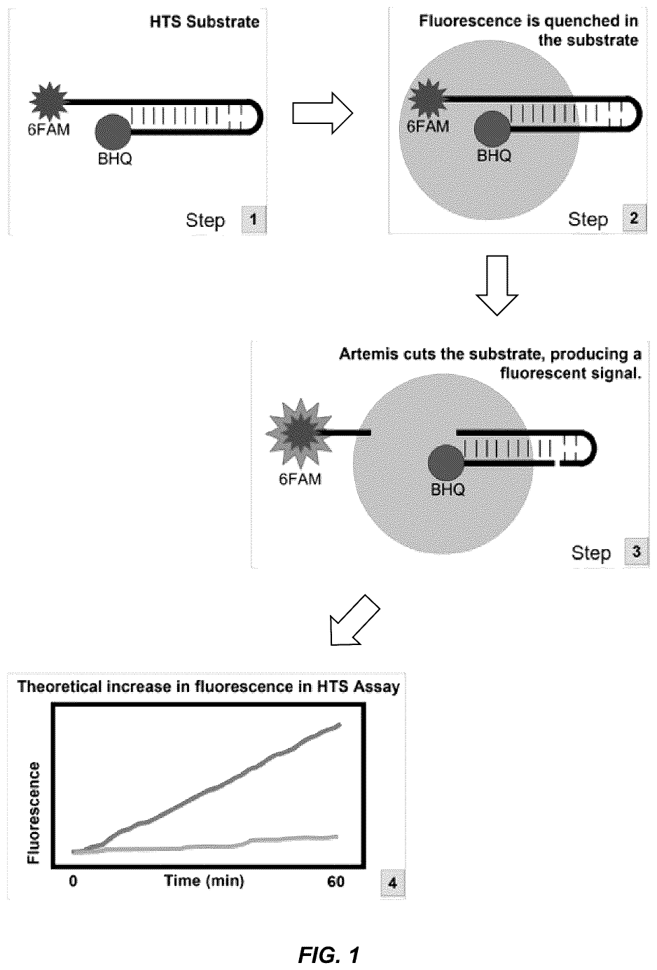

Artemis activity assay was developed using full length Artemis with DNA-PKcs to generate a fluorescent signal from the cleavage of the 5′ 6-FAM modified end of hairpin DNA with a black-hole quencher (3-BHQ) on the 3′ end. See FIG. 1. The assay buffer used was 25 mM MOPS, pH 7.5, 10 mM MgCl2, 10 mM KCl, 10 μM ATP, 1 mM DTT, 0.05% (w/v) CHAPS with 5 nM FL-Artemis, 5 nM DNA-PKcs, and 200 nM DNA FAM hairpin substrate. The fluorescence signal was monitored kinetically on a Perkin Elmer Envision microplate reader with FITC filters (λex=485 nm, λem=520 nm) with 1-min reads for 40 min. Compounds were tested in 10-point concentration (10 different concentrations with 3-fold dilutions) responses using the slopes of the enzyme readouts to determine percent inhibition which was fit to a 4-parameter logistic curve to calculate the IC₅₀ of each compound. The purity of the protein preparations were published previously (Ma et al., “A Biochemically Defined System for Mammalian Nonhomologous DNA End Joining,” Mol. Cell 16:701-713 (2004); Ma et al., “Hairpin Opening and Overhang Processing by an Artemis/DNA-dependent Protein Kinase Complex in Nonhomologous End Joining and V(D)J Recombination,” Cell 108:781-794 (2002); Lu et al., “A Biochemically Defined System for Coding Joint Formation in V(D)J Recombination,” Mol. Cell 31:485-497 (2008), each of which is hereby incorporated by reference in its entirety.

Table 3 below shows the structure of 100 compounds that were screened using this assay and the calculated IC₅₀ values for each.

TABLE 3

| Compound Structure | Calculated IC₅₀ (uM) |
|---|---|
| 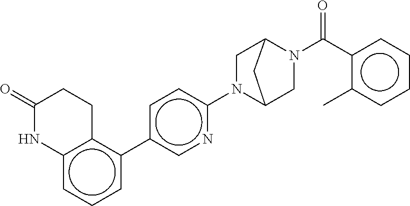 | 0.018 |
| 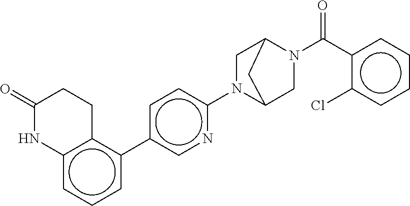 | 0.02 |
| 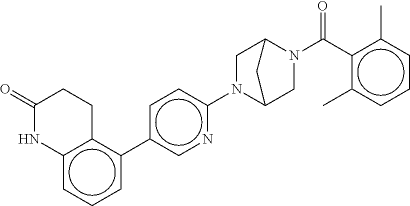 | 0.022 |
| 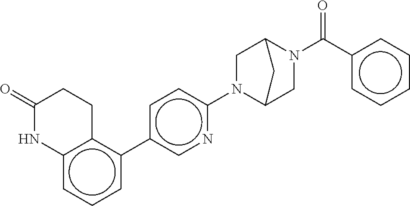 | 0.022 |
| 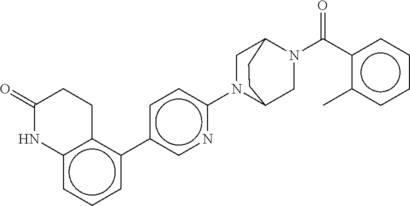 | 0.032 |
| 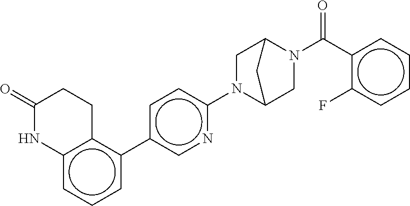 | 0.033 |
| 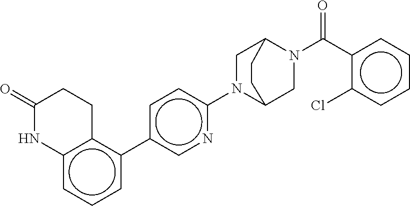 | 0.038 |
| 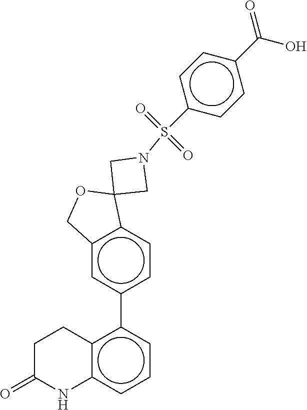 | 0.039 |
| 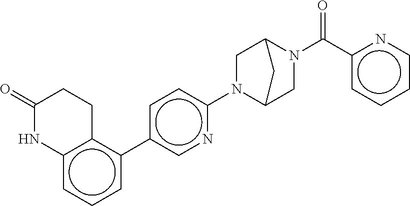 | 0.04 |
| 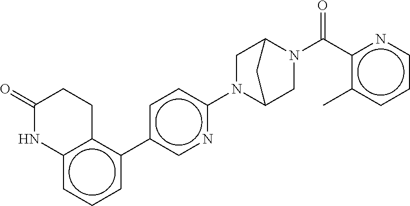 | 0.043 |
| 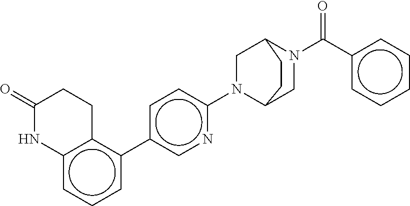 | 0.044 |
| 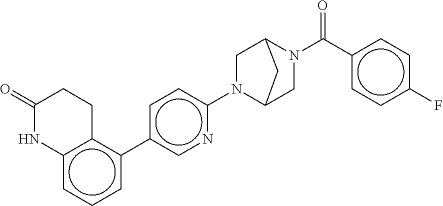 | 0.045 |
| 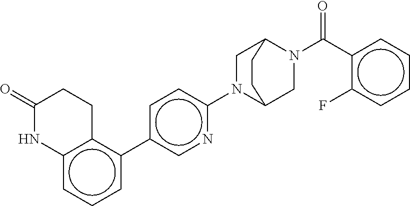 | 0.049 |
| 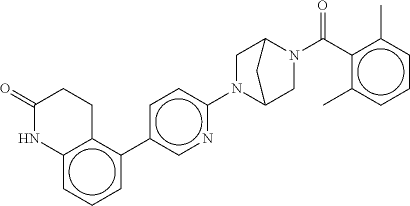 | 0.05 |
| 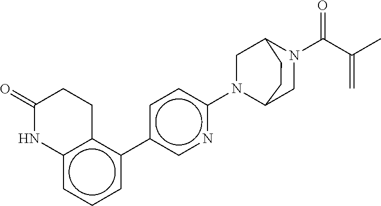 | 0.071 |
| 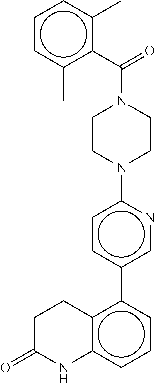 | 0.073 |
| 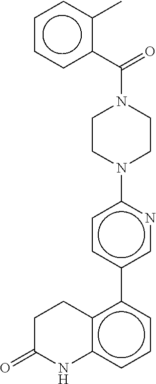 | 0.081 |
| 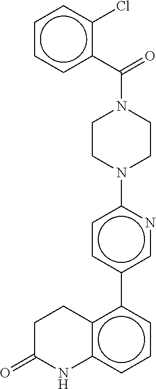 | 0.083 |
| 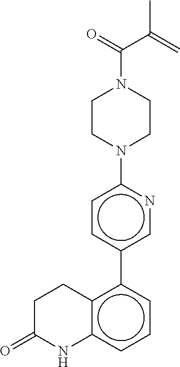 | 0.086 |
| 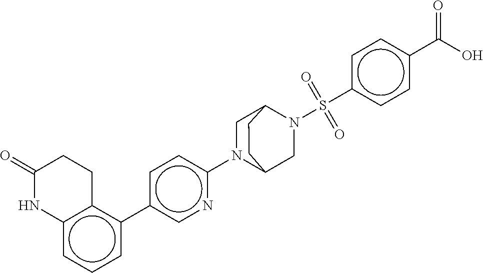 | 0.087 |
| 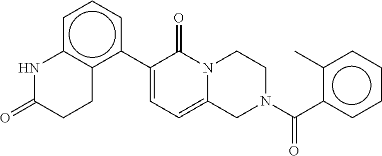 | 0.088 |
| 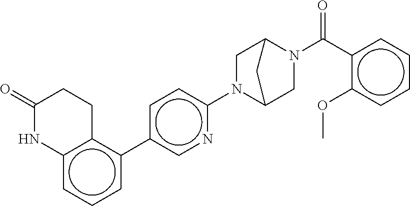 | 0.088 |
| 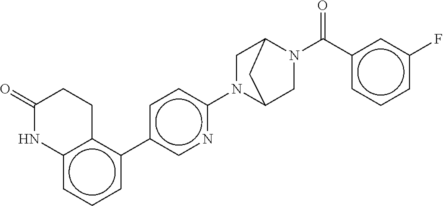 | 0.092 |
| 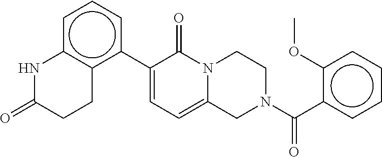 | 0.092 |
| 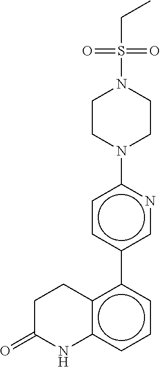 | 0.093 |
| 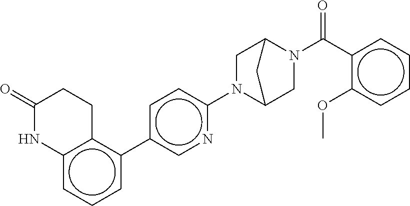 | 0.093 |
| 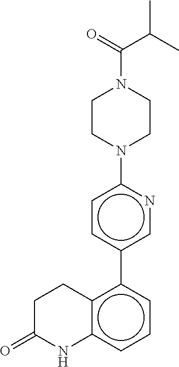 | 0.094 |
| 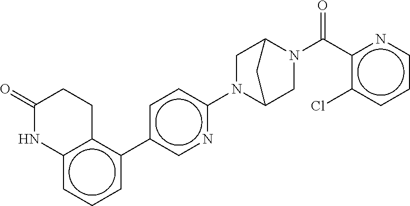 | 0.095 |
| 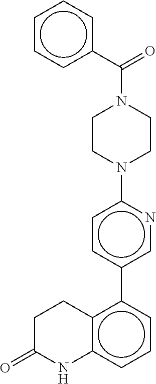 | 0.098 |
| 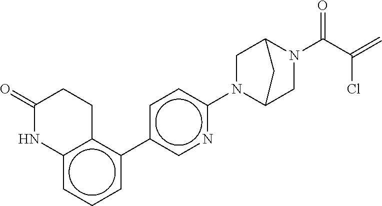 | 0.106 |
| 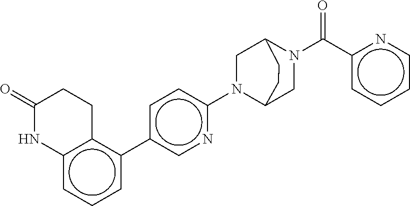 | 0.112 |
| 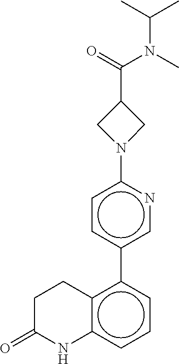 | 0.12 |
| 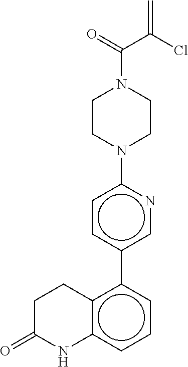 | 0.12 |
| 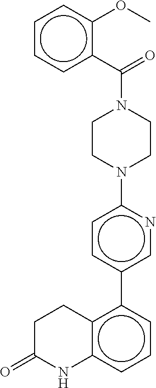 | 0.124 |
| 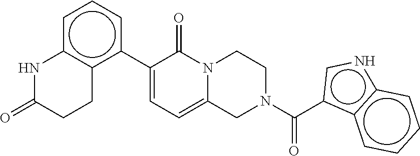 | 0.124 |
| 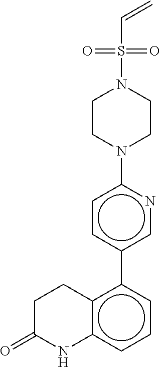 | 0.125 |
| 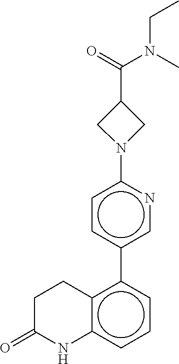 | 0.14 |
| 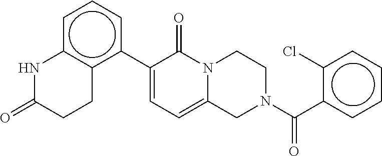 | 0.14 |
| 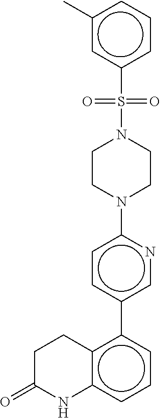 | 0.146 |
| 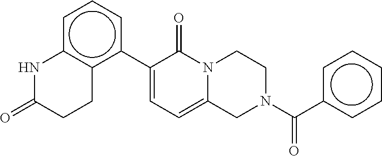 | 0.15 |
| 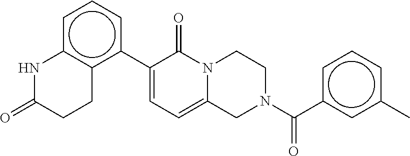 | 0.155 |
| 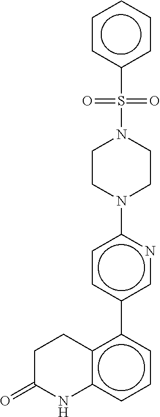 | 0.169 |
| 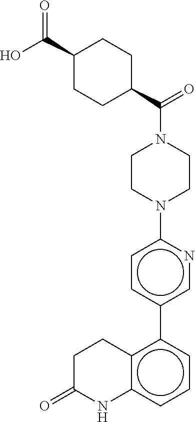 | 0.172 |
| 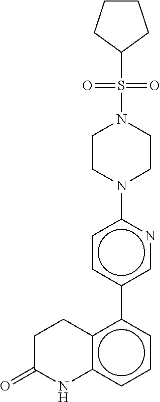 | 0.173 |
| 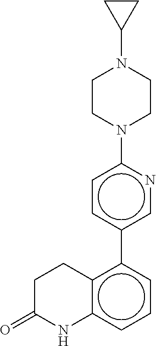 | 0.18 |
| 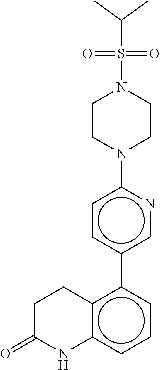 | 0.19 |
| 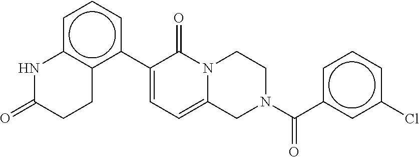 | 0.198 |
| 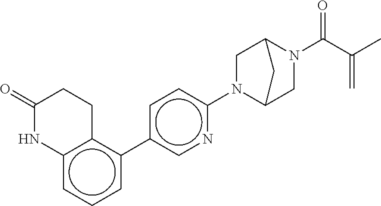 | 0.221 |
| 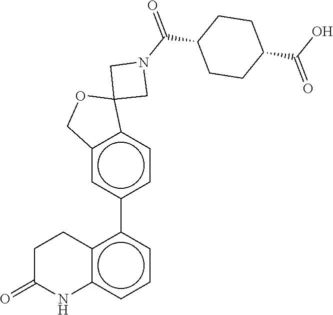 | 0.224 |
| 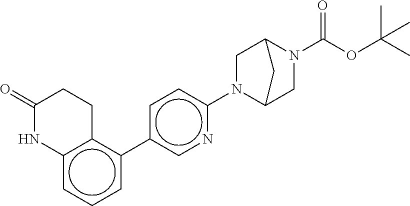 | 0.238 |
| 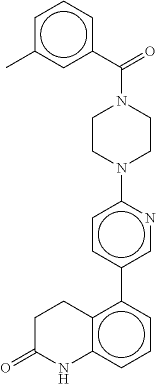 | 0.24 |
| 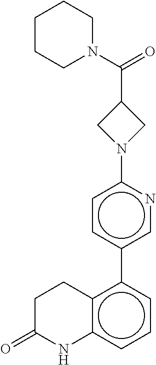 | 0.241 |
| 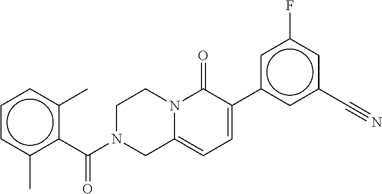 | 0.241 |
| 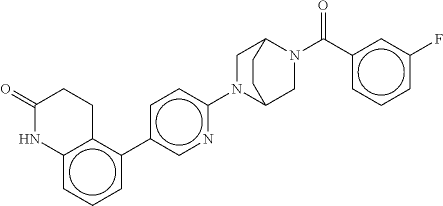 | 0.242 |
| 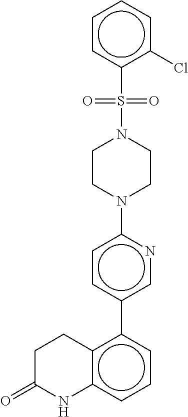 | 0.243 |
| 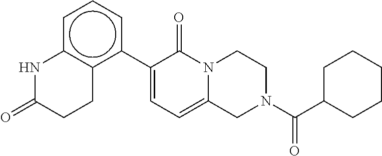 | 0.249 |
| 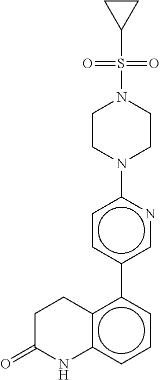 | 0.261 |
| 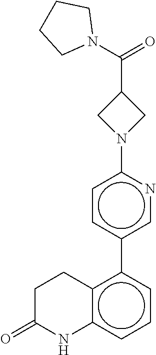 | 0.273 |
| 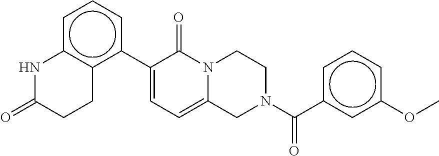 | 0.29 |
| 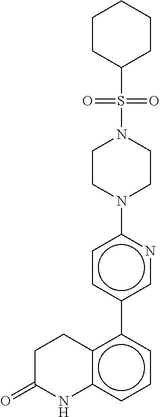 | 0.29 |
| 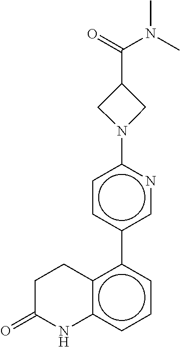 | 0.307 |
| 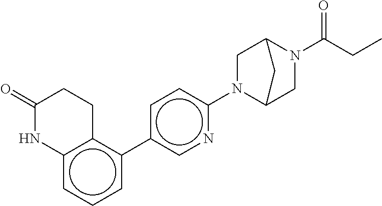 | 0.325 |
| 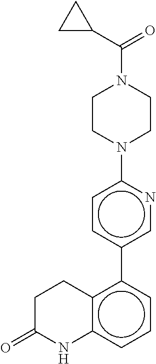 | 0.334 |
| 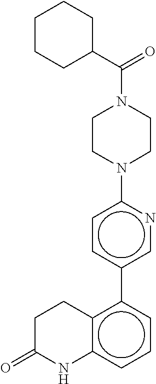 | 0.345 |
| 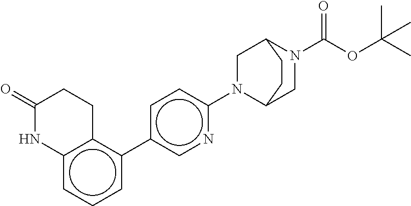 | 0.354 |
| 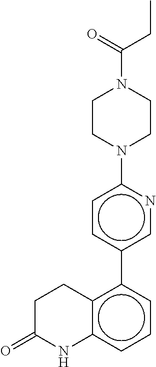 | 0.354 |
| 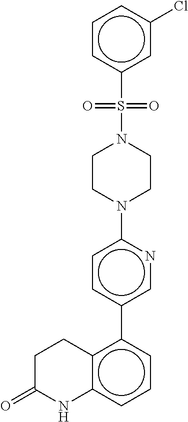 | 0.354 |
| 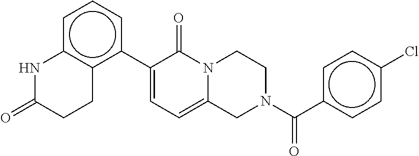 | 0.372 |
| 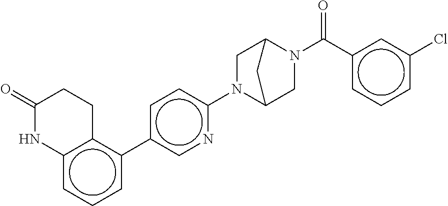 | 0.379 |
| 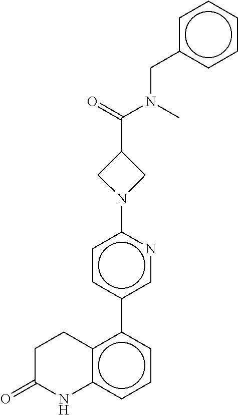 | 0.383 |
| 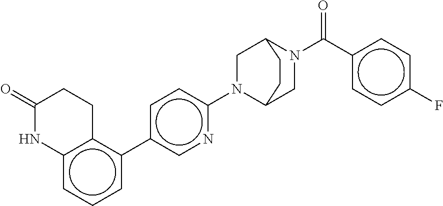 | 0.385 |
| 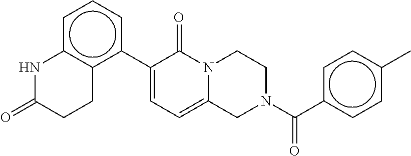 | 0.398 |
| 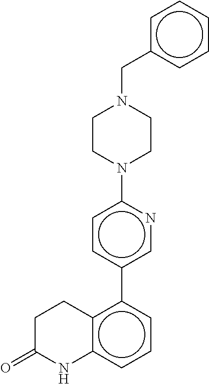 | 0.4 |
| 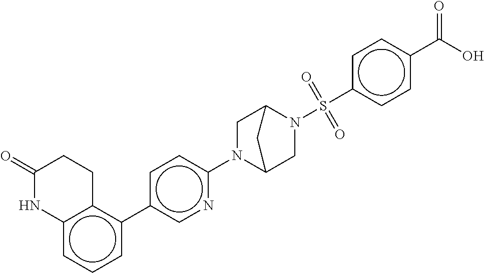 | 0.405 |
| 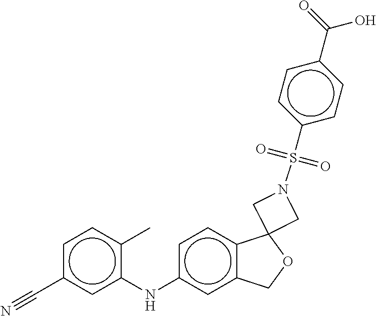 | 0.424 |
| 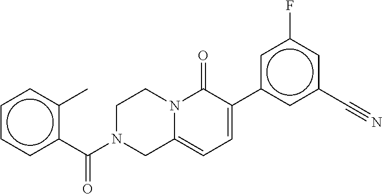 | 0.429 |
| 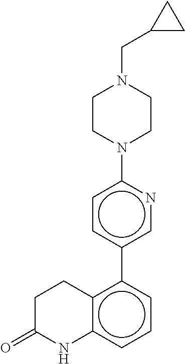 | 0.43 |
| 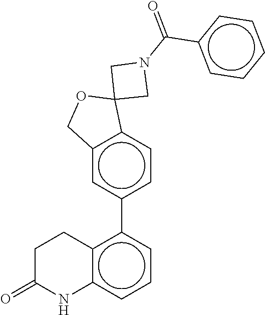 | 0.438 |
| 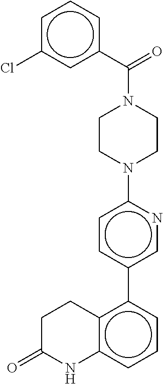 | 0.443 |
| 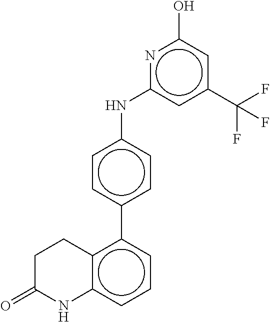 | 0.447 |
| 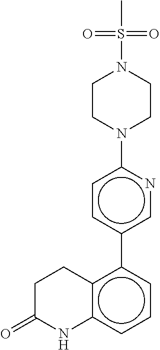 | 0.453 |
| 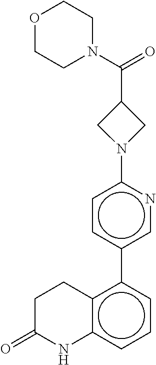 | 0.473 |
| 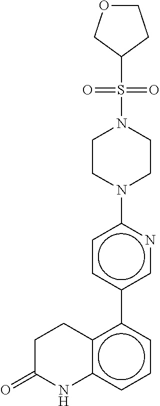 | 0.49 |
| 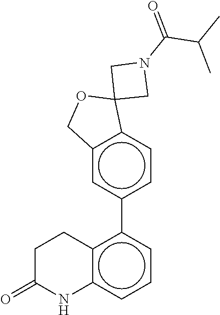 | 0.49 |
| 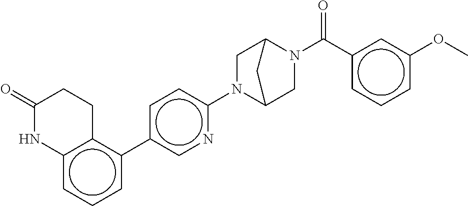 | 0.495 |
| 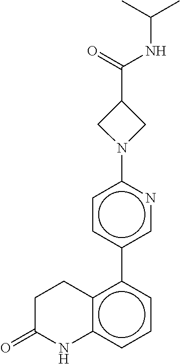 | 0.5 |
| 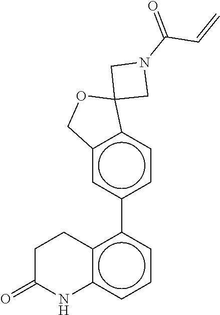 | 0.5 |
| 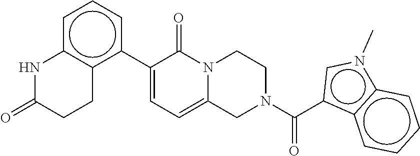 | 0.502 |
| 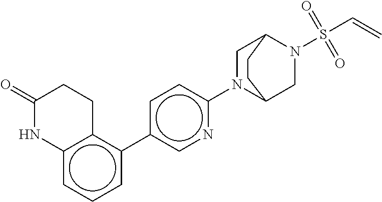 | 0.51 |
| 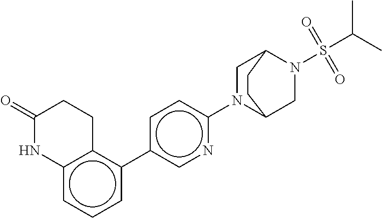 | 0.51 |
| 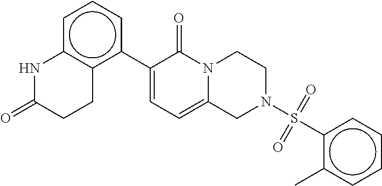 | 0.537 |
| 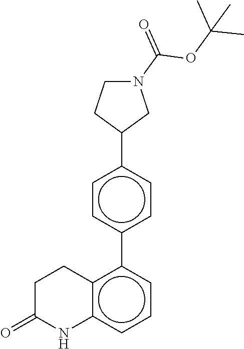 | 0.548 |
| 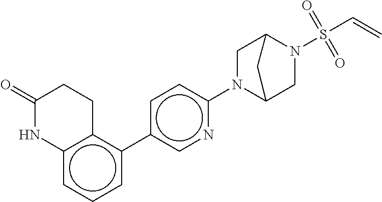 | 0.548 |
| 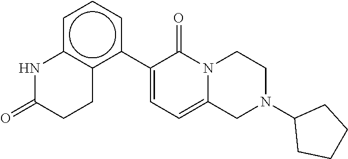 | 0.555 |
| 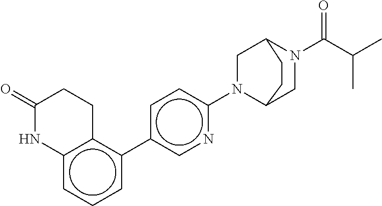 | 0.566 |
| 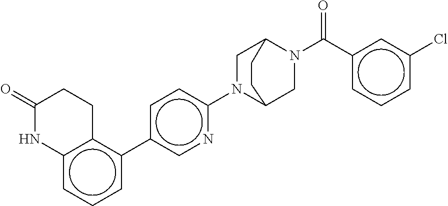 | 0.569 |
| 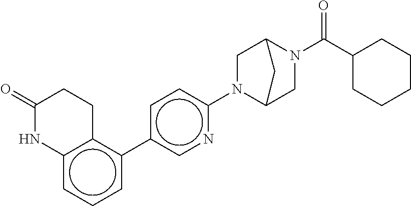 | 0.585 |
| 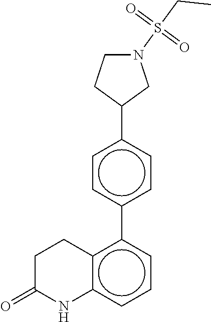 | 0.592 |
| 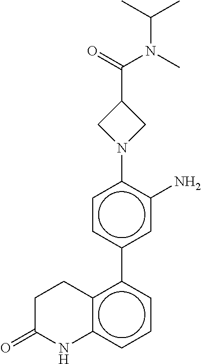 | 0.669 |
| 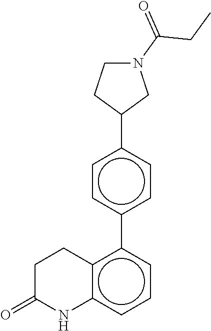 | 0.693 |

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A compound of Formula (I): 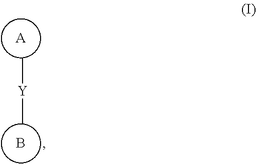
wherein 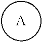
is selected from the group consisting of 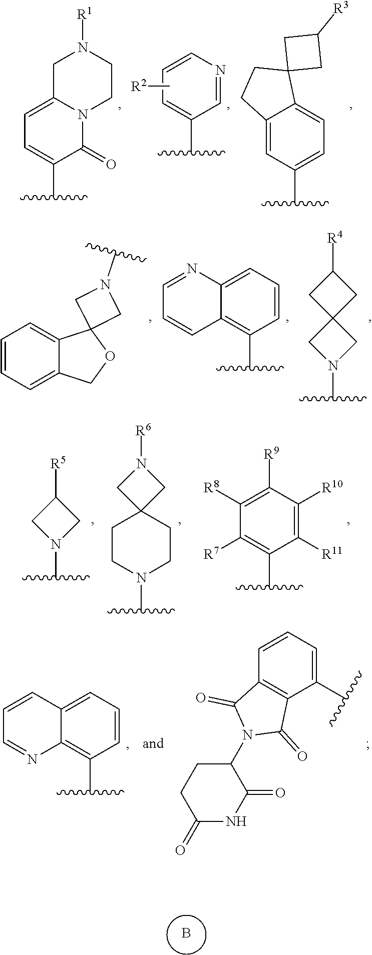
is selected from the group consisting of 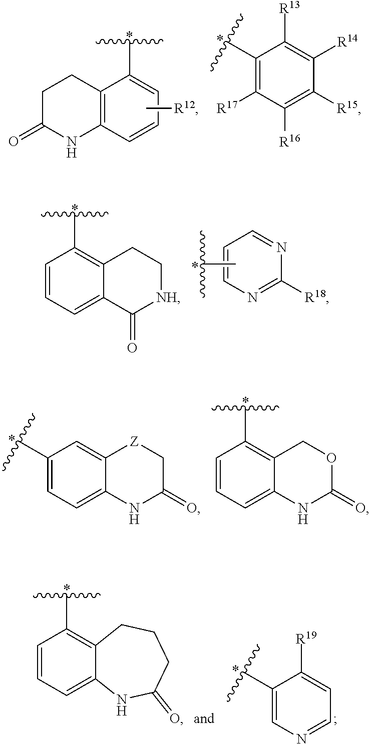
Y is absent, or, if present, is selected from the group consisting of —NH—, —NMe-, and 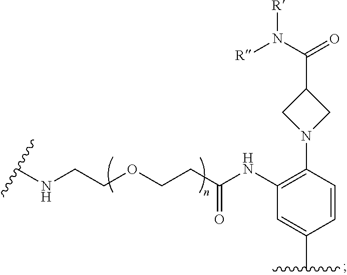
Z is CH₂ or O;
R′ is C₁₋₆ alkyl;
R″ is C₁₋₆ alkyl;
R¹ is selected from the group consisting of H, CN, C₁₋₆ alkyl, C₃₋₈ cycloalkyl, aryl, —C(O)C₁₋₆ alkyl, —C(O)C₂₋₆ alkenyl, —C(O)C₂₋₆ alkynyl, —C(O)aryl, —C(O)(CH₂)ₘbenzyl, —C(O)(CH₂)ₘNHMe, —C(O)(CH₂)ₘNMe₂, —C(O)(CH₂)ₘC₃₋₈ cycloalkyl, —C(O)(CH₂)ₘaryl, —C(O)(CH₂)ₘheteroaryl, —C(O)(CH₂)ₘheterocyclyl, —C(O)nonaromatic bicyclic compound, —C(O)CH(OMe)Ph, —S(O)₂C₁₋₆ alkyl, —S(O)₂C₃₋₈ cycloalkyl, and —S(O)₂aryl, wherein C₁₋₆ alkyl, C₂₋₆ alkenyl, C₃₋₈ cycloalkyl, aryl, benzyl, heteroaryl, and heterocyclyl, can be optionally substituted 1 to 3 times with a substituent independently selected at each occurrence from the group consisting of halogen, CN, CF₃, ═O, OH, COOH, —C₁₋₆ alkyl, C₃₋₈ cycloalkyl, aryl, heterocyclyl, and heteroaryl, —OC₁₋₆ alkyl, —O-aryl, —S(O)₂C₁₋₆ alkyl, —NHC(O)C₁₋₆ alkyl, —NHC(O)aryl, —C(O)C₁₋₆ alkyl, —C₁₋₆ alkylene-OH, —C₁₋₆ alkylene-C(O)OH, —NR^{a}R^{b}, and —C(O)NH₂;
R² is selected from the group consisting of halogen, —COOH, —C₁₋₆ alkyl, —OC₁₋₆ alkyl, —NR^{a}R^{b}, —NH(CH₂)ₖC(O)NR^{a}R^{b}, —NH(CH₂)ₖC(O)NR^{c}R^{d}, —C(O)NR^{a}R^{b}, and —S(O)₂C₁₋₆ alkyl;
R³ is selected from the group consisting of H, C₁₋₆ alkyl, C₃₋₈ cycloalkyl, C₄₋₁₄ cycloalkylalkyl, aryl, arylalkyl, —C(O)C₁₋₆ alkyl, —C(O)C₂₋₆ alkenyl, —C(O)C₃₋₈ cycloalkyl, —C(O)OC₁₋₆ alkyl, —C(O)(CH₂)_{q}aryl, —C(O)(CH₂)_{q}heteroaryl, —C(O)(CH₂)_{q}heterocyclyl, —S(O)₂C₁₋₆ alkyl, —S(O)₂C₂₋₆, —S(O)₂C₃₋₈ cycloalkyl, —S(O)₂(CH₂)_{q}aryl, —S(O)₂heteroaryl, and —S(O)₂heterocyclyl, wherein C₁₋₆ alkyl, C₃₋₈ cycloalkyl, aryl, arylalkyl, heteroaryl, and heterocyclyl can be optionally substituted 1 to 3 times with a substituent independently selected at each occurrence from the group consisting of OH, COOH, CF₃, halogen, C₁₋₆ alkyl, aryl, heteroaryl, heterocyclyl, NH₂, —OC₁₋₆ alkyl, —C(O)C₁₋₆ alkyl, —CH₂COOH, —CH₂OH, and —C(O)NH₂;
R⁴ is selected from the group consisting of —NR^{a}R^{b}, —NH—(CH₂)ₚ-heteroaryl, —NH—(CH₂)ₚ-heterocyclyl, —NH—C(O)C₁₋₆ alkyl, —NH—C(O)C₂₋₆ alkenyl, —NH—C(O)C₁₋₆ alkyl-aryl, —NH—CH₂—C(O)NR^{a}R^{b}, —NH—S(O)₂C₁₋₆ alkyl, and —NH—S(O)₂C₂₋₆ alkenyl, wherein C₁₋₆ alkyl, heteroaryl, and heterocyclyl can be substituted 1 to 3 times with C₁₋₆ alkyl;
R⁵ is selected from the group consisting of —NH—C(O)—CH₂—NH—C(O)-aryl, —NH—C(O)—CH₂—NH—C(O)—C₂₋₆ alkenyl, and —NH-heteroarylene-C(O)—NR^{a}R^{b};
R⁶ is selected from the group consisting of H, —C(O)C₁₋₆ alkyl, and —C(O)C₂₋₆ alkyl;
R⁷ is selected from the group consisting of H, CN, C₁₋₆ alkyl, —CH₂OH, and —C(O)NH₂;
R⁸ is selected from the group consisting of H, halogen, —C₁₋₆ alkyl, —CH₂OH, —OC₁₋₆ alkyl, —NMe₂, and —C(O)NHMe;
R⁹ is selected from the group consisting of H, halogen, C₁₋₆ alkyl, —OC₁₋₆ alkyl, heterocyclyl, —NR^{a}R^{b}, and —NH—CH₂—C(O)NH-i-Pr, wherein C₁₋₆ alkyl and heterocyclyl can be substituted with R²¹;
R¹⁰ is selected from the group consisting of H, C₁₋₆ alkyl, —OC₁₋₆ alkyl, —CH₂OH, —NH₂, —NH—C(O)C₁₋₆ alkyl, and —NH—C(O)OC₁₋₆ alkyl, wherein C₁₋₆ alkyl can be optionally substituted with —OC₁₋₆ alkyl;
R¹¹ is selected from the group consisting of H, halogen, and —C₁₋₆ alkyl;
R¹² is selected from the group consisting of H, halogen, C₁₋₆ alkyl, and —OC₁₋₆ alkyl;
R¹³ is selected from the group consisting of H, OH, —CH₂OH, and C₁₋₆ alkyl;
R¹⁴ is selected from the group consisting of H, C₁₋₆ alkyl, halogen, OH, —OC₁₋₆ alkyl, and CN;
R¹⁵ is selected from the group consisting of H, C₁₋₆ alkyl, —CH₂OH, CN, and —C(O)NH₂;
R¹⁶ is selected from the group consisting of H, CN, halogen, and —C(O)NH₂;
R¹⁷ is selected from the group consisting of H, OH, C₁₋₆ alkyl, —CH₂OH, and —C(O)NH₂;
R¹⁸ is selected from the group consisting of —OC₁₋₆ alkyl, 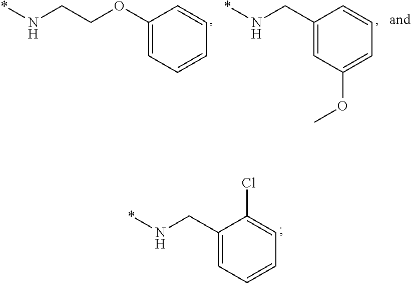
R¹⁹ is C₁₋₆ alkyl or —CH₂OH;
R²⁰ is independently selected at each occurrence from the group consisting of ═O, COOH, C₁₋₆ alkyl, C₃₋₈ cycloalkyl, —C(O)C₁₋₆ alkyl, —C(O)C₂₋₆ alkenyl, —C(O)—C₃₋₈ cycloalkyl, —C(O)aryl, —C(O)heteroaryl, —C(O)heterocyclyl, —C(O)NR^{aa}R^{bb}, —C(O)OC₁₋₆ alkyl, —NH—C(O)OC₁₋₆ alkyl, —NH—C(O)C₂₋₆ alkenyl, —NH—C(O)OC₂₋₆ alkenyl, —C₁₋₆ alkylene-arylene-NH—C(O)C₁₋₆ alkyl, —C₁₋₆ alkylene-arylene-NH—C(O)C₂₋₆ alkenyl, —S(O)₂C₁₋₆ alkyl, —S(O)₂C₂₋₆ alkenyl, —S(O)₂C₃₋₈ cycloalkyl, —S(O)₂(CH₂) aryl, —S(O)₂heterocyclyl, —(CH₂)ᵣheterocyclyl, wherein C₁₋₆ alkyl, C₁₋₆ alkylene, C₂₋₆ alkenyl, C₃₋₈ cycloalkyl, aryl, arylene, heteroaryl, and heterocyclyl can be optionally substituted 1 to 3 times with a substituent independently selected at each occurrence from the group consisting of halogen, —OH, —COOH, —CN, —C₁₋₆ alkyl, —OC₁₋₆ alkyl, —C₃₋₈ cycloalkyl, aryl, heterocyclyl, —C(O)C₁₋₆ alkyl, —C(O)OC₁₋₆ alkyl, —C(O)OC₁₋₆ alkylene-aryl, and —S(O)₂C₁₋₆ alkyl;
R²¹ is independently selected at each occurrence form the group consisting of —NR^{a}R^{b}, —C(O)C₁₋₆ alkyl, —C(O)OC₁₋₆ alkyl, and —S(O)₂C₁₋₆ alkyl, wherein —S(O)₂C₁₋₆ alkyl can be optionally substituted with -aryl-COOH;
R²² is independently selected at each occurrence form the group consisting of C₃₋₈ cycloalkyl, aryl, benzyl, —NR^{aa}R^{bb}, heteroaryl, and heterocyclyl, wherein heteroaryl and heterocyclyl can be optionally substituted 1 to 3 times with C₁₋₆ alkyl;
R^{a} is selected from the group consisting of H, C₁₋₆ alkyl, aryl, and heteroaryl, wherein C₁₋₆ alkyl, aryl, and heteroaryl can be optionally substituted 1 to 3 times with a substituent independently selected at each occurrence from the group consisting of H, OH, COOH, CF₃, —OC₁₋₆ alkyl, aryl, and —NR^{aa}R^{bb};
R^{b} is H or C₁₋₆ alkyl, wherein C₁₋₆ alkyl can be optionally substituted 1 to 3 times with —C(O)NR^{aa}R^{bb}; or
R^{a} and R^{b} combine with the nitrogen atom to which they are attached to form piperazine, azetidine, piperidine, pyrrolidine, diazabicyclo[2.2.1]heptane, diazabicyclo[2.2.2]octane, 1,4-diazepane, or 2,7-diazaspiro[3.5]nonane ring, wherein piperazine, azetidine, piperidine, pyrrolidine, diazabicyclo[2.2.1]heptane, diazabicyclo[2.2.2]octane, 1,4-diazepane, or 2,7-diazaspiro[3.5]nonane ring can be optionally substituted 1 to 3 times with R²⁰,
R^{c} is H, C₁₋₆ alkyl, or C₃₋₈ cycloalkyl, wherein C₁₋₆ alkyl can be optionally substituted 1 to 3 times with R²²;
R^{d} is H or C₁₋₆ alkyl; or
R^{c} and R^{d} combine with the nitrogen atom to which they are attached to form piperazine, piperidine, pyrrolidine, or morpholine ring, wherein piperazine or piperidine ring can be optionally substituted 1 to 3 times with a substituent independently selected at each occurrence from the group consisting of C₁₋₆ alkyl, heteroaryl, and heterocyclyl;
R^{aa} is H, C₁₋₆ alkyl, aryl, arylalkyl, or CN, wherein C₁₋₆ alkyl can be optionally substituted 1 to 3 times with—a substituent independently selected at each occurrence from the group consisting of C(O)OC₁₋₆ alkyl, COOH, and OH;
R^{bb} is H or C₁₋₆ alkyl;
m is 0, 1, 2, or 3;
n is 1, 2, 3, 4, or 5;
p is 1 or 2;
q is 0, 1, or 2;
r is 1;
s is 0 or 1; and
k is 0 or 1;
or an oxide thereof, a pharmaceutically acceptable salt thereof, a solvate thereof, or a prodrug thereof.

2. (canceled)

3. The compound according to claim 1, which has the Formula (IB):

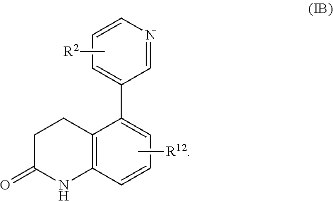

4.-5. (canceled)

6. The compound according to claim 1, which has the Formula (IE): 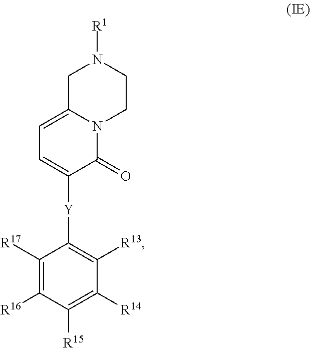
wherein Y is absent, or, if present, is —NH— or —NMe-.

7. The compound according to claim 1, which has the Formula (IF):

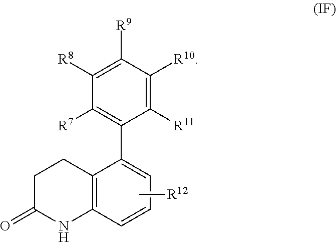

8.-21. (canceled)

22. The compound according to claim 3, which has the Formula (IB′): 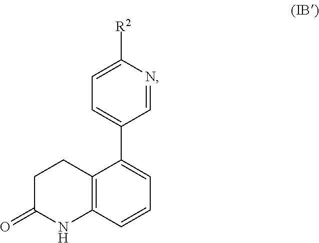
wherein
R² is —NR^{a}R^{b};
R^{a} and R^{b} combine with the nitrogen atom to which they are attached to form piperazine, azetidine, diazabicyclo[2.2.1]heptane, and diazabicyclo[2.2.2]octane, wherein piperazine, azetidine, diazabicyclo[2.2.1]heptane, and diazabicyclo[2.2.2]octane can be optionally substituted 1 to 3 times with R²⁰;
R²⁰ is independently selected at each occurrence form the group consisting of H, C₁₋₆ alkyl, C₃₋₈ cycloalkyl, —C(O)C₁₋₆ alkyl, —C(O)C₂₋₆ alkenyl, —C(O)—C₃₋₈ cycloalkyl, —C(O)aryl, —C(O)heteroaryl, —C(O)heterocyclyl, —C(O)NR^{aa}R^{bb}, —C(O)OC₁₋₆ alkyl, —S(O)₂C₁₋₆ alkyl, —S(O)₂C₂₋₆ alkenyl, —S(O)₂C₃₋₈ cycloalkyl, —S(O)₂aryl, and —S(O)₂heterocyclyl, wherein C₁₋₆ alkyl, C₂₋₆ alkenyl, C₃₋₈ cycloalkyl, aryl, and heteroaryl can be optionally substituted 1 to 3 times with a substituent independently selected at each occurrence from the group consisting of H, halogen, —COOH, —C₁₋₆ alkyl, —OC₁₋₆ alkyl, C₃₋₈ cycloalkyl, and aryl;
R^{aa} is selected at each occurrence form the group consisting of H, C₁₋₆ alkyl, and arylalkyl; and
R^{bb} is H or C₁₋₆ alkyl.

23. The compound according to claim 22, wherein R² is selected from the group consisting of

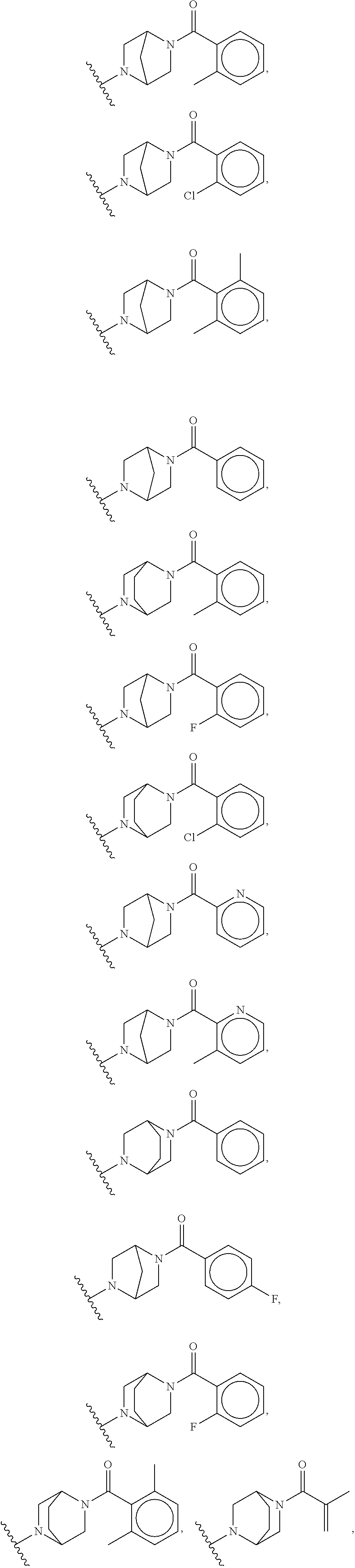

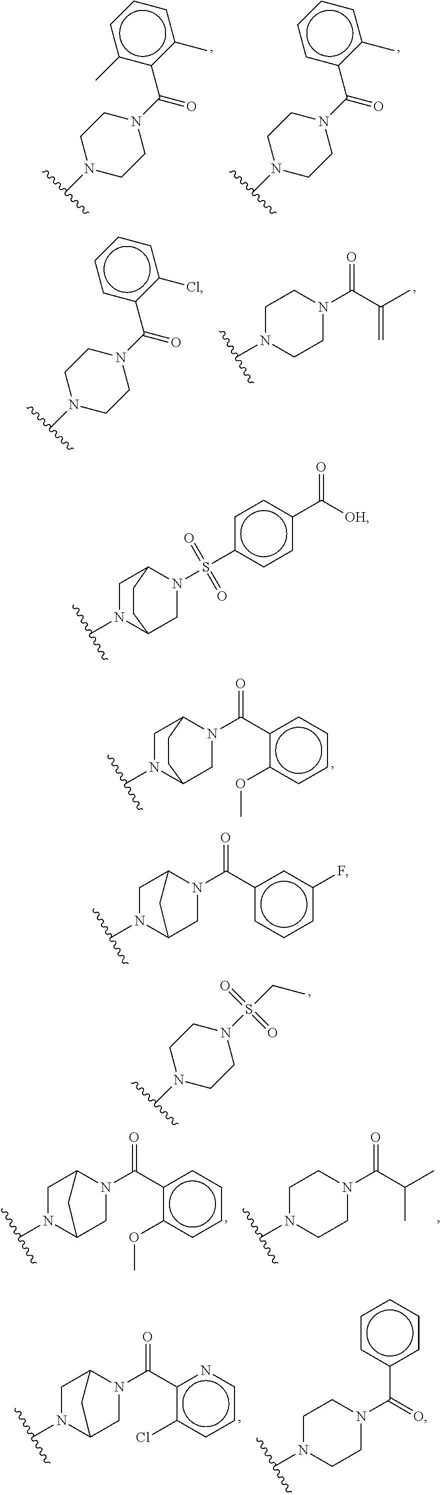

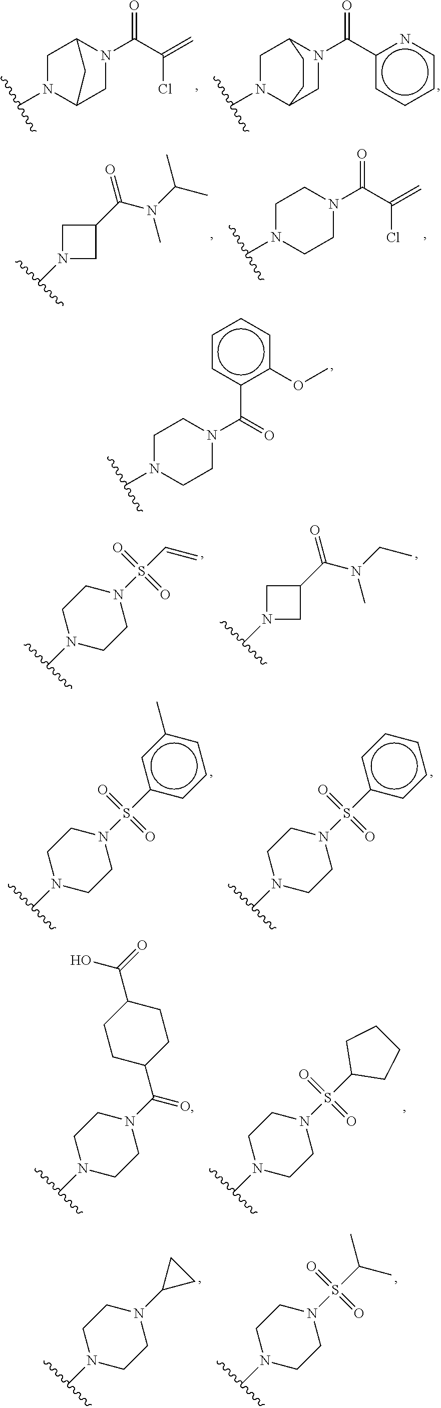

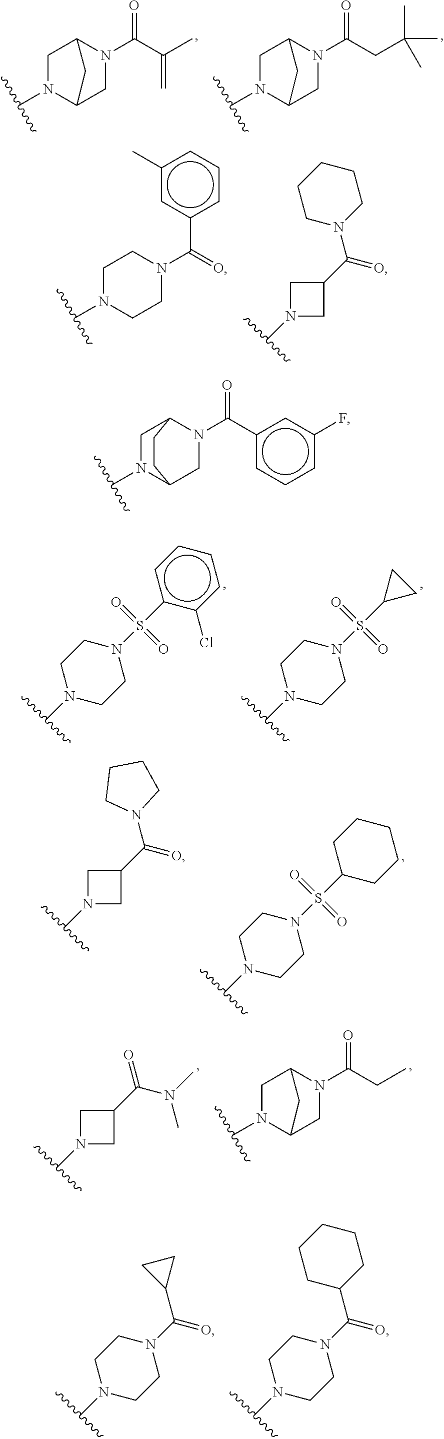

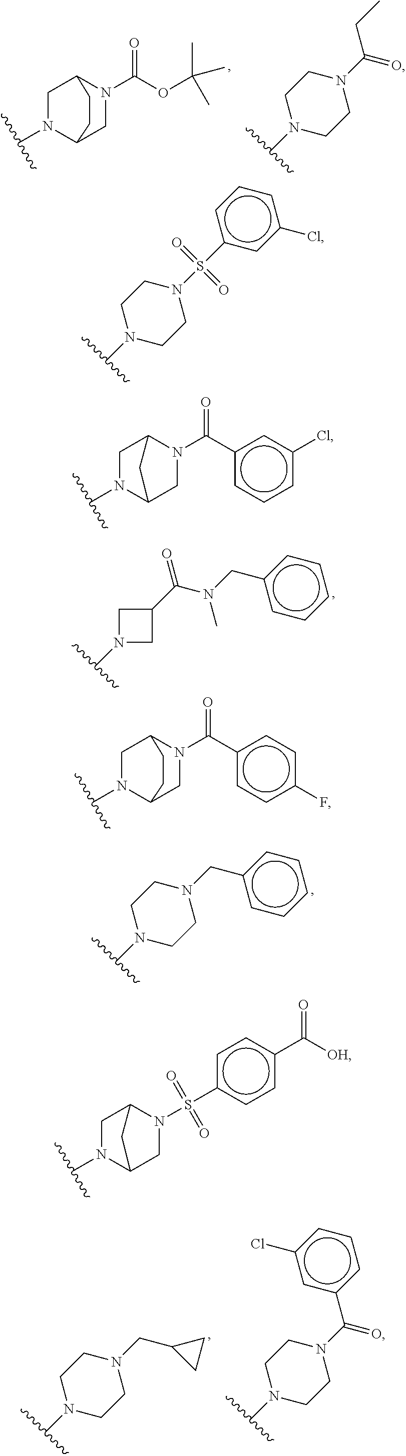

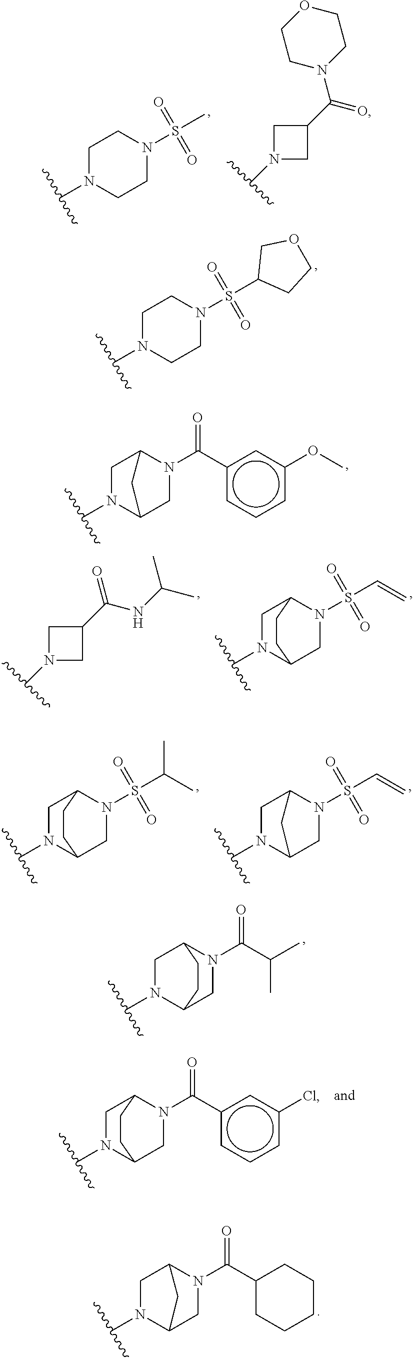

24.-30. (canceled)

31. The compound according to claim 6, which has the Formula (IE′): 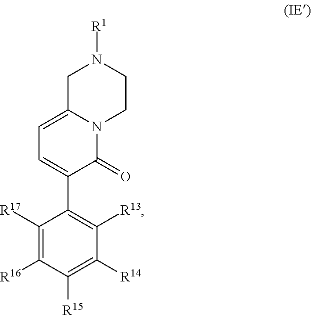
wherein
R¹ is —C(O)aryl, wherein aryl can be optionally substituted 1 to 3 times with —C₁₋₆ alkyl.

32. The compound according to claim 31, wherein R¹ is

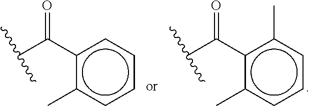

33. The compound according to claim 31, wherein R¹³ is H, R¹⁴ is F, R¹⁵ is H, R¹⁶ is CN, and R¹⁷ is H.

34. The compound according to claim 7, which has the Formula (IF′): 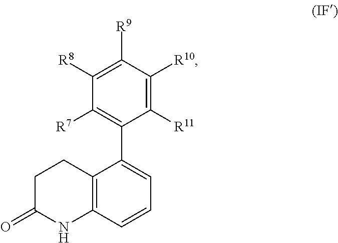
wherein
R⁹ is monocyclic heterocyclyl or —NR^{a}R^{b}, wherein monocyclic heterocyclyl can be optionally substituted 1 time with —C(O)C₁₋₆ alkyl, —C(O)OC₁₋₆ alkyl, or —S(O)₂C₁₋₆ alkyl;
R^{a} is heteroaryl optionally substituted 1 to 3 times with a substituent independently selected at each occurrence from the group consisting of H, OH, and CF₃;
R^{b} is H; or
R^{a} and R^{b} combine with the nitrogen atom to which they are attached to form azetidine optionally substituted 1 time with —C(O)NR^{aa}R^{bb};
R^{aa} is C₁₋₆ alkyl; and
R^{bb} is C₁₋₆ alkyl.

35. The compound according to claim 34, wherein R^{a} is pyridinyl substituted with OH and CF₃.

36. The compound according to claim 34, wherein R⁹ is selected from the group consisting of

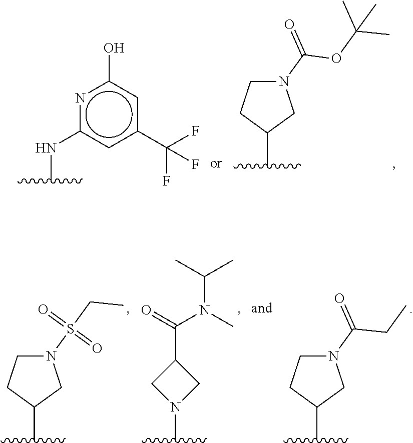

37. The compound according to claim 34, wherein R⁷ is H, R⁸ is H, R¹⁰ is H or NH₂, and R¹¹ is H.

38. A pharmaceutical composition comprising a therapeutically effective amount of the compound according to claim 1 and a pharmaceutically acceptable carrier.

39. A method of treating cancer or neoplastic condition in a subject, said method comprising:
administering to the subject in need thereof the compound according to claim 1.

40.-42. (canceled)

43. A method of inhibiting an Artemis nuclease activity, said method comprising:
contacting an Artemis nuclease with the compound according to claim 1 under conditions effective to inhibit the Artemis nuclease activity.

44. A method of ameliorating a condition caused by the activity of Artemis nuclease in a subject comprising:
administering to said subject the compound according to claim 1.

45. (canceled)

46. A method of enhancing cancer therapy in a subject comprising:
delivering the compound according to claim 1 to cancerous cells in said subject in an amount effective to inhibit Artemis nuclease, followed by administration of a traditional cancer therapy to said subject.

47. A method for the treatment of a severe combined immunodeficiency (SCID) comprising:
administering to a subject the compound according to claim 1.

48. A method of biotherapy in a subject comprising:
administering to the subject the compound according to claim 1.
